SEQUENCE LISTING (10 sequences)

SEQ ID NO: 1 - protein, 546 aa, Homo sapiens
MAEVVSPVPGAGRREPGEVGRARGPPVADPGVALSPQGEIIEGCRLPVLRRNQDNEDEWPLAEILSVKDISGRKLFYVHYIDFNKRLDEWVTHERLDLKKIQFPKKEAKTPTKNGLPGSRPGSPEREVPASAQASGKTLPIPVQITLRFNLPKEREAIPGGEPDQPLSSSSCLQPNHRSTKRKVEVVSPATPVPSETAPASVFPQNGAARRAVAAQPGRKRKSNCLGTDEDSQDSSDGIPSAPRMTGSLVSDRSHDDIVTRMKNIECIELGRHRLKPWYFSPYPQELTTLPVLYLCEFCLKYGRSLKCLQRHLTKCDLRHPPGNEIYRKGTISFFEIDGRKNKSYSQNLCLLAKCFLDHKTLYYDTDPFLFYVMTEYDCKGFHIVGYFSKEKESTEDYNVACILTLPPYQRRGYGKLLIEFSYELSKVEGKTGTPEKPLSDLGLLSYRSYWSQTILEILMGLKSESGERPQITINEISEITSIKKEDVISTLQYLNLINYYKGQYILTLSEDIVDGHERAMLKRLLRIDSKCLHFTPKDWSKRGKW

SEQ ID NO: 2 - protein, 513 aa, Homo sapiens
MAEVGEIIEGCRLPVLRRNQDNEDEWPLAEILSVKDISGRKLFYVHYIDFNKRLDEWVTHERLDLKKIQFPKKEAKTPTKNGLPGSRPGSPEREVPASAQASGKTLPIPVQITLRFNLPKEREAIPGGEPDQPLSSSSCLQPNHRSTKRKVEVVSPATPVPSETAPASVFPQNGAARRAVAAQPGRKRKSNCLGTDEDSQDSSDGIPSAPRMTGSLVSDRSHDDIVTRMKNIECIELGRHRLKPWYFSPYPQELTTLPVLYLCEFCLKYGRSLKCLQRHLTKCDLRHPPGNEIYRKGTISFFEIDGRKNKSYSQNLCLLAKCFLDHKTLYYDTDPFLFYVMTEYDCKGFHIVGYFSKEKESTEDYNVACILTLPPYQRRGYGKLLIEFSYELSKVEGKTGTPEKPLSDLGLLSYRSYWSQTILEILMGLKSESGERPQITINEISEITSIKKEDVISTLQYLNLINYYKGQYILTLSEDIVDGHERAMLKRLLRIDSKCLHFTPKDWSKRGKW

SEQ ID NO: 3 - protein, 461 aa, Homo sapiens
MAEVGEIIEGCRLPVLRRNQDNEDEWPLAEILSVKDISGRKLFYVHYIDFNKRLDEWVTHERLDLKKIQFPKKEAKTPTKNGLPGSRPGSPEREVKRKVEVVSPATPVPSETAPASVFPQNGAARRAVAAQPGRKRKSNCLGTDEDSQDSSDGIPSAPRMTGSLVSDRSHDDIVTRMKNIECIELGRHRLKPWYFSPYPQELTTLPVLYLCEFCLKYGRSLKCLQRHLTKCDLRHPPGNEIYRKGTISFFEIDGRKNKSYSQNLCLLAKCFLDHKTLYYDTDPFLFYVMTEYDCKGFHIVGYFSKEKESTEDYNVACILTLPPYQRRGYGKLLIEFSYELSKVEGKTGTPEKPLSDLGLLSYRSYWSQTILEILMGLKSESGERPQITINEISEITSIKKEDVISTLQYLNLINYYKGQYILTLSEDIVDGHERAMLKRLLRIDSKCLHFTPKDWSKRGKW

SEQ ID NO: 4 - protein, 494 aa, Homo sapiens
MAEVVSPVPGAGRREPGEVGRARGPPVADPGVALSPQGEIIEGCRLPVLRRNQDNEDEWPLAEILSVKDISGRKLFYVHYIDFNKRLDEWVTHERLDLKKIQFPKKEAKTPTKNGLPGSRPGSPEREVKRKVEVVSPATPVPSETAPASVFPQNGAARRAVAAQPGRKRKSNCLGTDEDSQDSSDGIPSAPRMTGSLVSDRSHDDIVTRMKNIECIELGRHRLKPWYFSPYPQELTTLPVLYLCEFCLKYGRSLKCLQRHLTKCDLRHPPGNEIYRKGTISFFEIDGRKNKSYSQNLCLLAKCFLDHKTLYYDTDPFLFYVMTEYDCKGFHIVGYFSKEKESTEDYNVACILTLPPYQRRGYGKLLIEFSYELSKVEGKTGTPEKPLSDLGLLSYRSYWSQTILEILMGLKSESGERPQITINEISEITSIKKEDVISTLQYLNLINYYKGQYILTLSEDIVDGHERAMLKRLLRIDSKCLHFTPKDWSKRGKW

SEQ ID NO: 5 - protein, 545 aa, Artificial Sequence
  Synthetic
MHHHHHHSSGVDLGTENLYFQSNAMAEVGEIIEGCRLPVLRRNQDNEDEWPLAEILSVKDISGRKLFYVHYIDFNKRLDEWVTHERLDLKKIQFPKKEAKTPTKNGLPGSRPGSPEREVPASAQASGKTLPIPVQITLRFNLPKEREAIPGGEPDQPLSSSSCLQPNHRSTKRKVEVVSPATPVPSETAPASVFPQNGAARRAVAAQPGRKRKSNCLGTDEDSQDSSDGIPSAPRMTGSLVSDRSHDDIVTRMKNIECIELGRHRLKPWYFSPYPQELTTLPVLYLCEFCLKYGRSLKCLQRHLTKCDLRHPPGNEIYRKGTISFFEIDGRKNKSYSQNLCLLAKCFLDHKTLYYDTDPFLFYVMTEYDCKGFHIVGYFSKEKESTEDYNVACILTLPPYQRRGYGKLLIEFSYELSKVEGKTGTPEKPLSDLGLLSYRSYWSQTILEILMGLKSESGERPQITINEISEITSIKKEDVISTLQYLNLINYYKGQYILTLSEDIVDGHERAMLKRLLRIDSKCLHFTPKDWSKRGKWDYKDDDDK

SEQ ID NO: 6 - protein, 524 aa, Artificial Sequence
  Synthetic
SNAMAEVGEIIEGCRLPVLRRNQDNEDEWPLAEILSVKDISGRKLFYVHYIDFNKRLDEWVTHERLDLKKIQFPKKEAKTPTKNGLPGSRPGSPEREVPASAQASGKTLPIPVQITLRFNLPKEREAIPGGEPDQPLSSSSCLQPNHRSTKRKVEVVSPATPVPSETAPASVFPQNGAARRAVAAQPGRKRKSNCLGTDEDSQDSSDGIPSAPRMTGSLVSDRSHDDIVTRMKNIECIELGRHRLKPWYFSPYPQELTTLPVLYLCEFCLKYGRSLKCLQRHLTKCDLRHPPGNEIYRKGTISFFEIDGRKNKSYSQNLCLLAKCFLDHKTLYYDTDPFLFYVMTEYDCKGFHIVGYFSKEKESTEDYNVACILTLPPYQRRGYGKLLIEFSYELSKVEGKTGTPEKPLSDLGLLSYRSYWSQTILEILMGLKSESGERPQITINEISEITSIKKEDVISTLQYLNLINYYKGQYILTLSEDIVDGHERAMLKRLLRIDSKCLHFTPKDWSKRGKWDYKDDDDK

SEQ ID NO: 7 - protein, 308 aa, Artificial Sequence
  Synthetic
MHHHHHHSSGVDLGTENLYFQSNAPPDPQVRCPSVIEFGKYEIHTWYSSPYPQEYSRLPKLYLCEFCLKYMKSRTILQQHMKKCGWFHPPANEIYRKNNISVFEVDGNVSTIYCQNLCLLAKLFLDHKTLYYDVEPFLFYVLTQNDVKGCHLVGYFSKEKHCQQKYNVSCIMILPQYQRKGYGRFLIDFSYLLSKREGQAGSPEKPLSDLGRLSYMAYWKSVILECLYHQNDKQISIKKLSKLTGICPQDITSTLHHLRMLDFRSDQFVIIRREKLIQDHMAKLQLNLRPVDVDPECLRWTPVIVSNS

SEQ ID NO: 8 - protein, 287 aa, Artificial Sequence
  Synthetic
SNAPPDPQVRCPSVIEFGKYEIHTWYSSPYPQEYSRLPKLYLCEFCLKYMKSRTILQQHMKKCGWFHPPANEIYRKNNISVFEVDGNVSTIYCQNLCLLAKLFLDHKTLYYDVEPFLFYVLTQNDVKGCHLVGYFSKEKHCQQKYNVSCIMILPQYQRKGYGRFLIDFSYLLSKREGQAGSPEKPLSDLGRLSYMAYWKSVILECLYHQNDKQISIKKLSKLTGICPQDITSTLHHLRMLDFRSDQFVIIRREKLIQDHMAKLQLNLRPVDVDPECLRWTPVIVSNS

SEQ ID NO: 9 - protein, 20 aa, Artificial Sequence
  Synthetic
SGRGRGGRGLGKGGARRHRK

SEQ ID NO: 10 - protein, 26 aa, Artificial Sequence
  Synthetic
SGRGKGGKGLGKGGAKRHRKVLRGGK

(19) United States
(12) Patent Application Publication
(10) Pub. No.: US 2022/0267260 A1
(43) Pub. Date: Aug. 25, 2022

(54) COMPOUNDS CONTAINING A SULFONIC GROUP AS KAT INHIBITORS

(71) Applicant: Epizyme, Inc., Cambridge, MA (US)

(72) Inventor: Darren Martin Harvey, Acton, MA (US)

(21) Appl. No.: 16/464,584

(22) PCT Filed: Nov. 29, 2017
(86) PCT No.: PCT/US2017/063721

§ 371 Date: May 28, 2019

Related U.S. Application Data
(60) Provisional application No. 62/427,732, filed on Nov. 29, 2016.
(60) Provisional application No. 62/434,356, filed on Dec. 14, 2016.

(51) Int. Cl. C07C 311/49; C07D 205/04; C07D 207/08; C07D 207/36; C07D 207/48; C07D 209/42; C07D 211/60; C07D 211/24; C07D 211/54; C07D 213/26; C07D 231/12; C07D 231/56; C07D 239/28; C07D 235/24; C07D 333/48; C07D 263/28; C07D 263/50; C07D 275/03; C07D 305/08; C07D 309/06; C07D 471/04; C07D 309/08; C07D 403/12; C07D 413/12; C07D 405/12; C07D 405/06; C07D 307/18; C07D 407/12; C07D 471/10

(52) U.S. Cl. CPC C07C 311/49; C07D 471/10; C07D 207/08; C07D 207/36; C07D 207/48; C07D 209/42; C07D 211/60; C07D 211/24; C07D 211/54; C07D 213/26; C07D 231/12; C07D 231/56; C07D 239/28; C07D 235/24; C07D 333/48; C07D 263/28; C07D 263/50; C07D 275/03; C07D 305/08; C07D 309/06; C07D 471/04; C07D 309/08; C07D 403/12; C07D 413/12; C07D 405/12; C07D 405/06; C07D 307/18; C07D 407/12; C07D 205/04

(57) ABSTRACT

The present invention provides compounds, pharmaceutically acceptable compositions thereof, and methods of using the same.

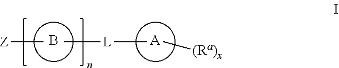

### RELATED APPLICATIONS

This Application is a national stage filing under 35 U.S.C. 371 of International Patent Application Serial No. PCT/US2017/063721, filed Nov. 29, 2017, which is a Non-Provisional of U.S. Application Ser. No. 62/434,356, filed Dec. 14, 2016, and U.S. Application Ser. No. 62/427,732, filed Nov. 29, 2016. The entire contents of these applications are incorporated herein by reference in their entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Mar. 26, 2021 is named E050170035US02-SEQ-DFC and is 33 kilobytes in size.

### SUMMARY

Protein acetylation is involved in several cellular processes. Lysine acetylation has been reported to modulate (e.g., inhibit) other protein modifications, such as methylation and ubiquitination, modify protein stability, alter subcellular localization, or change the spectrum of interacting proteins.

Some aspects of the present disclosure are based on the recognition of the importance of histone acetyl transferases, such as lysine acetyl transferases (KATs), and in particular KAT-5, in initiation and/or progression of some diseases and disorders, e.g., in cancer. Some aspects of the present disclosure encompass the recognition that KATs represent a valuable target for modulating activity in vitro and in vivo, including, for example, in a clinical context, such as cancer therapies. Some aspects of the present disclosure provide that certain KATs, e.g. KAT-5, are therapeutic targets in diseases and conditions characterized by an aberrant activity of KATs, e.g., an increased KAT-5 activity as compared to the activity observed in healthy cells, tissues, or under normal, non-pathological conditions.

Some aspects of the present disclosure provide that KAT-5 is a therapeutic target in various cancers. Some aspects of this disclosure are based on the recognition that KAT (e.g., KAT-5) activity in cancer cells is important for survival and/or proliferation of the cells.

Some aspects of this disclosure provide methods and strategies for inhibiting the survival and/or proliferation of cells, e.g., of neoplastic or malignant cells, comprising contacting such cells with a KAT inhibitor provided herein, by contacting such cells with a KAT-5 inhibitor in vitro, or in vivo, e.g., by administering a KAT (e.g., KAT-5) inhibitor to a subject harboring such cells or a tumor comprising such cells.

The present disclosure thus provides certain therapies useful for the treatment of diseases or conditions characterized by aberrant KAT (e.g., KAT-5) activity, such as various cancers. Methods and compositions provided by the present disclosure may be applicable, for example, to treatment of a wide range of solid tumors and/or to hematological malignancies.

Some aspects of this disclosure provide compounds, and pharmaceutically acceptable compositions thereof, that are inhibitors of lysine acetyl transferases (KATs). In some embodiments, the present invention provides inhibitors of KAT-5. Such KAT inhibitory compounds are of general formula I:

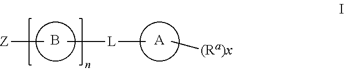

or a pharmaceutically acceptable salt thereof, wherein each of Ring A. Ring B, Z, L, R^{a}, n and x with respect to formula I above, is as defined and described in embodiments herein.

In some embodiments, compounds provided herein, and pharmaceutically acceptable compositions thereof, are useful for inhibiting KAT activity, e.g., KAT-5 activity, in vitro or in vivo, e.g., in a subject in need thereof, such as, for example, in a subject having a condition or disorder characterized by aberrant (e.g., increased) KAT activity. In some embodiments, compounds provided herein, and pharmaceutically acceptable compositions thereof, are useful for treating a variety of diseases, disorders or conditions, characterized by, associated with, or mediated by KAT activity, e.g., by KAT-5 activity. Such diseases, disorders, or conditions include those described herein.

Compounds provided by this invention are also useful for the study of KATs in biological and pathological phenomena and the comparative evaluation of new KAT inhibitors.

### Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B, and March, J., John Wiley & Sons, New York: 2001, the entire contents of Which are hereby incorporated by reference.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be systemic or local. In some embodiments, administration may be enteral or parenteral. In some embodiments, administration may be by injection (e.g., intramuscular, intravenous, or subcutaneous injection). In some embodiments, injection may involve bolus injection, drip, perfusion, or infusion. In some embodiments administration may be topical. Those skilled in the art will be aware of appropriate administration routes for use with particular therapies described herein, for example from among those listed on www.fda.gov, which include auricular (otic), buccal, conjunctival, cutaneous, dental, endocervical, endosinusial, endotracheal, enteral, epidural, extra-amniotic, extracorporeal, interstitial, intra-abdominal, intra-amniotic, intra-arterial, intra articular, intrabiliary, intrabronchial, intrabursal, intracardiac, intracartilaginous, intracaudal, intracavernous, intracavitary, intracerebral, intracisternal, intracorneal, intracoronal, intracorporus cavernosum, intradermal, intradiscal, intraductal, intraduodenal, intradural, intraepidermal, intraesophageal, intragastic, intragingival, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravitreal, laryngeal, nasal, nasogastric, ophthalmic, oral, orophalyngeal, parenteral, percutaneous, periarticular, peridural, perineural, periodontal, rectal, respiratory (e.g., inhalation), retrobulbar, soft tissue, subarachnoid, subconjunctival, subcutaneous, sublingual, submucosal, topical, transdermal, transmucosal, transplacental, transtracheal, ureteral, urethral, or vaginal. In some embodiments, administration may involve electro-osmosis, hemodialysis, infiltration, iontophoresis, irrigation, and/or occlusive dressing. In some embodiments, administration may involve dosing that is intermittent (e.g., a plurality of doses separated in time) and/or periodic (e.g., individual doses separated by a common period of time) dosing. In some embodiments, administration may involve continuous dosing.

Agent: As used herein, the term “agent”, may refer to a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Aliphatic: The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “carbocyclic” (or “cycloaliphatic” or “carbocycle” or “cycloalkyl”) refers to a monocyclic C₃-C₈ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkylene: The term “alkylene” refers to a bivalent alkyl group. Exemplary alkylenes include —CH₂—, —CH₂CH₂—, —CH(CH₃)—, —CH₂CH(CH₃)—, —CH(CH₃)CH₂—, etc. In some embodiments, an “alkylene chain” is a polymethylene group, i.e., —(CH₂)ₙ—, wherein n is a positive integer, preferably from 1 to 6, from 1 to 4, from 1 to 3, from 1 to 2, or from 2 to 3, A substituted alkylene chain is a bivalent alkyl group in which one or more hydrogen atoms are replaced with a substituent. Suitable substituents include those described below for a substituted aliphatic group.

Allele: As used herein, the term “allele” refers to one of two or more existing genetic variants of a specific polymorphic genomic locus.

Amino acid: As used herein, the term “amino acid” refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. As used herein, the term “standard amino acid” refers to any of the twenty L-amino acids commonly found in naturally occurring peptides. “Nonstandard amino acid” refers to any amino acid, other than the standard amino acids, regardless of whether it is or can be found in a natural source. In some embodiments, an amino acid, including a carboxy- and/or amino-terminal amino acid in a polypeptide, can contain a structural modification as compared to the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared to the general structure. In some embodiments, such modification may, for example, alter the stability or the circulating half-life of a polypeptide containing the modified amino acid as compared to one containing an otherwise identical unmodified amino acid. In some embodiments, such modification does not significantly alter a relevant activity of a polypeptide containing the modified amino acid, as compared to one containing an otherwise identical unmodified amino acid. As will be clear from context, in some embodiments, the term “amino acid” may be used to refer to a free amino acid; in some embodiments it may be used to refer to an amino acid residue of a polypeptide, e.g., an amino acid residue within a polypeptide.

Analog: As used herein, the term “analog” refers to a substance that shares one or more particular structural features; elements, components, or moieties with a reference substance. Typically, an “analog” shows significant structural similarity with the reference substance, for example sharing a core or consensus structure, but also differs in one or more certain discrete ways. In some embodiments, an analog is a substance that can be generated from the reference substance. e.g., by chemical manipulation of the reference substance, in some embodiments, an analog is a substance that can be generated through performance of a synthetic process substantially similar to (e.g., sharing a plurality of steps with) one that generates the reference substance. In some embodiments, an analog can be generated through performance of a synthetic process different from that used to generate the reference substance.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (for example when the one or more values of interest define a sufficiently narrow range that application of such a percentage variance would obviate the stated range).

Aryl: The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system and exemplary groups include phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Biological sample: The term “biological sample”, as used herein, includes, without limitation, cell cultures or extracts thereof, biopsied material obtained from a mammal or extracts thereof; and blood, saliva, urine, feces, semen, tears, or other body fluids or extracts thereof. Inhibition of activity of a lysine acetyl transferase, for example, KAT-5, in a biological sample is useful for a variety of purposes that are known to one of skill in the art. Examples of such purposes include, but are not limited to, blood transfusion, organ transplantation, biological specimen storage, and biological assays.

Bridged bicyclic: As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 04 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

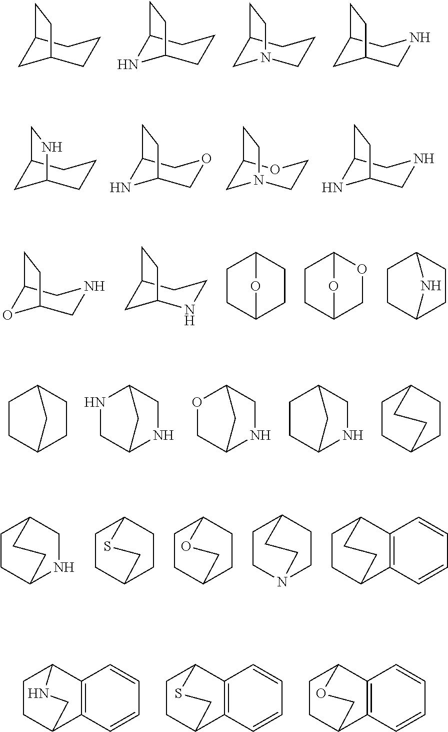

Cancer: As used herein, the term “cancer” refers to a disease, disorder, or condition in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer may be characterized by one or more tumors. Those skilled in the art are aware of a variety of types of cancer including, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, myeloid leukemia), lymphoma (e.g., Burkitt lymphoma [non-Hodgkin lymphoma], cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

Chromosome: As used herein, the term “chromosome” refers to a DNA molecule, optionally together with associated polypeptides and/or other entities, for example as found in the nucleus of eukaryotic cells. Typically, a chromosome carries genes and functions (e.g., origin of replication) that permit it to transmit hereditary information.

Combination therapy: As used herein, the term “combination therapy” refers to a clinical intervention in which a subject is simultaneously exposed to two or more therapeutic regimens (e.g. two or more therapeutic agents). In some embodiments, the two or more therapeutic regimens may be administered simultaneously. In some embodiments, the two or more therapeutic regimens may be administered sequentially (e.g., a first regimen administered prior to administration of any doses of a second regimen). In some embodiments, the two or more therapeutic regimens are administered in overlapping dosing regimens. In some embodiments, administration of combination therapy may involve administration of one or more therapeutic agents or modalities to a subject receiving the other agent(s) or modality. In some embodiments, combination therapy does not necessarily require that individual agents be administered together in a single composition (or even necessarily at the same time). In some embodiments, two or more therapeutic agents or modalities of a combination therapy are administered to a subject separately, e.g., in separate compositions, via separate administration routes (e.g., one agent orally and another agent intravenously), and/or at different time points. In some embodiments, two or more therapeutic agents may be administered together in a combination composition, or even in a combination compound (e.g., as part of a single chemical complex or covalent entity), via the same administration route, and/or at the same time.

Corresponding to: As used herein in the context of polypeptides, nucleic acids, and chemical compounds, the term “corresponding to”, designates the position/identity of a structural element, e.g., of an amino acid residue, a nucleotide residue, or a chemical moiety, in a compound or composition through comparison with an appropriate reference compound or composition.

Disease or disorder associated with KAT-5: As used herein, a “disease or disorder associated with KAT-5” or, alternatively, “a KAT-5-mediated disease or disorder” means any disease or other deleterious condition in which KAT-5, or a mutant thereof, is known or suspected to play a role.

Disease or disorder characterized by aberrant KAT activity: As used herein, a “disease or disorder characterized by aberrant KAT activity” means any disease or other deleterious condition in which an aberrant activity of a KAT, or a mutant thereof, is known or suspected to play a role. An aberrant activity includes, for example, an increased level of KAT activity as compared to a control or reference level. In some embodiments, the control or reference level is an activity level of KAT observed, measured, or expected in the absence of the disease or condition, e.g., in a normal cell, tissue, or sample.

Disease or disorder characterized by aberrant KAT-5 activity: As used herein, a “disease or disorder characterized by aberrant KAT-5 activity” means any disease or other deleterious condition in which an aberrant activity of KAT-5, or a mutant thereof, is known or suspected to play a role. An aberrant activity includes, for example, an increased level of KAT-5 activity as compared to a control or reference level. In some embodiments, the control or reference level is an activity level of KAT-5 observed, measured, or expected in the absence of the disease or condition, e.g., in a normal cell, tissue, or sample.

Domain: As used herein the term “domain” refers to a section or portion of a polypeptide. In some embodiments, a “domain” is associated with a particular structural and/or functional feature of the polypeptide so that, when the domain is physically separated from the rest of its parent polypeptide, it substantially or entirely retains the particular structural and/or functional feature. In some embodiments, a domain may include a portion of a polypeptide that, when separated from that (parent) polypeptide and linked with a different (recipient) polypeptide, substantially retains and/or imparts on the recipient polypeptide one or more structural and/or functional features that characterized it in the parent polypeptide. In some embodiments, a domain is a section of a polypeptide. In some such embodiments, a domain is characterized by a particular structural element (e.g., a particular amino acid sequence or sequence motif, α-helix character, β-sheet character, coiled-coil character, random coil character), and/or by a particular functional feature (e.g. binding activity, enzymatic activity, folding activity, signaling activity

Epigenetic Mark: As used herein, the term “epigenetic mark” refers to a feature of a nucleic acid or polypeptide not directly governed by genetic code. For example, in some embodiments, an epigenetic mark may represent or result from a modification to the nucleic acid or polypeptide. In some embodiments, such modification can include, for example, methylation, acetylation, ubiquitiniation, phosphorylation, ribosylation, amidation, glycosylation or combinations thereof.

Expression: As used herein, the term “expression” of a nucleic acid sequence refers to the generation of any gene product from the nucleic acid sequence, in some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and or (4) post-translational modification of a polypeptide or protein.

Gene: As used herein, the term “gene” refers to a DNA sequence in a chromosome that encodes a gene product (e.g., an RNA product and/or a polypeptide product). In some embodiments, a gene includes a coding sequence (e.g., a sequence that encodes a particular gene product); in some embodiments, a gene includes a non-coding sequence. In some particular embodiments, a gene may include both coding (e.g., exonic) and non-coding (e.g., intronic) sequences. In some embodiments, a gene may include one or more regulatory elements (e.g. promoters, enhancers, silencers, termination signals) that, for example, may control or impact one or more aspects of gene expression (e.g., cell-type-specific expression, inducible expression).

Halogen: The term “halogen” means F, Cl, Br, or I.

Heteroatom: The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

Heteroaryl: The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to live heteroatoms. The term “heteroatom” refers to nitrogen, oxygen, or sulfur, and includes any oxidized form of nitrogen or sulfur, and any quaternized form of a basic nitrogen, Exemplary heteroaryl groups include thienyl, furanyl, pyrrolyl, imidazolyl pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroalyl” and “heteroar-”, as used herein, also include groups in which a heteroaromatic ring is fused to one or more aryl, cycloaliphatic, or heterocyclyl rings, where the radical or point of attachment is on the heteroaromatic ring. Exemplary groups include indolyl, isoindolyl, benzothienyl, benzofuranyl, dibenzofuranyl, indazolyl, benzimidazolyl, benzthiazolyl, quinolyl, isoquinolyl cinnolinyl phthalazinyl, quinazolinyl, quinoxalinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

Heterocycle: As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl —

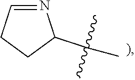 [SMILES: *C1CCC=N1]

NH (as in pyrrolidinyl —

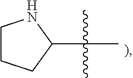 [SMILES: *C1CCCN1]

NR\hat{ } (as in N-substituted 2-pyrrolidinyl —

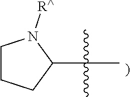

or ⁺NR\hat{ } (as in N-substituted 1-pyrrolidinyl —

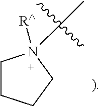

A heterocyclic ring can be attached to its pendant group at any heteroatom or carbon atom that results in a stable structure and any of the ring atoms can be optionally substituted. Examples of such saturated or partially unsaturated heterocyclic radicals include tetrahydrofuranyl, tetrahydrothiophenyl pyrrolidinyl, piperidinyl pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3 indolyl, isoindolinyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

Inhibitor: As used herein, the term “inhibitor” is defined as a compound that binds to and/or inhibits KAT-5 with measurable affinity. In certain embodiments, an inhibitor has an IC₅₀ and/or binding constant of less than about 50 μM, less than about 1 μM, less than about 500 nM, less than about 100 nM, or less than about 10 nM.

Lower alkyl: The term “lower alkyl” refers to a C₁₋₄ straight or branched alkyl group. Exemplary lower alkyl groups are methyl, ethyl, propyl, isopropyl, butyl, isobutyl, and tert-butyl.

Lower haloalkyl: The term “lower haloalkyl” refers to a C₁₋₄ straight or branched alkyl group that is substituted with one or more halogen atoms.

Measurable affinity: The terms “measurable affinity” and “measurably inhibit,” as used herein, means a measurable change in KAT, e.g., KAT-5 activity, between a sample comprising a compound of the present invention, or composition thereof, and the respective KAT, e.g., KAT-5, and an equivalent sample comprising KAT-5, in the absence of said compound, or composition thereof.

Mutant: As used herein, the term “mutant” refers to an organism, a cell, or a biomolecule (e.g., a nucleic acid or a protein) that comprises a genetic variation as compared to a reference organism, cell, or biomolecule. For example, a mutant nucleic acid may, in some embodiments, comprise a mutation, e.g., a nucleobase substitution, a deletion of one or more nucleobases, an insertion of one or more nucleobases, an inversion of two or more nucleobases, as, or a truncation, as compared to a reference nucleic acid molecule. Similarly, a mutant protein may comprise an amino acid substitution, insertion, inversion, or truncation, as compared to a reference polypeptide. Additional mutations, e.g., fusions and indels, are known to those of skill in the art. An organism or cell comprising or expressing a mutant nucleic acid or polypeptide is also sometimes referred to herein as a “mutant.” In some embodiments, a mutant comprises a genetic variant that is associated with a loss of function of a gene product. A loss of function may be a complete abolishment of function, e.g., an abolishment of the enzymatic activity of an enzyme, or a partial loss of function, e.g., a diminished enzymatic activity of an enzyme. In some embodiments, a mutant comprises a genetic variant that is associated with a gain of function, e.g., with a negative or undesirable alteration in a characteristic or activity in a gene product. In some embodiments, a mutant is characterized by a reduction or loss in a desirable level or activity as compared to a reference; in some embodiments, a mutant is characterized by an increase or gain of an undesirable level or activity as compared to a reference. In some embodiments, the reference organism, cell, or biomolecule is a wild-type organism, cell, or biomolecule.

Nucleic acid: As used herein, the term “nucleic acid” refers to a polymer of at least three nucleotides. In some embodiments, a nucleic acid comprises DNA. In some embodiments comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Parenteral: The term “parenteral” as used herein includes subcutaneous, intravenous, intramuscular, intra-articular, intra-synovial, intrasternal, intrathecal, intrahepatic, intralesional and intracranial injection or infusion techniques.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Peptide: As used herein, the term “peptide” refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to a composition that is suitable for administration to a human or animal subject. In some embodiments, a pharmaceutical composition comprises an active agent formulated together with one or more pharmaceutically acceptable carriers. In some embodiments, the active agent is present in a unit dose amount appropriate for administration in a therapeutic regimen. In some embodiments, a therapeutic regimen comprises one or more doses administered according to a schedule that has been determined to show a statistically significant probability of achieving a desired therapeutic effect when administered to a subject or population in need thereof. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, a pharmaceutical composition is intended and suitable for administration to a human subject. In some embodiments, a pharmaceutical composition is sterile and substantially pyrogen-free.

Pharmaceutically acceptable salt: As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Pharmaceutically acceptable carrier, adjuvant, or vehicle: The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The amount of compounds of the present invention that may be combined with the carrier materials to produce a composition in a single dosage form will vary depending upon the host treated, the particular mode of administration, etc. Preferably, provided compositions are formulated so that a dosage of between 0.01 to about 100 mg/kg, or about 0.1 mg/kg to about 50 mg/kg, and preferably from about 1 mg/kg to about 25 mg/kg, of subject body weight/day of the inhibitor can be administered to a patient receiving these compositions to obtain the desired therapeutic effect. The amount of a compound of the present invention in the composition will also depend upon the particular compound in the composition.

Polypeptides: As used herein, the term “polypeptide,” which is interchangeably used herein with the term “protein,” refers to a polymer of at least three amino acid residues. In some embodiments, a polypeptide comprises one or more, or all, natural amino acids. In some embodiments, a polypeptide comprises one or more, or all non-natural amino acids. In some embodiments, a polypeptide comprises one or more, or all, D-amino acids. In some embodiments, a polypeptide comprises one or more, or all, L-amino acids. In some embodiments, a polypeptide comprises one or more pendant groups or other modifications, e.g., modifying or attached to one or more amino acid side chains, at the polypeptide's N-terminus, at the polypeptide's C-terminus, or any combination thereof. In some embodiments, a polypeptide comprises one or more modifications such as acetylation, amidation, aminoethylation, biotinylation, carbamylation, carbonylation, citrullination, deamidation, deimination, glycosylation, lipidation, methylation, pegylation, phosphorylation, sumoylation, or combinations thereof. In some embodiments, a polypeptide may participate in one or more intra- or inter-molecular disulfide bonds. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may comprise a stapled polypeptide. In some embodiments, a polypeptide participates in non-covalent complex formation by non-covalent or covalent association with one or more other polypeptides (e.g., as in an antibody). In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, the term “polypeptide” may be appended to a name of a reference polypeptide, activity, or structure, in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise a fragment of a parent polypeptide. In some embodiments, a useful polypeptide as may comprise a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Reference: As used herein, the term “reference” refers to a standard or control relative to which a comparison is performed. For example, in some embodiments, an agent, animal, individual, population, sample, sequence, or value of interest is compared to a reference or control agent, animal, individual, population, sample, sequence, or value, in some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein, the term “sample” refers to a biological sample obtained or derived from a source of interest, as described herein. In some embodiments, a source of interest comprises an organism, such as a microbe, a plant, an animal or a human. In some embodiments, a biological sample comprises biological tissue or fluid. In some embodiments, a biological sample may comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; other body fluids, secretions, and/or excretions; and/or cells therefrom. In some embodiments, a biological sample comprises cells obtained from an individual, e.g., from a human or animal subject. In some embodiments, obtained cells are or include cells from an individual from whom the sample is obtained. In some embodiments; a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces). In some embodiments, as will be clear from context, the term “sample” refers to a preparation that is obtained by processing (e.g., by removing one or more components of and/or by adding one or more agents to) a primary sample. For example; filtering using a semi-permeable membrane. Such a “processed sample” may comprise, for example nucleic acids or polypeptides extracted from a sample or obtained by subjecting a primary sample to techniques such as amplification or reverse transcription of mRNA, isolation and/or purification of certain components.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments a human subject is an adult, adolescent, or pediatric subject. In some embodiments, a subject is suffering from a disease, disorder or condition, e.g., a disease, disorder or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease; disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Substituted or optionally substituted: As described herein, compounds of the invention may contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

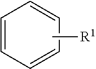

refers to at least

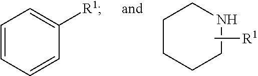

refers to at least

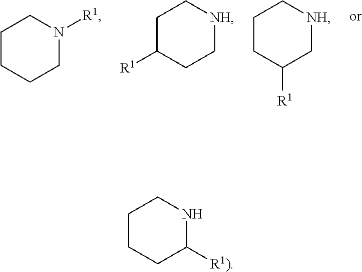

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}, —O(CH₂)₀₋₄R^{∘}, —O—(CH₂)₀₋₄C(O)OR^{∘}; —(CH₂)₀₋₄ CH(OR^{∘})₂; —(CH₂)₀₋₄SR^{∘}; —(CH₂)₀₋₄ Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂, —(CH₂)₀₋₄N(R^{∘})C(O)OR^{∘}; —N(R^{∘})N(R^{∘})C(O)R^{∘}; —N(R^{∘})N(R^{∘}C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}, —(CH₂)₀₋₄C(O)OSiR^{∘}₃; —(CH₂)₀₋₄OC(O)R^{∘};

—OC(O)(CH₂)₀₋₄SR^{∘}; —(CH₂)₀₋₄ SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}; —C(NOR^{∘})R^{∘}; —(CH₂)₀₋₄ SSR^{∘}; —(CH₂)₀₋₄ S(O)₂R^{∘}; —(CH₂)₀₋₄ S(O)₂OR^{∘}; —(CH₂)₀₋₄OS(O)₂R^{∘};
—S(O)₂NR^{∘}₂; —(CH₂)₀₋₄ S(O)R^{∘}; —N(R^{∘})S(O)₂NR^{∘}₂; —N(R^{∘})S(O)₂R^{∘}; —N(OR^{∘})R^{∘}, —C(NH)NR^{∘}₂; —P(O)₂R^{∘}; —P(O)R^{∘}₂, —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁ Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12 membered saturated, partially unsaturated, or aryl mono or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, (haloR^{●}), —(CH₂)₀₋₂ OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂ C(O)OR^{●},

—(CH₂)₀₋₂SR^{●}, —(CH₂)₀₋₂SH, —(CH₂)₀₋₂N₂, —(CH₂)₀₋₂NHR^{●}, —(CH₂)₀₋₂NR^{●}₂, —NO₂, —SiR^{●}₃,
—OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C s aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)NR^{†}₂, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a. “therapeutic agent” is an agent for which a medical prescription is required for administration to humans. In some embodiments, therapeutic agents may be KAT inhibitors, for example, KAT-5 inhibitors, as described herein.

Therapeutically effective amount: As used herein, the term “therapeutically effective amount” refers to an amount that produces a desired effect (e.g., a desired biological, clinical, or pharmacological effect) in a subject or population to which it is administered. In some embodiments, the term refers to an amount statistically likely to achieve the desired effect when administered to a subject in accordance with a particular dosing regimen (e.g., a therapeutic dosing regimen). In some embodiments, the term refers to an amount sufficient to produce the effect in at least a significant percentage (e.g., at least about 25%, about 30%, about 40%, about 50%, about 60%, about 70%, about 80%, about 90%, about 95%, or more) of a population that is suffering from and/or susceptible to a disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be an amount that provides a particular desired response in a significant number of subjects when administered to patients in need of such treatment, e.g., in at least about 25%, about 30%, about 40%, about 50%, about 60%, about 70%, about 80%, about 90%, about 95%, or more patients within a treated patient population. In some embodiments, reference to a therapeutically effective amount may be a reference to an amount sufficient to induce a desired effect as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treat, treatment or treating: As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

Tumor: As used herein, the term “tumor” refers to an abnormal growth of cells or tissue. In some embodiments, a tumor may comprise cells that are precancerous benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

Unit dosage form: The expression “unit dosage form” as used herein refers to a physically discrete unit of a provided compound and/or compositions thereof appropriate for the subject to be treated. It will be understood, however, that the total daily usage of the active agent (i.e., compounds and compositions of the present invention) will be decided by the attending physician within the scope of sound medical judgment. The specific effective dose level for any particular subject (i.e., patient) or organism will depend upon a variety of factors including the disorder being treated and the severity of the disorder; activity of specific active agent employed; specific composition employed; age, body weight, general health, sex and diet of the subject; time of administration, route of administration, and rate of excretion of the specific active agent employed; duration of the treatment; and like factors well known in the medical arts.

Unsaturated: The term “unsaturated,” as used herein, means that a moiety has one or more units of unsaturation.

Wild-type: As used herein, the term “wild-type” refers to a firm of an entity (e.g., a polypeptide or nucleic acid) that has a structure and/or activity as found in nature in a “normal” (as contrasted with mutant, diseased, altered) state or context. In some embodiments, more than one “wild type” form of a particular polypeptide or nucleic acid may exist in nature, for example as “alleles” of a particular gene or normal variants of a particular polypeptide. In some embodiments, that form (or those forms) of a particular polypeptide or nucleic acid that is most commonly observed in a population (e.g., in a human population) is the “wild type” form.

### DETAILED DESCRIPTION OF CERTAIN EMBODIMENTS

According to some aspects, the present invention provides a compound of formula I:

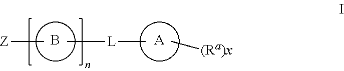

or a pharmaceutically acceptable salt thereof, wherein:
Ring A is selected from phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, an 8-10 membered bicyclic heterocyclic ring having 1-3 heteroatoms independently selected from nitrogen oxygen and sulfur, and an 8-10 membered bicyclic heteroaryl ring having 1-3 heteroatoms independently selected from nitrogen oxygen and sulfur;
L is a 3- to 6-atom linker comprising at least one —S(O)₂— group and 1-4 additional groups independently selected from —C(O)—, —NH—, —O—, and C₁₋₃ aliphatic; wherein:
   two atoms of L may, together with their intervening atoms, form a 4-6 membered saturated or partially unsaturated heterocyclic ring having 1-2 heteroatoms independently selected from nitrogen, oxygen and sulfur, or a 5-6 membered heteroaryl ring having 1-2 heteroatoms independently selected from nitrogen, oxygen and sulfur;
Ring B is an optionally substituted group selected from phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, and an 8-10 membered bicyclic heteroaryl ring having 1-3 heteroatoms independently selected from nitrogen oxygen and sulfur;
R^{a} is selected from halogen, —CN, —NO₂, —OR, —SR, —N(R)₂, —C(O)R, —C(O)₂R, —OC(O)R, —C(O)N(R)₂, —N(R)C(O)R, —Cy, or optionally substituted C₁₋₄ aliphatic;
Z is selected from halogen, —CN, —NO₂, —OR, —SR, —N(R)₂, —C(O)R, —C(O)₂R, —OC(O)R, —C(O)N(R)₂, —N(R)C(O)R, —Cy, —(C₁₋₃ aliphatic)-Cy or optionally substituted C₁₋₄ aliphatic;
Cy is an optionally substituted group selected from phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 6-8 membered bridged bicyclic heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, and an 8-10 membered bicyclic heteroaryl ring having 1-3 heteroatoms independently selected from nitrogen oxygen and sulfur;
each R is independently hydrogen or an optionally substituted group selected from C₁₋₄ aliphatic, phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, and an 8-10 membered bicyclic heteroaryl ring having 1-3 heteroatoms independently selected from nitrogen oxygen and sulfur;
n is 0 or 1; and
x is 0, 1, 2, or 3.

According to some aspects, the present invention provides a compound of formula I′:

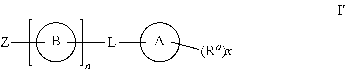

or a pharmaceutically acceptable salt thereof, wherein:
Ring A is selected from phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, an 8-10 membered bicyclic heterocyclic ring having 1-3 heteroatoms independently selected from nitrogen oxygen and sulfur, and an 8-10 membered bicyclic heteroaryl ring having 1-3 heteroatoms independently selected from nitrogen oxygen and sulfur;
L is a 2- to 6-atom linker comprising at least one group selected from —C(O)— and —S(O)_{y}— and 1-4 additional groups independently selected from —C(O)—, —NR—, —O—, and C₁₋₃ aliphatic; wherein:
   two atoms of L may, together with their intervening atoms, form a 4-6 membered saturated or partially unsaturated heterocyclic ring having 1-2 heteroatoms independently selected from nitrogen, oxygen and sulfur, or a 5-6 membered heteroaryl ring having 1-2 heteroatoms independently selected from nitrogen, oxygen and sulfur;
Ring B is an optionally substituted group selected from phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, and an 8-10 membered bicyclic heteroaryl ring having 1-3 heteroatoms independently selected from nitrogen oxygen and sulfur;
R^{a} is selected from halogen, —CN, —NO₂, —OR, —SR, —N(R)₂, —C(O)R, —C(O)₂R, —OC(O)R, —C(O)N(R)₂, —N(R)C(O)R, —Cy, or optionally substituted C₁₋₄ aliphatic;
Z is selected from halogen, —CN, —NO₂, —OR, —SR, —N(R)₂, —C(O)R, —C(O)₂R, —OC(O)R, —C(O)N(R)₂, —N(R)C(O)R, —Cy, —(C₁₋₃ aliphatic)-Cy or optionally substituted C₁₋₄ aliphatic;
Cy is an optionally substituted group selected from phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 6-8 membered bridged bicyclic heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, and an 8-10 membered bicyclic heteroaryl ring having 1-3 heteroatoms independently selected from nitrogen oxygen and sulfur;
each R is independently hydrogen or an optionally substituted group selected from C₁₋₄ aliphatic, phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic amyl ring, and an 8-10 membered bicyclic heteroaryl ring having 1-3 heteroatoms independently selected from nitrogen oxygen and sulfur;
y is 1 or 2;
n is 0 or 1; and
x is 0, 1, 2 or 3.

According to some aspects, the present invention provides a compound of formula I″:

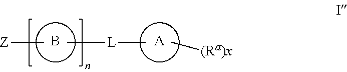

or a pharmaceutically acceptable salt thereof, wherein:
Ring A is selected from phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, an 8-10 membered bicyclic heterocyclic ring having 1-3 heteroatoms independently selected from nitrogen oxygen and sulfur, and an 8-10 membered bicyclic heteroaryl ring having 1-3 heteroatoms independently selected from nitrogen oxygen and sulfur;
L is a 2- to 6-atom linker comprising at least one group selected from —C(O)— and —S(O)_{y}— and 1-4 additional groups independently selected from —C(O)—, —NR—, —O—, and C₁₋₃ aliphatic; wherein:
   two atoms of L may, together with their intervening atoms, form a 4-6 membered saturated or partially unsaturated heterocyclic ring having 1-2 heteroatoms independently selected from nitrogen, oxygen and sulfur, or a 5-6 membered heteroaryl ring having 1-2 heteroatoms independently selected from nitrogen, oxygen and sulfur;
Ring B is an optionally substituted group selected from phenyl, a 3-7 membered saturated or partially unsaturated carbocyclic ring, a 3-7 membered saturated or partially unsaturated heterocyclic ring having 1-2 heteroatoms independently selected front nitrogen, oxygen and sulfur, a 5-6 membered heteroaryl ring having 1-2 heteroatoms independently selected from nitrogen, oxygen and sulfur, an 8-10 membered bicyclic aryl ring, and an 8-10 membered bicyclic heteroaryl ring having 1-3 heteroatoms independently selected from nitrogen oxygen and sulfur;
R^{a} is selected from halogen, —CN, —NO₂, —OR, —SR, —N(R)₂, —C(O)R, —C(O)₂R, —OC(O)R, —C(O)N(R)₂, —N(R)C(O)R, —Cy, or optionally substituted C₁₋₄ aliphatic;
Z is selected from halogen, —CN, —NO₂, —OR, —SR, —N(R)₂, —C(O)R, —C(O)₂R, —OC(O)R, —C(O)N(R)₂, —N(R)C(O)R, —N(R)C(O)₂R, —N(R)C(O)N(R)₂, —S(O)₂R, —Cy, —(C₁₋₃ aliphatic)-Cy or optionally substituted C₁₋₄ aliphatic;
Cy is an optionally substituted group selected from phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 6-8 membered bridged bicyclic heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, and an 8-10 membered bicyclic heteroaryl ring having 1-3 heteroatoms independently selected from nitrogen oxygen and sulfur;
each R is independently hydrogen or an optionally substituted group selected from C₁₋₄ aliphatic, phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, and an 8-10 membered bicyclic heteroaryl ring having 1-3 heteroatoms independently selected from nitrogen oxygen and sulfur;
y is 1 or 2;
n is 0 or 1; and
x is 0, 1, 2, or 3.

As defined above, Ring A is selected from phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, an 8-10 membered bicyclic heterocyclic ring having 1-3 heteroatoms independently selected from nitrogen oxygen and sulfur, and an 8-10 membered bicyclic heteroaryl ring having 1-3 heteroatoms independently selected from nitrogen oxygen and sulfur.

In some embodiments, Ring A is phenyl.

In some embodiments, Ring A is a 3-7 membered saturated or partially unsaturated carbocyclic ring. In some embodiments, Ring A is a 5-6 membered saturated or partially unsaturated carbocyclic ring. In some embodiments, Ring A is selected from cyclopropyl, cyclobutyl, cyclopentyl, cyclohexyl and cycloheptyl.

In some embodiments, Ring A is a 3-7 membered saturated or partially unsaturated heterocyclic ring having 1-2 heteroatoms independently selected from nitrogen, oxygen and sulfur. In some embodiments, Ring A is a 6-membered saturated or partially unsaturated heterocyclic ring having 1-2 heteroatoms independently selected from nitrogen, oxygen and sulfur. In some embodiments, Ring A is selected from piperazinyl or morpholinyl. In some embodiments, Ring A is a 6-membered saturated or partially unsaturated heterocyclic ring having 1 heteroatom independently selected from nitrogen, oxygen and sulfur. In some embodiments, Ring A is piperidinyl.

In some embodiments, Ring A is a 5-6 membered heteroaryl ring having 1-2 heteroatoms independently selected from nitrogen, oxygen and sulfur. In some embodiments, Ring A is selected from pyrrolyl, furanyl, thiophenyl, pyrazolyl, imidazolyl, oxazolyl, thiazolyl, pyridyl, and pyrimidinyl.

In some embodiments, Ring A is a 8-10 membered bicyclic aryl ring. In some embodiments, Ring A is naphthyl.

In some embodiments, Ring A is an 8-10 membered bicyclic heterocyclic ring having 1-3 heteroatoms independently selected from nitrogen oxygen and sulfur. In some embodiments, Ring A is indolinyl, 3H-indolyl or isoindolinyl.

In some embodiments, Ring A is a 8-10 membered bicyclic heteroaryl ring having 1-3 heteroatoms independently selected from nitrogen oxygen and sulfur. In some embodiments, Ring A is a 9-membered bicyclic heteroaryl ring having 1-3 heteroatoms independently selected from nitrogen oxygen and sulfur. In some embodiments, Ring A is a 9-membered bicyclic heteroaryl ring having 1-2 heteroatoms independently selected from nitrogen oxygen and sulfur. In some embodiments, A is selected from indazolyl, benzimidazolyl, indolyl, or isoindolyl. In some embodiments, Ring A is a 10-membered bicyclic heteroaryl ring having 1-2 heteroatoms independently selected from nitrogen oxygen and sulfur. In some embodiments, A is selected from quinolyl, isoquinolyl, or quinazolinyl.

In some embodiments, Ring A-(R^{a})ₓ is selected from the group consisting of

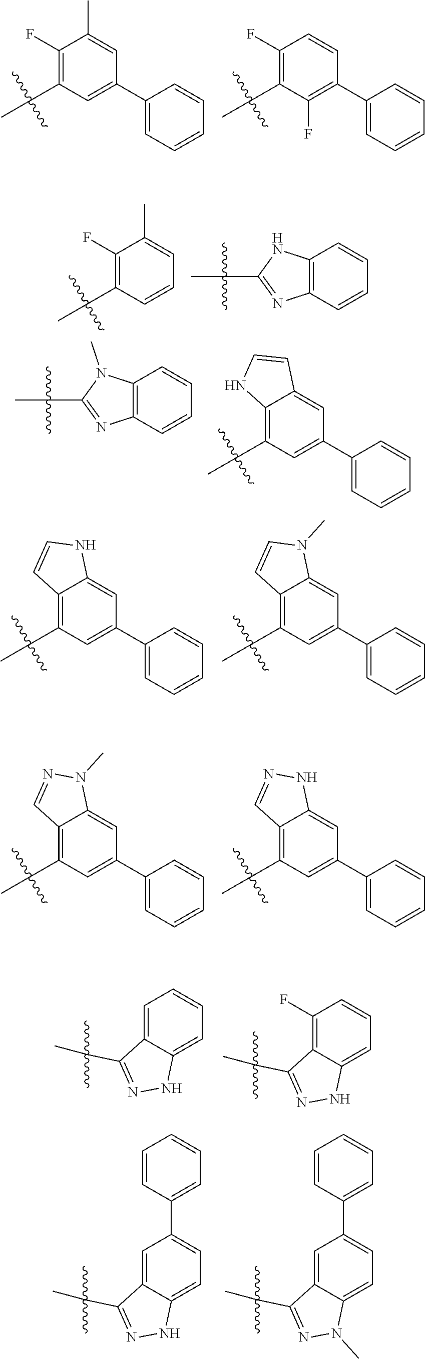

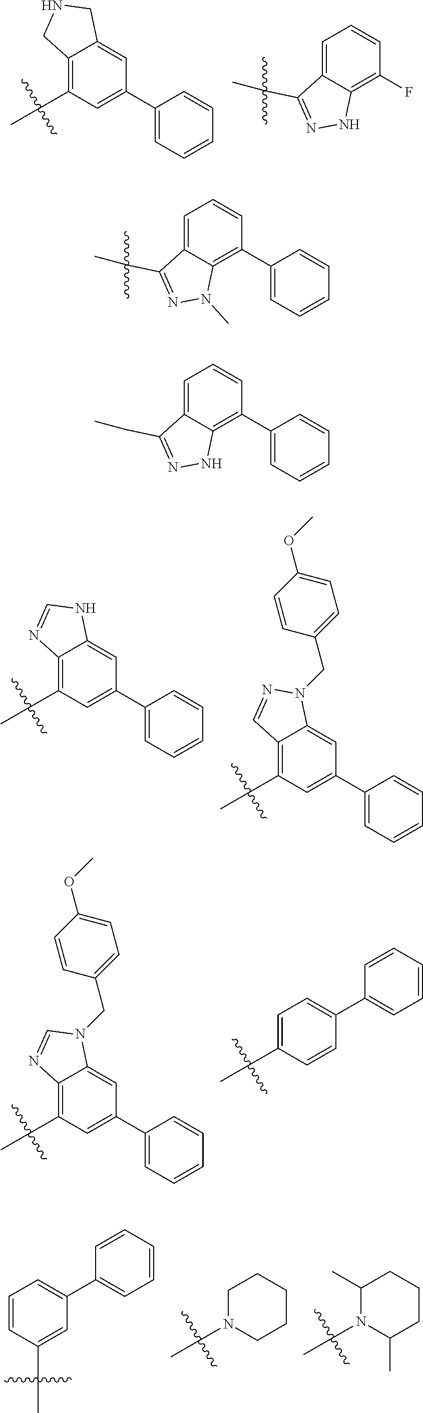

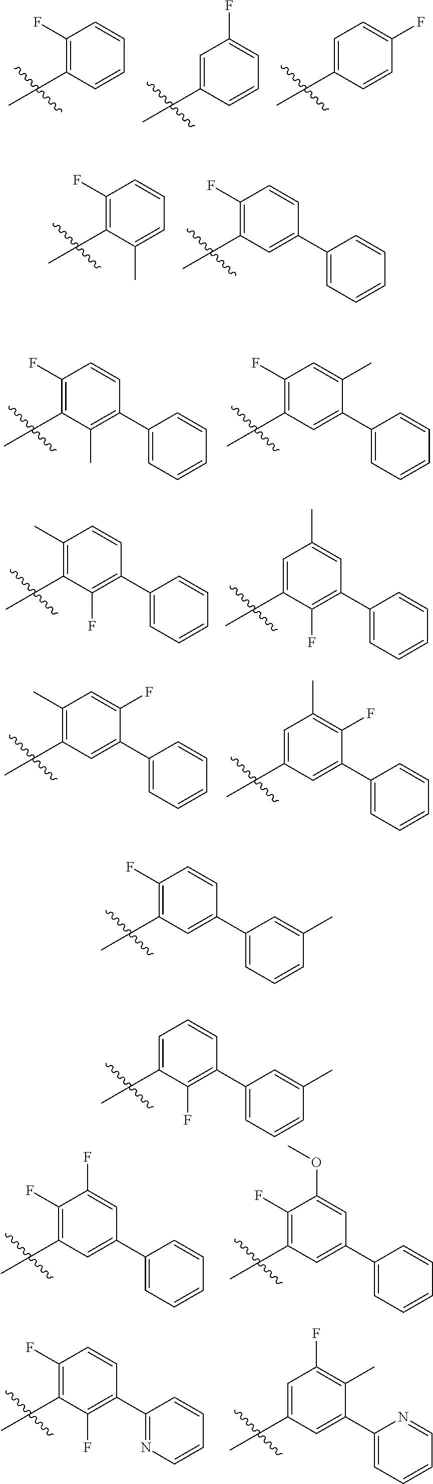

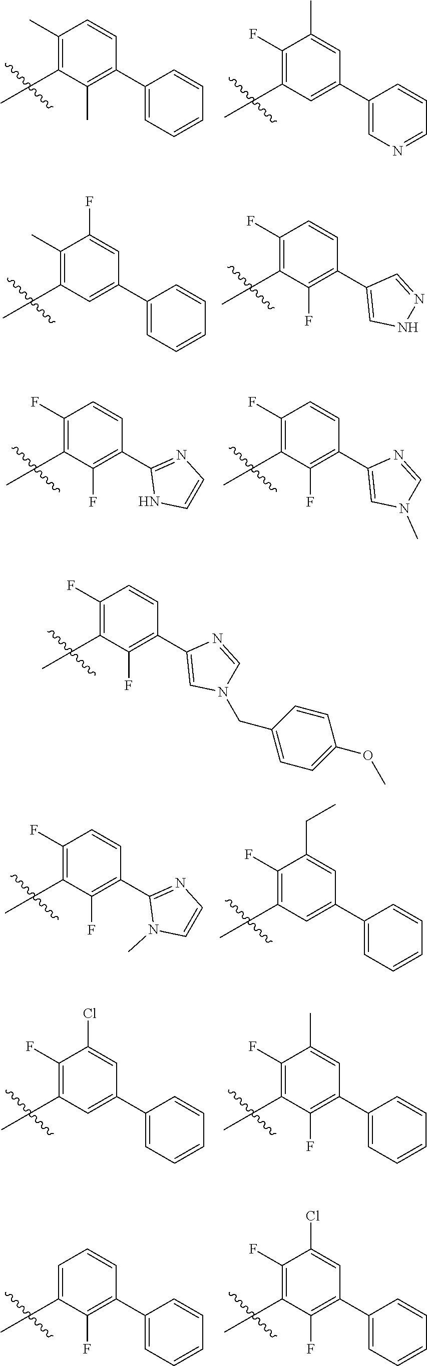

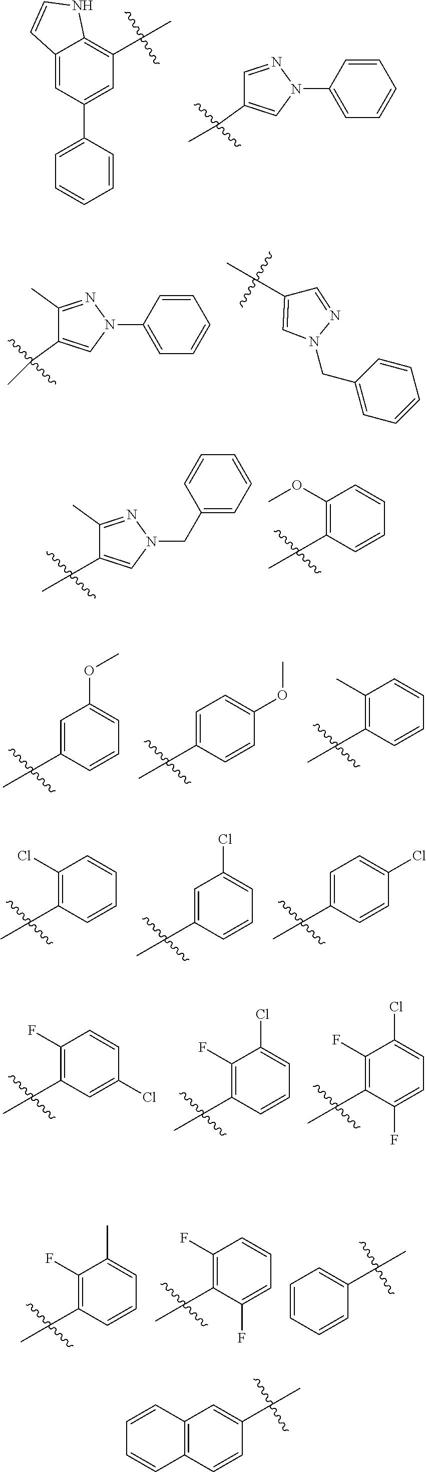

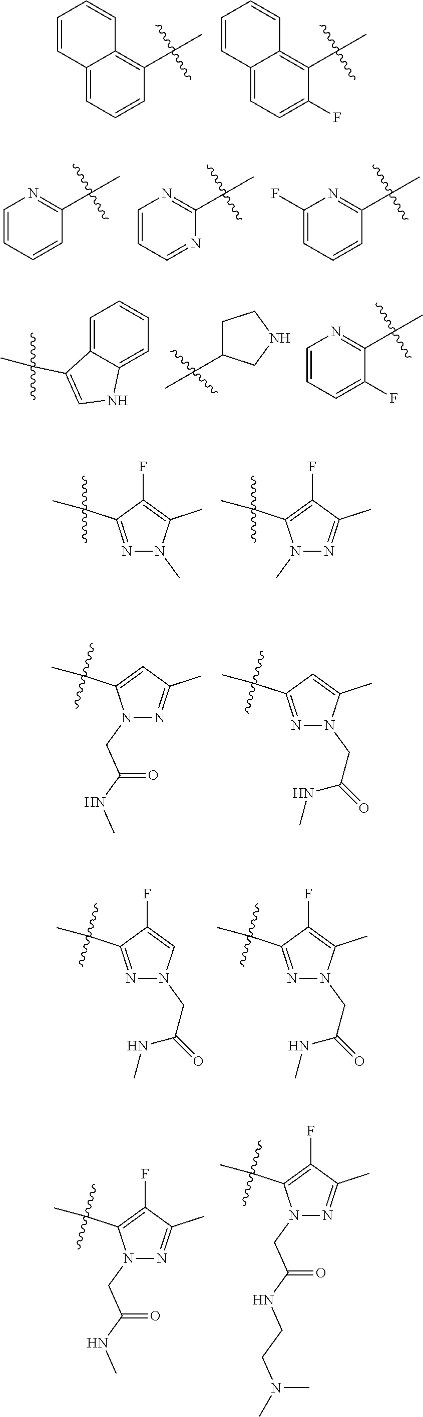

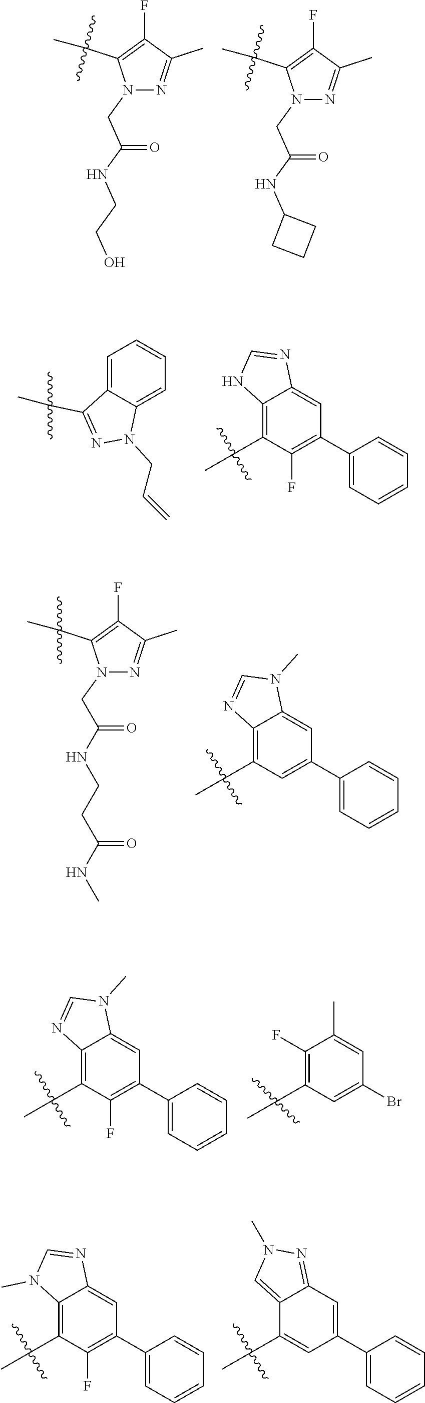

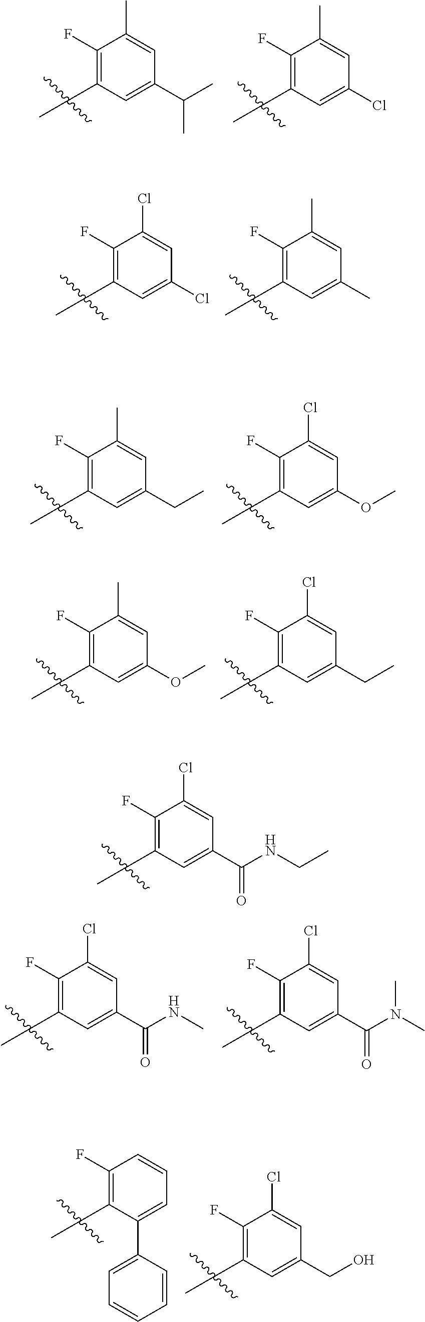

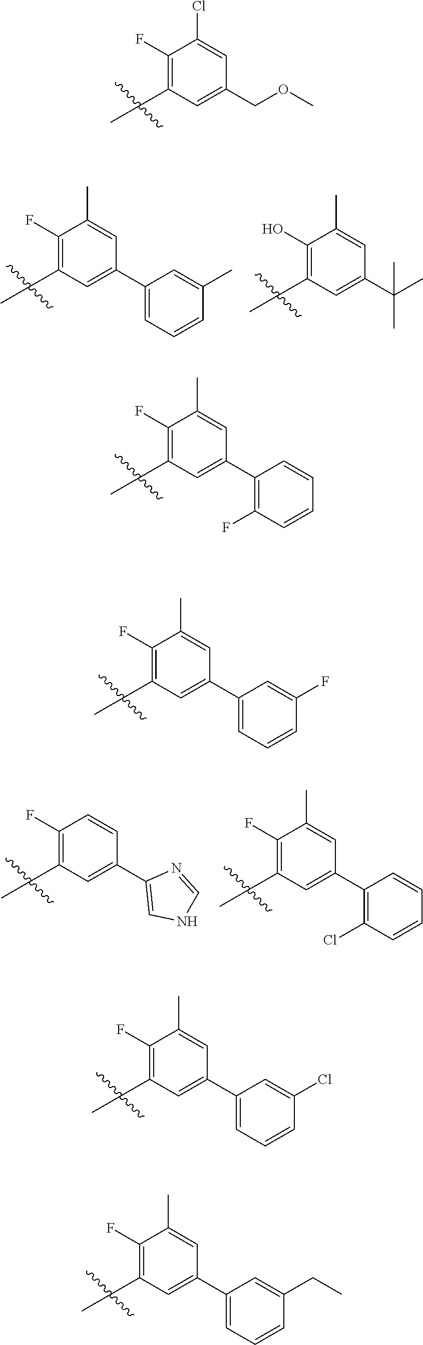

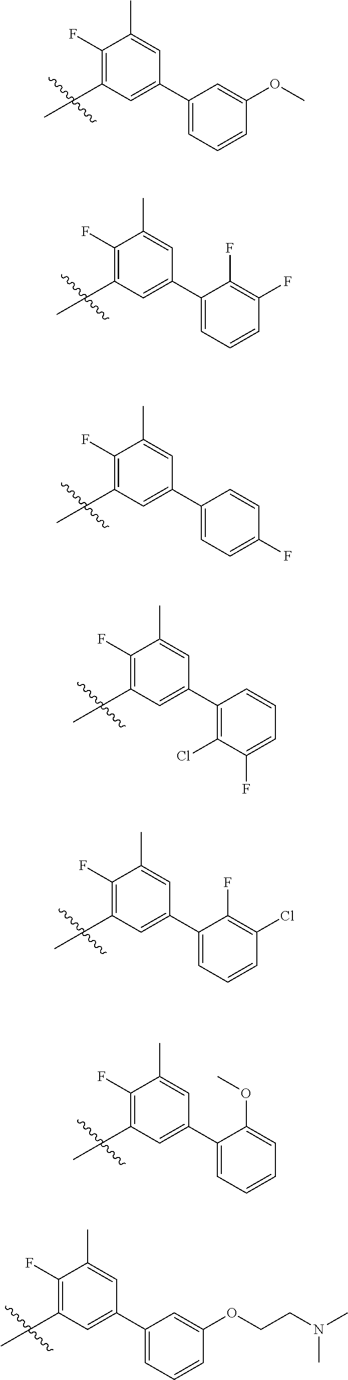

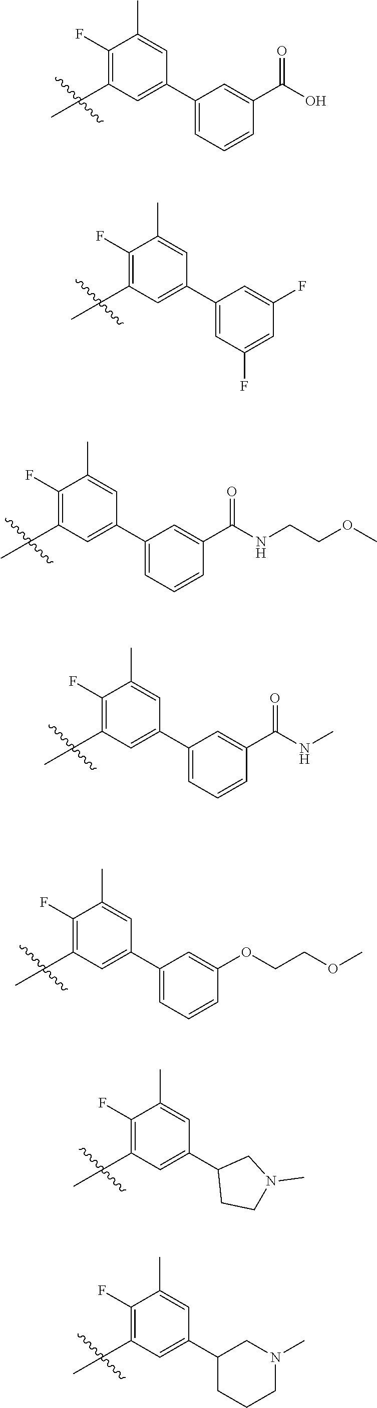

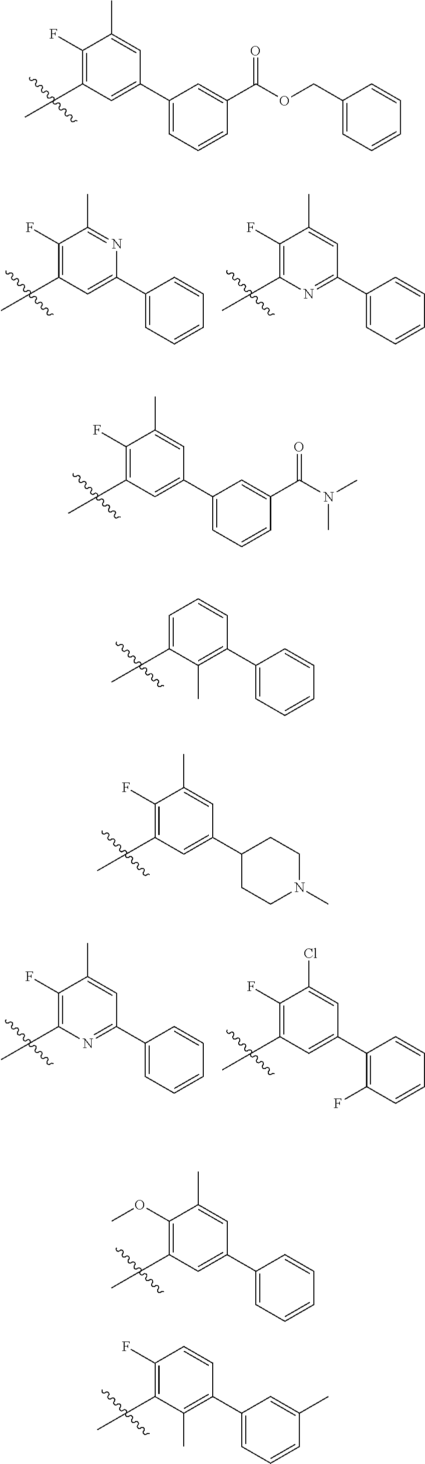

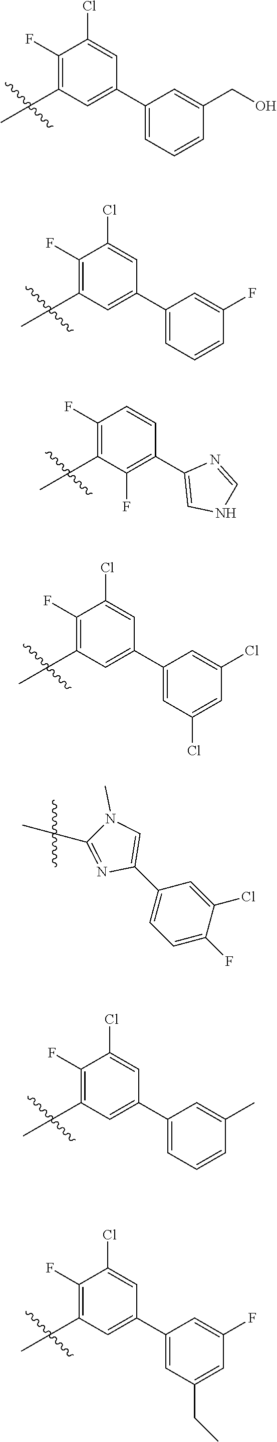

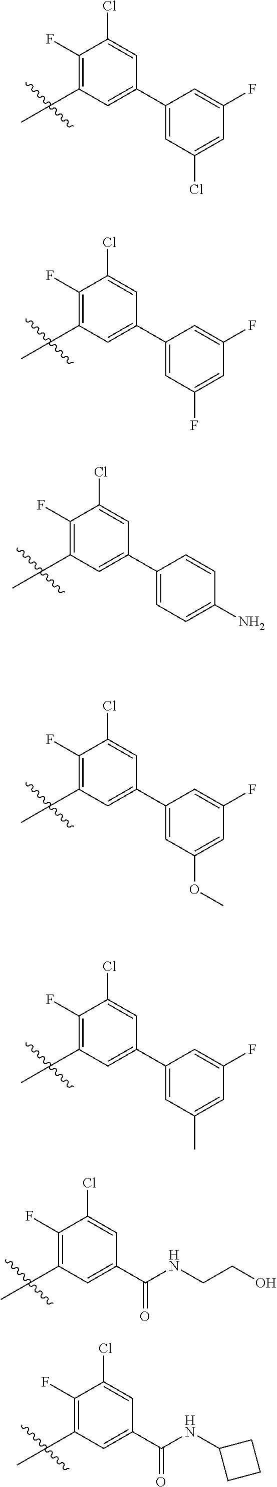

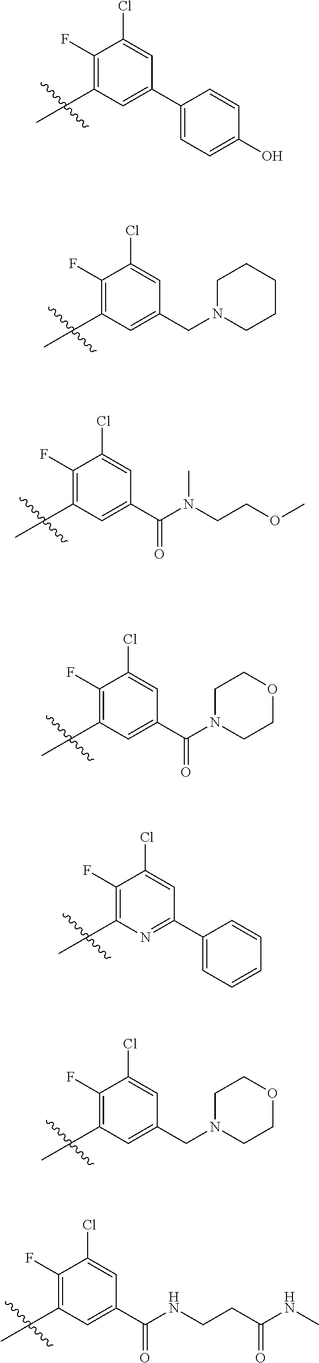

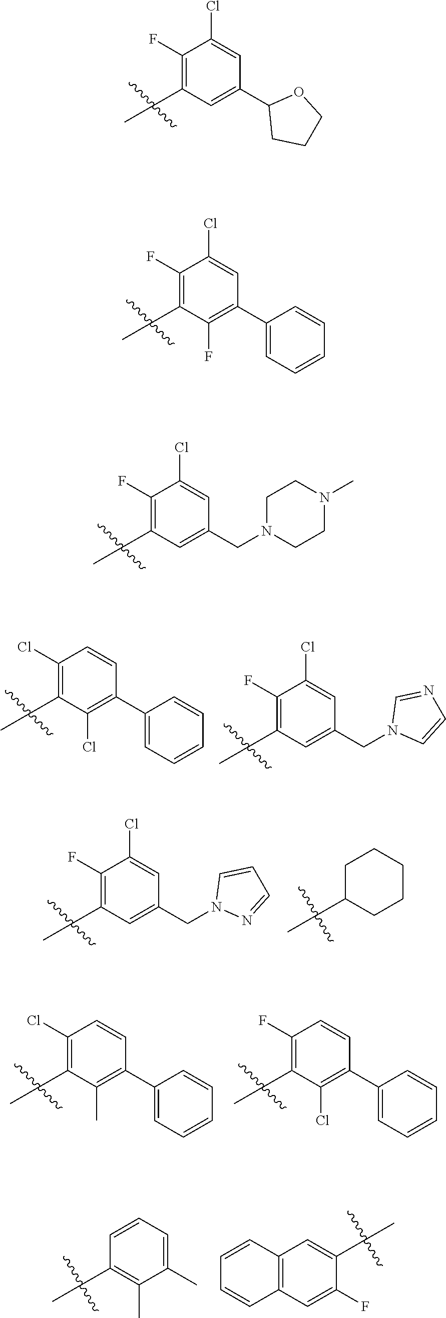

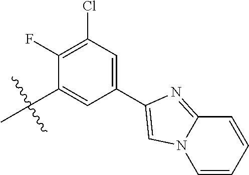 [SMILES: *c1cc(-c2cn3ccccc3n2)cc(Cl)c1F]

As defined above, Ring B is an optionally substituted group selected from phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, and an 8-10 membered bicyclic heteroaryl ring having 1-3 heteroatoms independently selected from nitrogen oxygen and sulfur.

In some embodiments, Ring B is optionally substituted phenyl.

In some embodiments, Ring B is an optionally substituted 3-7 membered saturated or partially unsaturated carbocyclic ring. In some embodiments, Ring B is an optionally substituted cyclopropyl, cyclobutyl, cyclopentyl, cyclohexyl or cycloheptyl.

In some embodiments, Ring B is an optionally substituted 3-7 membered saturated or partially unsaturated heterocyclic ring having 1-2 heteroatoms independently selected from nitrogen, oxygen and sulfur. In some embodiments, Ring B is an optionally substituted 3-4 membered saturated heterocyclic ring having 1 heteroatom independently selected from nitrogen, oxygen and sulfur. In some embodiments, Ring B is an optionally substituted 5-6 membered saturated or partially unsaturated heterocyclic ring having 1 heteroatom independently selected from nitrogen, oxygen and sulfur. In some embodiments, Ring B is selected from optionally substituted azetidinyl, pyrrolidinyl and piperidinyl.

In some embodiments, Ring B is an optionally substituted 5-6 membered heteroaryl ring having 1-2 heteroatoms independently selected from nitrogen, oxygen and sulfur. In some embodiments, Ring B is an optionally substituted pyrrolyl, thiophenyl, pyrazolyl, imidazolyl, oxazolyl, thiazolyl, pyridyl, or pyrimidinyl.

In some embodiments, Ring B is an optionally substituted 8-10 membered bicyclic aryl ring. In some embodiments, Ring B is optionally substituted naphthyl.

In some embodiments, Ring B is an optionally substituted 8-10 membered bicyclic heteroaryl ring having 1-3 heteroatoms independently selected from nitrogen oxygen and sulfur. In some embodiments, Ring B is an optionally substituted 8-10 membered bicyclic heteroaryl ring having 1-3 heteroatoms independently selected from nitrogen oxygen and sulfur. In some embodiments, Ring B is an optionally substituted 9-membered bicyclic heteroaryl ring having 1-3 heteroatoms independently selected from nitrogen oxygen and sulfur. In some embodiments, Ring B is an optionally substituted 9-membered bicyclic heteroaryl ring having 1-2 heteroatoms independently selected from nitrogen oxygen and sulfur. In some embodiments, Ring B is optionally substituted indazolyl, benzimidazolyl, indolyl, or isoindolyl. In some embodiments, Ring B is an optionally substituted 10-membered bicyclic heteroaryl ring having 1-2 heteroatoms independently selected from nitrogen oxygen and sulfur. In some embodiments, Ring B is optionally substituted quinolyl, isoquinolyl, or quinazolinyl.

In some embodiments, Ring B is selected from the group consisting of

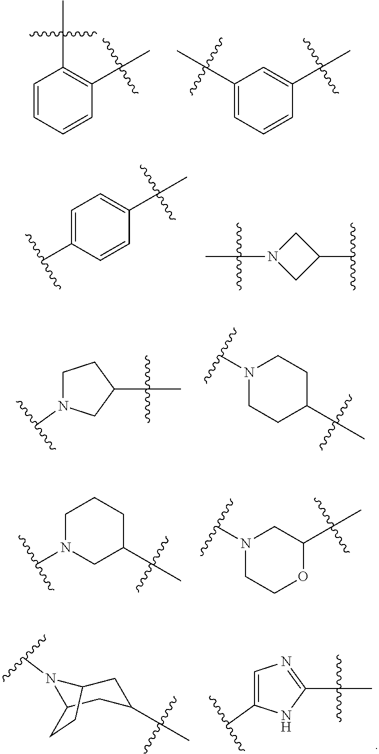

In some embodiments, Ring B is not

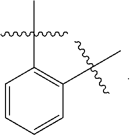 [SMILES: *c1ccccc1*]

As defined above for formula I, L is a 3- to 6-atom linker comprising at least one —S(O)₂— group and 1-4 additional groups independently selected from —C(O)—, —NH—, —O—, and C₁₋₃ aliphatic; wherein:
two atoms of L may, together with their intervening atoms, form a 4-6 membered saturated or partially unsaturated heterocyclic ring having 1-2 heteroatoms independently selected from nitrogen, oxygen and sulfur, or a 5-6 membered heteroaryl ring having 1-2 heteroatoms independently selected from nitrogen, oxygen and sulfur.

As defined above for formulae I′ and I″, L is a 2- to 6-atom linker comprising at least one group selected from —C(O)— and —S(O)_{y}— and 1-4 additional groups independently selected from —C(O)—, —NR—, —O—, and C₁₋₃ aliphatic; wherein:
two atoms of L may, together with their intervening atoms, form a 4-6 membered saturated or partially unsaturated heterocyclic ring having 1-2 heteroatoms independently selected from nitrogen, oxygen and sulfur, or a 5-6 membered heteroaryl ring having 1-2 heteroatoms independently selected from nitrogen, oxygen and sulfur

As used herein, the terms “2- to 6-atom linker” and “3- to 6-atom linker”; or any of variation thereof (e.g., “3- to 5-atom linker”, “4-atom linker”, etc.), mean a bivalent moiety which is 2- to 6-atoms in linear length or 3- to 6-atoms in linear length, respectively. Exemplary 4-atom linkers include, by way of example,

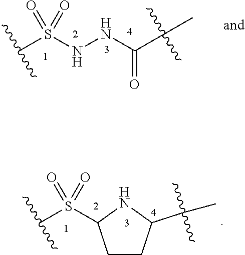 [SMILES: *C(=O)NNS(*)(=O)=O ; *C1CCC(S(*)(=O)=O)N1]

It will be appreciated that when a L comprises a C₁₋₃ aliphatic, such C₁₋₃ aliphatic may be unsubstituted or substituted as defined above for an “optionally substituted group”.

In some embodiments of formulae I′ and I″, L is a 2- to 6-atom linker comprising at least one group selected from —C(O)— and —S(O)_{y}— and 1-4 additional groups independently selected from —C(O)—, —NR—, —O—, and C₁₋₃ aliphatic. In some embodiments of formulae I′ and I″, L is a 2- to 6-atom linker comprising at least one —C(O)— group and 1-4 additional groups independently selected from —C(O)—, —NR—, —O—, and C₁₋₃ aliphatic. In some embodiments of formulae I′ and I″, L is a 2- to 4-atom linker comprising at least one —C(O)— group and 1-3 additional groups independently selected from —C(O)—, —NH—, —O—, and C₁₋₃ aliphatic. In some embodiments of formulae I′ and I″, L is a 2- to 4-atom linker comprising at least one —C(O)— group and 2-3 additional groups independently selected from —C(O)—, —NH—, —O—, and C₁₋₃ aliphatic. In some embodiments of formulae I′ and I″, L is a 3- to 4-atom linker comprising at least one —C(O)— group and 2-3 additional groups independently selected from —C(O)—, —NH—, —O—, and C₁₋₃ aliphatic.

In some embodiments of formulae I′ and I″, L is a 3- to 4-atom linker comprising at least one group selected from —C(O)— and —S(O)_{y}— and 1-2 additional groups independently selected from —C(O)—, —NR—, —O—, and C₁₋₃ aliphatic. In some such embodiments, y is 1. Accordingly, in some embodiments of formulae I′ and I″, L is a 3- to 4-atom linker comprising at least one group selected from —C(O)— and —S(O)— and 1-4 additional groups independently selected from —C(O)—, —NR—, —O—, and C₁₋₃ aliphatic.

In some embodiments of formulae I′ and I″, L is a 2- to 6-atom linker comprising at least one —S(O)— group and 1-4 additional groups independently selected from —C(O)—, —NH—, —O—, and C₁₋₃ aliphatic. In some embodiments of formulae I′ and I″, L is a 4-atom linker comprising at least one —S(O)— group and 2-3 additional groups independently selected from —C(O)—, —NH—, —O—, and C₁₋₃ aliphatic. In some embodiments of formulae I′ and I″, L is a 4-atom linker comprising at least one —S(O)— group and 2-3 additional groups independently selected from —C(O)—. —NR—, —O—, and C₁₋₃ aliphatic.

In some embodiments of formulae I′ and I″, L is a 2- to 6-atom linker comprising at least one group selected from —C(O)— and —S(O)_{y}— and 1-4 additional groups independently selected from —C(O)—, —NR—, —O—, and C₁₋₃ aliphatic. In some embodiments of formulae I′ and I″, L is a 2-atom linker comprising at least one group selected from —C(O)— and —S(O)_{y}— and 1 additional group independently selected from —C(O)—, —NR—, —O—, and C₁₋₃ aliphatic.

In some embodiments, L is a 3- to 6-atom linker comprising at least one —S(O)₂— group and 1-4 additional groups independently selected from —C(O)—, —NH—, —O—, and C₁₋₃ aliphatic. In some embodiments of formulae I′ and I″, L is a 3- to 6-atom linker comprising at least one group selected from —C(O)— and —S(O)_{y}— and 1-4 additional groups independently selected from —C(O)—, —NR—, —O—, and C₁₋₃ aliphatic.

In some embodiments, L is a 3- to 6-atom linker comprising one —S(O)₂— group and 1 additional group selected from —C(O)—, —NH—, —O—, and C₁₋₃ aliphatic. In some embodiments of formulae I′ and I″, L is a 3- to 6-atom linker comprising one group selected from —C(O)— and —S(O)_{y}— and 1 additional group selected from —C(O)—, —NR—, and C₁₋₃ aliphatic. In some such embodiments, y is 2 and R is hydrogen.

In some embodiments, L is a 3- to 6-atom linker comprising one —S(O)₂— group and 1 additional group selected from —C(O)—, —NH—, —O—, and C₁₋₂ aliphatic. In some embodiments of formulae I′ and I″, L is a 3- to 6-atom linker comprising one group selected from —C(O)— and —S(O)_{y}— and 1 additional group selected from —C(O)—, —NR—, —O—, and C₁₋₂ aliphatic. In some such embodiments, y is 2 and R is hydrogen.

In some embodiments, L is a 3- to 6-atom linker comprising one —S(O)₂— group and 1 additional group selected from —C(O)—, —NH—, —O—, and —CH₂—. In some embodiments of formulae I′ and I″, L is a 3- to 6-atom linker comprising one group selected from —C(O)— and —S(O)_{y}— and 1 additional group selected from —C(O)—, —NR—, —O—, and —CH₂—. In some such embodiments, y is 2 and R is hydrogen.

In some embodiments, L is a 3- to 6-atom linker comprising one —S(O)₂— group and 2 additional groups independently selected from —C(O)—, —NH—, —O—, and C₁₋₃ aliphatic. In some embodiments of formulae I′ and I″, L is a 3- to 6-atom linker comprising one group selected from —C(O)— and —S(O)_{y}— and 2 additional groups independently selected from —C(O)—, —NR—, —O—, and C₁₋₃ aliphatic. In some such embodiments, y is 2 and R is hydrogen.

In some embodiments, L is a 3- to 6-atom linker comprising one —S(O)₂— group and 2 additional groups independently selected from —C(O)—, —NH—, —O—, and C₁₋₂ aliphatic. In some embodiments of formulae I′ and I″, L is a 3- to 6-atom linker comprising one group selected from —C(O)— and —S(O)_{y}— and 2 additional groups independently selected from —C(O)—, —NR—, —O—, and C₁₋₂ aliphatic. In some such embodiments, y is 2 and R is hydrogen.

In some embodiments, L is a 3- to 6-atom linker comprising one —S(O)₂— group and 2 additional groups independently selected from —C(O)—, —NH—, —O—, and —CH₂—. In some embodiments of formulae I′ and I″, L is a 3- to 6-atom linker comprising one group selected from —C(O)— and —S(O)_{y}— and 2 additional groups independently selected from —C(O)—, —NR—, —O—, and —CH₂—. In some such embodiments, y is 2 and R is hydrogen.

In some embodiments, L is a 4- to 6-atom linker comprising one —S(O)₂— group and 3 additional groups independently selected from —C(O)—, —NH—, —O—, and C₁₋₃ aliphatic. In some embodiments of formulae I′ and I″, L is a 4- to 6-atom linker comprising one group selected from —C(O)— and —S(O)_{y}— and 3 additional groups independently selected from —C(O)—, —NR—, —O—, and C₁₋₃ aliphatic. In some such embodiments, y is 2 and R is hydrogen.

In some embodiments of formulae I′ and I″, L is a 4- to 6-atom linker comprising one group selected from —C(O)— and —S(O)_{y}— and 3 additional groups independently selected from —C(O)—, —NR—, —O—, and C₁₋₃ aliphatic. In some such embodiments, y is 1 and R is hydrogen.

In some embodiments, L is a 4- to 6-atom linker comprising one —S(O)₂— group and 3 additional groups independently selected from —C(O)—, —NH—, —O—, and C₁₋₂ aliphatic. In some embodiments of formulae I′ and I″, L is a 4- to 6-atom linker comprising one group selected from —C(O)— and —S(O)_{y}— and 3 additional groups independently selected from —C(O)—, —NR—, —O—, and C₁₋₂ aliphatic. In some such embodiments, y is 2 and R is hydrogen.

In some embodiments of formulae I′ and I″, L is a 4- to 6-atom linker comprising one group selected from —C(O)— and —S(O)_{y}— and 3 additional groups independently selected from —C(O)—, —NR—, —O—, and C₁₋₂ aliphatic. In some such embodiments, y is 1 and R is hydrogen.

In some embodiments, L is a 4- to 6-atom linker comprising one —S(O)₂— group and 3 additional groups independently selected from —C(O)—, —NH—, —O—, and —CH₂—. In some embodiments of formulae I′ and I″, L is a 4- to 6-atom linker comprising one group selected from —C(O)— and —S(O)_{y}— and 3 additional groups independently selected from —C(O)—, —NR—, —O—, and —CH₂—. In some such embodiments, y is 2 and R is hydrogen.

In some embodiments of formulae I′ and I″, L is a 4- to 6-atom linker comprising one group selected from —C(O)— and —S(O)_{y}— and 3 additional groups independently selected from —C(O)—, —NR—, —O—, and —CH₂—. In some such embodiments, y is 1 and R is hydrogen.

In some embodiments, L is a 5- to 6-atom linker comprising one —S(O)₂— group and 4 additional groups independently selected from —C(O)—, —NH—, —O—, and C₁₋₃ aliphatic. In some embodiments of formulae I′ and I″, L is a 5- to 6-atom linker comprising one group selected from —C(O)— and —S(O)_{y}— and 4 additional groups independently selected from —C(O)—, —NR—, —O—, and C₁₋₃ aliphatic. In some such embodiments, y is 2 and R is hydrogen.

In some embodiments, L is a 5- to 6-atom linker comprising one —S(O)₂— group and 4 additional groups independently selected from —C(O)—, —NH—, —O—, and C₁₋₂ aliphatic. In some embodiments of formulae I′ and I″, L is a 5- to 6-atom linker comprising one group selected from —C(O)— and —S(O)_{y}— and 4 additional groups independently selected from —C(O)—, —NR—, —O—, and C₁₋₂ aliphatic. In some such embodiments, y is 2 and R is hydrogen.

In some embodiments, L is a 5- to 6-atom linker comprising one —S(O)₂— group and 4 additional groups independently selected from —C(O)—, —NH—, —O—, and —CH₂—. In some embodiments of formulae I′ and I″, L is a 5- to 6-atom linker comprising one group selected from —C(O)— and —S(O)_{y}— and 4 additional groups independently selected from —C(O)—, —NR—, —O—, and —CH₂—. In some such embodiments, y is 2 and R is hydrogen.

In some embodiments, L is a 3-atom linker comprising at least one —S(O)₂— group and 1-2 additional groups independently selected from —C(O)—, —NH—, —O—, and C₁₋₃ aliphatic. In some embodiments of formulae I′ and I″, L is a 3-atom linker comprising at least one group selected from —C(O)— and —S(O)_{y}— and 1-2 additional groups independently selected from —C(O)—, —NR—, —O—, and C₁₋₃ aliphatic. In some such embodiments, y is 2 and R is hydrogen.

In some embodiments, L is a 3-atom linker comprising at least one —S(O)₂— group and 1-2 additional groups independently selected from —C(O)—, —NH—, —O—, and C₁₋₂ aliphatic. In some embodiments of formulae I′ and I″, L is a 3-atom linker comprising at least one group selected from —C(O)— and —S(O)_{y}— and 1-2 additional groups independently selected from —C(O)—, —NR—, —O—, and C₁₋₂ aliphatic. In some such embodiments, y is 2 and R is hydrogen.

In some embodiments, L is a 3-atom linker comprising at least one —S(O)₂— group and 1-2 additional groups independently selected from —C(O)—, —NH—, —O—, and —CH₂—. In some embodiments of formulae I′ and I″, L is a 3-atom linker comprising at least one group selected from —C(O)— and —S(O)— and 1-2 additional groups independently selected from —C(O)—, —NR—, —O—, and —CH₂—. In some such embodiments, y is 2 and R is hydrogen.

In some embodiments, L is a 4-atom linker comprising at least one —S(O)₂— group and 1-3 additional groups independently selected from —C(O)—, —NH—, and C₁₋₃ aliphatic. In some embodiments of formulae I′ and I″, L is a 4-atom linker comprising al least one group selected from —C(O)— and —S(O)_{y}— and 1-3 additional groups independently selected from —C(O)—, —NR—, —O—, and C₁₋₃ aliphatic. In some such embodiments, y is 2 and R is hydrogen.

In some embodiments, L is a 4-atom linker comprising at least one —S(O)₂— group and 2-3 additional groups independently selected from —C(O)—, —NH—, —O—, and C₁₋₃ aliphatic. In some embodiments of formulae I′ and I″, L is a 4-atom linker comprising at least one group selected from —C(O)— and —S(O)_{y}— and 2-3 additional groups independently selected from —C(O)—, —NR—, —O—, and C₁₋₃ aliphatic. In some such embodiments, y is 2 and R is hydrogen.

In some embodiments, L is a 4-atom linker comprising at least one —S(O)₂— group and 2-3 additional groups independently selected from —C(O)—, —NH—, —O—, and C₁₋₃ aliphatic. In some embodiments of formulae I′ and I″, L is a 4-atom linker comprising at least one group selected from —C(O)— and —S(O)_{y}— and 2-3 additional groups independently selected from —C(O)—, —NR—, —O—, and C₁₋₂ aliphatic. In some such embodiments, y is 2 and R is hydrogen.

In some embodiments, L is a 4-atom linker comprising at least one —S(O)₂— group and 2-3 additional groups independently selected from —C(O)—, —NH—, —O—, and —CH₂—. In some embodiments of formulae I′ and I″, L is a 4-atom linker comprising at least one group selected from —C(O)— and —S(O)_{y}— and 2-3 additional groups independently selected from —C(O)—, —NR—, —O—, and —CH₂—. In some such embodiments, y is 2 and R is hydrogen.

In some embodiments, L is a 5-atom linker comprising at least one —S(O)₂— group and 1-4 additional groups independently selected from —C(O)—, —NH—, —O—, and C₁₋₃ aliphatic. In some embodiments of formulae I′ and I″, L is a 5-atom linker comprising at least one group selected from —C(O)— and —S(O)_{y}— and 1-4 additional groups independently selected from —C(O)—, —NR—, and C₁₋₂ aliphatic. In some such embodiments, y is 2 and R is hydrogen.

In some embodiments, L is a 5-atom linker comprising at least one —S(O)₂— group and 1-4 additional groups independently selected from —C(O)—, —NH—, —O—, and C₁₋₂ aliphatic. In some embodiments of formulae I′ and I″, L is a 5-atom linker comprising at least one group selected from —C(O)— and —S(O)_{y}— and 1-4 additional groups independently selected from —C(O)—, —NR—, —O—, and C₁₋₂ aliphatic. In some such embodiments, y is 2 and R is hydrogen.

In some embodiments, L is a 5-atom linker comprising at least one —S(O)₂— group and 1-4 additional groups independently selected from —C(O)—, —NH—, —O—, and —CH₂—. In some embodiments of formulae I′ and I″, L is a 5-atom linker comprising at least one group selected from —C(O)— and —S(O)— and 1-4 additional groups independently selected from —C(O)—, —NR—, —O—, and —CH₂—. In some such embodiments, y is 2 and R is hydrogen.

In some embodiments, L is a 6-atom linker comprising at least one —S(O)₂— group and 1-4 additional groups independently selected from —C(O)—, —NH—, —O—, and C₁₋₃ aliphatic. In some embodiments of formulae I′ and I″, L is a 6-atom linker comprising at least one group selected from —C(O)— and —S(O)_{y}— and 1-4 additional groups independently selected from —C(O)—, —NR—, —O—, and C₁₋₃ aliphatic. In some such embodiments, y is 2 and R is hydrogen.

In some embodiments, L is a 6-atom linker comprising at least one —S(O)₂— group and 1-4 additional groups independently selected from —C(O)—, —NH—, —O—, and C₁₋₂ aliphatic. In some embodiments of formulae I′ and I″, L is a 6-atom linker comprising at least one group selected from —C(O)— and —S(O)_{y}— and 1-4 additional groups independently selected from —C(O)—, —NR—, —O—, and —C₁₋₂ aliphatic. In some such embodiments, y is 2 and R is hydrogen.

In some embodiments, L is a 6-atom linker comprising at least one —S(O)₂— group and 1-4 additional groups independently selected from —C(O)—, —NH—, —O—, and —CH₂—. In some embodiments of formulae I′ and I″, L is a 6-atom linker comprising at least one group selected from —C(O)— and —S(O)_{y}— and 1-4 additional groups independently selected from —C(O)—, —NR—, —O—, and —CH₂—. In some such embodiments, y is 2 and R is hydrogen.

In some embodiments, L is selected from the group consisting of

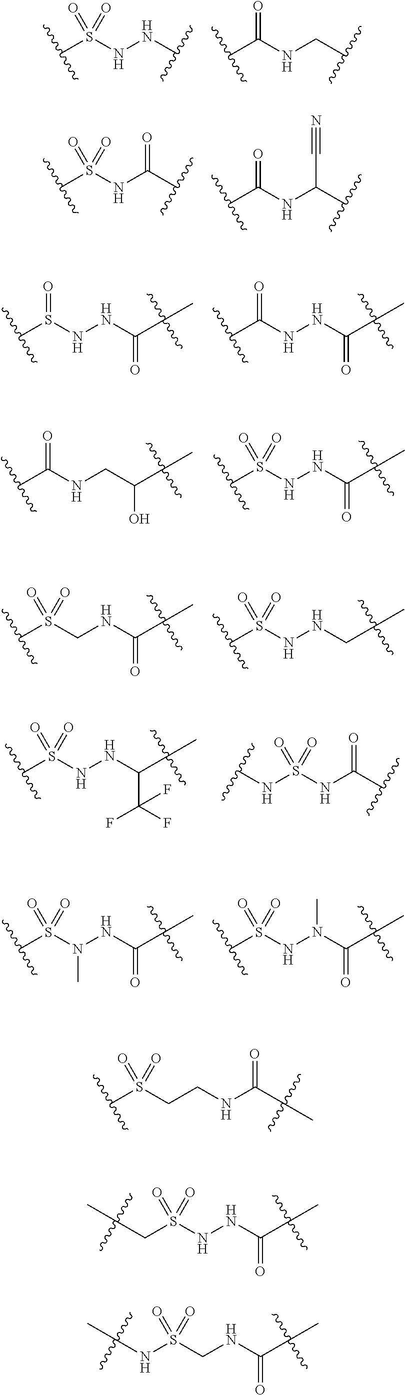

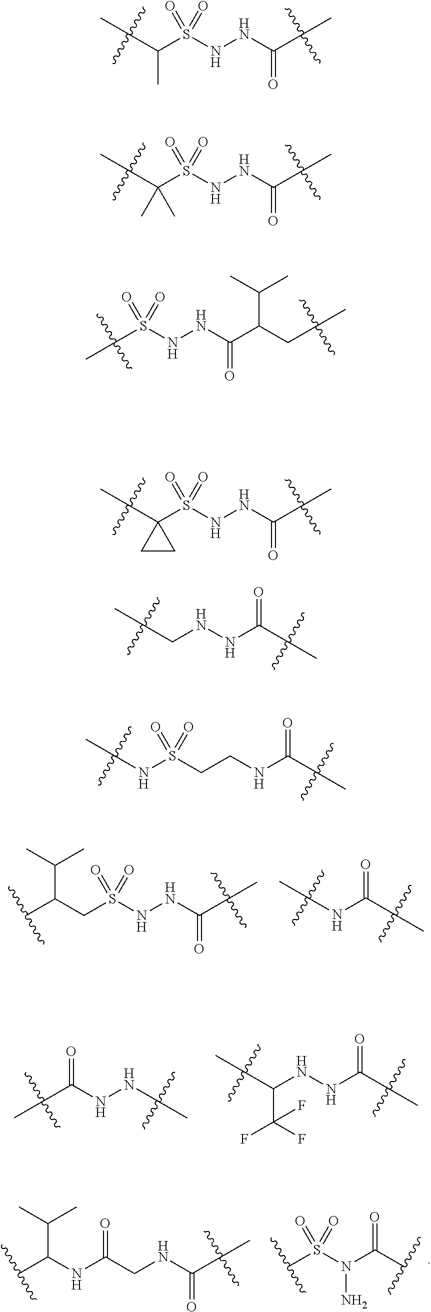

In some embodiments, L is selected from the group consisting of

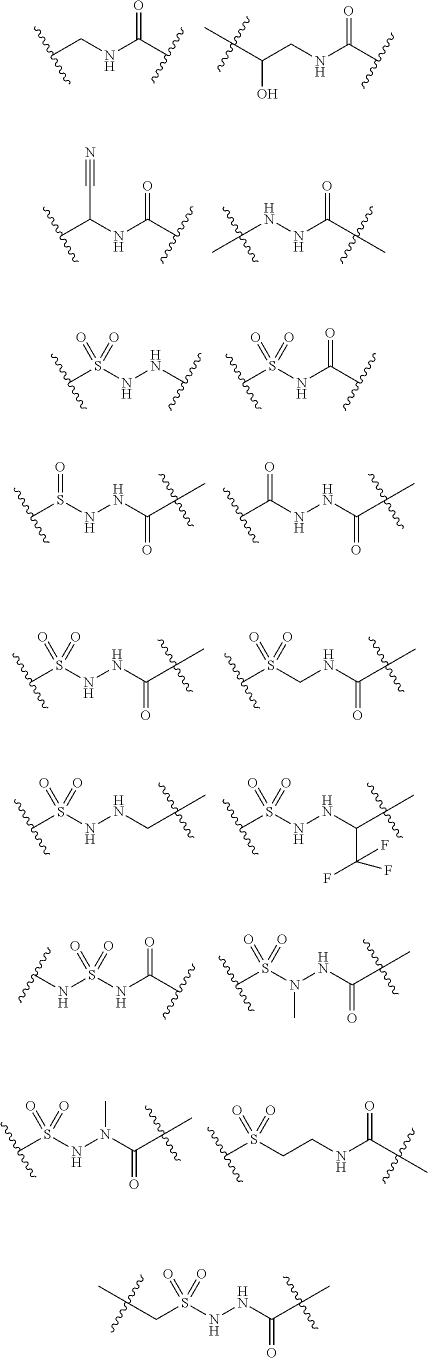

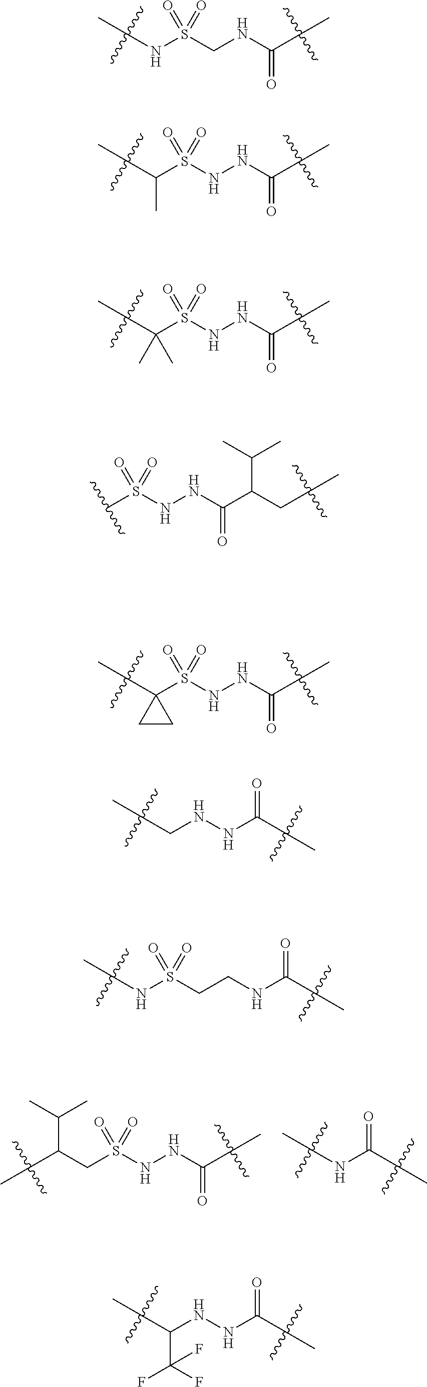

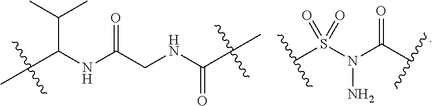 [SMILES: *C(=O)NCC(=O)NC(*)C(C)C ; *C(=O)N(N)S(*)(=O)=O]

In some embodiments, L is a 3- to 6-atom linker comprising at least one —S(O)₂— group and 1-4 additional groups independently selected from —C(O)—, —NH—, —O—, and C₁₋₃ aliphatic, wherein two atoms of L, together with their intervening atoms, form a 4-6 membered saturated or partially unsaturated heterocyclic ring having 1-2 heteroatoms independently selected from nitrogen, oxygen and sulfur, or a 5-6 membered heteroaryl ring having 1-2 heteroatoms independently selected from nitrogen, oxygen and sulfur.

In some embodiments, L is a 4- to 5-atom linker comprising at least one —S(O)₂— group and 1-4 additional groups independently selected from —C(O)—, —NH—, —O—, and C₁₋₃ aliphatic, wherein two atoms of L, together with their intervening atoms, form a 4-6 membered saturated or partially unsaturated heterocyclic ring having 1-2 heteroatoms independently selected from nitrogen, oxygen and sulfur, or a 5-6 membered heteroaryl ring having 1-2 heteroatoms independently selected from nitrogen, oxygen and sulfur.

In some embodiments, L is a 4- to 5-atom linker comprising at least one —S(O)₂— group and 1-4 additional groups independently selected from —C(O)—, —NH—, —O—, and C₁₋₃ aliphatic, wherein two atoms of L, together with their intervening atoms, form a 4-6 membered saturated or partially unsaturated heterocyclic ring having 1-2 heteroatoms independently selected from nitrogen, oxygen and sulfur.

In some embodiments, L is a 4-atom linker comprising at least one —S(O)₂— group and 1-3 additional groups independently selected from —C(O)—, —NH—, —O—, and C₁₋₃ aliphatic, wherein two atoms of L, together with their intervening atoms, form a 4-6 membered saturated or partially unsaturated heterocyclic ring having 1-2 heteroatoms independently selected from nitrogen, oxygen and sulfur.

In some embodiments, L is a 4-atom linker comprising at least one —S(O)₂— group and 1-3 additional groups independently selected from —C(O)—, —NH—, —O—, and C₁₋₂ aliphatic, wherein two atoms of L, together with their intervening atoms, form a 4-6 membered saturated or partially unsaturated heterocyclic ring having 1-2 heteroatoms independently selected from nitrogen, oxygen and sulfur.

In some embodiments, L is a 5-atom linker comprising at least one —S(O)₂— group and 1-4 additional groups independently selected from —C(O)—, —NH—, —O—, and C₁₋₃ aliphatic, wherein two atoms of L, together with their intervening atoms, form a 4-6 membered saturated or partially unsaturated heterocyclic ring having 1-2 heteroatoms independently selected from nitrogen, oxygen and sulfur.

In some embodiments, L is a 5-atom linker comprising at least one —S(O)₂— group and 1-4 additional groups independently selected from —C(O)—, —NH—, —O—, and C₁₋₂ aliphatic, wherein two atoms of L, together with their intervening atoms, form a 4-6 membered saturated or partially unsaturated heterocyclic ring having 1-2 heteroatoms independently selected from nitrogen, oxygen and sulfur.

In some embodiments, L is a 4- to 5-atom linker comprising at least one —S(O)₂— group and 1-4 additional groups independently selected from —C(O)—, —NH—, —O—, and C₁₋₃ aliphatic, wherein two atoms of L, together with their intervening atoms, form a 5-6 membered heteroaryl ring having 1-2 heteroatoms independently selected from nitrogen, oxygen and sulfur.

In some embodiments, L is a 5-atom linker comprising at least one —S(O)₂— group and 1-4 additional groups independently selected from —C(O)—, —NH—, —O—, and C₁₋₃ aliphatic, wherein two atoms of L, together with their intervening atoms, form a 5-6 membered heteroaryl ring having 1-2 heteroatoms independently selected from nitrogen, oxygen and sulfur.

In some embodiments, L is a 5-atom linker comprising at least one —S(O)₂— group and 1-4 additional groups independently selected from —C(O)—, —NH—, —O—, and C₁₋₂ aliphatic, wherein two atoms of L, together with their intervening atoms, forth a 5-6 membered heteroaryl ring having 1-2 heteroatoms independently selected from nitrogen, oxygen and sulfur.

In some embodiments, L is selected from the group consisting of

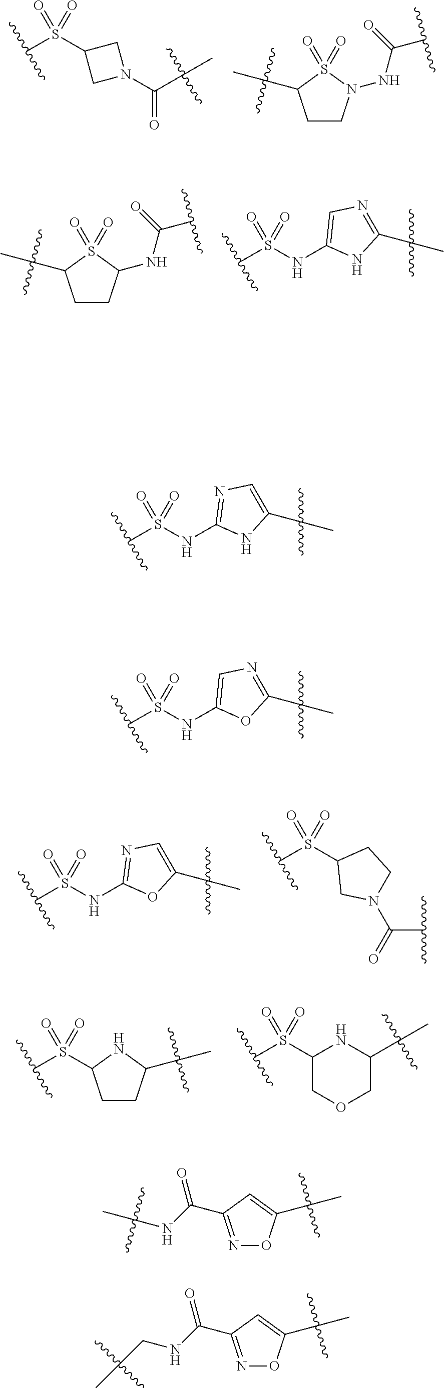

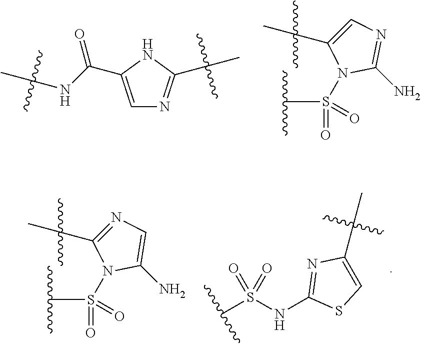 [SMILES: *NC(=O)c1cnc(*)[nH]1 ; *c1cnc(N)n1S(*)(=O)=O ; *c1ncc(N)n1S(*)(=O)=O ; *c1csc(NS(*)(=O)=O)n1]

As defined above, R^{a} is selected from halogen, —CN, —NO₂, —OR, —SR, —N(R)₂, —C(O)R, —C(O)₂R, —CO(O)R, —C(O)N(R)₂, —N(R)C(O)R, —Cy, or optionally substituted C₁₋₄ aliphatic.

In some embodiments, R^{a} is halogen.

In some embodiments, R^{a} is selected from —CN, —NO₂, —C(O)R, —C(O)₂R, and —C(O)N(R)₂.

In some embodiments, R^{a} is selected from —OR, —SR, and —N(R)₂.

In some embodiments, R^{a} is selected from —OC(O)R and —N(R)C(O)R.

In some embodiments, R^{a} is Cy.

In some embodiments, R^{a} is optionally substituted C₁₋₄ aliphatic. In some embodiments, R^{a} is methyl.

In some embodiments, R^{a} is —Cy.

As defined above, Z is selected from halogen, —CN, —NO₂, —OR, —SR, —N(R)₂, —C(O)R, —C(O)₂R, —OC(O)R, —C(O)N(R)₂, —N(R)C(O)R, —Cy, —(C₁₋₃ aliphatic)-Cy or optionally substituted C₁₋₄ aliphatic.

In some embodiments, Z is optionally substituted C₁₋₄ aliphatic. In some embodiments, Z is optionally substituted C₁₋₂ aliphatic. In some embodiments, Z is optionally substituted methyl. In some embodiments, Z is optionally substituted ethyl. In some embodiments, Z is optionally substituted i-propyl. In some embodiments, Z is optionally substituted t-butyl.

In some embodiments, Z is C₁₋₄ aliphatic optionally substituted with halogen. In some such embodiments, Z is —CF₃. In some embodiments, Z is —CH₂CF₃.

In some embodiments, Z is C₁₋₄ aliphatic optionally substituted with one or more groups selected from oxo, —(CH₂)₀₋₄R^{∘}, and —(CH₂)₀₋₄OR^{∘}. In some such embodiments, R^{∘} is selected from hydrogen, C₁₋₆ aliphatic, or a 5-6-membered saturated, partially unsaturated, or aryl ring having heteroatoms independently selected from nitrogen, oxygen, or sulfur optionally substituted with halogen or —(CH₂)₀₋₂OR.

In some embodiments, Z is C₁₋₄ aliphatic optionally substituted with oxo.

In some embodiments, Z is C₁₋₄ aliphatic optionally substituted with —(CH₂)₀₋₄R^{∘}. In some such embodiments, R^{∘} is a 5-6-membered saturated, partially unsaturated, or aryl ring having 0-4 heteroatoms independently selected from nitrogen, oxygen, or sulfur. In some embodiments, Z is C₁₋₄ aliphatic optionally substituted with —(CH₂)₀₋₄R^{∘}, wherein R^{∘} is phenyl optionally substituted with halogen or —(CH₂)₀₋₂OR^{●}.

In some embodiments, Z is C₁₋₄ aliphatic optionally substituted with —(CH₂)₀₋₄OR^{∘}. In some such embodiments, R^{∘} is hydrogen or C₁₋₆ aliphatic. In some embodiments, Z is C₁₋₄ aliphatic optionally substituted with —(CH₂)₀₋₂OR^{∘}. In some such embodiments, R^{∘} is hydrogen or C₁₋₆ aliphatic. In some embodiments, Z is C₁₋₄ aliphatic optionally substituted with —OR^{∘}, —CH₂OR^{∘} or —CH₂CH₂OR^{∘}.

In some embodiments, Z is —Cy.

In some embodiments, Z is —(C₁₋₃ aliphatic)-Cy. In some embodiments, Z is —(C₁₋₂ aliphatic)-Cy. In some embodiments, Z is —(C₃ aliphatic)-Cy. In some embodiments, Z is —CH₂-Cy, —CH₂CH₂—Cy, —CH(CH₃)—Cy, —C(CH₃)₂—Cy, or

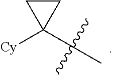

In some embodiments, Z is selected from halogen, —CN, and —NO₂. In some embodiments, Z is selected from halogen.

In some embodiments, Z is selected from —OR, —SR, and —N(R)₂. In some embodiments, Z is —N(R)₂. In some embodiments, Z is —OR.

In some embodiments, Z is selected from —C(O)R, —C(O)₂R, and —C(O)N(R)₂. In some embodiments, Z is —C(O)R. In some embodiments, Z is —C(O)₂R. In some embodiments, Z is —C(O)N(R)₂.

In some embodiments, Z is selected from —OC(O)R and —N(R)C(O)R. In some embodiments, Z is —N(R)C(O)R.

As defined above formula I″, Z is selected from halogen, —CN, —NO₂, —OR, —SR, —N(R)₂, —C(O)R, —C(O)₂R, —OC(O)R, —C(O)N(R)₂, —N(R)C(O)R, —N(R)C(O)₂R, —N(R)C(O)N(R)₂, —S(O)₂R, —Cy, —(C₁₋₃ aliphatic)-Cy or optionally substituted C₁₋₄ aliphatic.

In some embodiments of formula I″, Z is —C(O)R, —C(O)₂R, —C(O)N(R)₂, or —S(O)₂R. In some embodiments of formula I″, Z is —OC(O)R, —N(R)C(O)R, —N(R)C(O)₂R, or —N(R)C(O)N(R)₂.

In some embodiments of formula I″, Z is —N(R)C(O)₂R.

In some embodiments of formula I″, Z is —N(R)C(O)N(R)₂.

In some embodiments of formula Z is —S(O)₂R.

In some embodiments, Z is selected from the group consisting of fluoro, chloro, methyl, ethyl, isopropyl, tert-butyl, phenyl, —OH, —OCH₃, —CH₂OH, or the groups in Table 1:

TABLE 1

| | |
|---|---|
| 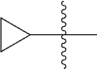 [SMILES: *C1CC1] | 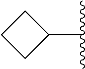 [SMILES: *C1CCC1] |
| 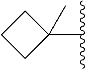 [SMILES: *C1(C)CCC1] | 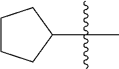 [SMILES: *C1CCCC1] |
| 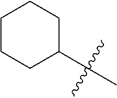 [SMILES: *C1CCCCC1] | 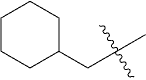 [SMILES: *CC1CCCCC1] |
| 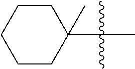 [SMILES: *C1(C)CCCCC1] | 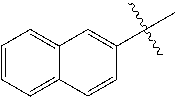 [SMILES: *c1ccc2ccccc2c1] |
| 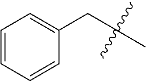 [SMILES: *Cc1ccccc1] | 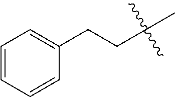 [SMILES: *CCc1ccccc1] |
| 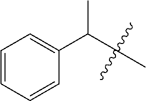 [SMILES: *C(C)c1ccccc1] | 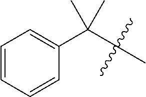 [SMILES: *C(C)(C)c1ccccc1] |
| 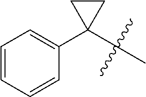 [SMILES: *C1(c2ccccc2)CC1] | 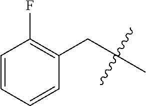 [SMILES: *Cc1ccccc1F] |
| 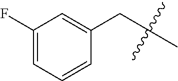 [SMILES: *Cc1cccc(F)c1] | 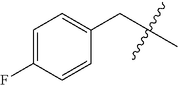 [SMILES: *Cc1ccc(F)cc1] |
| 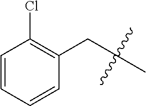 [SMILES: *Cc1ccccc1Cl] | 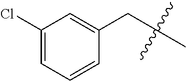 [SMILES: *Cc1cccc(Cl)c1] |
| 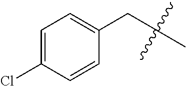 [SMILES: *Cc1ccc(Cl)cc1] | 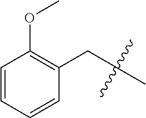 [SMILES: *Cc1ccccc1OC] |

| | |
|---|---|
| 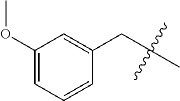 [SMILES: *Cc1cccc(OC)c1] | 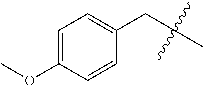 [SMILES: *Cc1ccc(OC)cc1] |
| 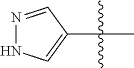 [SMILES: *c1cn[nH]c1] | 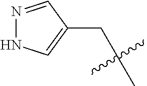 [SMILES: *Cc1cn[nH]c1] |
| 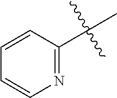 [SMILES: *c1ccccn1] | 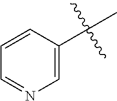 [SMILES: *c1cccnc1] |
| 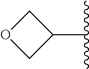 [SMILES: *C1COC1] | 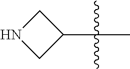 [SMILES: *C1CNC1] |
| 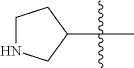 [SMILES: *C1CCNC1] | 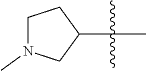 [SMILES: *C1CCN(C)C1] |
| 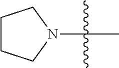 [SMILES: *N1CCCC1] | 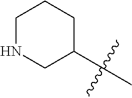 [SMILES: *C1CCCNC1] |
| 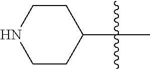 [SMILES: *C1CCNCC1] | 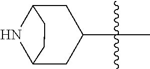 [SMILES: *C1CC2CCC(C1)N2] |

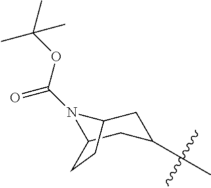 [SMILES: *C1CC2CCC(C1)N2C(=O)OC(C)(C)C]

| | |
|---|---|
| 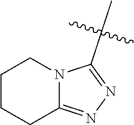 [SMILES: *c1nnc2n1CCCC2] | 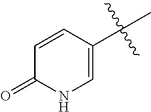 [SMILES: *c1ccc(=O)[nH]c1] |
| 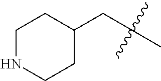 [SMILES: *CC1CCNCC1] | 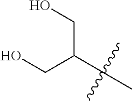 [SMILES: *C(CO)CO] |
| 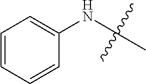 [SMILES: *Nc1ccccc1] | 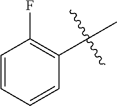 [SMILES: *c1ccccc1F] |

| | |
|---|---|
| 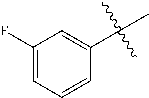 [SMILES: *c1cccc(F)c1] | 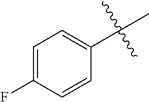 [SMILES: *c1ccc(F)cc1] |
| 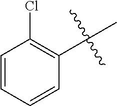 [SMILES: *c1ccccc1Cl] | 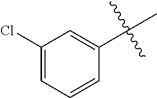 [SMILES: *c1cccc(Cl)c1] |
| 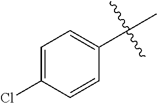 [SMILES: *c1ccc(Cl)cc1] | 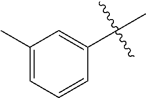 [SMILES: *c1cccc(C)c1] |
| 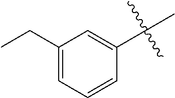 [SMILES: *c1cccc(CC)c1] | 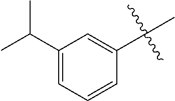 [SMILES: *c1cccc(C(C)C)c1] |
| 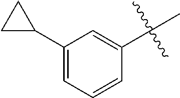 [SMILES: *c1cccc(C2CC2)c1] | 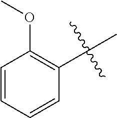 [SMILES: *c1ccccc1OC] |
| 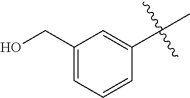 [SMILES: *c1cccc(CO)c1] | 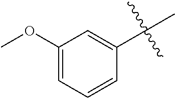 [SMILES: *c1cccc(OC)c1] |
| 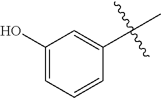 [SMILES: *c1cccc(O)c1] | 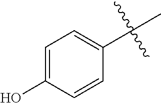 [SMILES: *c1ccc(O)cc1] |
| 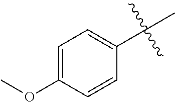 [SMILES: *c1ccc(OC)cc1] | 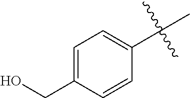 [SMILES: *c1ccc(CO)cc1] |

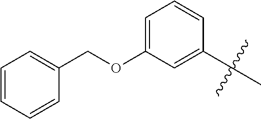 [SMILES: *c1cccc(OCc2ccccc2)c1]

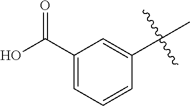 [SMILES: *c1cccc(C(=O)O)c1]

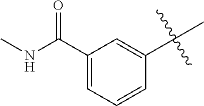 [SMILES: *c1cccc(C(=O)NC)c1]

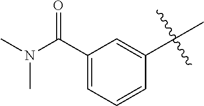 [SMILES: *c1cccc(C(=O)N(C)C)c1]

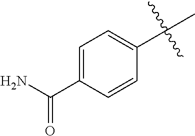 [SMILES: *c1ccc(C(N)=O)cc1]

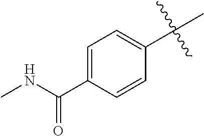 [SMILES: *c1ccc(C(=O)NC)cc1]

| | |
|---|---|
| 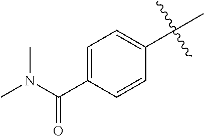 [SMILES: *c1ccc(C(=O)N(C)C)cc1] | 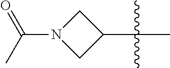 [SMILES: *C1CN(C(C)=O)C1] |

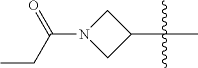 [SMILES: *C1CN(C(=O)CC)C1]

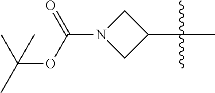 [SMILES: *C1CN(C(=O)OC(C)(C)C)C1]

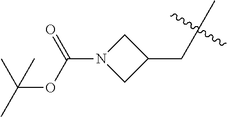 [SMILES: *CC1CN(C(=O)OC(C)(C)C)C1]

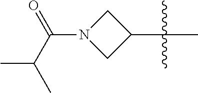 [SMILES: *C1CN(C(=O)C(C)C)C1]

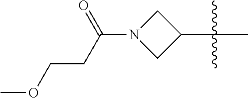 [SMILES: *C1CN(C(=O)CCOC)C1]

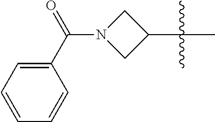 [SMILES: *C1CN(C(=O)c2ccccc2)C1]

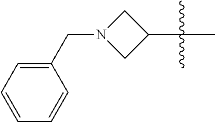 [SMILES: *C1CN(Cc2ccccc2)C1]

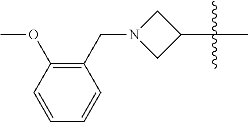 [SMILES: *C1CN(Cc2ccccc2OC)C1]

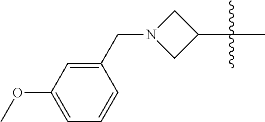 [SMILES: *C1CN(Cc2cccc(OC)c2)C1]

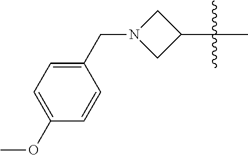 [SMILES: *C1CN(Cc2ccc(OC)cc2)C1]

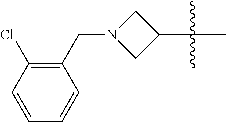 [SMILES: *C1CN(Cc2ccccc2Cl)C1]

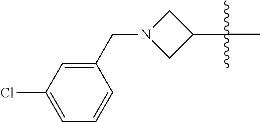 [SMILES: *C1CN(Cc2cccc(Cl)c2)C1]

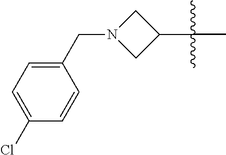 [SMILES: *C1CN(Cc2ccc(Cl)cc2)C1]

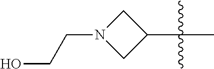 [SMILES: *C1CN(CCO)C1]

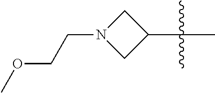 [SMILES: *C1CN(CCOC)C1]

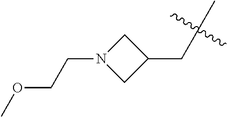 [SMILES: *CC1CN(CCOC)C1]

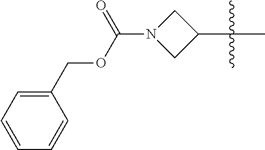 [SMILES: *C1CN(C(=O)OCc2ccccc2)C1]

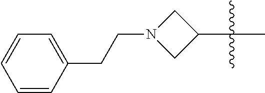 [SMILES: *C1CN(CCc2ccccc2)C1]

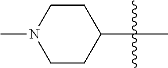 [SMILES: *C1CCN(C)CC1]

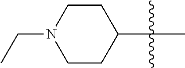 [SMILES: *C1CCN(CC)CC1]

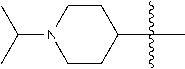 [SMILES: *C1CCN(C(C)C)CC1]

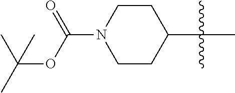 [SMILES: *C1CCN(C(=O)OC(C)(C)C)CC1]

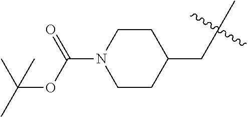 [SMILES: *CC1CCN(C(=O)OC(C)(C)C)CC1]

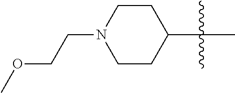 [SMILES: *C1CCN(CCOC)CC1]

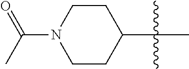 [SMILES: *C1CCN(C(C)=O)CC1]

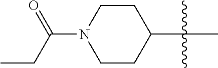 [SMILES: *C1CCN(C(=O)CC)CC1]

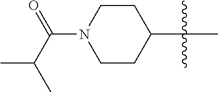 [SMILES: *C1CCN(C(=O)C(C)C)CC1]

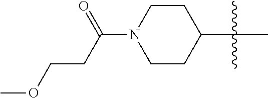 [SMILES: *C1CCN(C(=O)CCOC)CC1]

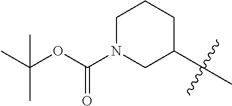 [SMILES: *C1CCCN(C(=O)OC(C)(C)C)C1]

| | |
|---|---|
| 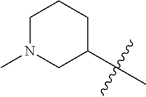 [SMILES: *C1CCCN(C)C1] | 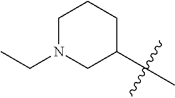 [SMILES: *C1CCCN(CC)C1] |

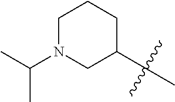 [SMILES: *C1CCCN(C(C)C)C1]

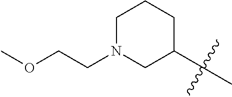 [SMILES: *C1CCCN(CCOC)C1]

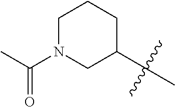 [SMILES: *C1CCCN(C(C)=O)C1]

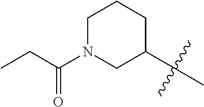 [SMILES: *C1CCCN(C(=O)CC)C1]

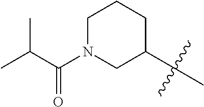 [SMILES: *C1CCCN(C(=O)C(C)C)C1]

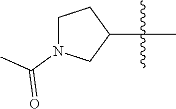 [SMILES: *C1CCN(C(C)=O)C1]

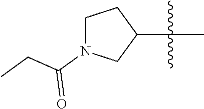 [SMILES: *C1CCN(C(=O)CC)C1]

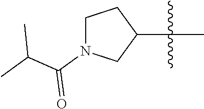 [SMILES: *C1CCN(C(=O)C(C)C)C1]

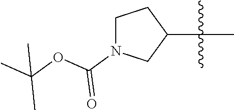 [SMILES: *C1CCN(C(=O)OC(C)(C)C)C1]

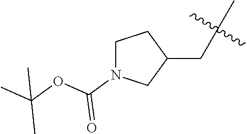 [SMILES: *CC1CCN(C(=O)OC(C)(C)C)C1]

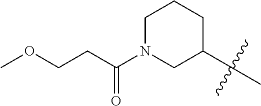 [SMILES: *C1CCCN(C(=O)CCOC)C1]

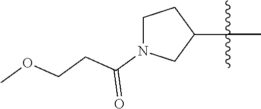 [SMILES: *C1CCN(C(=O)CCOC)C1]

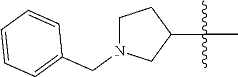 [SMILES: *C1CCN(Cc2ccccc2)C1]

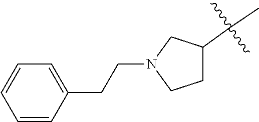 [SMILES: *C1CCN(CCc2ccccc2)C1]

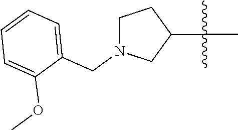 [SMILES: *C1CCN(Cc2ccccc2OC)C1]

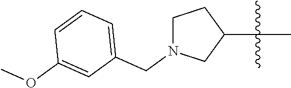 [SMILES: *C1CCN(Cc2cccc(OC)c2)C1]

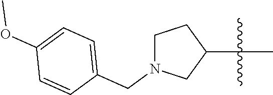 [SMILES: *C1CCN(Cc2ccc(OC)cc2)C1]

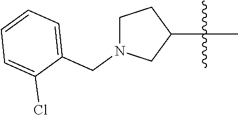 [SMILES: *C1CCN(Cc2ccccc2Cl)C1]

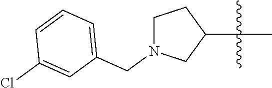 [SMILES: *C1CCN(Cc2cccc(Cl)c2)C1]

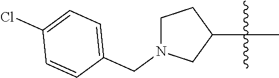 [SMILES: *C1CCN(Cc2ccc(Cl)cc2)C1]

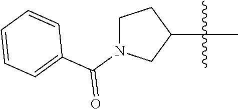 [SMILES: *C1CCN(C(=O)c2ccccc2)C1]

| | |
|---|---|
| 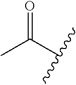 [SMILES: *C(C)=O] | 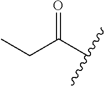 [SMILES: *C(=O)CC] |
| 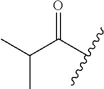 [SMILES: *C(=O)C(C)C] | 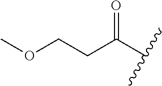 [SMILES: *C(=O)CCOC] |
| 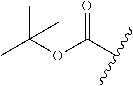 [SMILES: *C(=O)OC(C)(C)C] | 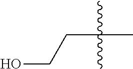 [SMILES: *CCO] |
| 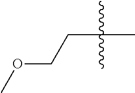 [SMILES: *CCOC] | 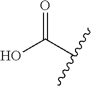 [SMILES: *C(=O)O] |
| 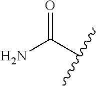 [SMILES: *C(N)=O] | 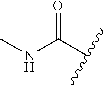 [SMILES: *C(=O)NC] |
| 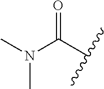 [SMILES: *C(=O)N(C)C] | 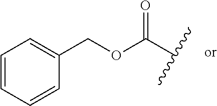 [SMILES: *C(=O)OCc1ccccc1] |

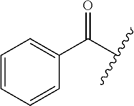 [SMILES: *C(=O)c1ccccc1]

In some embodiments, Z is selected from the group consisting of fluoro, chloro, methyl, ethyl, isopropyl, —OH, —OCH₃, —CH₂CH₂OCH₃, —CH₂OH, —CH₂CH₂OH, —CO₂H, or the groups in Table 2:

TABLE 2

| | | |
|---|---|---|
| 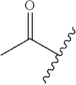 [SMILES: *C(C)=O] | 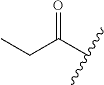 [SMILES: *C(=O)CC] | 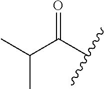 [SMILES: *C(=O)C(C)C] |
| 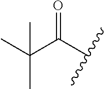 [SMILES: *C(=O)C(C)(C)C] | 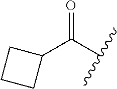 [SMILES: *C(=O)C1CCC1] | 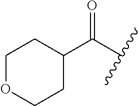 [SMILES: *C(=O)C1CCOCC1] |
| 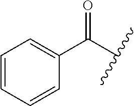 [SMILES: *C(=O)c1ccccc1] | 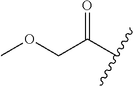 [SMILES: *C(=O)COC] | 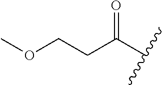 [SMILES: *C(=O)CCOC] |
| 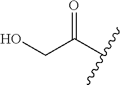 [SMILES: *C(=O)CO] | 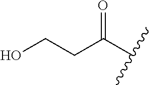 [SMILES: *C(=O)CCO] | 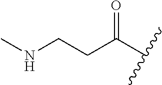 [SMILES: *C(=O)CCNC] |
| 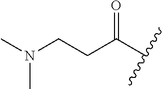 [SMILES: *C(=O)CCN(C)C] | 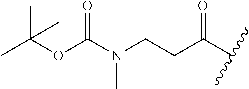 [SMILES: *C(=O)CCN(C)C(=O)OC(C)(C)C] | 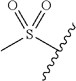 [SMILES: *S(C)(=O)=O] |
| 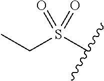 [SMILES: *S(=O)(=O)CC] | 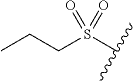 [SMILES: *S(=O)(=O)CCC] | 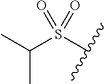 [SMILES: *S(=O)(=O)C(C)C] |
| 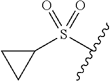 [SMILES: *S(=O)(=O)C1CC1] | 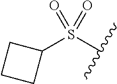 [SMILES: *S(=O)(=O)C1CCC1] | 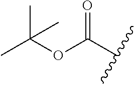 [SMILES: *C(=O)OC(C)(C)C] |
| 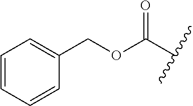 [SMILES: *C(=O)OCc1ccccc1] | 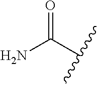 [SMILES: *C(N)=O] | 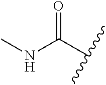 [SMILES: *C(=O)NC] |
| 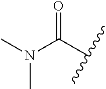 [SMILES: *C(=O)N(C)C] | 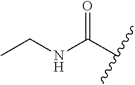 [SMILES: *C(=O)NCC] | 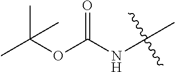 [SMILES: *NC(=O)OC(C)(C)C] |
| 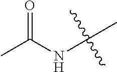 [SMILES: *NC(C)=O] | 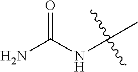 [SMILES: *NC(N)=O] | 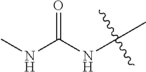 [SMILES: *NC(=O)NC] |
| 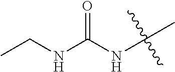 [SMILES: *NC(=O)NCC] | 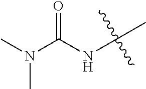 [SMILES: *NC(=O)N(C)C] | 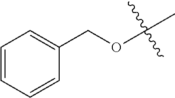 [SMILES: *OCc1ccccc1] |
| 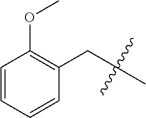 [SMILES: *Cc1ccccc1OC] | 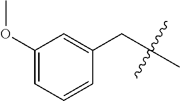 [SMILES: *Cc1cccc(OC)c1] | 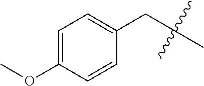 [SMILES: *Cc1ccc(OC)cc1] |
| 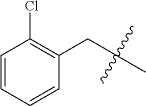 [SMILES: *Cc1ccccc1Cl] | 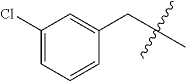 [SMILES: *Cc1cccc(Cl)c1] | 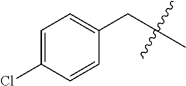 [SMILES: *Cc1ccc(Cl)cc1] |
| 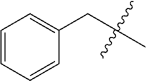 [SMILES: *Cc1ccccc1] | 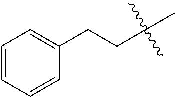 [SMILES: *CCc1ccccc1] | 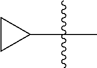 [SMILES: *C1CC1] |

In some embodiments, Z is selected from the group consisting of the groups in Table 3:

TABLE 3

| | | |
|---|---|---|
| 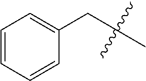 [SMILES: *Cc1ccccc1] | 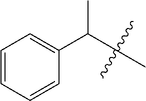 [SMILES: *C(C)c1ccccc1] | 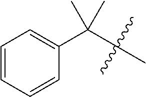 [SMILES: *C(C)(C)c1ccccc1] |
| 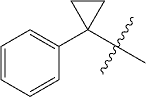 [SMILES: *C1(c2ccccc2)CC1] | 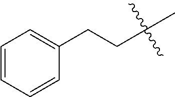 [SMILES: *CCc1ccccc1] | 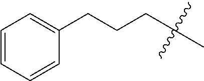 [SMILES: *CCCc1ccccc1] |
| 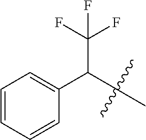 [SMILES: *C(c1ccccc1)C(F)(F)F] | 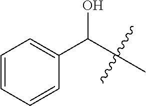 [SMILES: *C(O)c1ccccc1] | 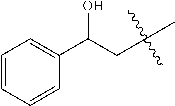 [SMILES: *CC(O)c1ccccc1] |
| 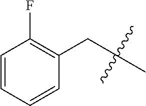 [SMILES: *Cc1ccccc1F] | 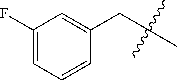 [SMILES: *Cc1cccc(F)c1] | 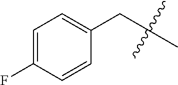 [SMILES: *Cc1ccc(F)cc1] |
| 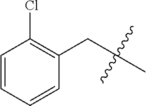 [SMILES: *Cc1ccccc1Cl] | 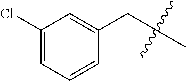 [SMILES: *Cc1cccc(Cl)c1] | 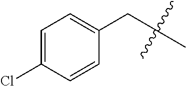 [SMILES: *Cc1ccc(Cl)cc1] |
| 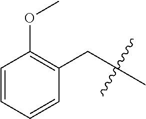 [SMILES: *Cc1ccccc1OC] | 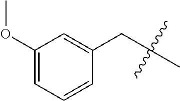 [SMILES: *Cc1cccc(OC)c1] | 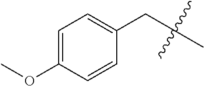 [SMILES: *Cc1ccc(OC)cc1] |
| 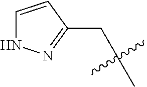 [SMILES: *Cc1cc[nH]n1] | 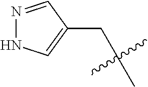 [SMILES: *Cc1cn[nH]c1] | 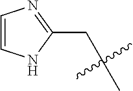 [SMILES: *Cc1ncc[nH]1] |
| 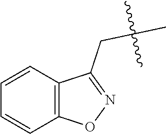 [SMILES: *Cc1noc2ccccc12] | 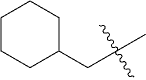 [SMILES: *CC1CCCCC1] | 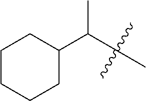 [SMILES: *C(C)C1CCCCC1] |
| 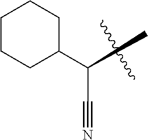 | 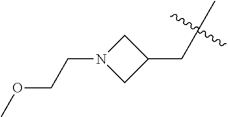 [SMILES: *CC1CN(CCOC)C1] | 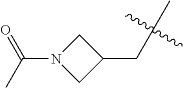 [SMILES: *CC1CN(C(C)=O)C1] |
| 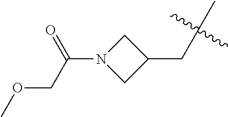 [SMILES: *CC1CN(C(=O)COC)C1] | 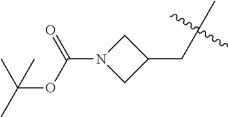 [SMILES: *CC1CN(C(=O)OC(C)(C)C)C1] | 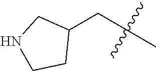 [SMILES: *CC1CCNC1] |
| 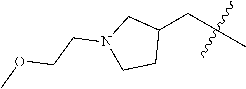 [SMILES: *CC1CCN(CCOC)C1] | 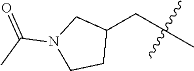 [SMILES: *CC1CCN(C(C)=O)C1] | 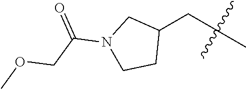 [SMILES: *CC1CCN(C(=O)COC)C1] |
| 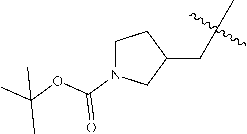 [SMILES: *CC1CCN(C(=O)OC(C)(C)C)C1] | 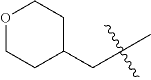 [SMILES: *CC1CCOCC1] | 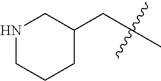 [SMILES: *CC1CCCNC1] |
| 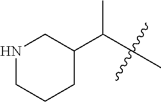 [SMILES: *C(C)C1CCCNC1] | 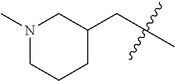 [SMILES: *CC1CCCN(C)C1] | 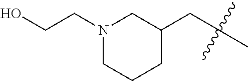 [SMILES: *CC1CCCN(CCO)C1] |
| 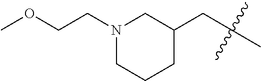 [SMILES: *CC1CCCN(CCOC)C1] | 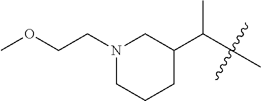 [SMILES: *C(C)C1CCCN(CCOC)C1] | 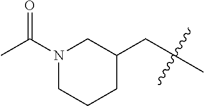 [SMILES: *CC1CCCN(C(C)=O)C1] |
| 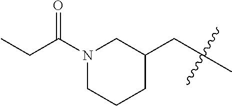 [SMILES: *CC1CCCN(C(=O)CC)C1] | 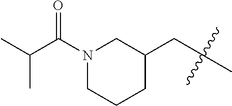 [SMILES: *CC1CCCN(C(=O)C(C)C)C1] | 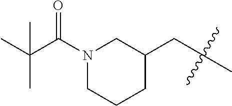 [SMILES: *CC1CCCN(C(=O)C(C)(C)C)C1] |
| 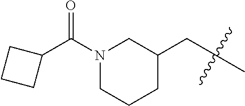 [SMILES: *CC1CCCN(C(=O)C2CCC2)C1] | 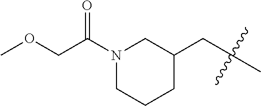 [SMILES: *CC1CCCN(C(=O)COC)C1] | 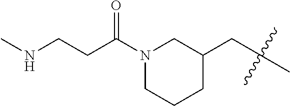 [SMILES: *CC1CCCN(C(=O)CCNC)C1] |
| 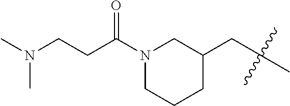 [SMILES: *CC1CCCN(C(=O)CCN(C)C)C1] | 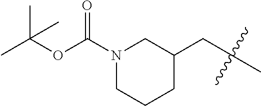 [SMILES: *CC1CCCN(C(=O)OC(C)(C)C)C1] | 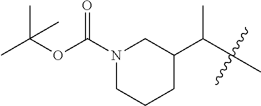 [SMILES: *C(C)C1CCCN(C(=O)OC(C)(C)C)C1] |
| 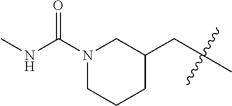 [SMILES: *CC1CCCN(C(=O)NC)C1] | 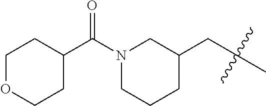 [SMILES: *CC1CCCN(C(=O)C2CCOCC2)C1] | 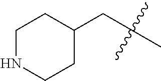 [SMILES: *CC1CCNCC1] |
| 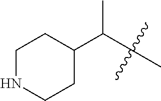 [SMILES: *C(C)C1CCNCC1] | 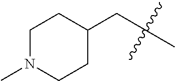 [SMILES: *CC1CCN(C)CC1] | 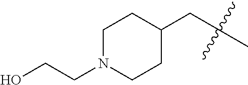 [SMILES: *CC1CCN(CCO)CC1] |
| 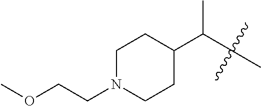 [SMILES: *C(C)C1CCN(CCOC)CC1] | 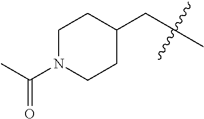 [SMILES: *CC1CCN(C(C)=O)CC1] | 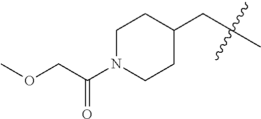 [SMILES: *CC1CCN(C(=O)COC)CC1] |
| 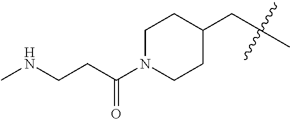 [SMILES: *CC1CCN(C(=O)CCNC)CC1] | 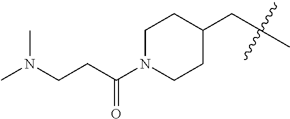 [SMILES: *CC1CCN(C(=O)CCN(C)C)CC1] | 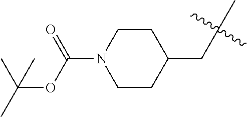 [SMILES: *CC1CCN(C(=O)OC(C)(C)C)CC1] |
| 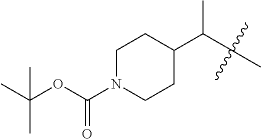 [SMILES: *C(C)C1CCN(C(=O)OC(C)(C)C)CC1] | 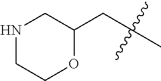 [SMILES: *CC1CNCCO1] | 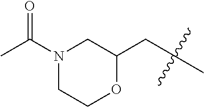 [SMILES: *CC1CN(C(C)=O)CCO1] |
| 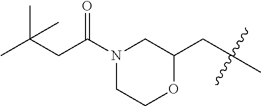 [SMILES: *CC1CN(C(=O)CC(C)(C)C)CCO1] | | |

In some embodiments, Z is selected from the group consisting of methyl isopropyl, tert-butyl, phenyl, —CF₃, —CH₂CF₃, or the groups in Table 4:

TABLE 4

| | | |
|---|---|---|
| 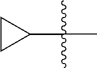 [SMILES: *C1CC1] | 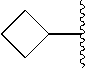 [SMILES: *C1CCC1] | 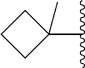 [SMILES: *C1(C)CCC1] |
| 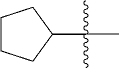 [SMILES: *C1CCCC1] | 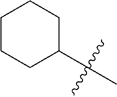 [SMILES: *C1CCCCC1] | 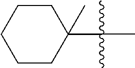 [SMILES: *C1(C)CCCCC1] |
| 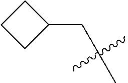 [SMILES: *CC1CCC1] | 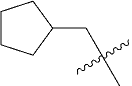 [SMILES: *CC1CCCC1] | 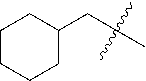 [SMILES: *CC1CCCCC1] |
| 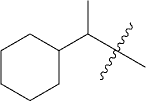 [SMILES: *C(C)C1CCCCC1] | 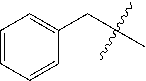 [SMILES: *Cc1ccccc1] | 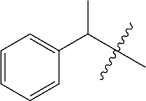 [SMILES: *C(C)c1ccccc1] |
| 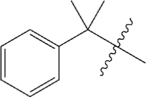 [SMILES: *C(C)(C)c1ccccc1] | 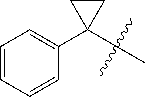 [SMILES: *C1(c2ccccc2)CC1] | 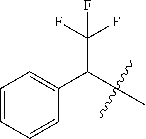 [SMILES: *C(c1ccccc1)C(F)(F)F] |
| 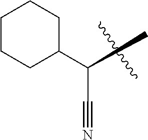 | 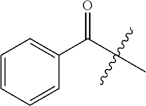 [SMILES: *C(=O)c1ccccc1] | 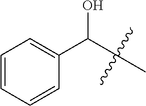 [SMILES: *C(O)c1ccccc1] |
| 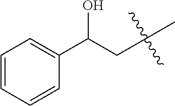 [SMILES: *CC(O)c1ccccc1] | 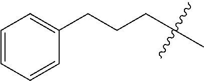 [SMILES: *CCCc1ccccc1] | 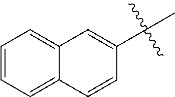 [SMILES: *c1ccc2ccccc2c1] |
| 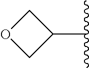 [SMILES: *C1COC1] | 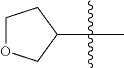 [SMILES: *C1CCOC1] | 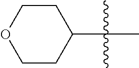 [SMILES: *C1CCOCC1] |
| 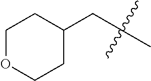 [SMILES: *CC1CCOCC1] | 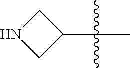 [SMILES: *C1CNC1] | 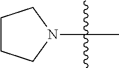 [SMILES: *N1CCCC1] |
| 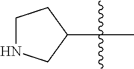 [SMILES: *C1CCNC1] | 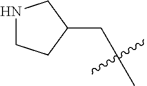 [SMILES: *CC1CCNC1] | 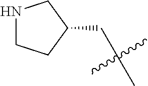 [SMILES: *C[C@@H]1CCNC1] |
| 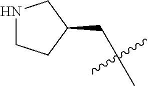 [SMILES: *C[C@H]1CCNC1] | 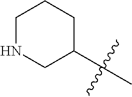 [SMILES: *C1CCCNC1] | 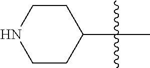 [SMILES: *C1CCNCC1] |
| 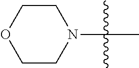 [SMILES: *N1CCOCC1] | 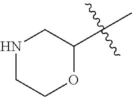 [SMILES: *C1CNCCO1] | 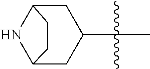 [SMILES: *C1CC2CCC(C1)N2] |
| 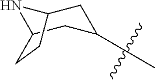 [SMILES: *C1CC2CCC(C1)N2] | 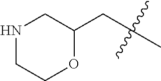 [SMILES: *CC1CNCCO1] | 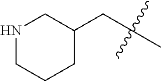 [SMILES: *CC1CCCNC1] |
| 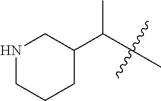 [SMILES: *C(C)C1CCCNC1] | 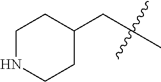 [SMILES: *CC1CCNCC1] | 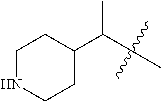 [SMILES: *C(C)C1CCNCC1] |
| 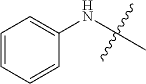 [SMILES: *Nc1ccccc1] | 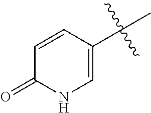 [SMILES: *c1ccc(=O)[nH]c1] | 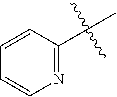 [SMILES: *c1ccccn1] |
| 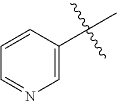 [SMILES: *c1cccnc1] | 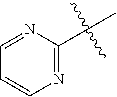 [SMILES: *c1ncccn1] | 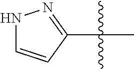 [SMILES: *c1cc[nH]n1] |
| 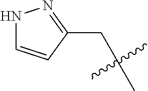 [SMILES: *Cc1cc[nH]n1] | 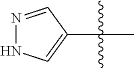 [SMILES: *c1cn[nH]c1] | 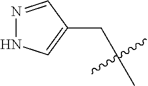 [SMILES: *Cc1cn[nH]c1] |
| 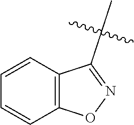 [SMILES: *c1noc2ccccc12] | 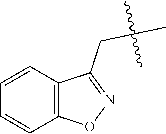 [SMILES: *Cc1noc2ccccc12] | 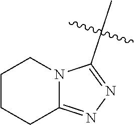 [SMILES: *c1nnc2n1CCCC2] |

| | |
|---|---|
| 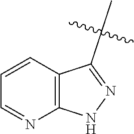 [SMILES: *c1n[nH]c2ncccc12] | 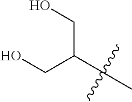 [SMILES: *C(CO)CO] |

In some embodiments, Z is selected from the groups in Table 5:

TABLE 5

| | | |
|---|---|---|
| 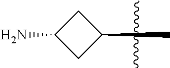 | 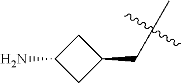 [SMILES: *C[C@H]1C[C@H](N)C1] | 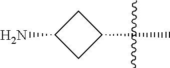 |
| 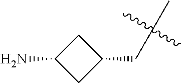 [SMILES: *C[C@H]1C[C@@H](N)C1] | 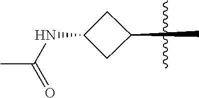 | 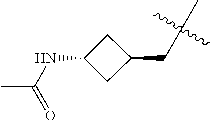 [SMILES: *C[C@H]1C[C@H](NC(C)=O)C1] |
| 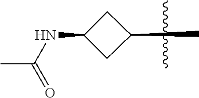 | 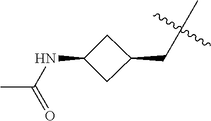 [SMILES: *C[C@H]1C[C@@H](NC(C)=O)C1] | 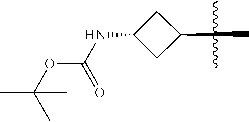 |
| 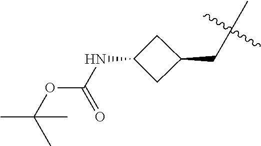 [SMILES: *C[C@H]1C[C@H](NC(=O)OC(C)(C)C)C1] | 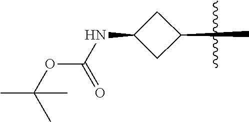 | 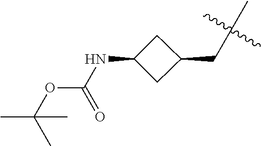 [SMILES: *C[C@H]1C[C@@H](NC(=O)OC(C)(C)C)C1] |
| 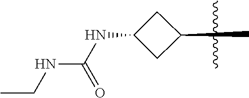 | 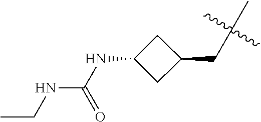 [SMILES: *C[C@H]1C[C@H](NC(=O)NCC)C1] | 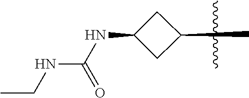 |
| 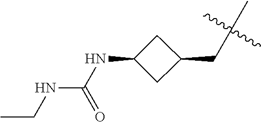 [SMILES: *C[C@H]1C[C@@H](NC(=O)NCC)C1] | 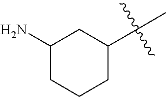 [SMILES: *C1CCCC(N)C1] | 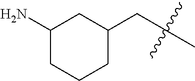 [SMILES: *CC1CCCC(N)C1] |
| 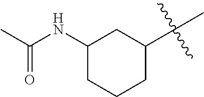 [SMILES: *C1CCCC(NC(C)=O)C1] | 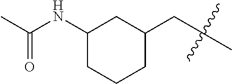 [SMILES: *CC1CCCC(NC(C)=O)C1] | 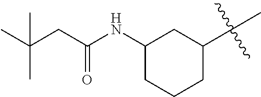 [SMILES: *C1CCCC(NC(=O)CC(C)(C)C)C1] |
| 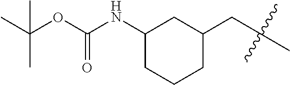 [SMILES: *CC1CCCC(NC(=O)OC(C)(C)C)C1] | 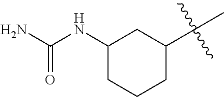 [SMILES: *C1CCCC(NC(N)=O)C1] | 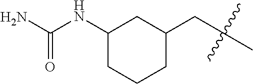 [SMILES: *CC1CCCC(NC(N)=O)C1] |
| 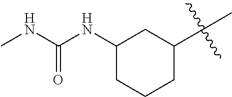 [SMILES: *C1CCCC(NC(=O)NC)C1] | 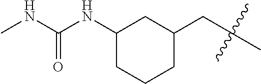 [SMILES: *CC1CCCC(NC(=O)NC)C1] | 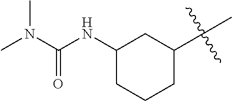 [SMILES: *C1CCCC(NC(=O)N(C)C)C1] |
| 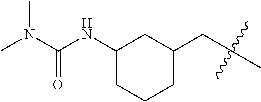 [SMILES: *CC1CCCC(NC(=O)N(C)C)C1] | 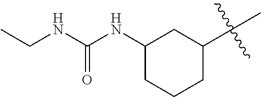 [SMILES: *C1CCCC(NC(=O)NCC)C1] | 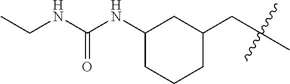 [SMILES: *CC1CCCC(NC(=O)NCC)C1] |
| 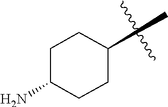 | 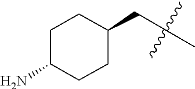 [SMILES: *C[C@H]1CC[C@H](N)CC1] | 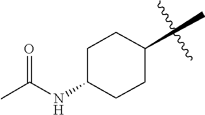 |
| 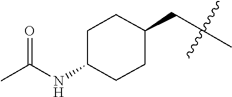 [SMILES: *C[C@H]1CC[C@H](NC(C)=O)CC1] | 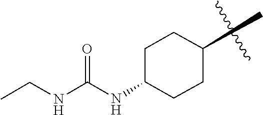 | 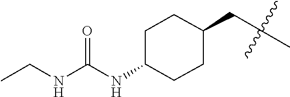 [SMILES: *C[C@H]1CC[C@H](NC(=O)NCC)CC1] |
| 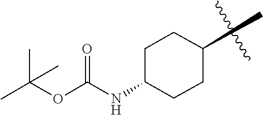 | 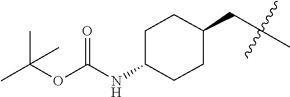 [SMILES: *C[C@H]1CC[C@H](NC(=O)OC(C)(C)C)CC1] | 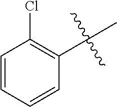 [SMILES: *c1ccccc1Cl] |
| 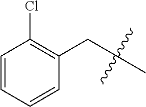 [SMILES: *Cc1ccccc1Cl] | 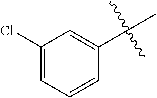 [SMILES: *c1cccc(Cl)c1] | 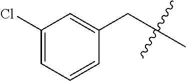 [SMILES: *Cc1cccc(Cl)c1] |
| 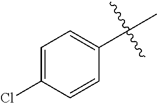 [SMILES: *c1ccc(Cl)cc1] | 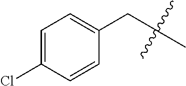 [SMILES: *Cc1ccc(Cl)cc1] | 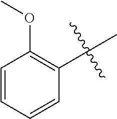 [SMILES: *c1ccccc1OC] |
| 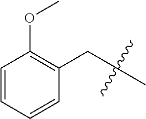 [SMILES: *Cc1ccccc1OC] | 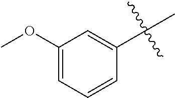 [SMILES: *c1cccc(OC)c1] | 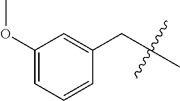 [SMILES: *Cc1cccc(OC)c1] |
| 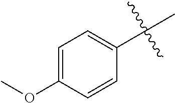 [SMILES: *c1ccc(OC)cc1] | 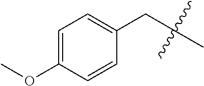 [SMILES: *Cc1ccc(OC)cc1] | 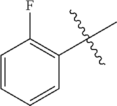 [SMILES: *c1ccccc1F] |
| 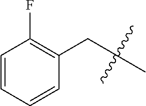 [SMILES: *Cc1ccccc1F] | 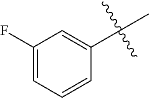 [SMILES: *c1cccc(F)c1] | 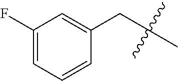 [SMILES: *Cc1cccc(F)c1] |
| 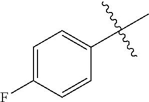 [SMILES: *c1ccc(F)cc1] | 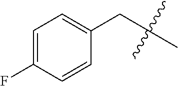 [SMILES: *Cc1ccc(F)cc1] | 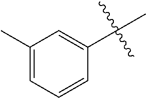 [SMILES: *c1cccc(C)c1] |
| 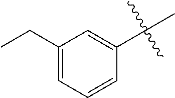 [SMILES: *c1cccc(CC)c1] | 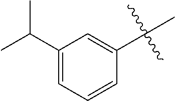 [SMILES: *c1cccc(C(C)C)c1] | 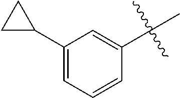 [SMILES: *c1cccc(C2CC2)c1] |
| 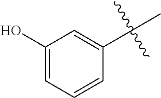 [SMILES: *c1cccc(O)c1] | 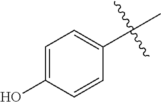 [SMILES: *c1ccc(O)cc1] | 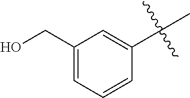 [SMILES: *c1cccc(CO)c1] |
| 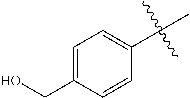 [SMILES: *c1ccc(CO)cc1] | 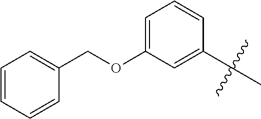 [SMILES: *c1cccc(OCc2ccccc2)c1] | 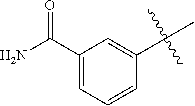 [SMILES: *c1cccc(C(N)=O)c1] |
| 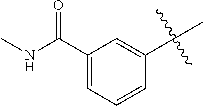 [SMILES: *c1cccc(C(=O)NC)c1] | 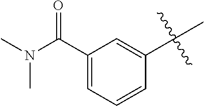 [SMILES: *c1cccc(C(=O)N(C)C)c1] | 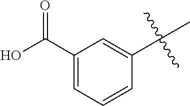 [SMILES: *c1cccc(C(=O)O)c1] |
| 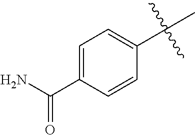 [SMILES: *c1ccc(C(N)=O)cc1] | 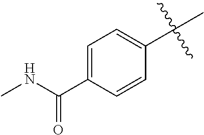 [SMILES: *c1ccc(C(=O)NC)cc1] | 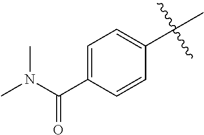 [SMILES: *c1ccc(C(=O)N(C)C)cc1] |
| 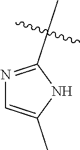 [SMILES: *c1ncc(C)[nH]1] | 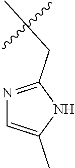 [SMILES: *Cc1ncc(C)[nH]1] | 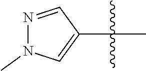 [SMILES: *c1cnn(C)c1] |
| 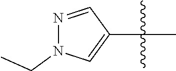 [SMILES: *c1cnn(CC)c1] | 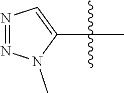 [SMILES: *c1cnnn1C] | 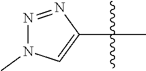 [SMILES: *c1cn(C)nn1] |
| 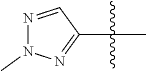 [SMILES: *c1cnn(C)n1] | 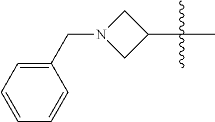 [SMILES: *C1CN(Cc2ccccc2)C1] | 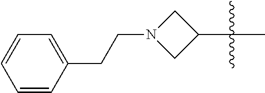 [SMILES: *C1CN(CCc2ccccc2)C1] |
| 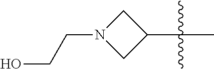 [SMILES: *C1CN(CCO)C1] | 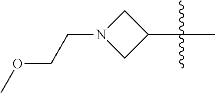 [SMILES: *C1CN(CCOC)C1] | 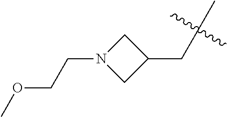 [SMILES: *CC1CN(CCOC)C1] |
| 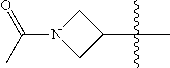 [SMILES: *C1CN(C(C)=O)C1] | 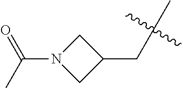 [SMILES: *CC1CN(C(C)=O)C1] | 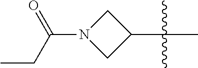 [SMILES: *C1CN(C(=O)CC)C1] |
| 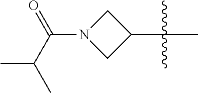 [SMILES: *C1CN(C(=O)C(C)C)C1] | 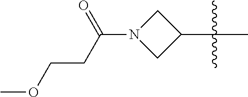 [SMILES: *C1CN(C(=O)CCOC)C1] | 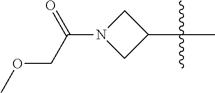 [SMILES: *C1CN(C(=O)COC)C1] |
| 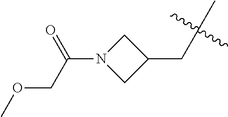 [SMILES: *CC1CN(C(=O)COC)C1] | 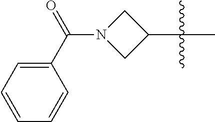 [SMILES: *C1CN(C(=O)c2ccccc2)C1] | 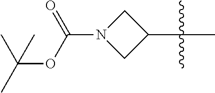 [SMILES: *C1CN(C(=O)OC(C)(C)C)C1] |
| 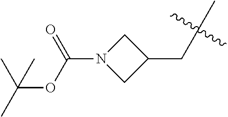 [SMILES: *CC1CN(C(=O)OC(C)(C)C)C1] | 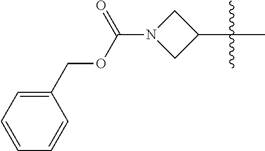 [SMILES: *C1CN(C(=O)OCc2ccccc2)C1] | 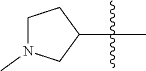 [SMILES: *C1CCN(C)C1] |
| 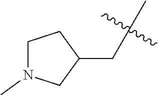 [SMILES: *CC1CCN(C)C1] | 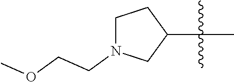 [SMILES: *C1CCN(CCOC)C1] | 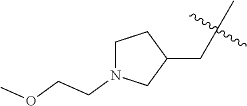 [SMILES: *CC1CCN(CCOC)C1] |
| 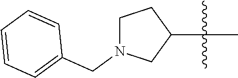 [SMILES: *C1CCN(Cc2ccccc2)C1] | 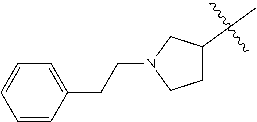 [SMILES: *C1CCN(CCc2ccccc2)C1] | 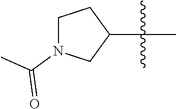 [SMILES: *C1CCN(C(C)=O)C1] |
| 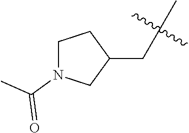 [SMILES: *CC1CCN(C(C)=O)C1] | 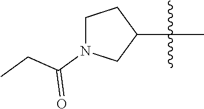 [SMILES: *C1CCN(C(=O)CC)C1] | 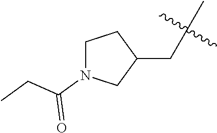 [SMILES: *CC1CCN(C(=O)CC)C1] |
| 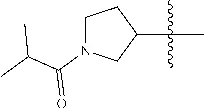 [SMILES: *C1CCN(C(=O)C(C)C)C1] | 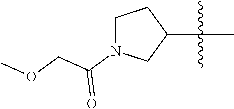 [SMILES: *C1CCN(C(=O)COC)C1] | 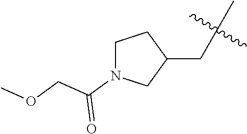 [SMILES: *CC1CCN(C(=O)COC)C1] |
| 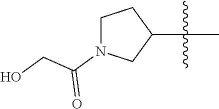 [SMILES: *C1CCN(C(=O)CO)C1] | 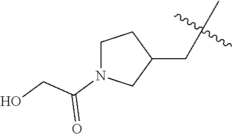 [SMILES: *CC1CCN(C(=O)CO)C1] | 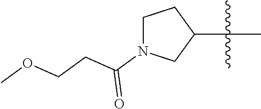 [SMILES: *C1CCN(C(=O)CCOC)C1] |
| 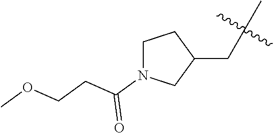 [SMILES: *CC1CCN(C(=O)CCOC)C1] | 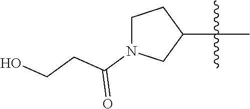 [SMILES: *C1CCN(C(=O)CCO)C1] | 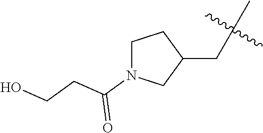 [SMILES: *CC1CCN(C(=O)CCO)C1] |
| 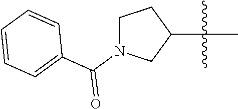 [SMILES: *C1CCN(C(=O)c2ccccc2)C1] | 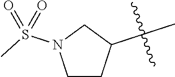 [SMILES: *C1CCN(S(C)(=O)=O)C1] | 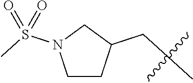 [SMILES: *CC1CCN(S(C)(=O)=O)C1] |
| 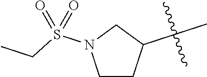 [SMILES: *C1CCN(S(=O)(=O)CC)C1] | 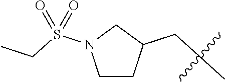 [SMILES: *CC1CCN(S(=O)(=O)CC)C1] | 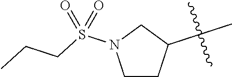 [SMILES: *C1CCN(S(=O)(=O)CCC)C1] |
| 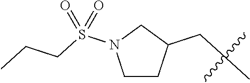 [SMILES: *CC1CCN(S(=O)(=O)CCC)C1] | 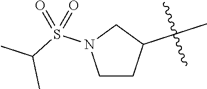 [SMILES: *C1CCN(S(=O)(=O)C(C)C)C1] | 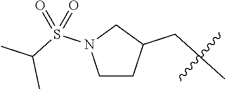 [SMILES: *CC1CCN(S(=O)(=O)C(C)C)C1] |
| 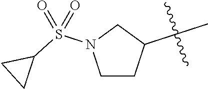 [SMILES: *C1CCN(S(=O)(=O)C2CC2)C1] | 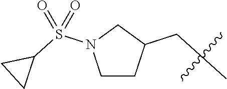 [SMILES: *CC1CCN(S(=O)(=O)C2CC2)C1] | 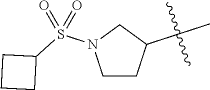 [SMILES: *C1CCN(S(=O)(=O)C2CCC2)C1] |
| 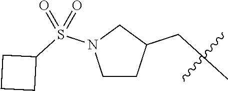 [SMILES: *CC1CCN(S(=O)(=O)C2CCC2)C1] | 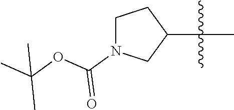 [SMILES: *C1CCN(C(=O)OC(C)(C)C)C1] | 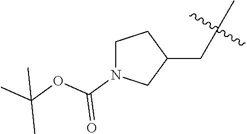 [SMILES: *CC1CCN(C(=O)OC(C)(C)C)C1] |
| 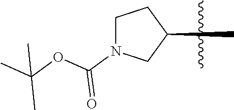 | 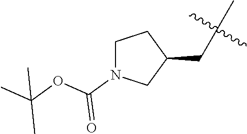 [SMILES: *C[C@@H]1CCN(C(=O)OC(C)(C)C)C1] | 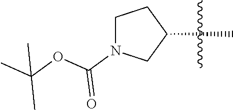 |
| 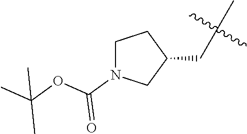 [SMILES: *C[C@H]1CCN(C(=O)OC(C)(C)C)C1] | 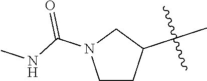 [SMILES: *C1CCN(C(=O)NC)C1] | 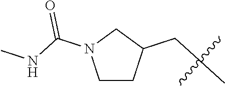 [SMILES: *CC1CCN(C(=O)NC)C1] |
| 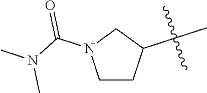 [SMILES: *C1CCN(C(=O)N(C)C)C1] | 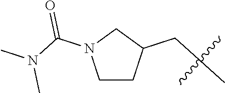 [SMILES: *CC1CCN(C(=O)N(C)C)C1] | 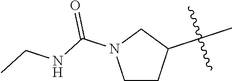 [SMILES: *C1CCN(C(=O)NCC)C1] |
| 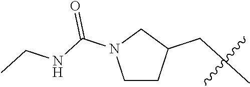 [SMILES: *CC1CCN(C(=O)NCC)C1] | 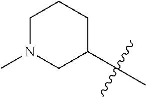 [SMILES: *C1CCCN(C)C1] | 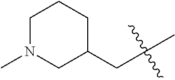 [SMILES: *CC1CCCN(C)C1] |
| 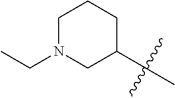 [SMILES: *C1CCCN(CC)C1] | 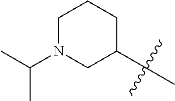 [SMILES: *C1CCCN(C(C)C)C1] | 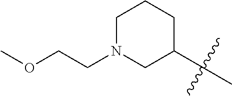 [SMILES: *C1CCCN(CCOC)C1] |
| 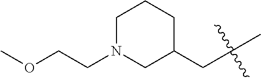 [SMILES: *CC1CCCN(CCOC)C1] | 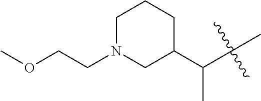 [SMILES: *C(C)C1CCCN(CCOC)C1] | 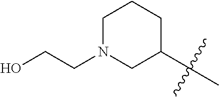 [SMILES: *C1CCCN(CCO)C1] |
| 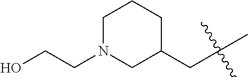 [SMILES: *CC1CCCN(CCO)C1] | 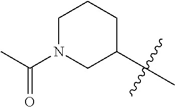 [SMILES: *C1CCCN(C(C)=O)C1] | 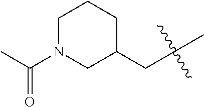 [SMILES: *CC1CCCN(C(C)=O)C1] |
| 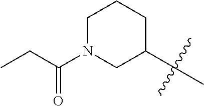 [SMILES: *C1CCCN(C(=O)CC)C1] | 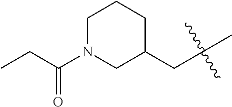 [SMILES: *CC1CCCN(C(=O)CC)C1] | 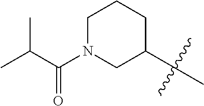 [SMILES: *C1CCCN(C(=O)C(C)C)C1] |
| 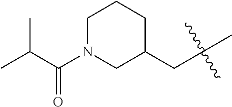 [SMILES: *CC1CCCN(C(=O)C(C)C)C1] | 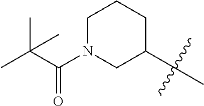 [SMILES: *C1CCCN(C(=O)C(C)(C)C)C1] | 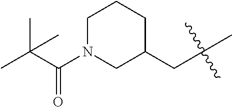 [SMILES: *CC1CCCN(C(=O)C(C)(C)C)C1] |
| 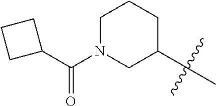 [SMILES: *C1CCCN(C(=O)C2CCC2)C1] | 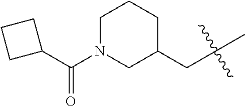 [SMILES: *CC1CCCN(C(=O)C2CCC2)C1] | 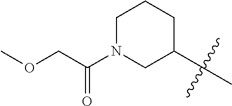 [SMILES: *C1CCCN(C(=O)COC)C1] |
| 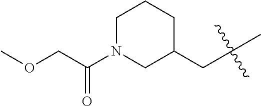 [SMILES: *CC1CCCN(C(=O)COC)C1] | 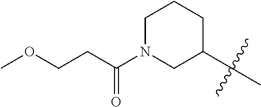 [SMILES: *C1CCCN(C(=O)CCOC)C1] | 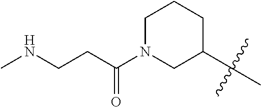 [SMILES: *C1CCCN(C(=O)CCNC)C1] |
| 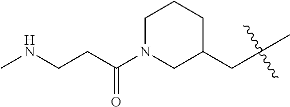 [SMILES: *CC1CCCN(C(=O)CCNC)C1] | 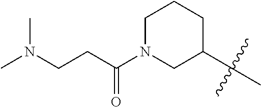 [SMILES: *C1CCCN(C(=O)CCN(C)C)C1] | 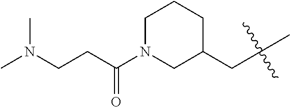 [SMILES: *CC1CCCN(C(=O)CCN(C)C)C1] |
| 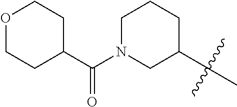 [SMILES: *C1CCCN(C(=O)C2CCOCC2)C1] | 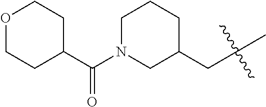 [SMILES: *CC1CCCN(C(=O)C2CCOCC2)C1] | 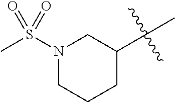 [SMILES: *C1CCCN(S(C)(=O)=O)C1] |
| 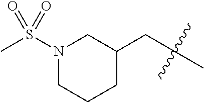 [SMILES: *CC1CCCN(S(C)(=O)=O)C1] | 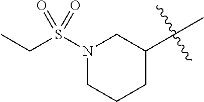 [SMILES: *C1CCCN(S(=O)(=O)CC)C1] | 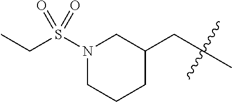 [SMILES: *CC1CCCN(S(=O)(=O)CC)C1] |
| 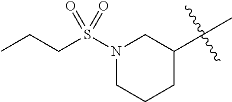 [SMILES: *C1CCCN(S(=O)(=O)CCC)C1] | 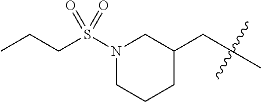 [SMILES: *CC1CCCN(S(=O)(=O)CCC)C1] | 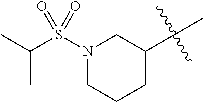 [SMILES: *C1CCCN(S(=O)(=O)C(C)C)C1] |
| 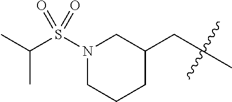 [SMILES: *CC1CCCN(S(=O)(=O)C(C)C)C1] | 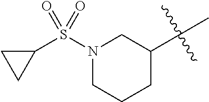 [SMILES: *C1CCCN(S(=O)(=O)C2CC2)C1] | 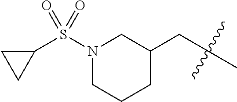 [SMILES: *CC1CCCN(S(=O)(=O)C2CC2)C1] |
| 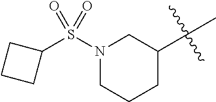 [SMILES: *C1CCCN(S(=O)(=O)C2CCC2)C1] | 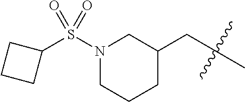 [SMILES: *CC1CCCN(S(=O)(=O)C2CCC2)C1] | 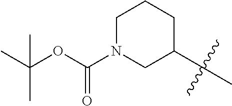 [SMILES: *C1CCCN(C(=O)OC(C)(C)C)C1] |
| 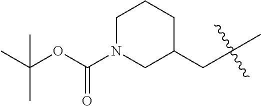 [SMILES: *CC1CCCN(C(=O)OC(C)(C)C)C1] | 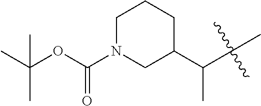 [SMILES: *C(C)C1CCCN(C(=O)OC(C)(C)C)C1] | 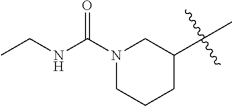 [SMILES: *C1CCCN(C(=O)NCC)C1] |
| 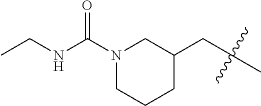 [SMILES: *CC1CCCN(C(=O)NCC)C1] | 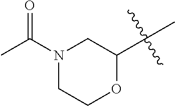 [SMILES: *C1CN(C(C)=O)CCO1] | 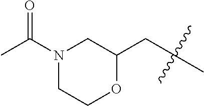 [SMILES: *CC1CN(C(C)=O)CCO1] |
| 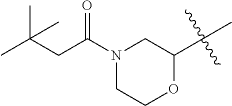 [SMILES: *C1CN(C(=O)CC(C)(C)C)CCO1] | 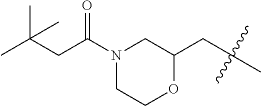 [SMILES: *CC1CN(C(=O)CC(C)(C)C)CCO1] | 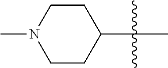 [SMILES: *C1CCN(C)CC1] |
| 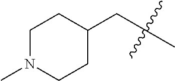 [SMILES: *CC1CCN(C)CC1] | 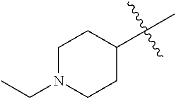 [SMILES: *C1CCN(CC)CC1] | 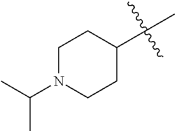 [SMILES: *C1CCN(C(C)C)CC1] |
| 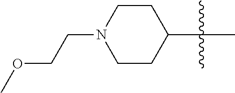 [SMILES: *C1CCN(CCOC)CC1] | 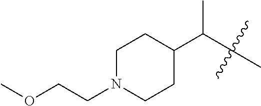 [SMILES: *C(C)C1CCN(CCOC)CC1] | 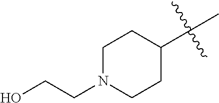 [SMILES: *C1CCN(CCO)CC1] |
| 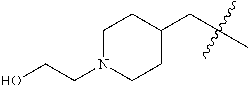 [SMILES: *CC1CCN(CCO)CC1] | 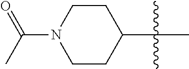 [SMILES: *C1CCN(C(C)=O)CC1] | 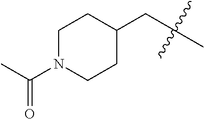 [SMILES: *CC1CCN(C(C)=O)CC1] |
| 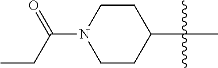 [SMILES: *C1CCN(C(=O)CC)CC1] | 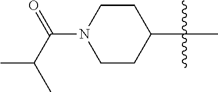 [SMILES: *C1CCN(C(=O)C(C)C)CC1] | 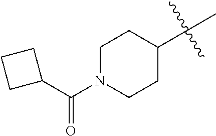 [SMILES: *C1CCN(C(=O)C2CCC2)CC1] |
| 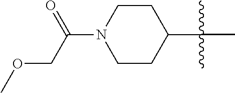 [SMILES: *C1CCN(C(=O)COC)CC1] | 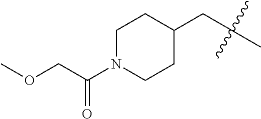 [SMILES: *CC1CCN(C(=O)COC)CC1] | 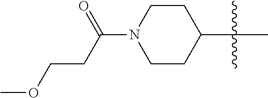 [SMILES: *C1CCN(C(=O)CCOC)CC1] |
| 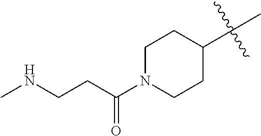 [SMILES: *C1CCN(C(=O)CCNC)CC1] | 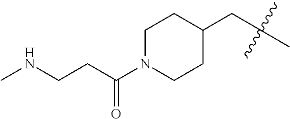 [SMILES: *CC1CCN(C(=O)CCNC)CC1] | 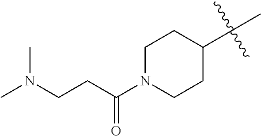 [SMILES: *C1CCN(C(=O)CCN(C)C)CC1] |
| 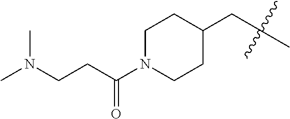 [SMILES: *CC1CCN(C(=O)CCN(C)C)CC1] | 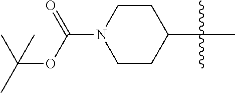 [SMILES: *C1CCN(C(=O)OC(C)(C)C)CC1] | 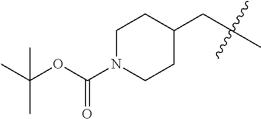 [SMILES: *CC1CCN(C(=O)OC(C)(C)C)CC1] |
| 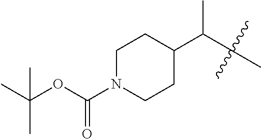 [SMILES: *C(C)C1CCN(C(=O)OC(C)(C)C)CC1] | 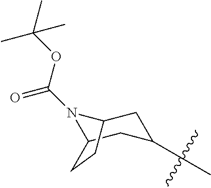 [SMILES: *C1CC2CCC(C1)N2C(=O)OC(C)(C)C] | 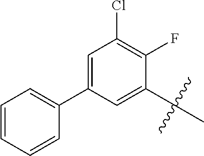 [SMILES: *c1cc(-c2ccccc2)cc(Cl)c1F] |
| 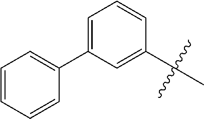 [SMILES: *c1cccc(-c2ccccc2)c1] | 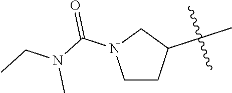 [SMILES: *C1CCN(C(=O)N(C)CC)C1] | 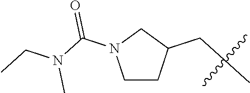 [SMILES: *CC1CCN(C(=O)N(C)CC)C1] |

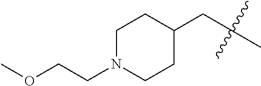 [SMILES: *CC1CCN(CCOC)CC1]

In some embodiments, Z is selected from the group consisting of methyl, ethyl, isopropyl, tert-butyl, phenyl,

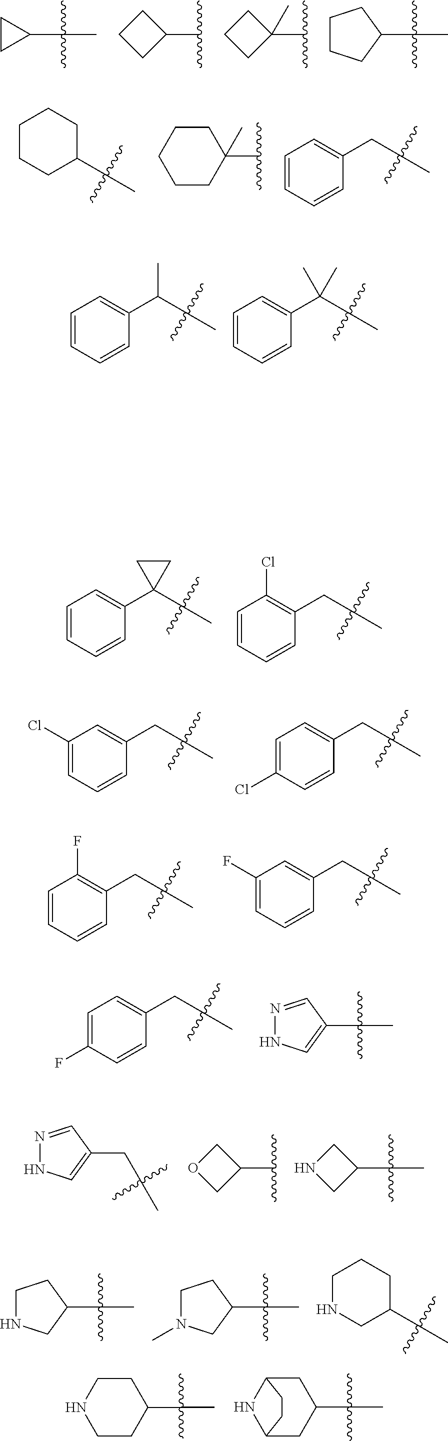

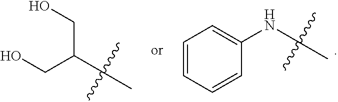 [SMILES: *C(CO)CO ; *Nc1ccccc1]

In some embodiments, Z is selected from the group consisting of

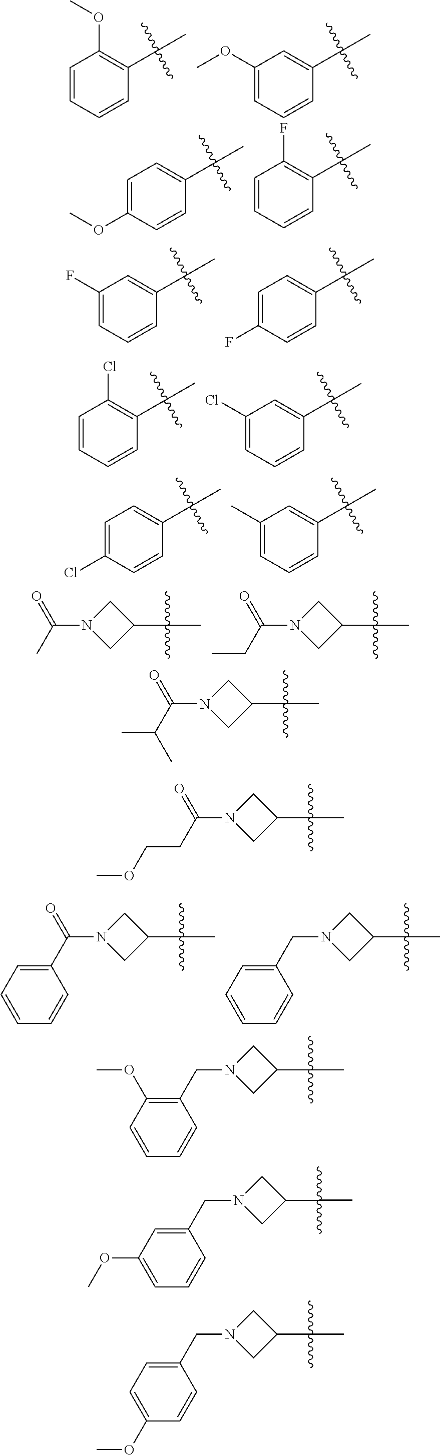

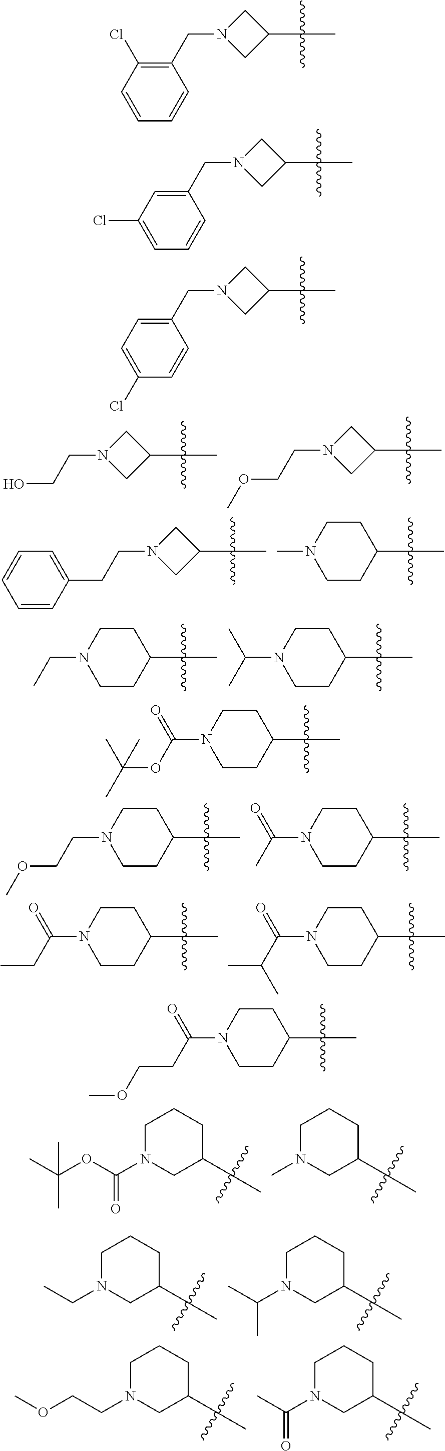

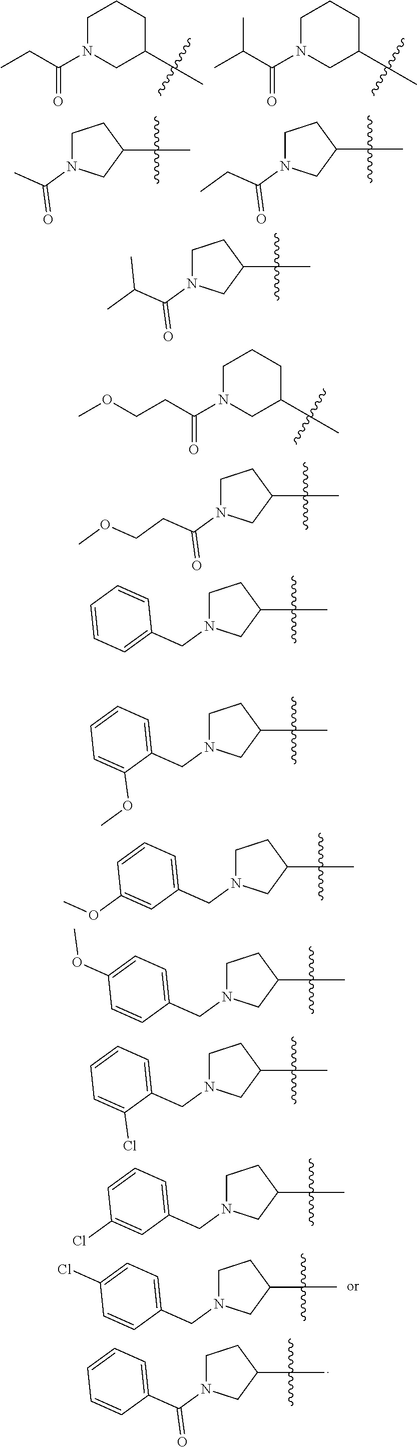

In some embodiments, Z is selected from the group consisting of fluoro, chloro, —OCH₃, methyl, ethyl, isopropyl,

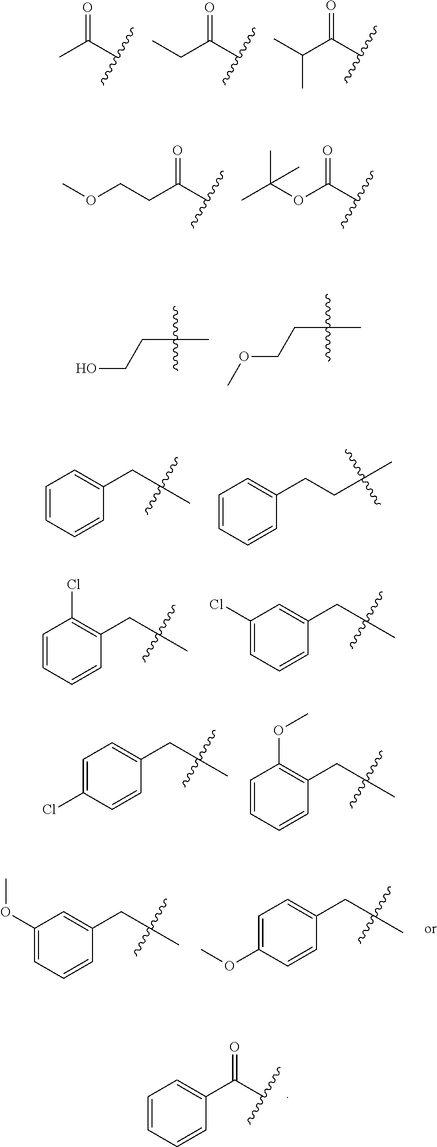

In some embodiments, Z is selected from the group consisting of chloro, fluoro, methyl, ethyl, isopropyl, tert-butyl, phenyl, —OCH₃,

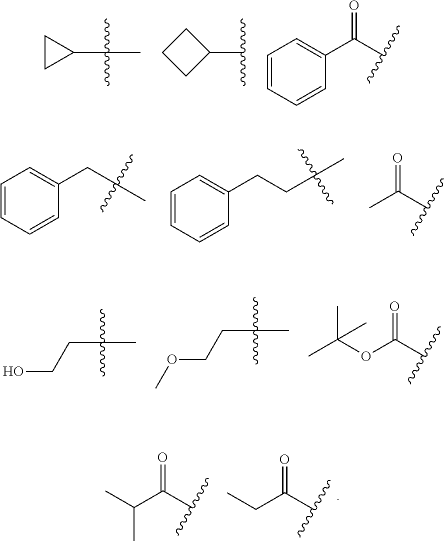

In some embodiments, Z is selected from the group consisting of methyl, ethyl, isopropyl, tert-butyl, —CH(CH₂OH)₂, —CF₃CH₂CH₃.

In some embodiments, Z is selected from the group consisting of methyl, ethyl, isopropyl, tert-butyl, —CH(CH₂OH)₂, —CF₃ or —CH₂CF₃, or the groups in any of Table 1, Table 2, Table 3, Table 4 or Table 5.

In some embodiments, Z is not phenyl. In some embodiments, Z is not

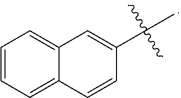 [SMILES: *c1ccc2ccccc2c1]

In some embodiments, Z is not

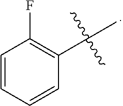 [SMILES: *c1ccccc1F]

In some embodiments, Z is not phenyl,

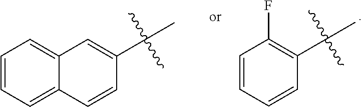 [SMILES: *c1ccc2ccccc2c1 ; *c1ccccc1F]

As defined above, Cy is an optionally substituted group selected from phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 6-8 membered bridged bicyclic heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic amyl ring, and an 8-10 membered bicyclic heteroaryl ring having 1-3 heteroatoms independently selected from nitrogen oxygen and sulfur.

In some embodiments, Cy is an optionally substituted phenyl.

In some embodiments, Cy is phenyl optionally substituted with a group selected from halogen, —NO₂, —CN, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄ CH(OR^{∘)}₂, —(CH₂)₀₋₄ SR^{∘}, —(CH₂)₀₋₄ Ph which may be substituted with R^{∘}, —(CH₂)₀₋₄O(CH₂)₀₋₁Ph which may be substituted with —(CH₂)₀₋₄N(R^{∘})₂, —(CH₂)₀₋₄N(R^{∘})C(O)R^{∘}, —(CH₂)₀₋₄N(R^{∘})C(O)OR^{∘}, —(CH₂)₀₋₄C(O)R^{∘}, —(CH₂)₀₋₄C(O)OR^{∘}, —(CH₂)₀₋₄OC(O)R^{∘}, —(CH₂)₀₋₄C(O)NR^{∘}₂, —(CH₂)₀₋₄ OC(O)NR^{∘}₂, —(CH₂)₀₋₄ S(O)₂R^{∘}, —(CH₂)₀₋₄S(O)₂OR^{∘}, —(CH₂)₀₋₄OS(O)₂R^{∘}, —S(O)₂NR^{∘}₂, —(CH₂)₀₋₄ S(O)R^{∘}, and —N(R^{∘}S(O)₂R^{∘}, wherein each R^{∘} is independently hydrogen, C₁₋₆ aliphatic. —CH₂Ph, —O(CH₂)₀₋₁Ph, —CH₂— (5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having heteroatoms independently selected from nitrogen, oxygen, or sulfur. In some embodiments, Cy is phenyl optionally substituted with halogen or —(CH₂)₀₋₄OR^{∘}.

In some embodiments, Cy is an optionally substituted 3-7 membered saturated or partially unsaturated carbocyclic ring. In some embodiments, Cy is an optionally substituted 3-membered saturated or partially unsaturated carbocyclic ring. In some embodiments, Cy is an optionally substituted 4-membered saturated or partially unsaturated carbocyclic ring. In some embodiments, Cy is an optionally substituted 5-membered saturated or partially unsaturated carbocyclic ring. In some embodiments, Cy is an optionally substituted 6-membered saturated or partially unsaturated carbocyclic ring. In some embodiments, Cy is selected from optionally substituted cyclopropyl, cyclobutyl, cyclopentyl, and cyclohexyl.

In some embodiments, Cy is a 3-7 membered saturated or partially unsaturated carbocyclic ring optionally substituted with —(CH₂)₀₋₄R^{∘}.

In some embodiments, Cy is an optionally substituted 3-7 membered saturated or partially unsaturated heterocyclic ring having 1-2 heteroatoms independently selected from nitrogen, oxygen and sulfur. In some embodiments, Cy is an optionally substituted 4-membered saturated or partially unsaturated heterocyclic ring having 1 heteroatom selected from nitrogen, oxygen and sulfur. In some embodiments, Cy is an optionally substituted group selected from optionally substituted azetidinyl or oxetanyl.

In some embodiments, Cy is azetidinyl optionally substituted with —(CH₂)₀₋₄R^{∘}, —(CH₂)₀₋₄ OR^{∘}, or —(CH₂)₀₋₄C(O)R^{∘}. In some such embodiments, R^{∘} is selected from hydrogen, C₁₋₆ aliphatic or a 5-6-membered saturated, partially unsaturated, or aryl ring having 0-4 heteroatoms independently selected from nitrogen, oxygen, or sulfur optionally substituted with halogen or —(CH₂)₀₋₂OR^{●}.

In some embodiments, Cy is azetidinyl optionally substituted with —(CH₂)₀₋₄R^{∘}, —(CH₂)₀₋₄OR^{∘}, —(CH₂)₀₋₄C(O)R^{∘} or —(CH₂)₀₋₄C(O)OR^{∘}. In some such embodiments, R^{∘} is selected from hydrogen, C₁₋₆ aliphatic or a 5-6-membered saturated, partially unsaturated, or aryl ring having 0-4 heteroatoms independently selected from nitrogen, oxygen, or sulfur optionally substituted with halogen or —(CH₂)₀₋₂ OR^{●}.

In some embodiments, Cy is azetidinyl optionally substituted with —(CH₂)₀₋₄C(O)R^{∘}. In some such embodiments, R^{∘} is C₁₋₆ aliphatic optionally substituted with —(CH₂)₀₋₂OR^{●}.

In some embodiments, Cy is azetidinyl optionally substituted with —(CH₂)₀₋₄C(O)OR^{∘}.

In some embodiments, Cy is azetidinyl optionally substituted on the nitrogen atom with —R^{†}, —C(O)R^{†}, —C(O)OR^{†},

In some such embodiments, R^{†} is optionally substituted with —OH, —OR^{●},

In some embodiments, Cy is an optionally substituted 5-membered saturated or partially unsaturated heterocyclic ring having 1-2 heteroatoms independently selected from nitrogen, oxygen and sulfur. In some embodiments, Cy is an optionally substituted pyrrolidinyl.

In some embodiments, Cy is pyrrolidinyl optionally substituted with —(CH₂)₀₋₄R^{∘} or —(CH₂)₀₋₄C(O)R^{∘}. In some such embodiments, R^{∘} is C₁₋₆ aliphatic or a 5-6-membered saturated, partially unsaturated, or aryl ring having 0-4 heteroatoms independently selected from nitrogen, oxygen, or sulfur optionally substituted with halogen or —(CH₂)₀₋₂OR^{●}.

In some embodiments, Cy is pyrrolidinyl optionally substituted with —(CH₂)₀₋₄R^{∘}, —(CH₂)₀₋₄OR^{∘}, —(CH₂)₀₋₄C(O)R^{∘}, or —(CH₂)₀₋₄C(O)OR^{∘}. In some such embodiments, R^{∘} is hydrogen, C₁₋₆ aliphatic or a 5-6-membered saturated, partially unsaturated, or aryl ring having 0-4 heteroatoms independently selected from nitrogen, oxygen, or sulfur optionally substituted with halogen, —(CH₂)₀₋₂OR^{∘}, —(CH₂)₀₋₂ NHR^{∘}, or —(CH₂)₀₋₂NR^{∘}₂.

In some embodiments, Cy is pyrrolidinyl optionally substituted with —(CH₂)₀₋₄OR^{∘}.

In some embodiments, Cy is pyrrolidinyl optionally substituted with —(CH₂)₀₋₄C(O)R^{∘}. In some such embodiments, R^{∘} is C₁₋₆ aliphatic optionally substituted with —(CH₂)₀₋₂OR^{∘} or —(CH₂)₀₋₂OH.

In some embodiments, Cy is pyrrolidinyl optionally substituted with —(CH₂)₀₋₄C(O)OR^{∘}. In some such embodiments, R^{∘} is C₁₋₆ aliphatic.

In some embodiments, Cy is pyrrolidinyl optionally substituted with —(CH₂)₀₋₄C(O)N(R^{∘}₂, wherein R^{∘} is hydrogen or C₁₋₆ aliphatic.

In some embodiments, Cy is pyrrolidinyl optionally substituted with —(CH₂)₀₋₄SO₂R^{∘}. In some such embodiments, R^{∘} is C₁₋₆ aliphatic.

In some embodiments, Cy is pyrrolidinyl optionally substituted on the nitrogen atom with —R^{†}, —C(O)R^{†}, —C(O)OR^{†}, —C(O)NR^{†}₂, or —S(O)₂R^{†}. In some such embodiments, R^{†} is optionally substituted with —OR^{●}, —NH₂, —NHR^{●}, or —NR^{●}₂.

In some embodiments, Cy is an optionally substituted 6-membered saturated or partially unsaturated heterocyclic ring having 1-2 heteroatoms independently selected from nitrogen, oxygen and sulfur. In some embodiments, Cy is an optionally substituted piperidinyl. In some embodiments, Cy is tetrahydro-2H-pyranyl. In some such embodiments, Cy is tetrahydro-2H-pyran-4-yl.

In some embodiments, Cy is piperidinyl optionally substituted with —(CH₂)₀₋₄R^{∘}, —(CH₂)₀₋₄OR^{∘}, —(CH₂)₀₋₄C(O)R^{∘}, or —(CH₂)₀₋₄C(O)OR^{∘}. In some such embodiments, R^{∘} is C₁₋₆ aliphatic or a 5-6-membered saturated, partially unsaturated, or aryl ring having 0-4 heteroatoms independently selected from nitrogen, oxygen, or sulfur optionally substituted with halogen or —(CH₂)₀₋₂OR^{●}.

In some embodiments, Cy is piperidinyl optionally substituted with —(CH₂)₀₋₄R^{∘}, —(CH₂)₀₋₄OR^{∘}, —(CH₂)₀₋₄C(O)R^{∘}, —(CH₂)₀₋₄C(O)OR^{∘}, or —(CH₂)₀₋₄C(O)N(R^{∘})₂. In some such embodiments, R^{∘} is hydrogen, C₁₋₆ aliphatic or a 5-6-membered saturated, partially unsaturated, or aryl ring having 0-4 heteroatoms independently selected from nitrogen, oxygen, or sulfur optionally substituted with halogen, —(CH₂)₀₋₂OR^{∘}, —(CH₂)₀₋₂NHR^{●}, or —(CH₂)₀₋₂NR^{●}₂.

In some embodiments, is piperidinyl optionally substituted with —(CH₂)₀₋₄OR^{∘}.

In some embodiments, Cy is piperidinyl optionally substituted with —(CH₂)₀₋₄C(O)R^{∘}. In some such embodiments, R^{∘} is C₁₋₆ aliphatic optionally substituted with —(CH₂)₀₋₂OR^{●}.

In some embodiments, is piperidinyl optionally substituted with —(CH₂)₀₋₄C(O)R^{∘}, wherein R^{∘} is C₁₋₆ aliphatic optionally substituted with —(CH₂)₀₋₂ NHR^{●} or —(CH₂)₀₋₂NR^{●}₂.

In some embodiments, Cy is piperidinyl optionally substituted with —(CH₂)₀₋₄C(O)R^{∘}, wherein R^{∘} is a 5-6-membered saturated, partially unsaturated, or aryl ring having 0-4 heteroatoms independently selected from nitrogen, oxygen, or sulfur. In some embodiments, R^{∘} is a 6-membered saturated ring having 0-4 heteroatoms independently selected from nitrogen, oxygen, or sulfur. In some such embodiments, R^{∘} is tetrahydropyranyl.

In some embodiments, Cy is piperidinyl optionally substituted with —(CH₂)₀₋₄C(O)OR^{∘}. In some such embodiments, R^{∘} is C₁₋₆ aliphatic.

In some embodiments, Cy is piperidinyl optionally substituted with —(CH₂)₀₋₄C(O)N(R^{∘})₂.

In some embodiments, Cy is piperidinyl optionally substituted with —(CH₂)₀₋₄ SO₂R^{∘}. In some such embodiments, R^{∘} is C₁₋₆ aliphatic.

In some embodiments, Cy is piperidinyl optionally substituted on the nitrogen atom with —R^{†}, —C(O)R^{†}, —C(O)OR^{†}, —C(O)NR^{†}₂, or —S(O)₂R^{†}. In some such embodiments, R^{†} is optionally substituted with —OH, —OR^{●}, —NH₂, —NHR^{●}, or —NR^{●}₂.

In some embodiments, Cy is morpholinyl optionally substituted with —(CH₂)₀₋₄R^{∘}, —(CH₂)₀₋₄OR^{∘}, —(CH₂)₀₋₄C(O)R^{∘}, or —(CH₂)₀₋₄C(O)OR^{∘}. In some such embodiments, R^{∘} is C₁₋₆ aliphatic or a 5-6-membered saturated, partially unsaturated, or aryl ring having 0-4 heteroatoms independently selected from nitrogen, oxygen, or sulfur optionally substituted with halogen or —(CH₂)₀₋₂OR^{●}.

In some embodiments, Cy is morpholinyl optionally substituted with —(CH₂)₀₋₄C(O)OR^{∘}. In some such embodiments, R^{∘} is C₁₋₆ aliphatic.

In some embodiments, Cy is morpholinyl optionally substituted with —(CH₂)₀₋₄C(O)R^{∘}. In some such embodiments, R^{∘} is C₁₋₆ aliphatic.

In some embodiments, Cy is morpholinyl optionally substituted on the nitrogen atom with —R^{†}, —C(O)R^{†}, or —C(O)OR^{†}.

In some embodiments, Cy is an optionally substituted 6-8 membered bridged bicyclic heterocyclic ring having 1-2 heteroatoms independently selected from nitrogen, oxygen and sulfur. In some embodiments, Cy is an optionally substituted 8-membered bridged bicyclic heterocyclic ring having 1-2 heteroatoms independently selected from nitrogen, oxygen and sulfur. In some embodiments, Cy is an 8-membered bridged bicyclic heterocyclic ring having 1-2 heteroatoms independently selected from nitrogen, oxygen and sulfur optionally substituted on a nitrogen atom with —R^{†}, —C(O)R^{†}, or —C(O)OR^{†}.

In some embodiments, Cy is an optionally substituted 5-6 membered heteroaryl ring having 1-2 heteroatoms independently selected from nitrogen, oxygen and sulfur. In some embodiments, Cy is an optionally substituted 5-membered heteroaryl ring having 1-2 heteroatoms independently selected from nitrogen, oxygen and sulfur. In some embodiments, Cy is an optionally substituted 5-membered heteroaryl ring having 1 heteroatom selected from nitrogen, oxygen and sulfur. In some embodiments, Cy is optionally substituted pyrazolyl.

In some embodiments, Cy is optionally substituted imidazolyl. In some such embodiments, Cy is imidazolyl optionally substituted with —(CH₂)₀₋₄R^{∘}.

In some embodiments, Cy is imidazolyl optionally substituted on a nitrogen atom with —R^{†}. In some such embodiments, —R^{†} is optionally substituted with —OH or —OR^{●}.

In some embodiments, Cy is an optionally substituted 6-membered heteroaryl ring having 1-2 heteroatoms independently selected from nitrogen, oxygen and sulfur. In some embodiments, Cy is an optionally substituted 6-membered heteroaryl ring having 1 heteroatom selected from nitrogen, oxygen and sulfur. In some embodiments, Cy is optionally substituted pyridinyl or pyrimdinyl.

In some embodiments, Cy is an optionally substituted 8-10 membered bicyclic aryl ring. In some embodiments, Cy is optionally substituted naphthyl.

In some embodiments, Cy is an optionally substituted 8-10 membered bicyclic heteroaryl ring having 1-3 heteroatoms independently selected from nitrogen oxygen and sulfur. In some embodiments, Cy is an optionally substituted 8-10 membered bicyclic heteroaryl ring having 1-3 heteroatoms independently selected from nitrogen oxygen and sulfur. In some embodiments, Cy is an optionally substituted 9-membered bicyclic heteroaryl ring having 1-3 heteroatoms independently selected from nitrogen oxygen and sulfur. In some embodiments, Cy is an optionally substituted 1H-pyrazolo[3,4-H]pyridinyl. In some embodiments, Cy is an optionally substituted benzo[d]isoxazolyl. In some embodiments, Cy is an optionally substituted 9-membered bicyclic heteroaryl ring having 1-2 heteroatoms independently selected from nitrogen oxygen and sulfur. In some embodiments, Cy is an optionally substituted indazolyl, benzimidazolyl, indolyl, or isoindolyl. In some embodiments, Cy is an optionally substituted 10-membered bicyclic heteroaryl ring having 1-2 heteroatoms independently selected from nitrogen oxygen and sulfur. In some embodiments, Cy is an optionally substituted quinolyl, isoquinolyl, or quinazolinyl.

In some embodiments, Cy is selected from the group consisting of phenyl,

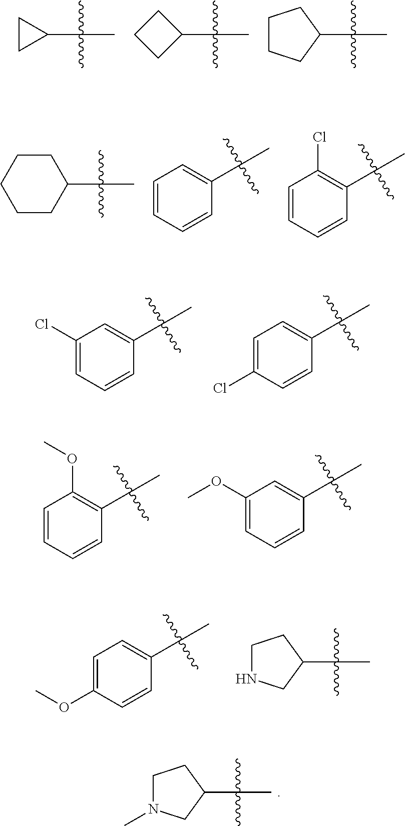

In some embodiments, Cy is selected from the group consisting of:

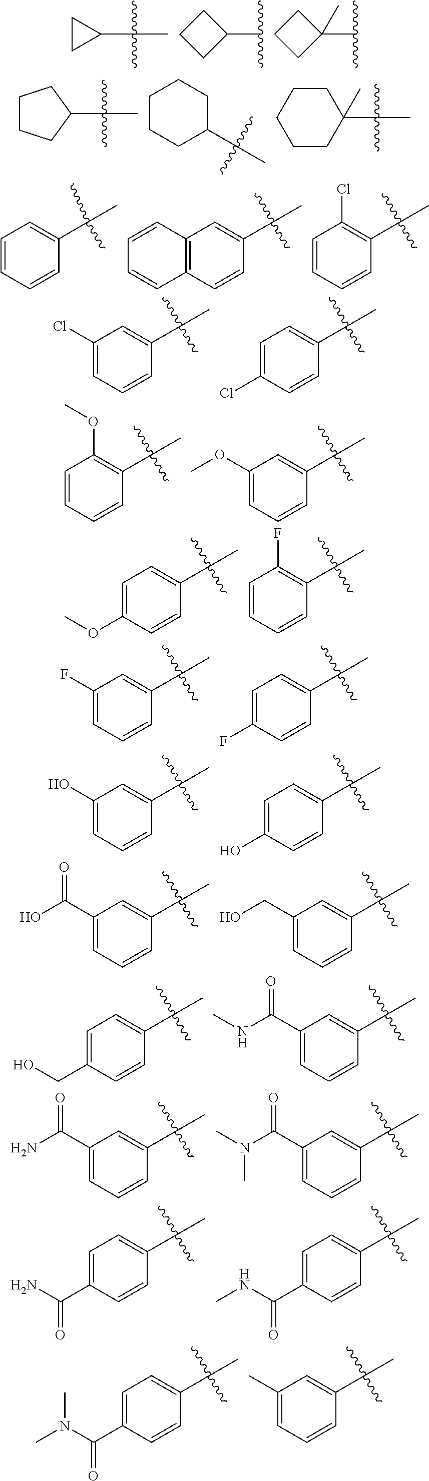

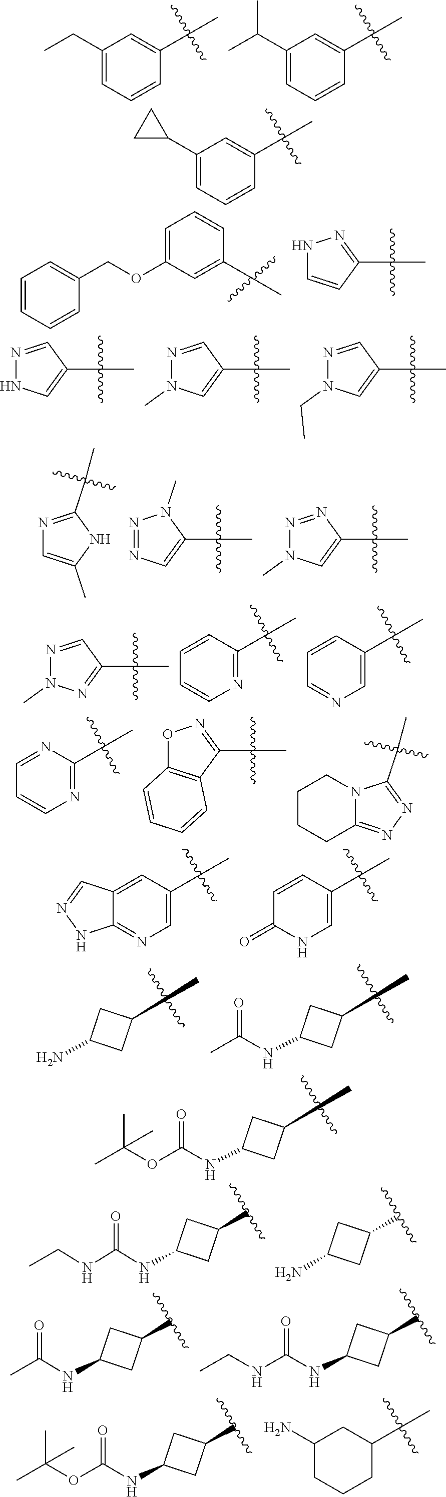

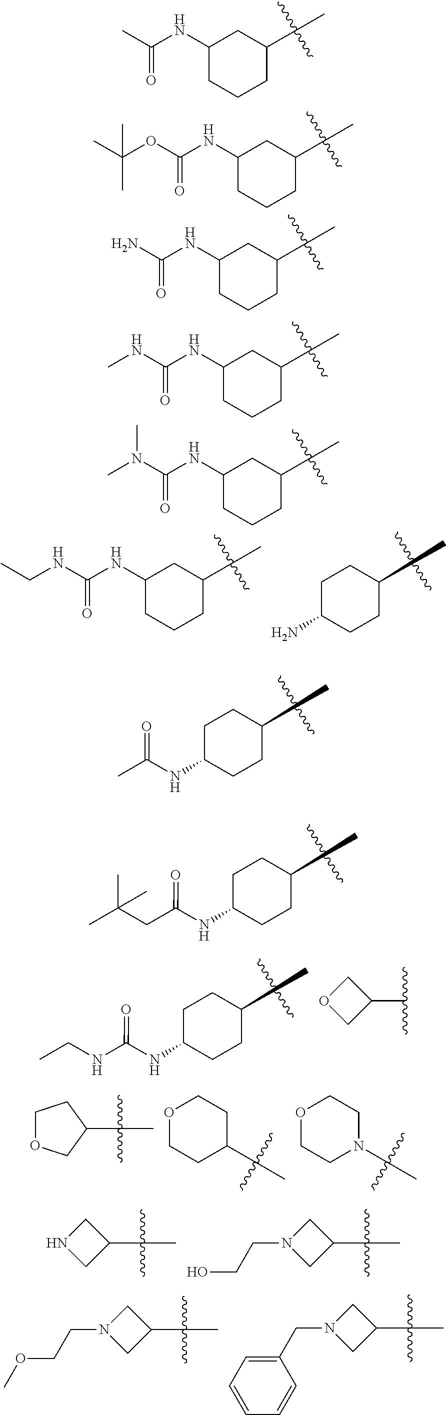

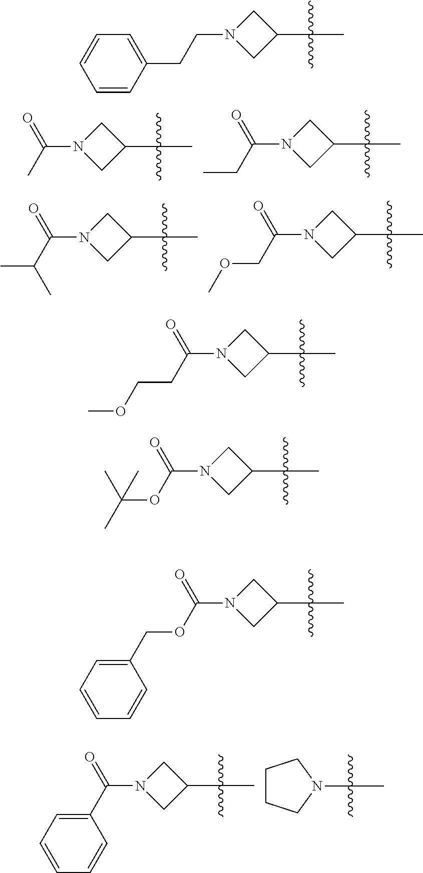

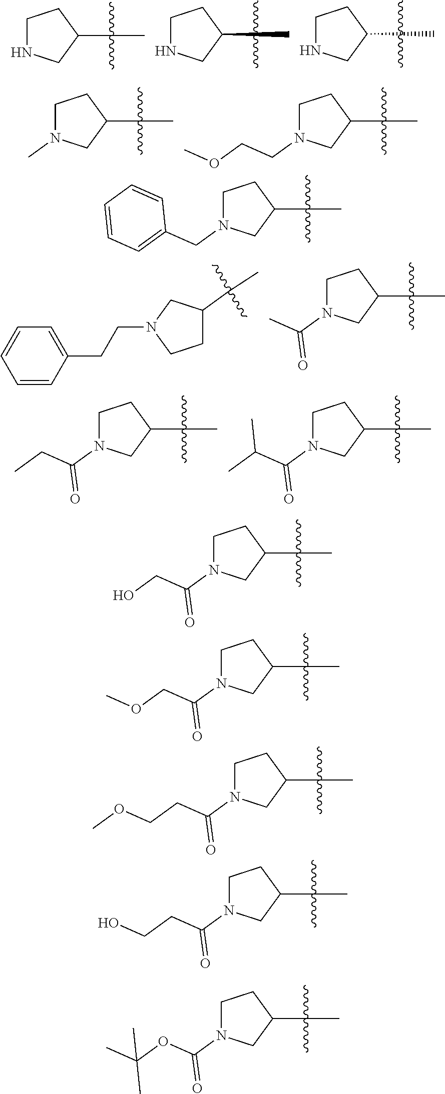

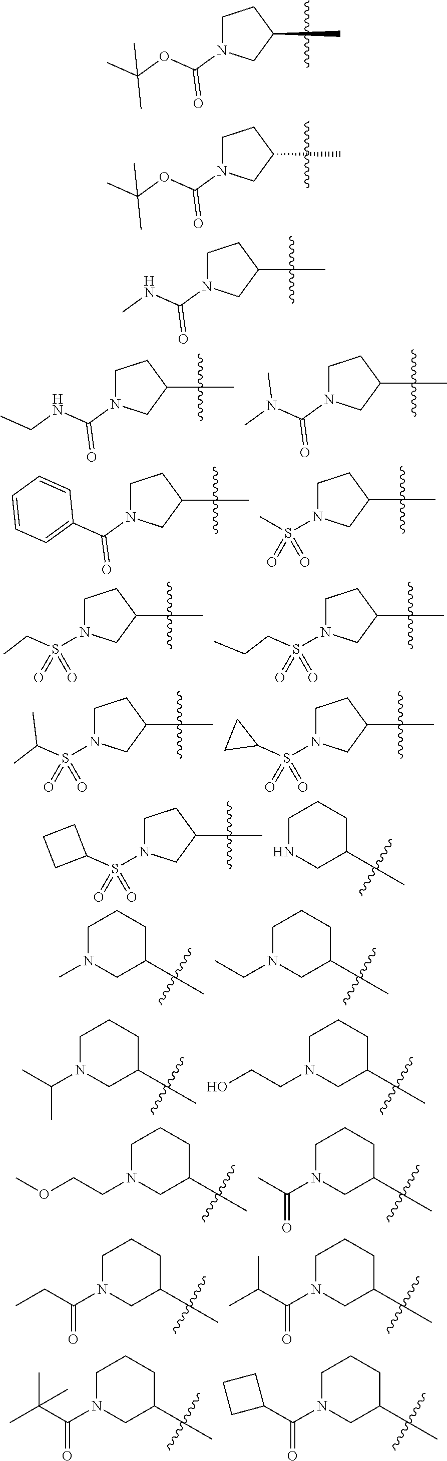

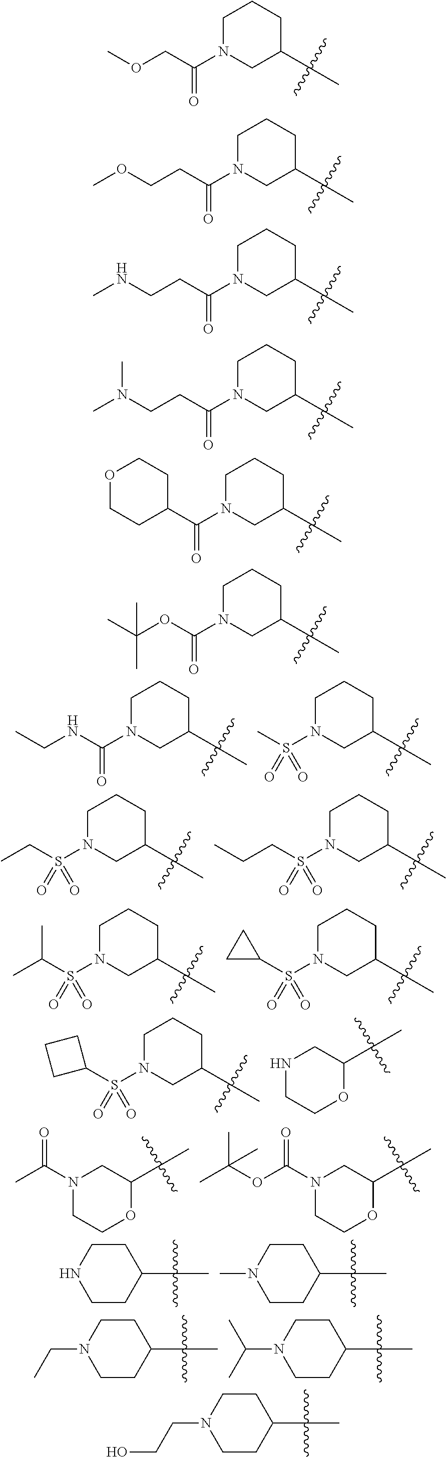

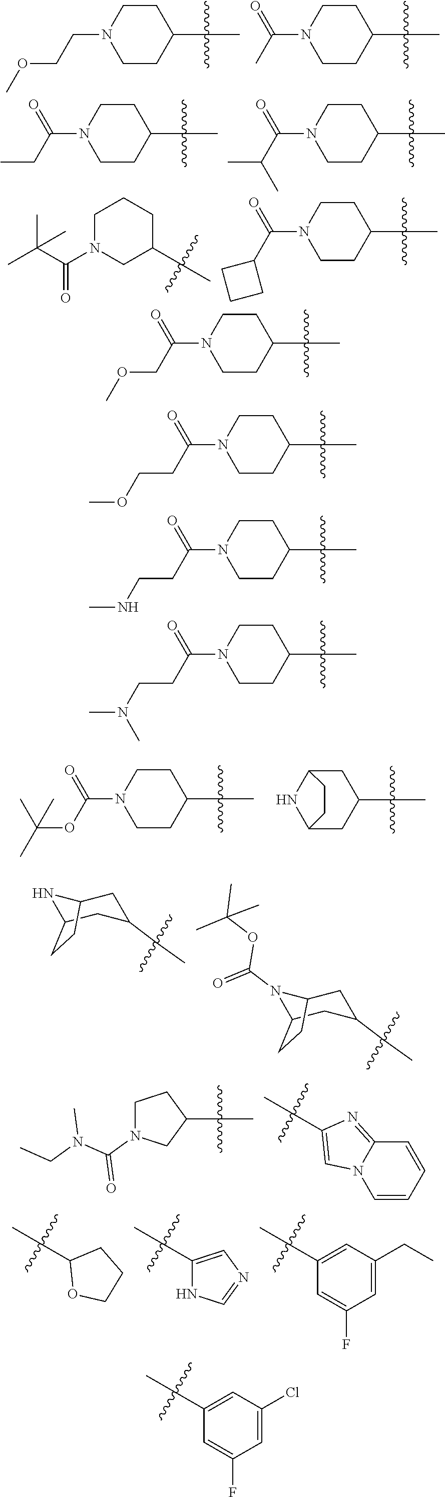

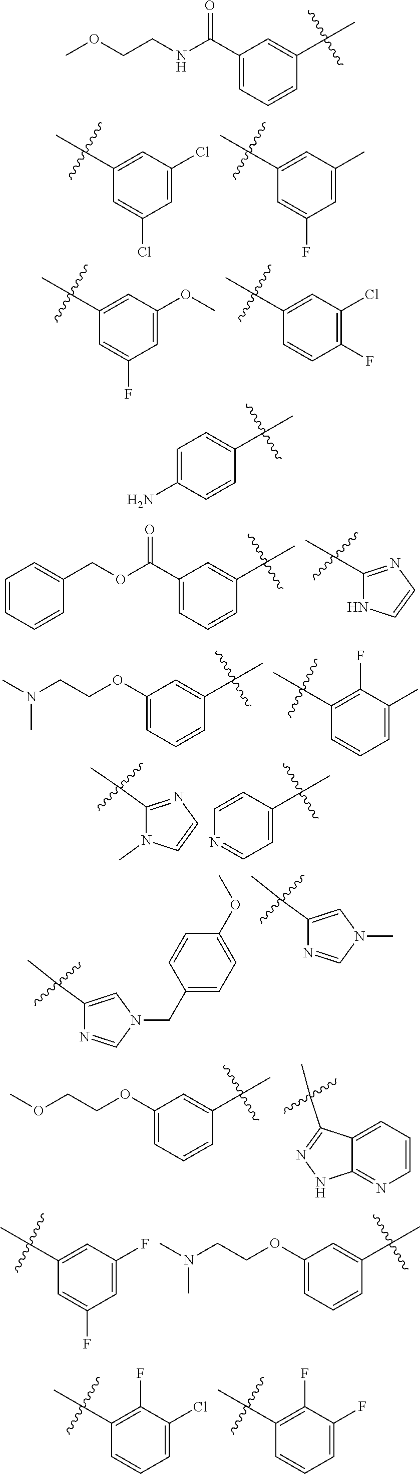

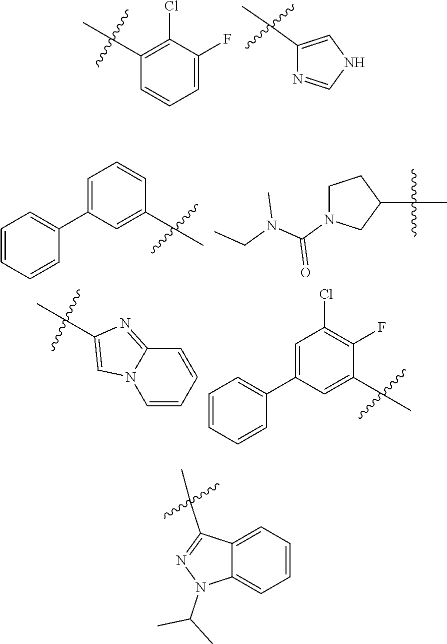

As defined above, each R is independently hydrogen or an optionally substituted group selected from C₁₋₄ aliphatic, phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, and an 8-10 membered bicyclic heteroaryl ring having 1-3 heteroatoms independently selected from nitrogen oxygen and sulfur.

In some embodiments, R is hydrogen.

In some embodiments, R is an optionally substituted group selected from C₁₋₄ aliphatic, phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, and an 8-10 membered bicyclic heteroaryl ring having 1-3 heteroatoms independently selected from nitrogen oxygen and sulfur,

In some embodiments, R is an optionally substituted C₁₋₄ aliphatic. In some embodiments, R is optionally substituted methyl. In some embodiments, R is optionally substituted ethyl. In some embodiments, R is optionally substituted i-propyl. In some embodiments, R is optionally substituted t-butyl.

In some embodiments, R is an optionally substituted phenyl.

In some embodiments, R is an optionally substituted 3-7 membered saturated or partially unsaturated carbocyclic ring. In some embodiments, R is an optionally substituted 5-6 membered saturated or partially unsaturated carbocyclic ring. In some embodiments, R is selected from optionally substituted cyclopropyl, cyclobutyl, cyclopentyl, cyclohexyl and cycloheptyl.

In some embodiments, R is an optionally substituted 3-7 membered saturated or partially unsaturated heterocyclic ring having 1-2 heteroatoms independently selected from nitrogen, oxygen and sulfur. In some embodiments, R is azetidinyl, pyrrolidinyl, piperidinyl, piperazinyl, morpholinyl, or azepinyl.

In some embodiments, R is an optionally substituted 5-6 membered heteroaryl ring having 1-2 heteroatoms independently selected from nitrogen, oxygen and sulfur. In some embodiments, R is an optionally substituted pyrrolyl, furanyl, thiophenyl, oxazolyl, thiazolyl, pyridyl, or pyrimidinyl.

In some embodiments, R is an optionally substituted 8-10 membered bicyclic aryl ring. In some embodiments, R is optionally substituted naphthyl.

In some embodiments, R is an optionally substituted 8-10 membered bicyclic heteroaryl ring having 1-3 heteroatoms independently selected from nitrogen oxygen and sulfur. In some such embodiments, R is an optionally substituted 9-10 bicyclic heteroaryl ring having 1-3 heteroatoms independently selected from nitrogen oxygen and sulfur. In some embodiments, R is indolyl, isoindolyl, indazolyl, benzimidazolyl, benzoxazolyl, benzothiazolyl, quinolyl, or isoquinolyl.

As defined above, y is 1 or 2. In some embodiments, y is 1. In some embodiments, y is 2.

As defined above, n is 0 or 1. In some embodiments, n is 0. In some embodiments, n is 1.

As defined above, x is 0, 1, 2, or 3. In some embodiments, x is 0. In some embodiments, x is 1. In some embodiments, x is 2. In some embodiments, x is 3.

In some embodiments, the present invention provides a compound of formulae I-a, I-b, I-c, I-d, I-e, I-g, I-h, I-j, I-k, I-l, I-m, I-n, I-o, I-p, I-q, I-r, or I-s:

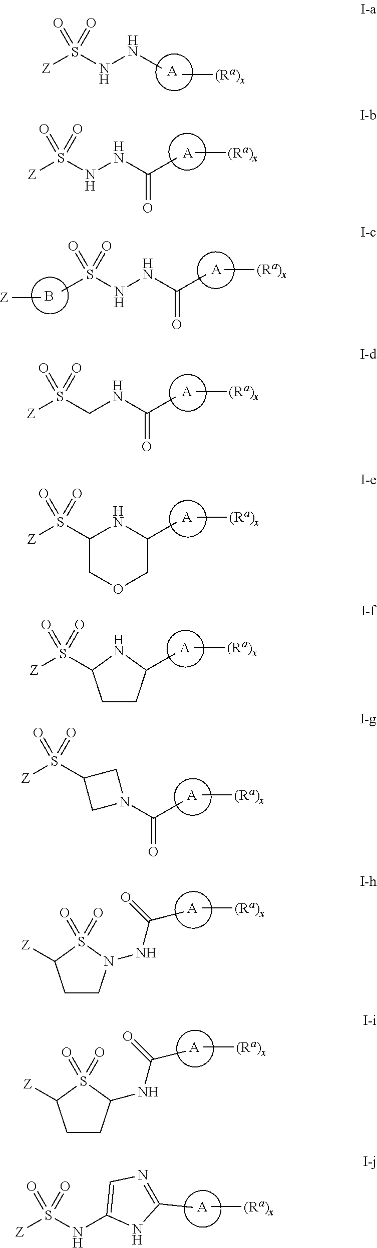

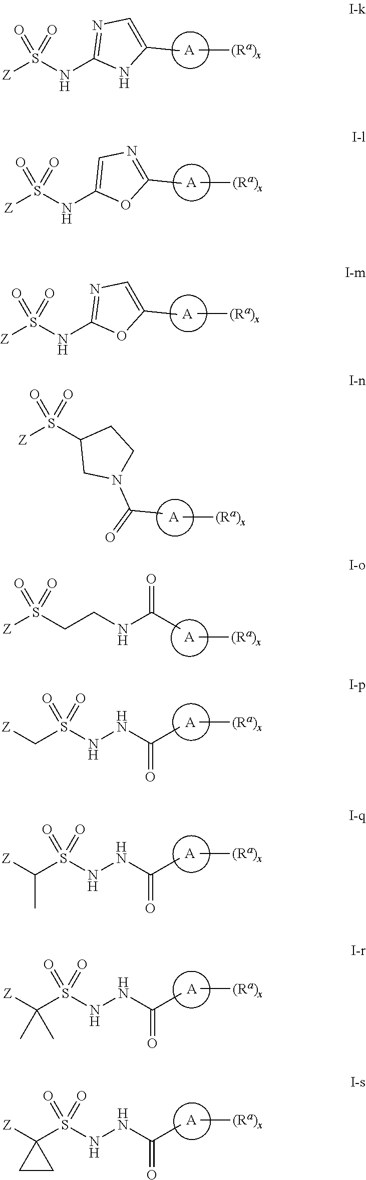

or a pharmaceutically acceptable salt thereof.

In some embodiments, the present invention provides a compound of formulae II, III, IV or V:

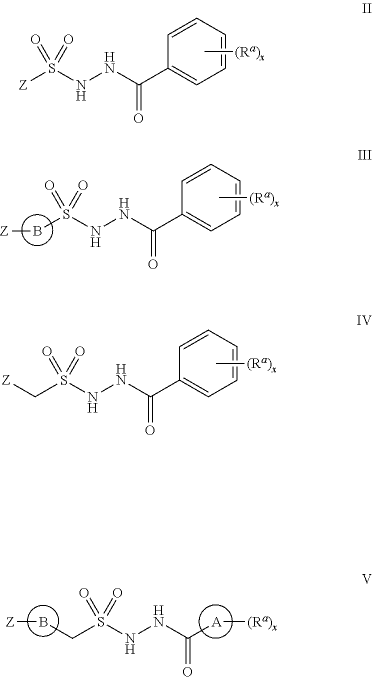

or a pharmaceutically acceptable salt thereof.

In some embodiments, the compound of formula I is selected from the group consisting of

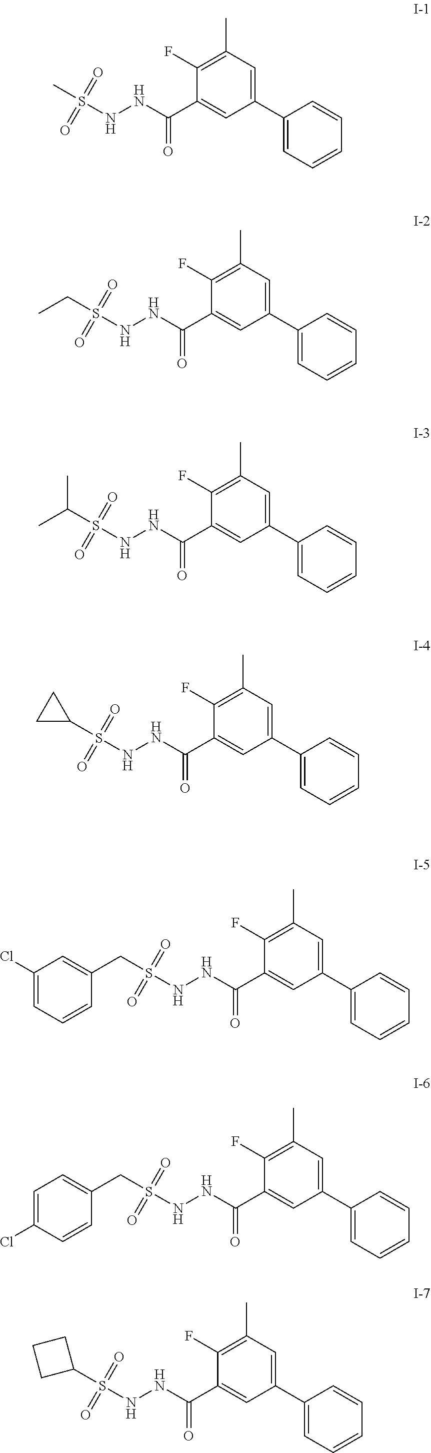

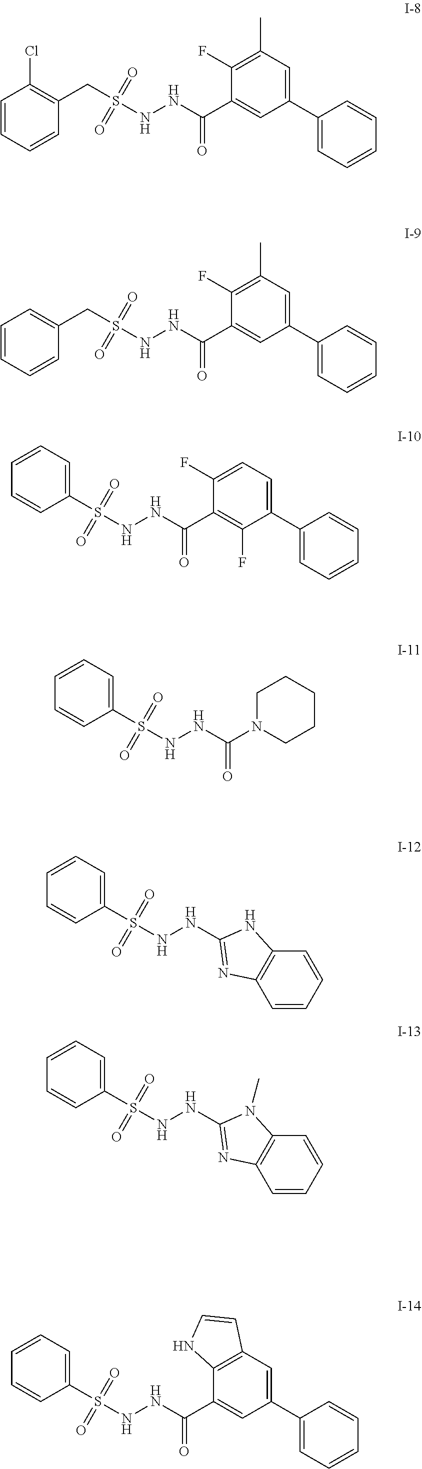

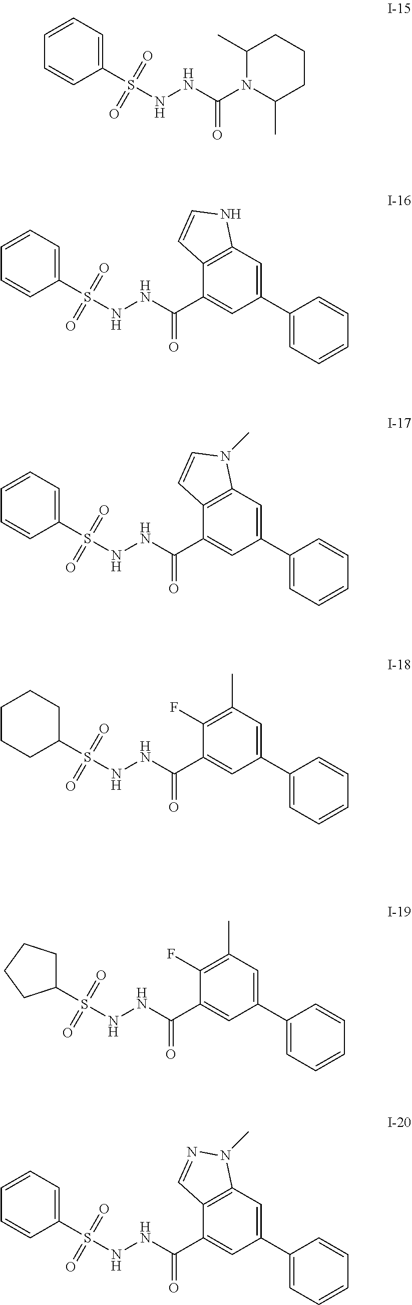 [SMILES: CC1CCCC(C)N1C(=O)NNS(=O)(=O)c1ccccc1 ; O=C(NNS(=O)(=O)c1ccccc1)c1cc(-c2ccccc2)cc2[nH]ccc12 ; Cn1ccc2c(C(=O)NNS(=O)(=O)c3ccccc3)cc(-c3ccccc3)cc21 ; Cc1cc(-c2ccccc2)cc(C(=O)NNS(=O)(=O)C2CCCCC2)c1F ; Cc1cc(-c2ccccc2)cc(C(=O)NNS(=O)(=O)C2CCCC2)c1F ; Cn1ncc2c(C(=O)NNS(=O)(=O)c3ccccc3)cc(-c3ccccc3)cc21]

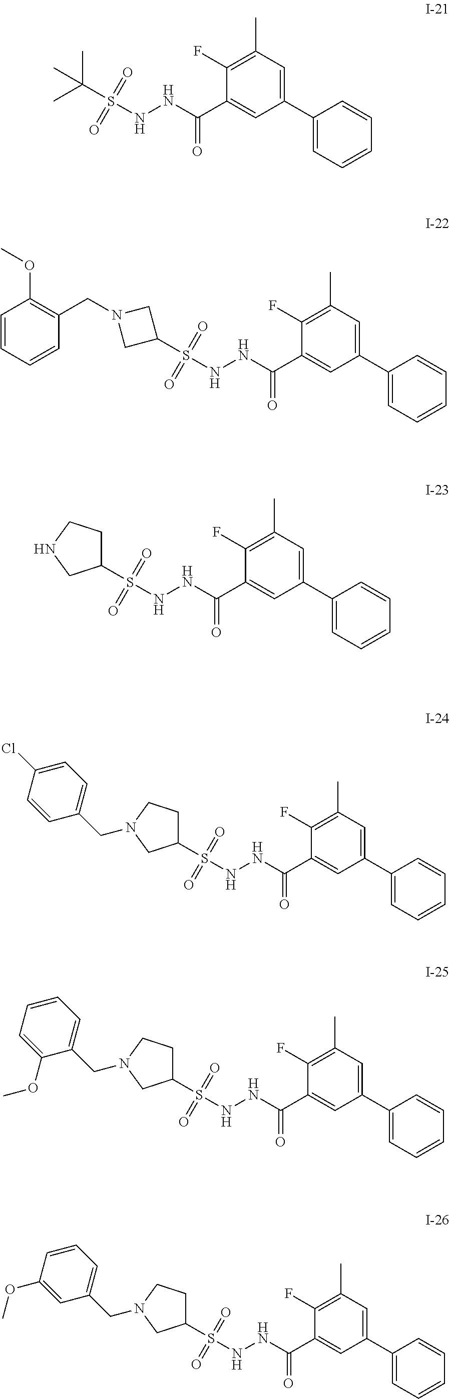 [SMILES: Cc1cc(-c2ccccc2)cc(C(=O)NNS(=O)(=O)C(C)(C)C)c1F ; COc1ccccc1CN1CC(S(=O)(=O)NNC(=O)c2cc(-c3ccccc3)cc(C)c2F)C1 ; Cc1cc(-c2ccccc2)cc(C(=O)NNS(=O)(=O)C2CCNC2)c1F ; Cc1cc(-c2ccccc2)cc(C(=O)NNS(=O)(=O)C2CCN(Cc3ccc(Cl)cc3)C2)c1F ; COc1ccccc1CN1CCC(S(=O)(=O)NNC(=O)c2cc(-c3ccccc3)cc(C)c2F)C1 ; COc1cccc(CN2CCC(S(=O)(=O)NNC(=O)c3cc(-c4ccccc4)cc(C)c3F)C2)c1]

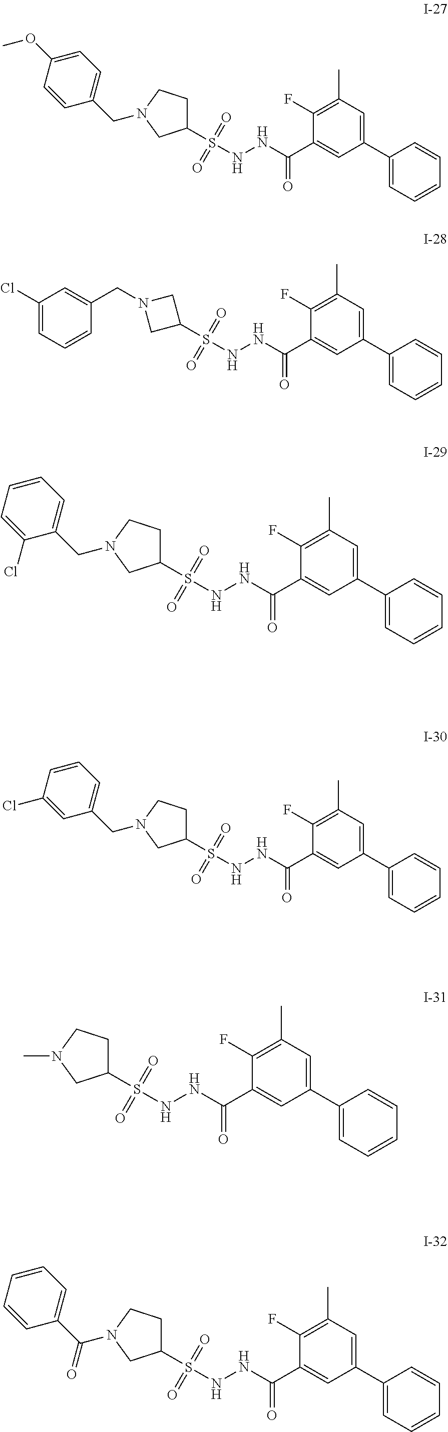 [SMILES: COc1ccc(CN2CCC(S(=O)(=O)NNC(=O)c3cc(-c4ccccc4)cc(C)c3F)C2)cc1 ; Cc1cc(-c2ccccc2)cc(C(=O)NNS(=O)(=O)C2CN(Cc3cccc(Cl)c3)C2)c1F ; Cc1cc(-c2ccccc2)cc(C(=O)NNS(=O)(=O)C2CCN(Cc3ccccc3Cl)C2)c1F ; Cc1cc(-c2ccccc2)cc(C(=O)NNS(=O)(=O)C2CCN(Cc3cccc(Cl)c3)C2)c1F ; Cc1cc(-c2ccccc2)cc(C(=O)NNS(=O)(=O)C2CCN(C)C2)c1F ; Cc1cc(-c2ccccc2)cc(C(=O)NNS(=O)(=O)C2CCN(C(=O)c3ccccc3)C2)c1F]

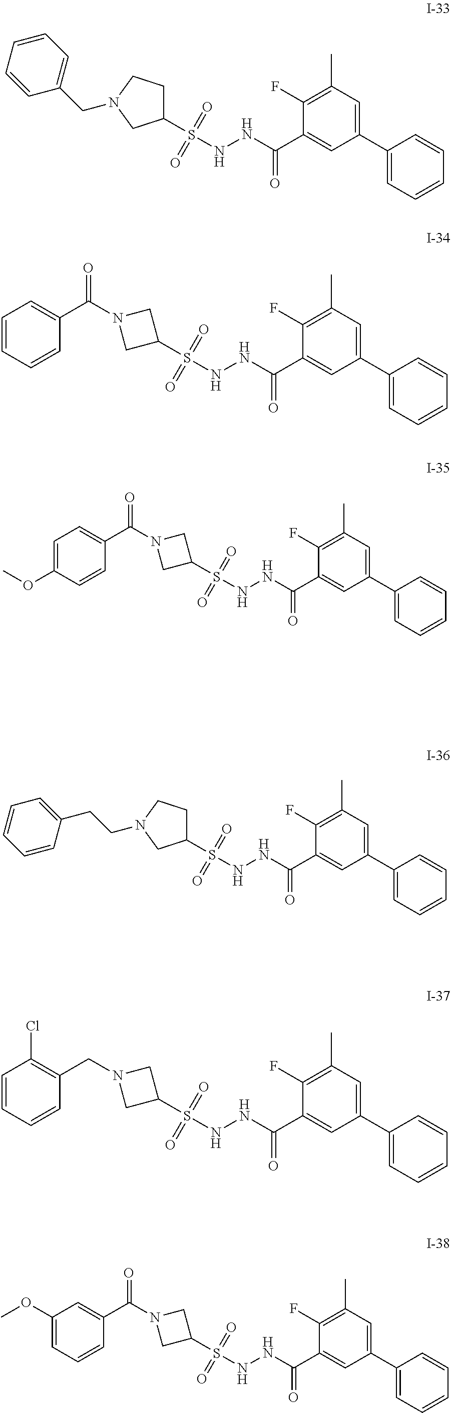 [SMILES: Cc1cc(-c2ccccc2)cc(C(=O)NNS(=O)(=O)C2CCN(Cc3ccccc3)C2)c1F ; Cc1cc(-c2ccccc2)cc(C(=O)NNS(=O)(=O)C2CN(C(=O)c3ccccc3)C2)c1F ; COc1ccc(C(=O)N2CC(S(=O)(=O)NNC(=O)c3cc(-c4ccccc4)cc(C)c3F)C2)cc1 ; Cc1cc(-c2ccccc2)cc(C(=O)NNS(=O)(=O)C2CCN(CCc3ccccc3)C2)c1F ; Cc1cc(-c2ccccc2)cc(C(=O)NNS(=O)(=O)C2CN(Cc3ccccc3Cl)C2)c1F ; COc1cccc(C(=O)N2CC(S(=O)(=O)NNC(=O)c3cc(-c4ccccc4)cc(C)c3F)C2)c1]

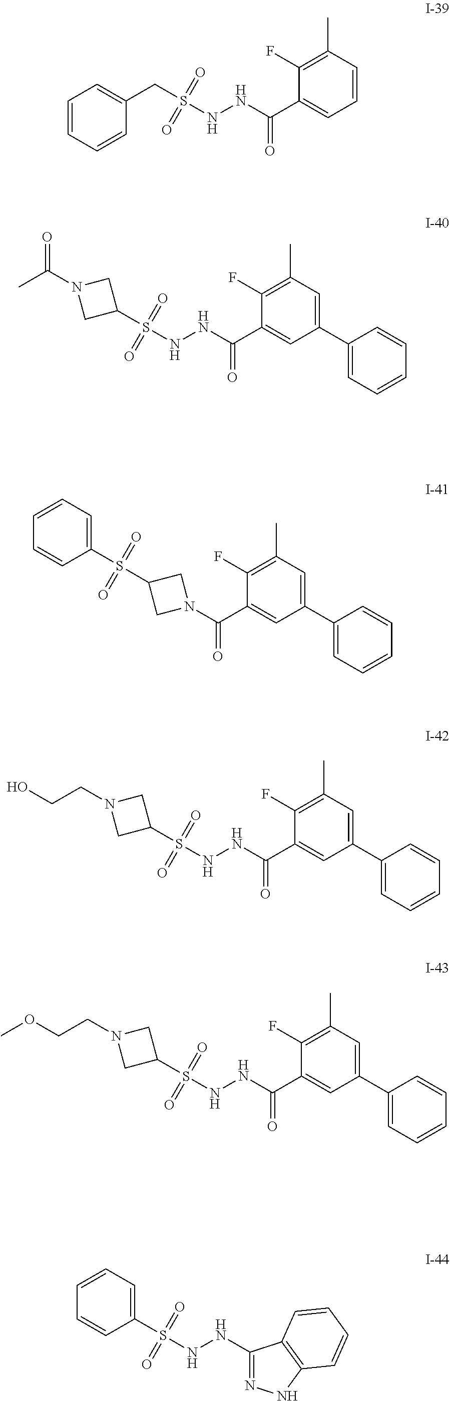 [SMILES: Cc1cccc(C(=O)NNS(=O)(=O)Cc2ccccc2)c1F ; CC(=O)N1CC(S(=O)(=O)NNC(=O)c2cc(-c3ccccc3)cc(C)c2F)C1 ; Cc1cc(-c2ccccc2)cc(C(=O)N2CC(S(=O)(=O)c3ccccc3)C2)c1F ; Cc1cc(-c2ccccc2)cc(C(=O)NNS(=O)(=O)C2CN(CCO)C2)c1F ; COCCN1CC(S(=O)(=O)NNC(=O)c2cc(-c3ccccc3)cc(C)c2F)C1 ; O=S(=O)(NNc1n[nH]c2ccccc12)c1ccccc1]

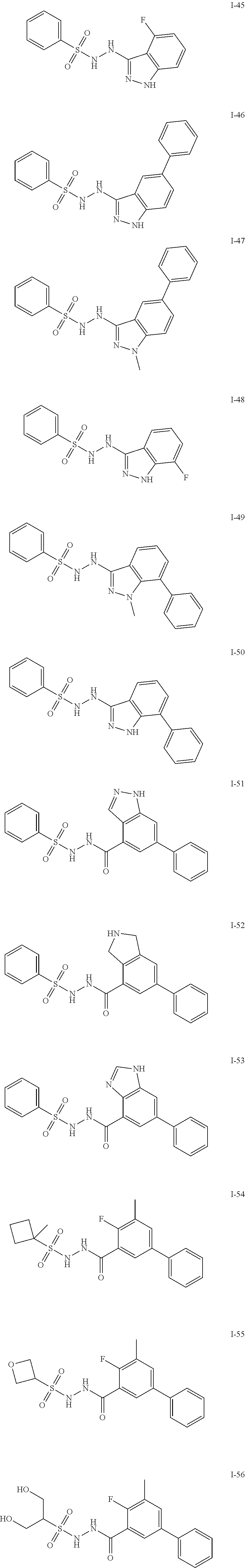

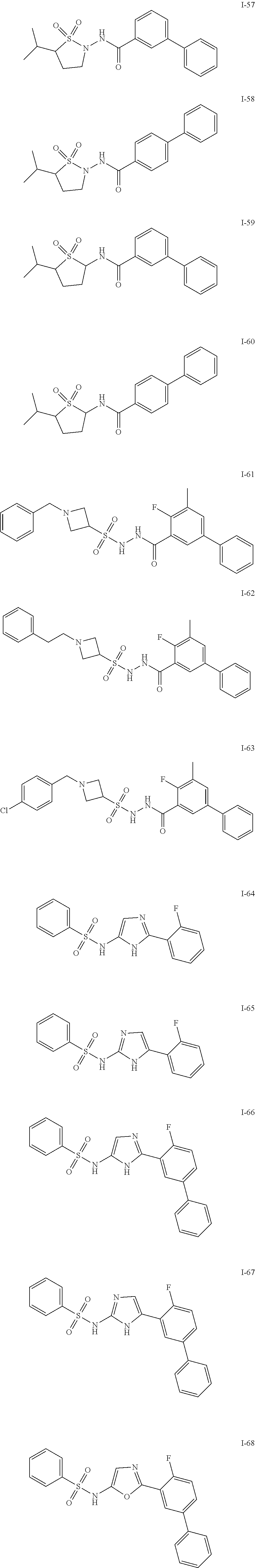

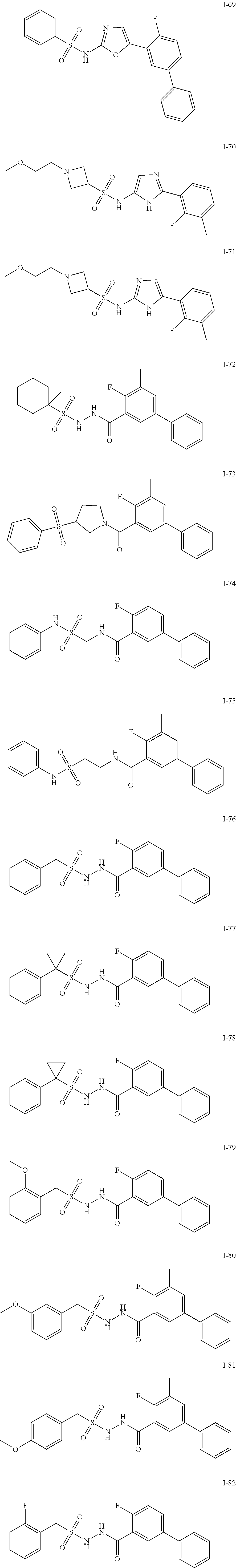

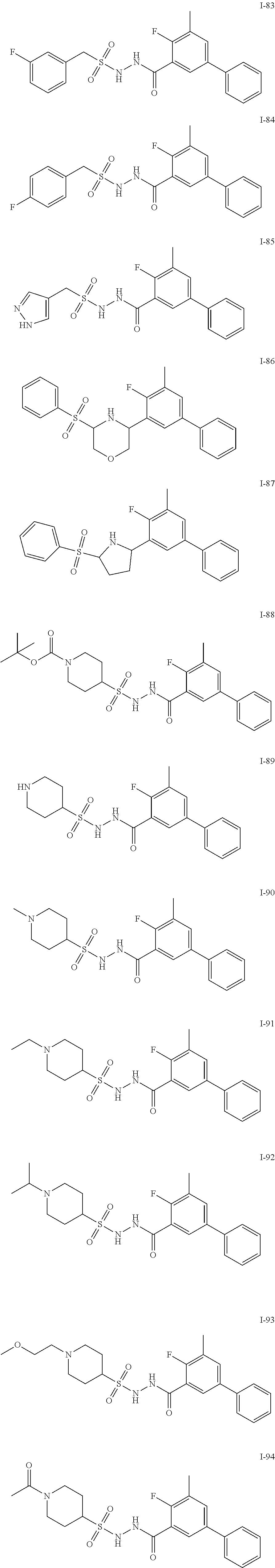

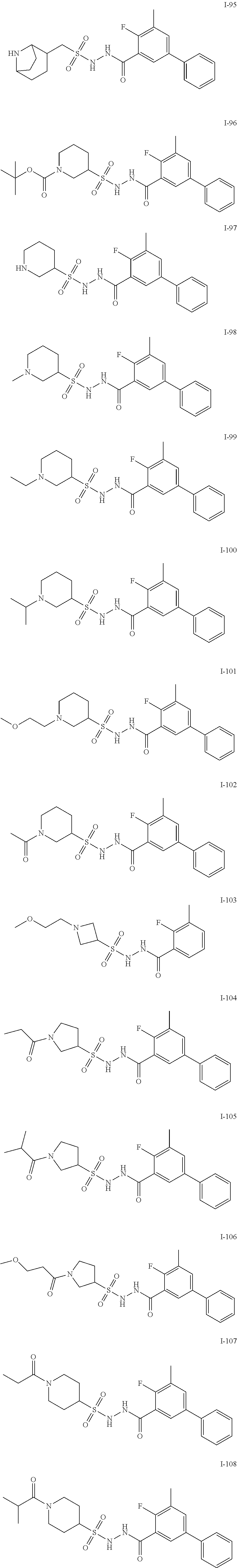

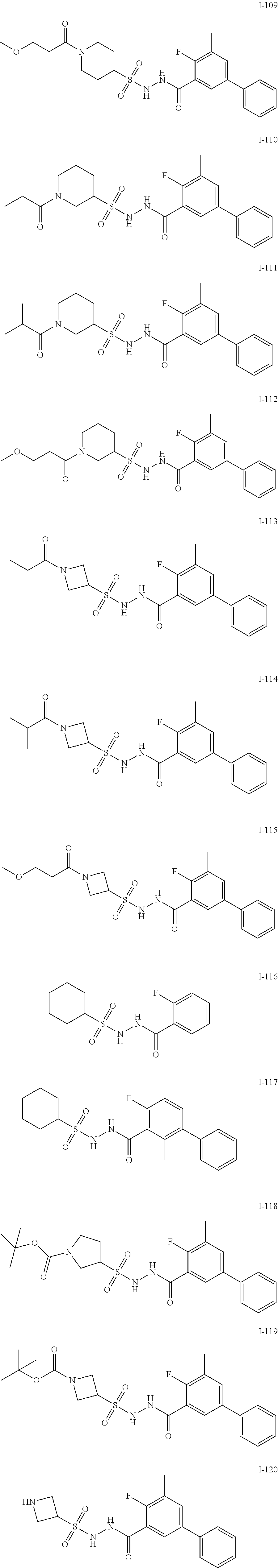

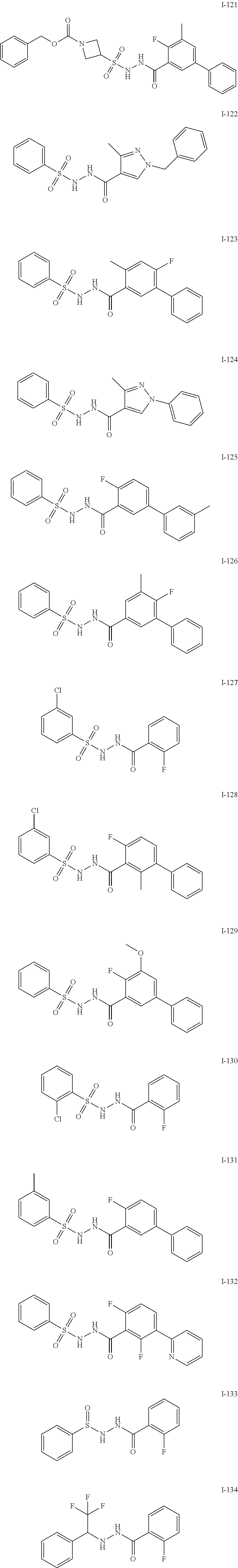

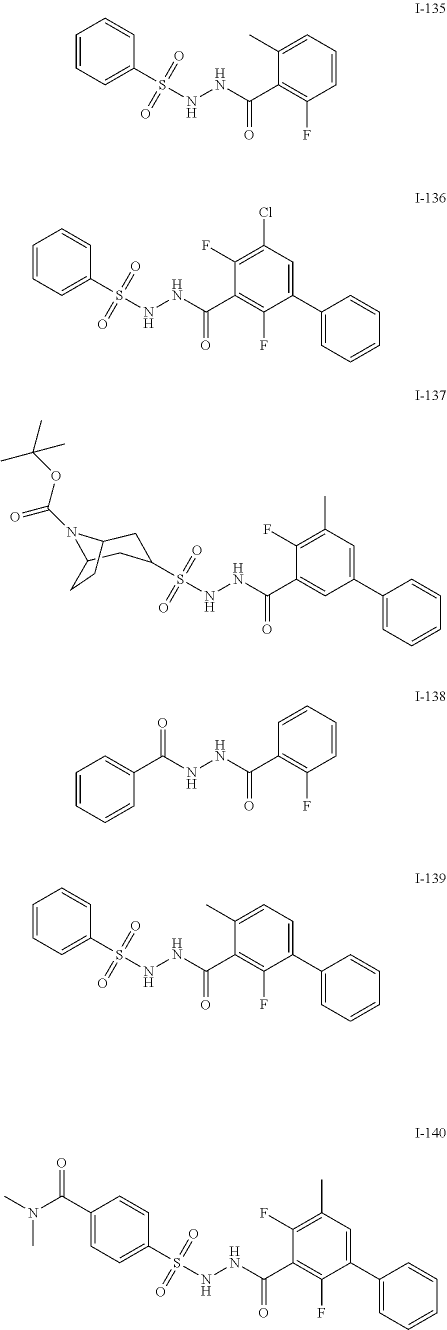 [SMILES: Cc1cccc(F)c1C(=O)NNS(=O)(=O)c1ccccc1 ; O=C(NNS(=O)(=O)c1ccccc1)c1c(F)c(Cl)cc(-c2ccccc2)c1F ; Cc1cc(-c2ccccc2)cc(C(=O)NNS(=O)(=O)C2CC3CCC(C2)N3C(=O)OC(C)(C)C)c1F ; O=C(NNC(=O)c1ccccc1F)c1ccccc1 ; Cc1ccc(-c2ccccc2)c(F)c1C(=O)NNS(=O)(=O)c1ccccc1 ; Cc1cc(-c2ccccc2)c(F)c(C(=O)NNS(=O)(=O)c2ccc(C(=O)N(C)C)cc2)c1F]

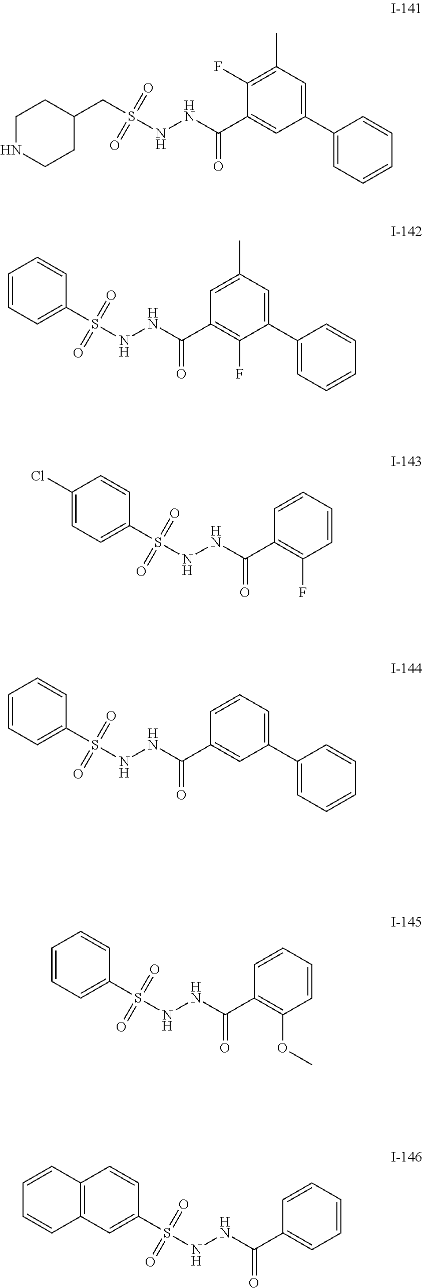 [SMILES: Cc1cc(-c2ccccc2)cc(C(=O)NNS(=O)(=O)CC2CCNCC2)c1F ; Cc1cc(C(=O)NNS(=O)(=O)c2ccccc2)c(F)c(-c2ccccc2)c1 ; O=C(NNS(=O)(=O)c1ccc(Cl)cc1)c1ccccc1F ; O=C(NNS(=O)(=O)c1ccccc1)c1cccc(-c2ccccc2)c1 ; COc1ccccc1C(=O)NNS(=O)(=O)c1ccccc1 ; O=C(NNS(=O)(=O)c1ccc2ccccc2c1)c1ccccc1]

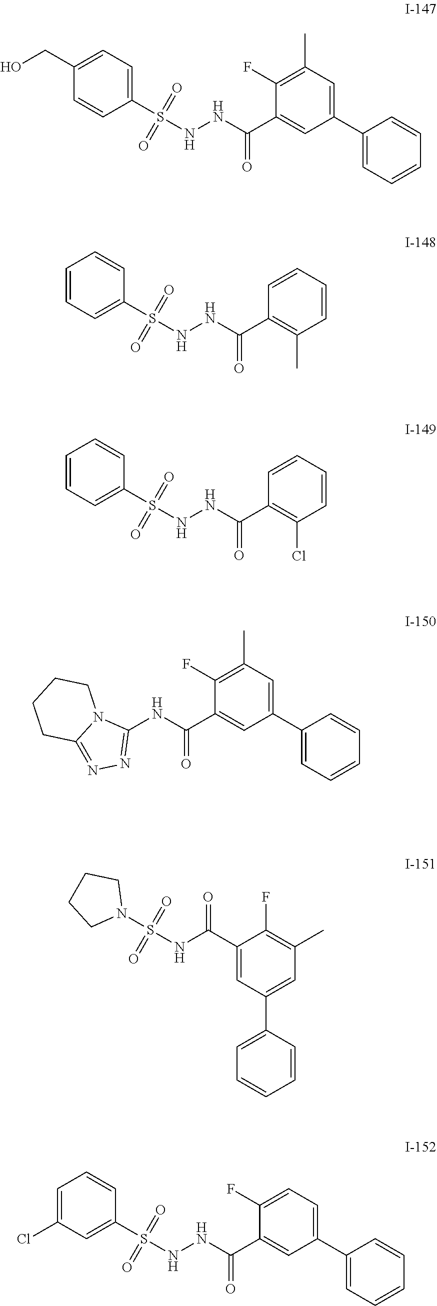 [SMILES: Cc1cc(-c2ccccc2)cc(C(=O)NNS(=O)(=O)c2ccc(CO)cc2)c1F ; Cc1ccccc1C(=O)NNS(=O)(=O)c1ccccc1 ; O=C(NNS(=O)(=O)c1ccccc1)c1ccccc1Cl ; Cc1cc(-c2ccccc2)cc(C(=O)Nc2nnc3n2CCCC3)c1F ; Cc1cc(-c2ccccc2)cc(C(=O)NS(=O)(=O)N2CCCC2)c1F ; O=C(NNS(=O)(=O)c1cccc(Cl)c1)c1cc(-c2ccccc2)ccc1F]

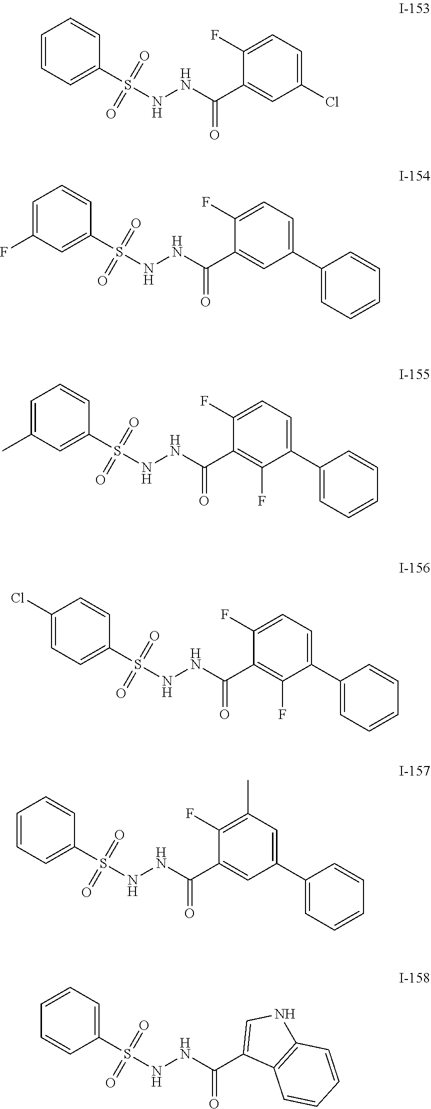 [SMILES: O=C(NNS(=O)(=O)c1ccccc1)c1cc(Cl)ccc1F ; O=C(NNS(=O)(=O)c1cccc(F)c1)c1cc(-c2ccccc2)ccc1F ; Cc1cccc(S(=O)(=O)NNC(=O)c2c(F)ccc(-c3ccccc3)c2F)c1 ; O=C(NNS(=O)(=O)c1ccc(Cl)cc1)c1c(F)ccc(-c2ccccc2)c1F ; Cc1cc(-c2ccccc2)cc(C(=O)NNS(=O)(=O)c2ccccc2)c1F ; O=C(NNS(=O)(=O)c1ccccc1)c1c[nH]c2ccccc12]

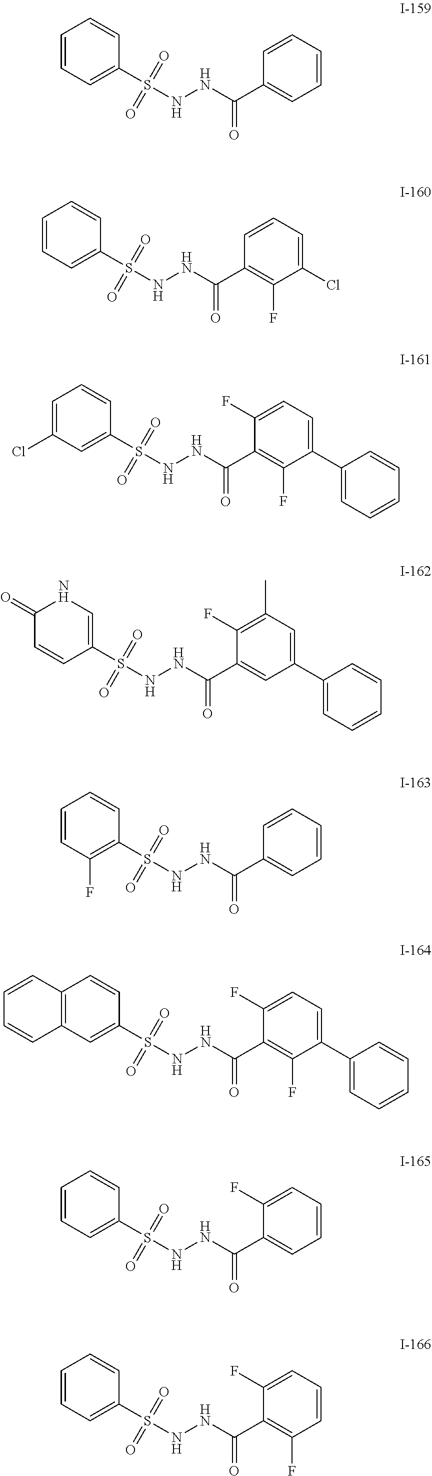

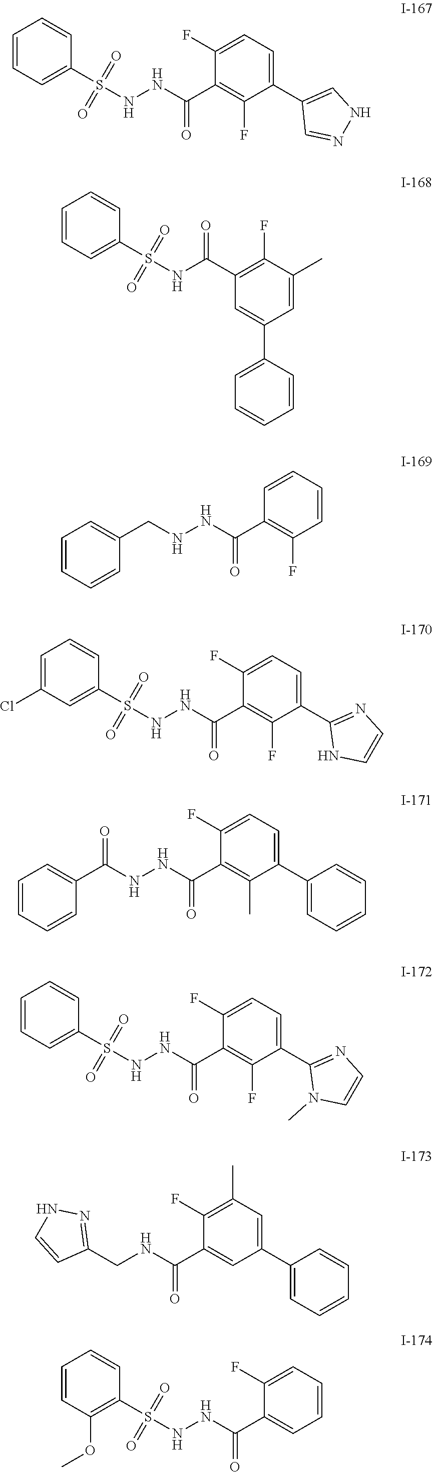

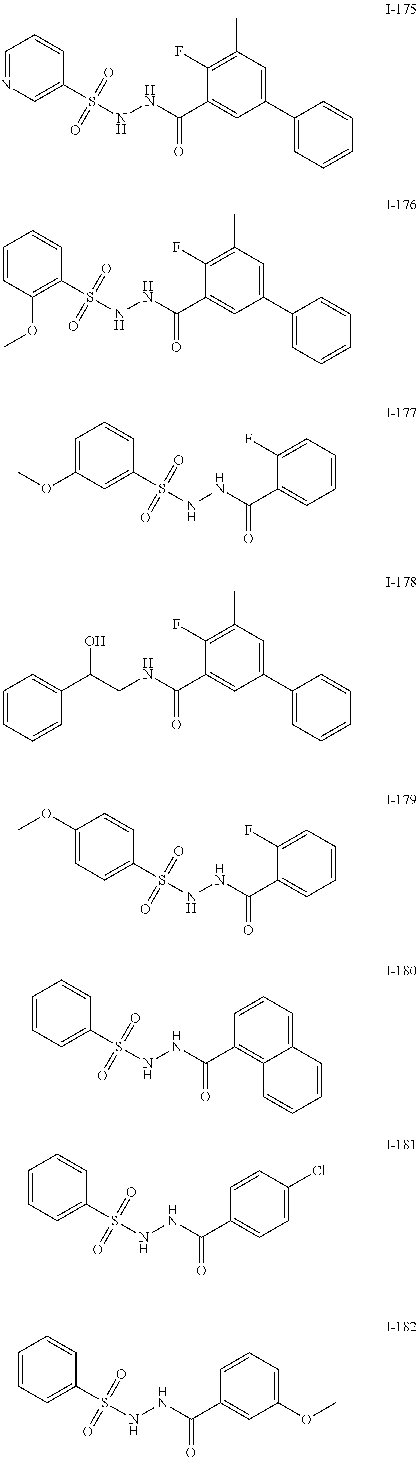

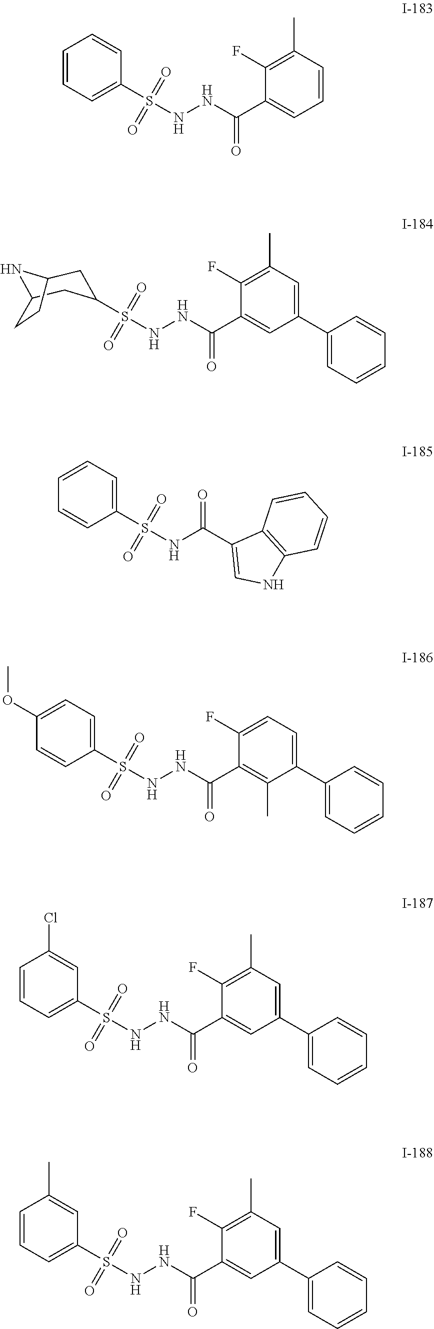 [SMILES: Cc1cccc(C(=O)NNS(=O)(=O)c2ccccc2)c1F ; Cc1cc(-c2ccccc2)cc(C(=O)NNS(=O)(=O)C2CC3CCC(C2)N3)c1F ; O=C(NS(=O)(=O)c1ccccc1)c1c[nH]c2ccccc12 ; COc1ccc(S(=O)(=O)NNC(=O)c2c(F)ccc(-c3ccccc3)c2C)cc1 ; Cc1cc(-c2ccccc2)cc(C(=O)NNS(=O)(=O)c2cccc(Cl)c2)c1F ; Cc1cccc(S(=O)(=O)NNC(=O)c2cc(-c3ccccc3)cc(C)c2F)c1]

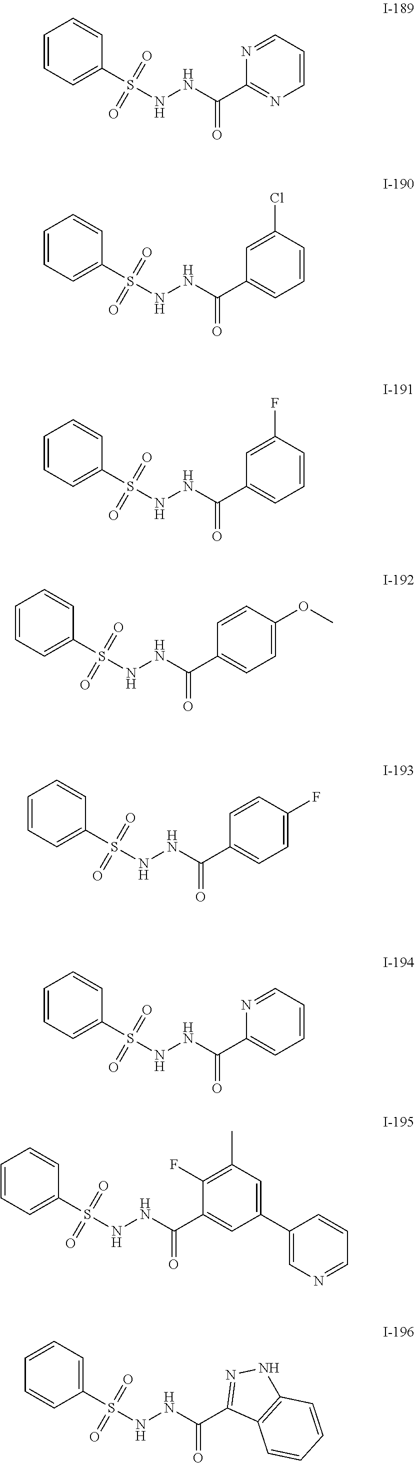

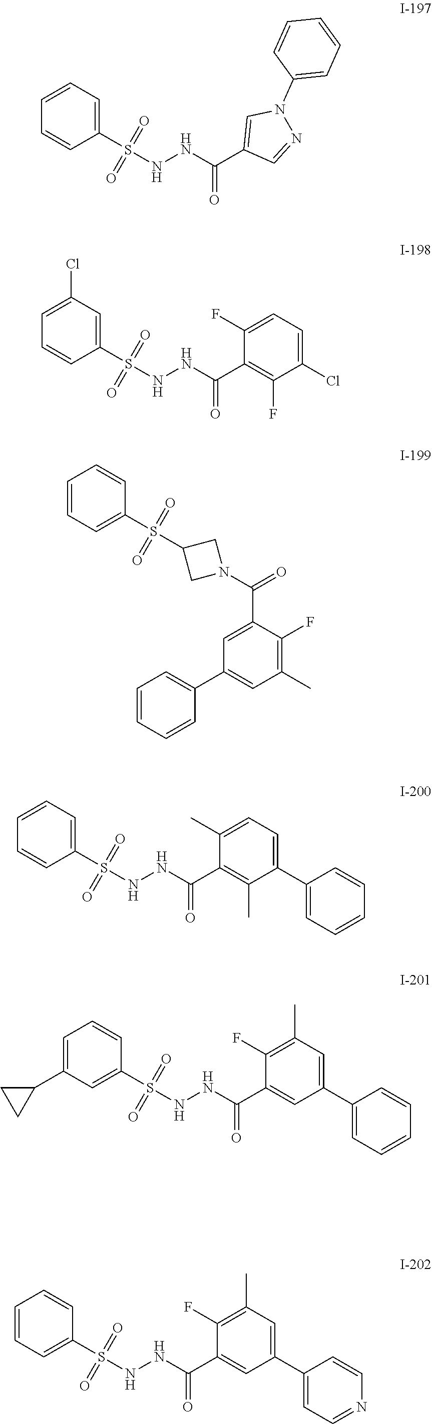 [SMILES: O=C(NNS(=O)(=O)c1ccccc1)c1cnn(-c2ccccc2)c1 ; O=C(NNS(=O)(=O)c1cccc(Cl)c1)c1c(F)ccc(Cl)c1F ; Cc1cc(-c2ccccc2)cc(C(=O)N2CC(S(=O)(=O)c3ccccc3)C2)c1F ; Cc1ccc(-c2ccccc2)c(C)c1C(=O)NNS(=O)(=O)c1ccccc1 ; Cc1cc(-c2ccccc2)cc(C(=O)NNS(=O)(=O)c2cccc(C3CC3)c2)c1F ; Cc1cc(-c2ccncc2)cc(C(=O)NNS(=O)(=O)c2ccccc2)c1F]

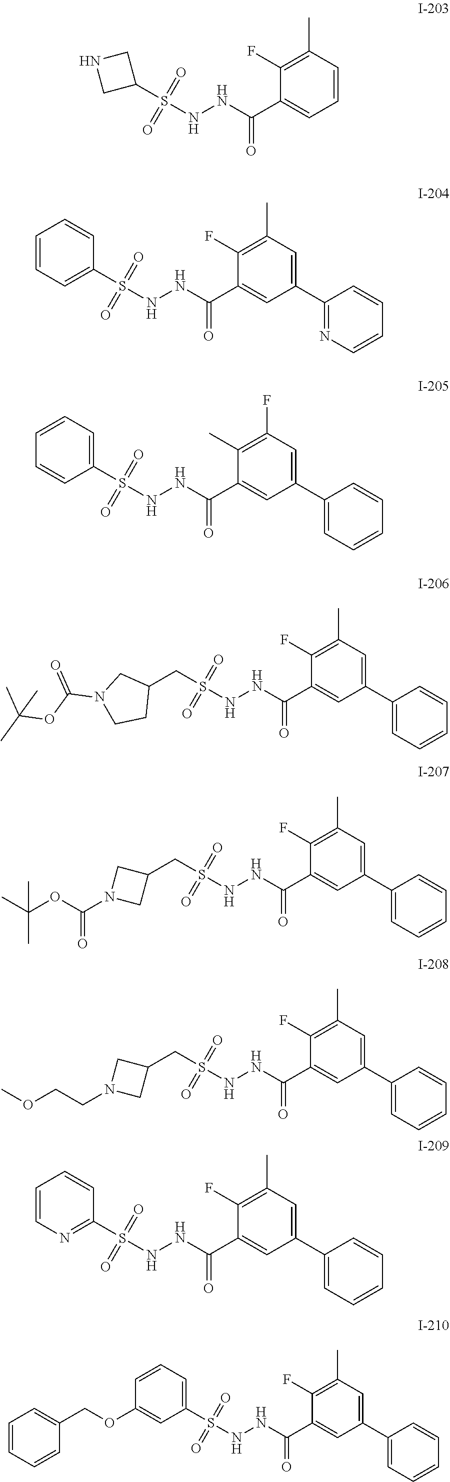

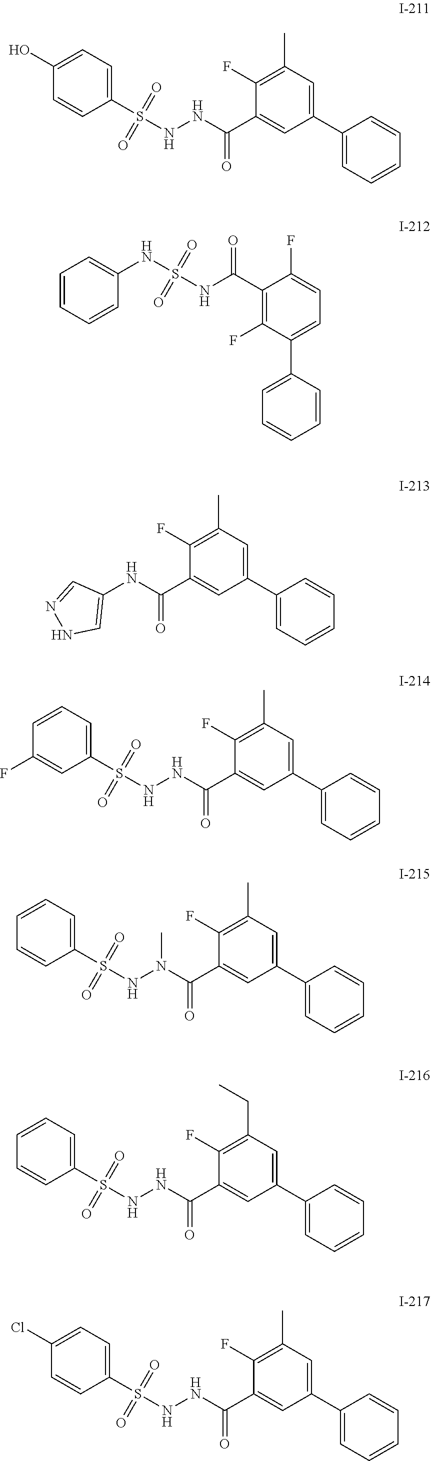

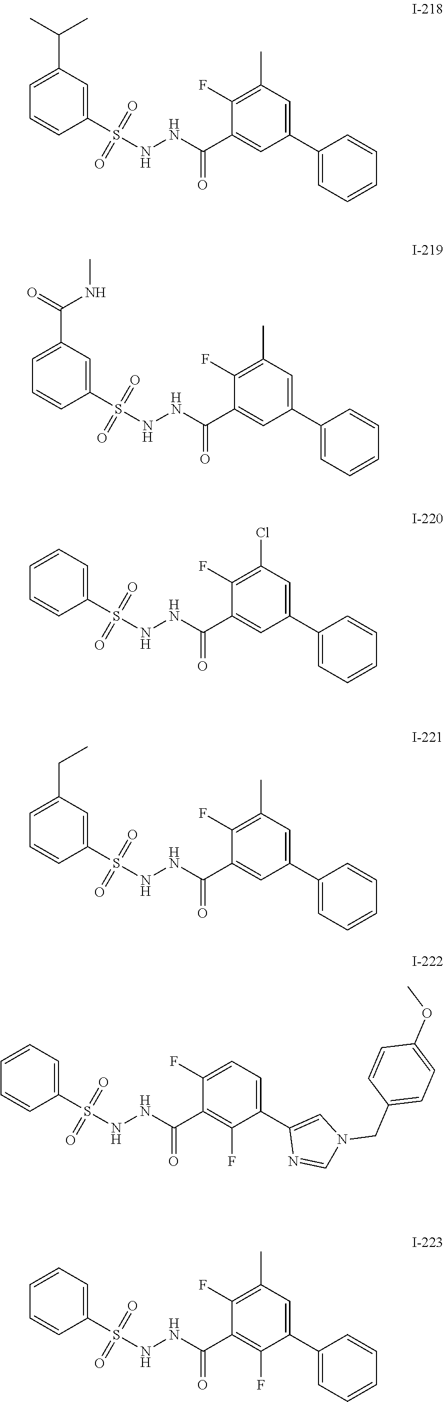 [SMILES: Cc1cc(-c2ccccc2)cc(C(=O)NNS(=O)(=O)c2cccc(C(C)C)c2)c1F ; CNC(=O)c1cccc(S(=O)(=O)NNC(=O)c2cc(-c3ccccc3)cc(C)c2F)c1 ; O=C(NNS(=O)(=O)c1ccccc1)c1cc(-c2ccccc2)cc(Cl)c1F ; CCc1cccc(S(=O)(=O)NNC(=O)c2cc(-c3ccccc3)cc(C)c2F)c1 ; COc1ccc(Cn2cnc(-c3ccc(F)c(C(=O)NNS(=O)(=O)c4ccccc4)c3F)c2)cc1 ; Cc1cc(-c2ccccc2)c(F)c(C(=O)NNS(=O)(=O)c2ccccc2)c1F]

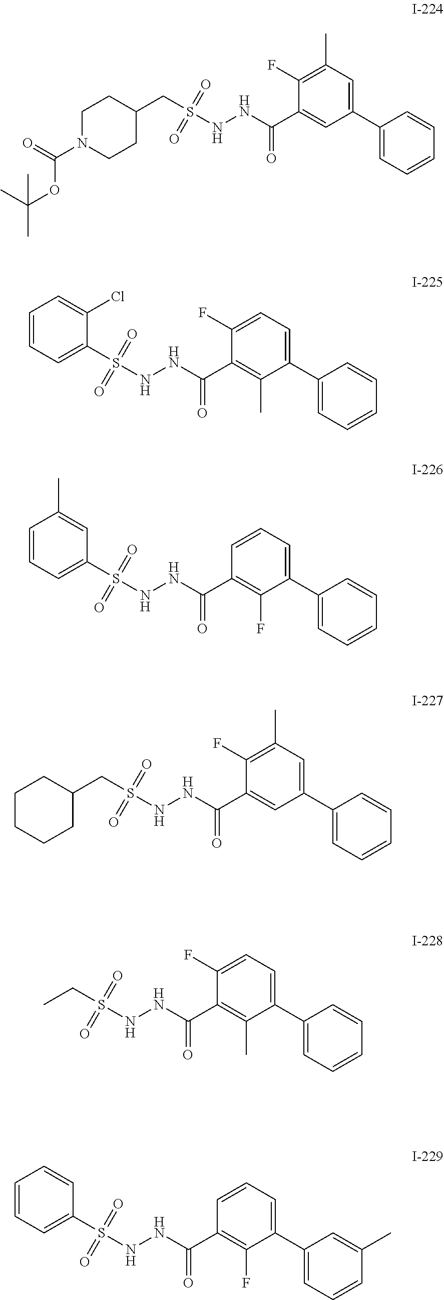 [SMILES: Cc1cc(-c2ccccc2)cc(C(=O)NNS(=O)(=O)CC2CCN(C(=O)OC(C)(C)C)CC2)c1F ; Cc1c(-c2ccccc2)ccc(F)c1C(=O)NNS(=O)(=O)c1ccccc1Cl ; Cc1cccc(S(=O)(=O)NNC(=O)c2cccc(-c3ccccc3)c2F)c1 ; Cc1cc(-c2ccccc2)cc(C(=O)NNS(=O)(=O)CC2CCCCC2)c1F ; CCS(=O)(=O)NNC(=O)c1c(F)ccc(-c2ccccc2)c1C ; Cc1cccc(-c2cccc(C(=O)NNS(=O)(=O)c3ccccc3)c2F)c1]

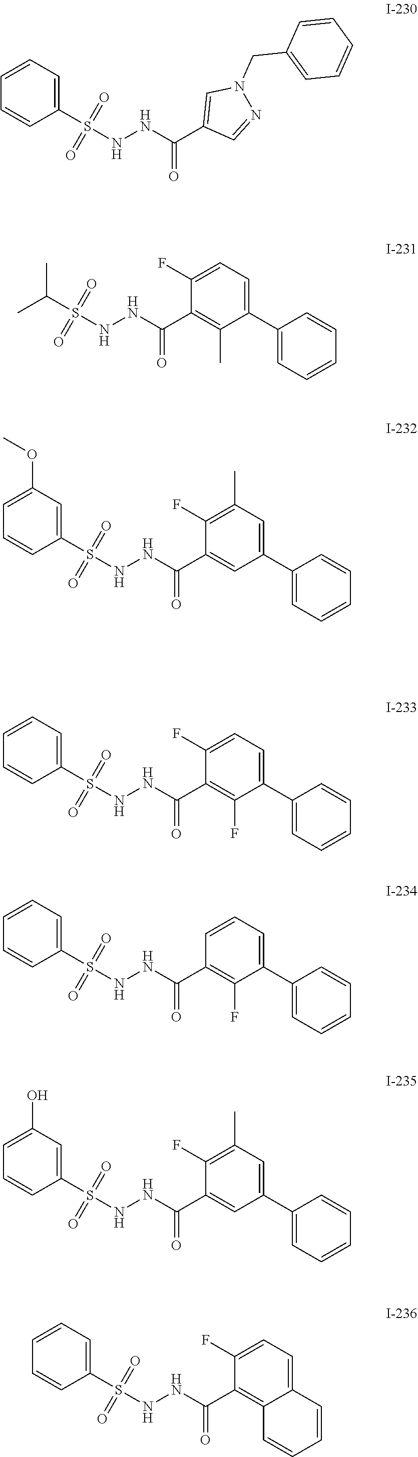

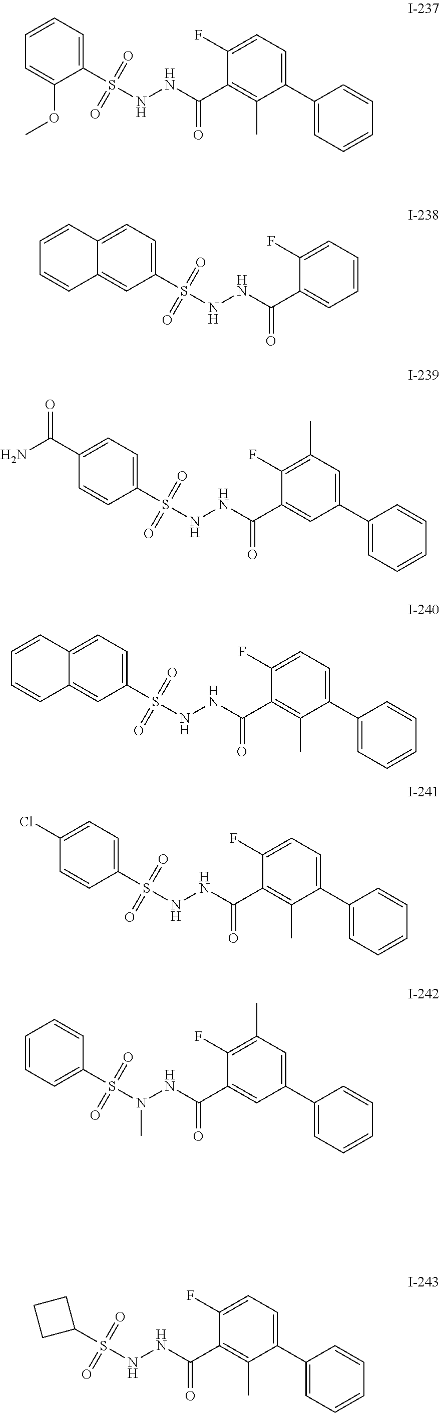

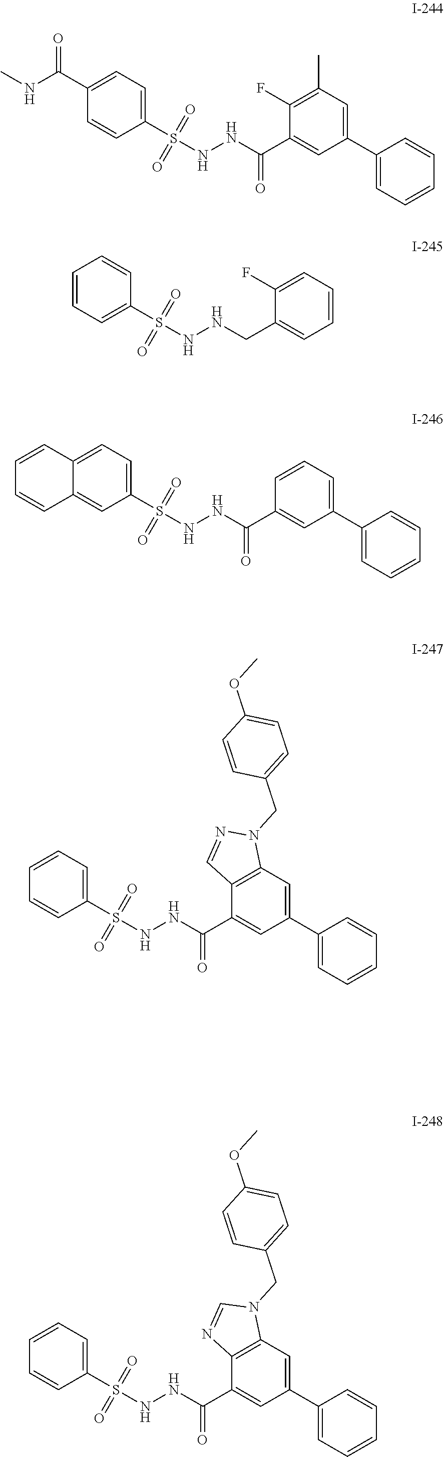 [SMILES: CNC(=O)c1ccc(S(=O)(=O)NNC(=O)c2cc(-c3ccccc3)cc(C)c2F)cc1 ; O=S(=O)(NNCc1ccccc1F)c1ccccc1 ; O=C(NNS(=O)(=O)c1ccc2ccccc2c1)c1cccc(-c2ccccc2)c1 ; COc1ccc(Cn2ncc3c(C(=O)NNS(=O)(=O)c4ccccc4)cc(-c4ccccc4)cc32)cc1 ; COc1ccc(Cn2cnc3c(C(=O)NNS(=O)(=O)c4ccccc4)cc(-c4ccccc4)cc32)cc1]

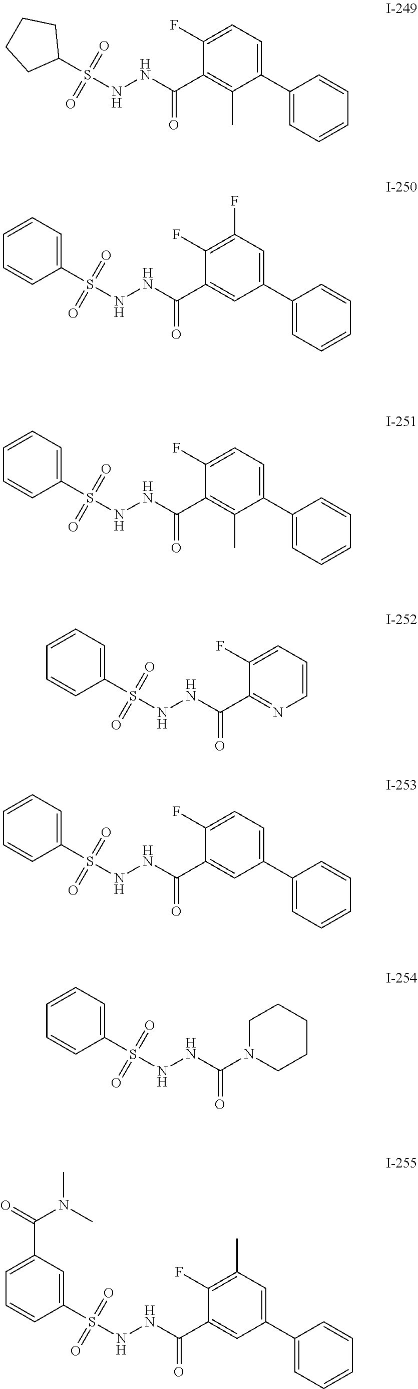

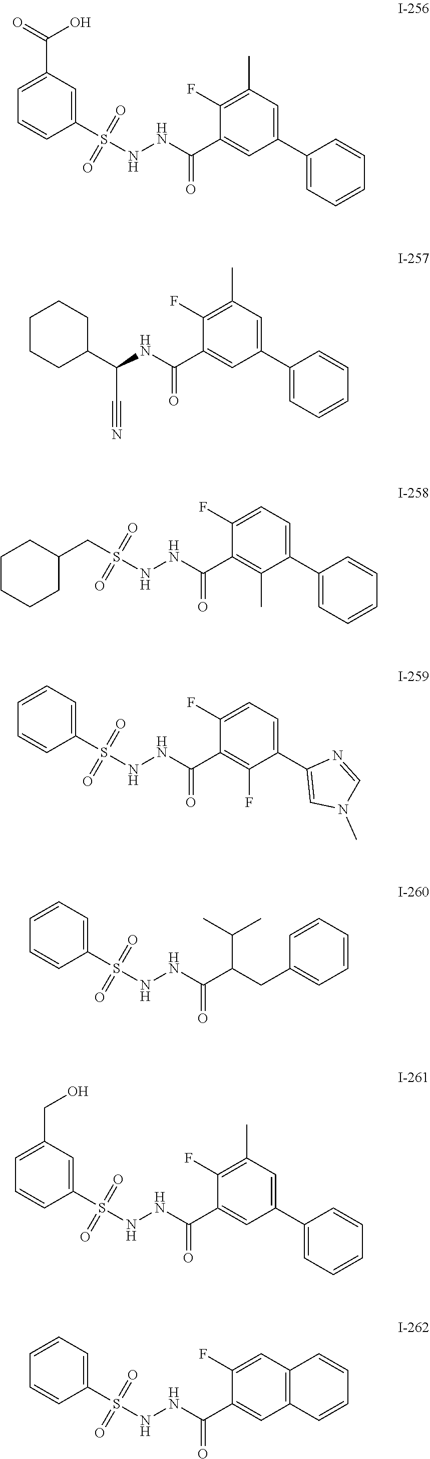

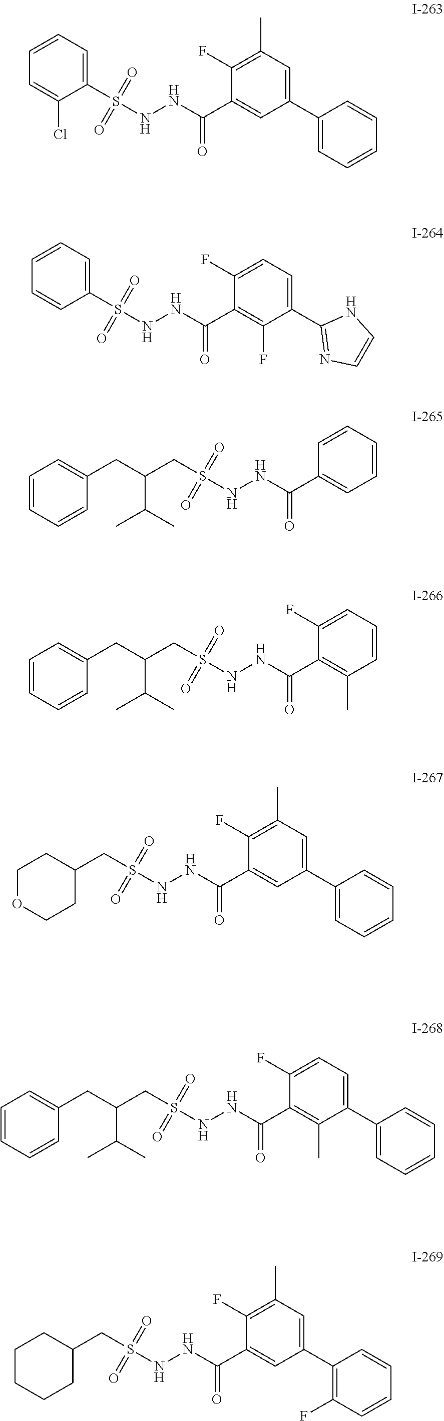

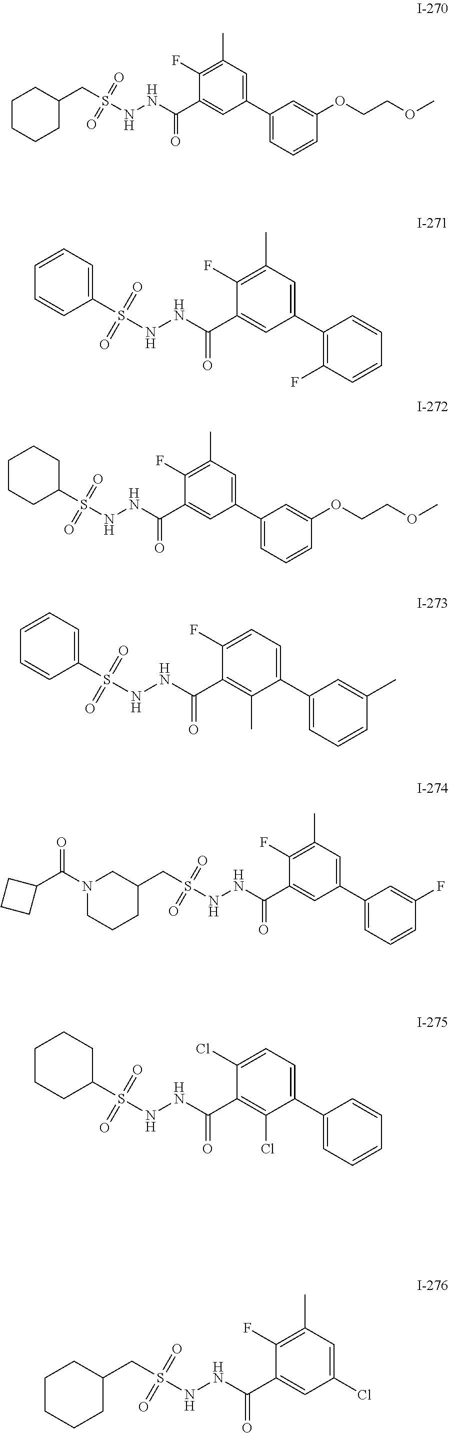

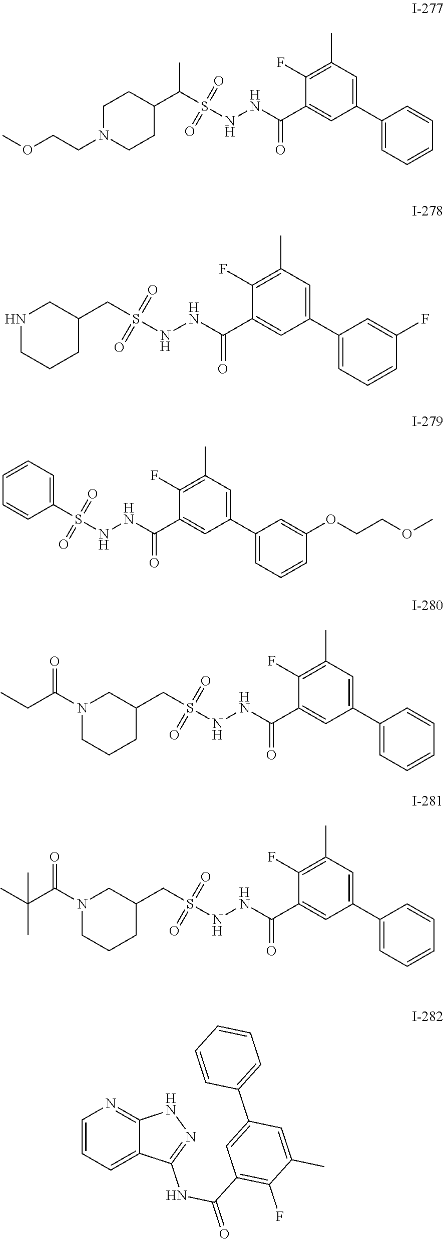

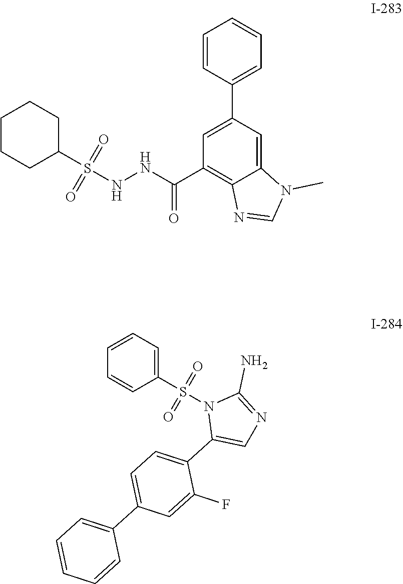 [SMILES: Cn1cnc2c(C(=O)NNS(=O)(=O)C3CCCCC3)cc(-c3ccccc3)cc21 ; Nc1ncc(-c2ccc(-c3ccccc3)cc2F)n1S(=O)(=O)c1ccccc1]

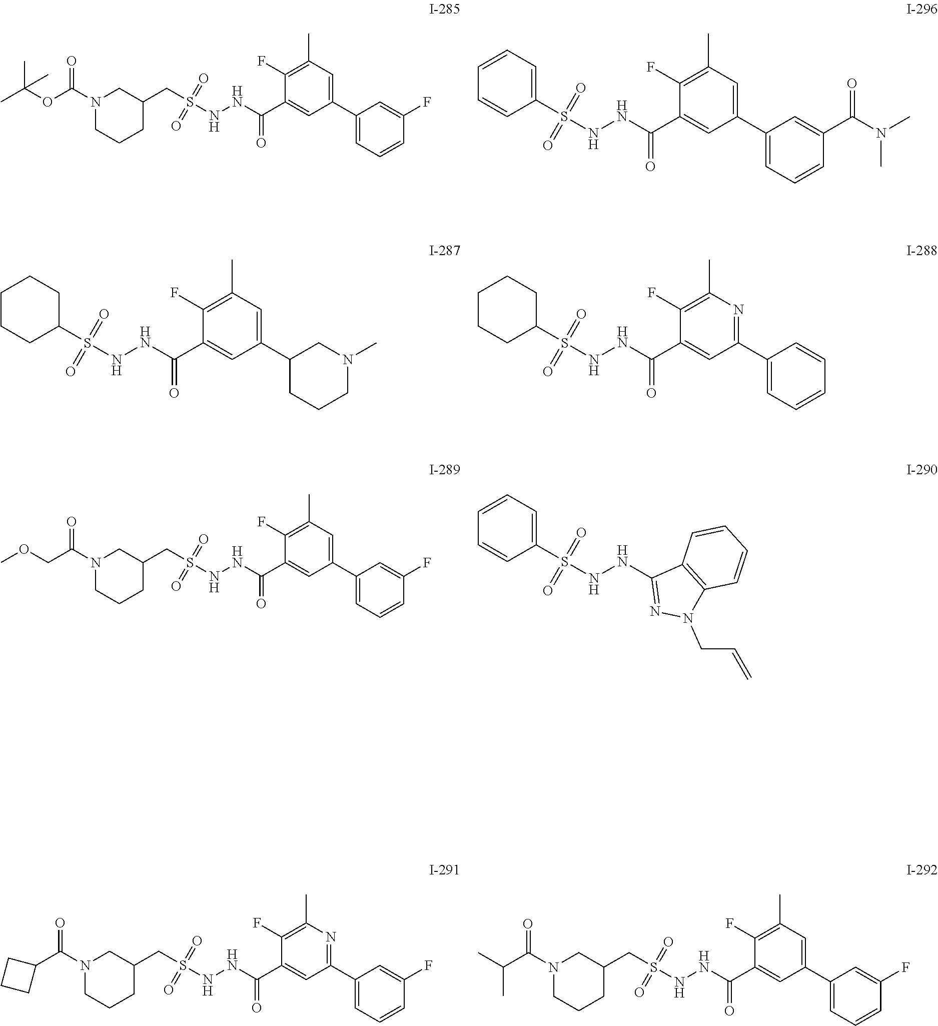

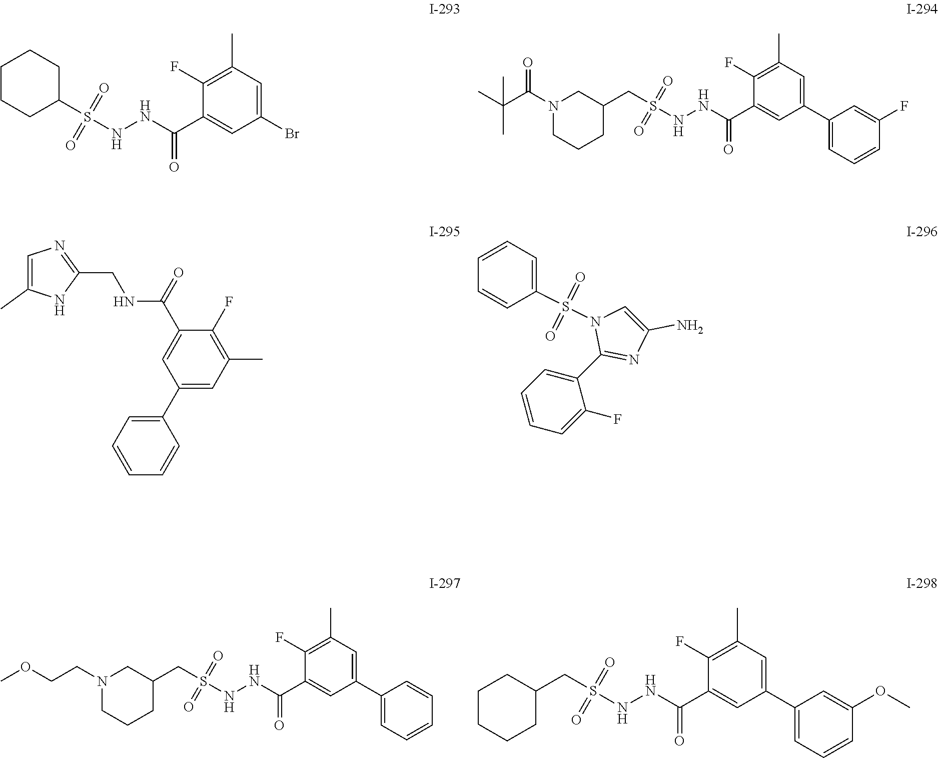

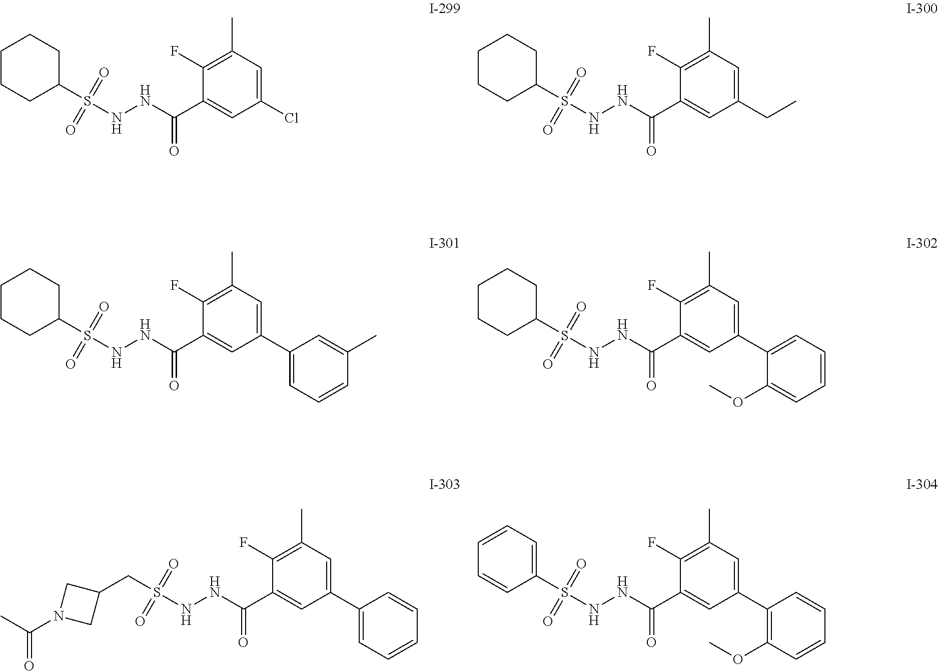 [SMILES: Cc1cc(Cl)cc(C(=O)NNS(=O)(=O)C2CCCCC2)c1F ; CCc1cc(C)c(F)c(C(=O)NNS(=O)(=O)C2CCCCC2)c1 ; Cc1cccc(-c2cc(C)c(F)c(C(=O)NNS(=O)(=O)C3CCCCC3)c2)c1 ; COc1ccccc1-c1cc(C)c(F)c(C(=O)NNS(=O)(=O)C2CCCCC2)c1 ; CC(=O)N1CC(CS(=O)(=O)NNC(=O)c2cc(-c3ccccc3)cc(C)c2F)C1 ; COc1ccccc1-c1cc(C)c(F)c(C(=O)NNS(=O)(=O)c2ccccc2)c1]

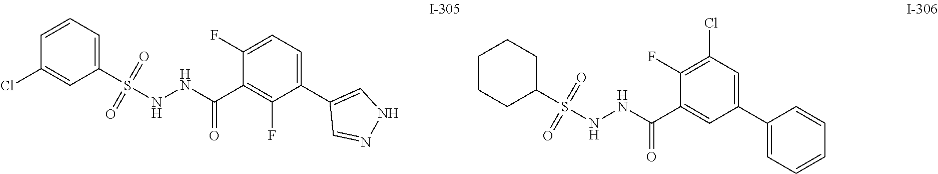 [SMILES: O=C(NNS(=O)(=O)c1cccc(Cl)c1)c1c(F)ccc(-c2cn[nH]c2)c1F ; O=C(NNS(=O)(=O)C1CCCCC1)c1cc(-c2ccccc2)cc(Cl)c1F]

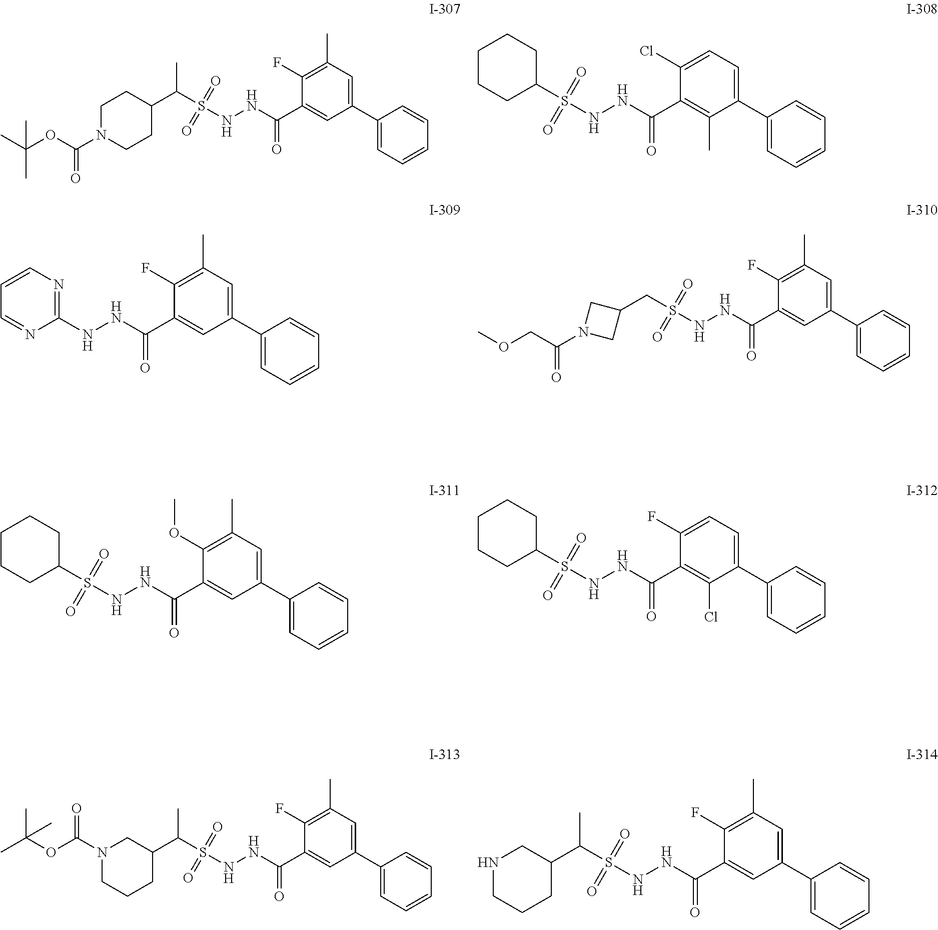

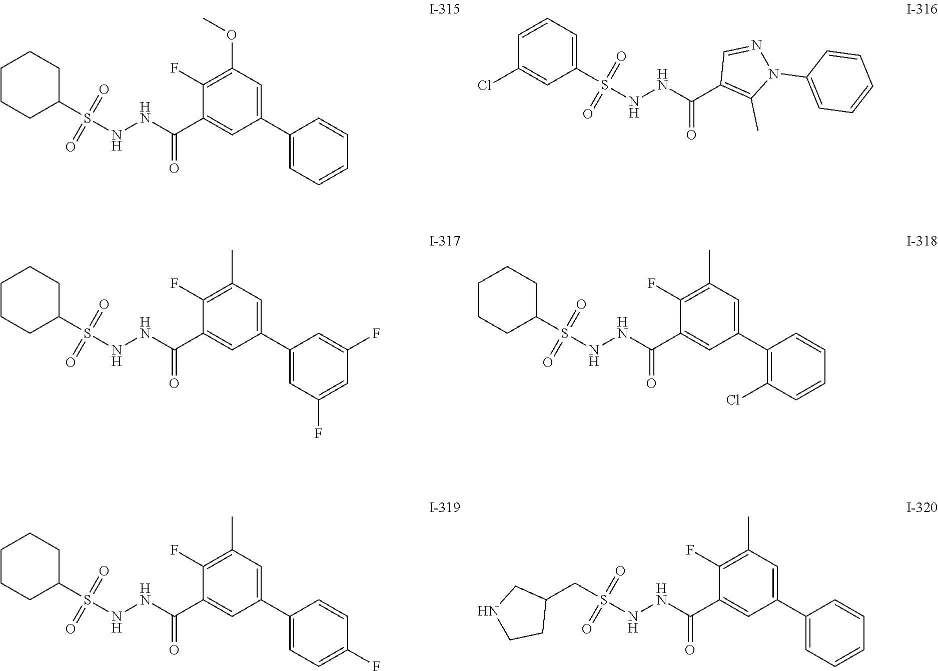 [SMILES: COc1cc(-c2ccccc2)cc(C(=O)NNS(=O)(=O)C2CCCCC2)c1F ; Cc1c(C(=O)NNS(=O)(=O)c2cccc(Cl)c2)cnn1-c1ccccc1 ; Cc1cc(-c2cc(F)cc(F)c2)cc(C(=O)NNS(=O)(=O)C2CCCCC2)c1F ; Cc1cc(-c2ccccc2Cl)cc(C(=O)NNS(=O)(=O)C2CCCCC2)c1F ; Cc1cc(-c2ccc(F)cc2)cc(C(=O)NNS(=O)(=O)C2CCCCC2)c1F ; Cc1cc(-c2ccccc2)cc(C(=O)NNS(=O)(=O)CC2CCNC2)c1F]

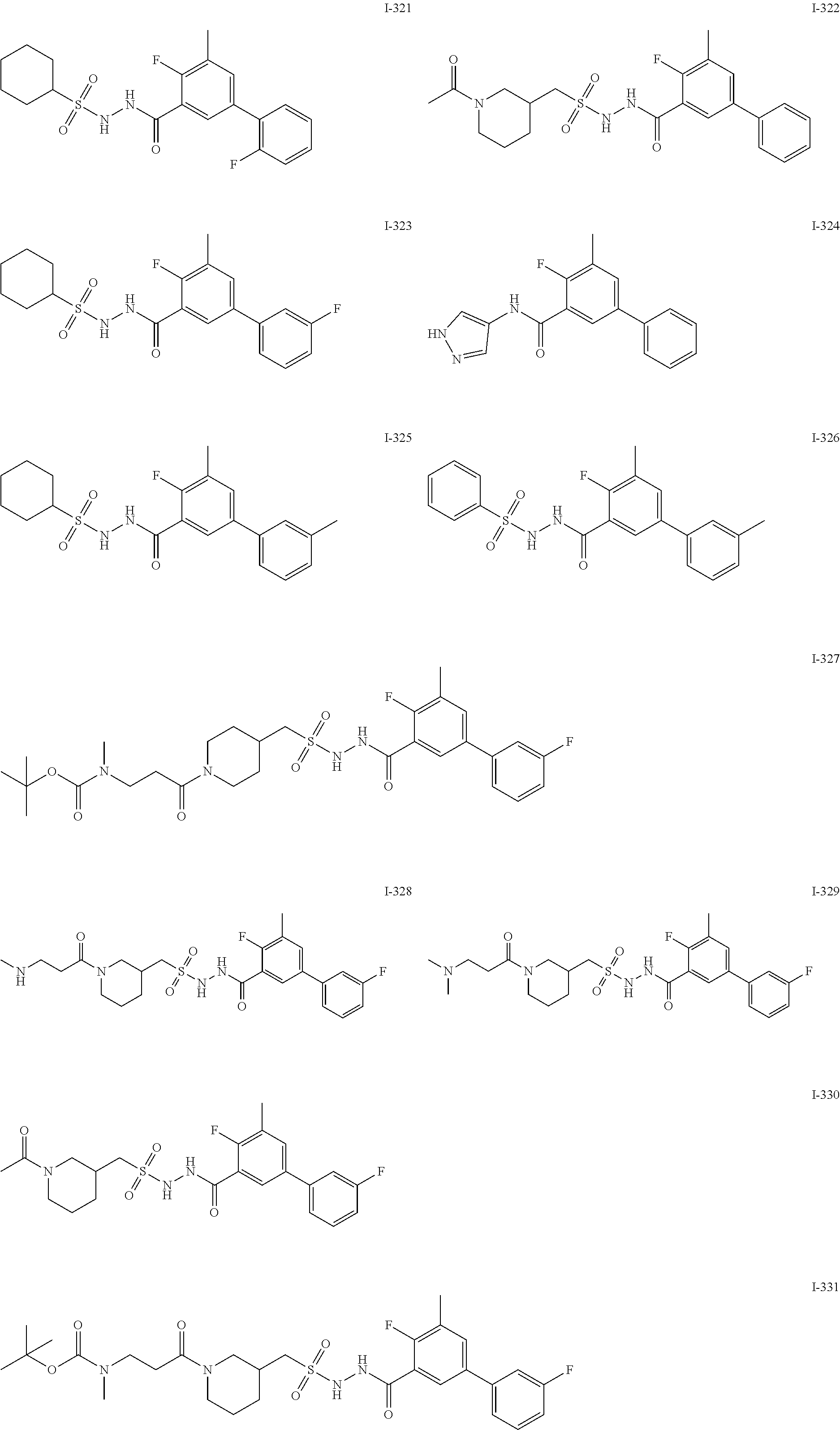

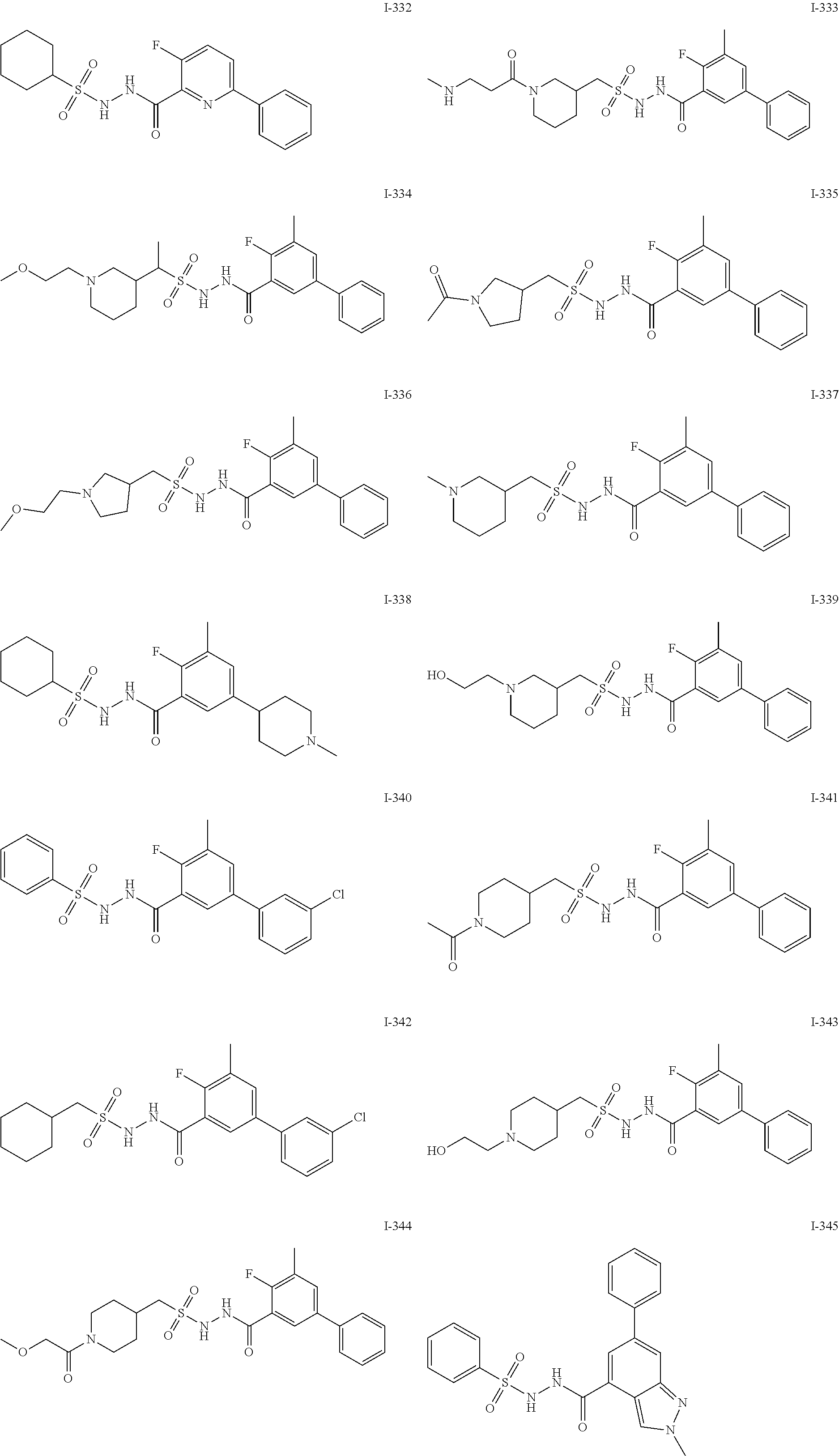

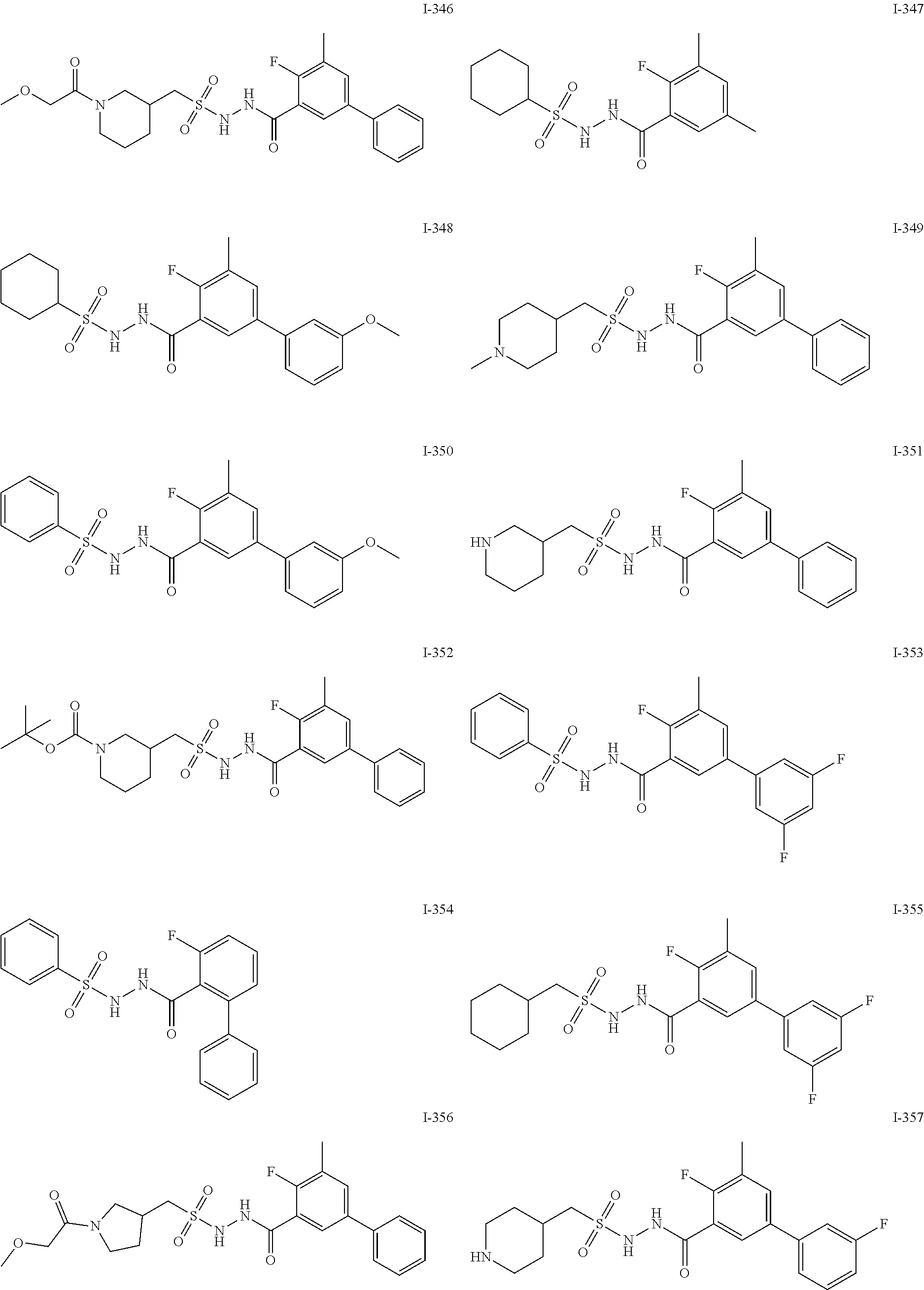

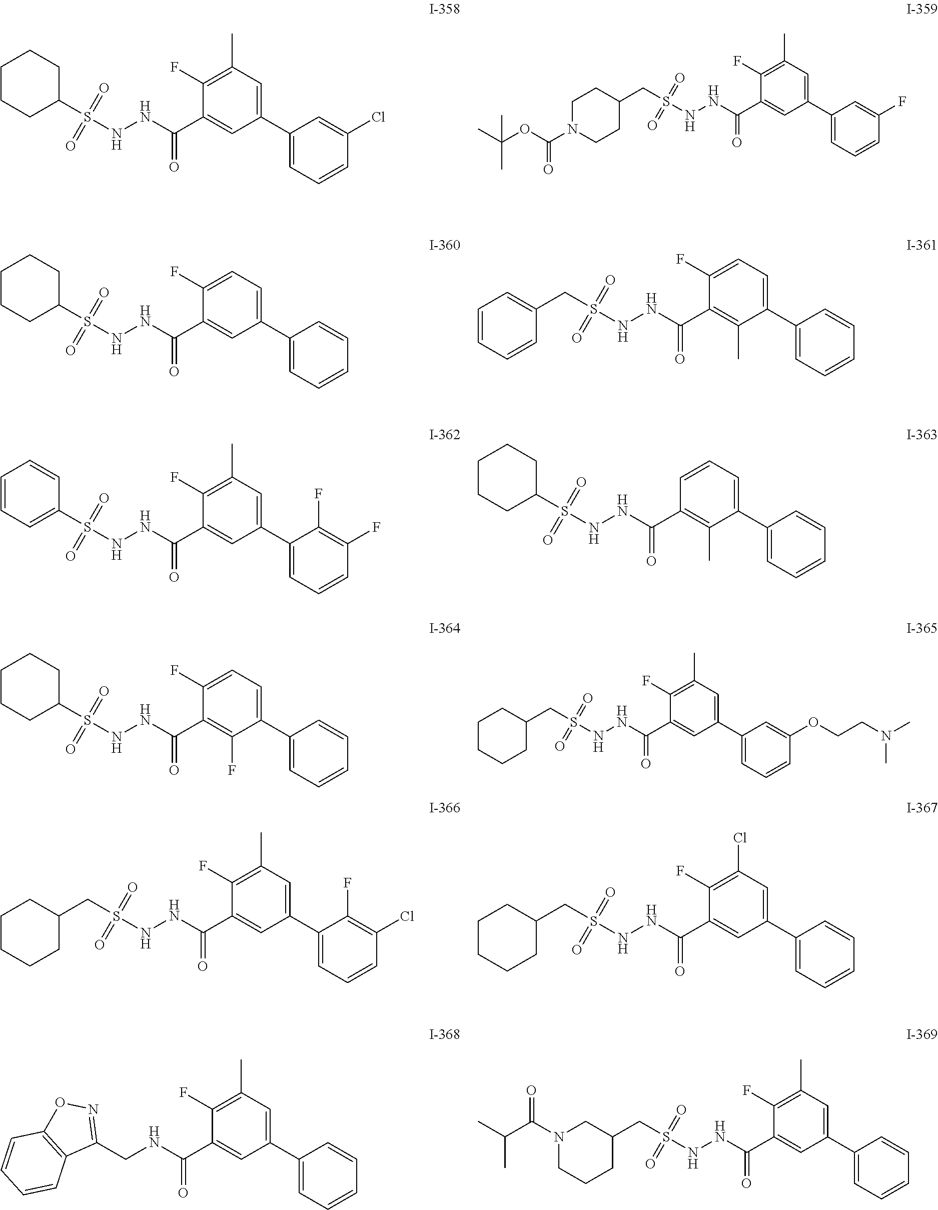

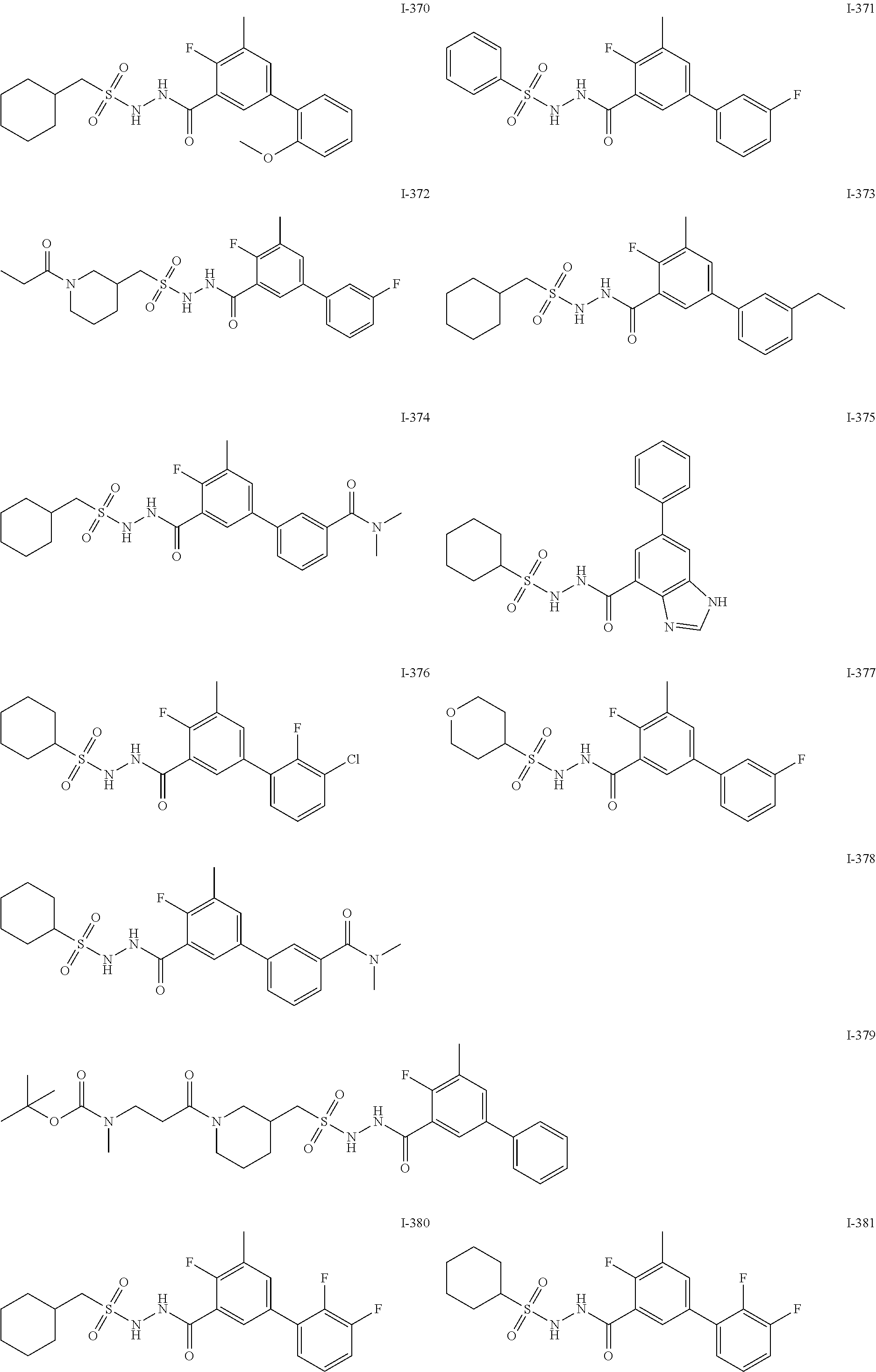

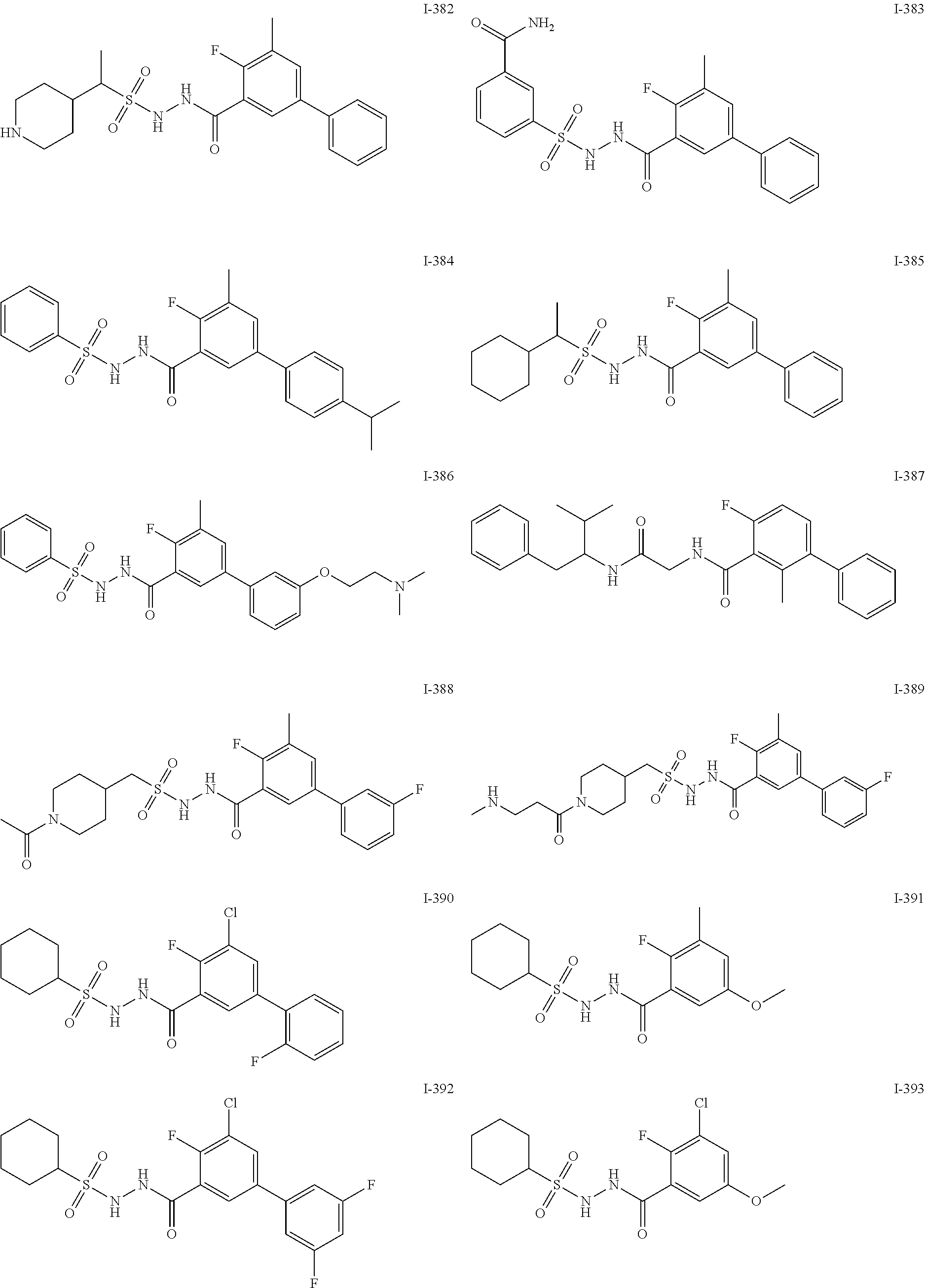

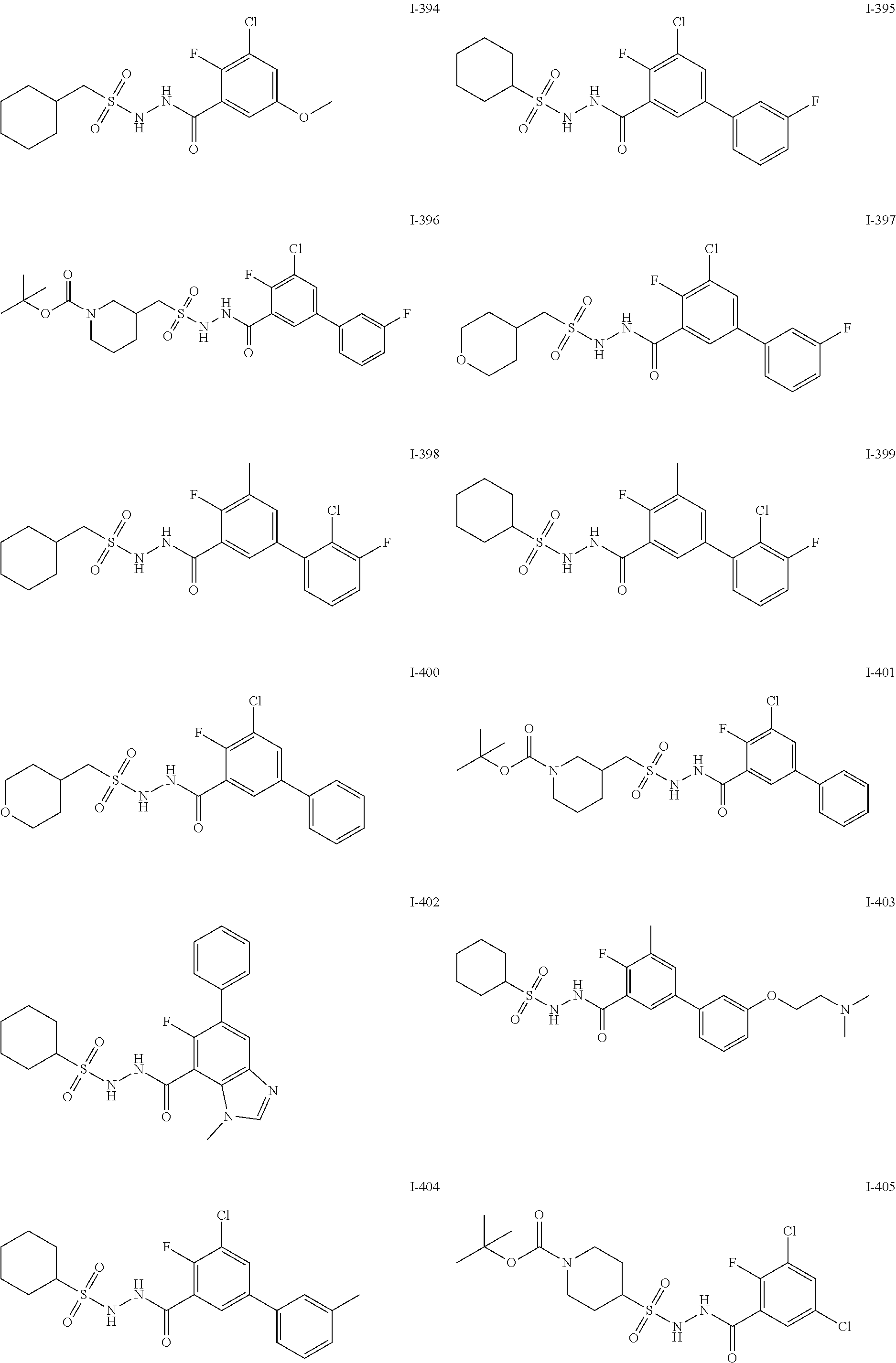

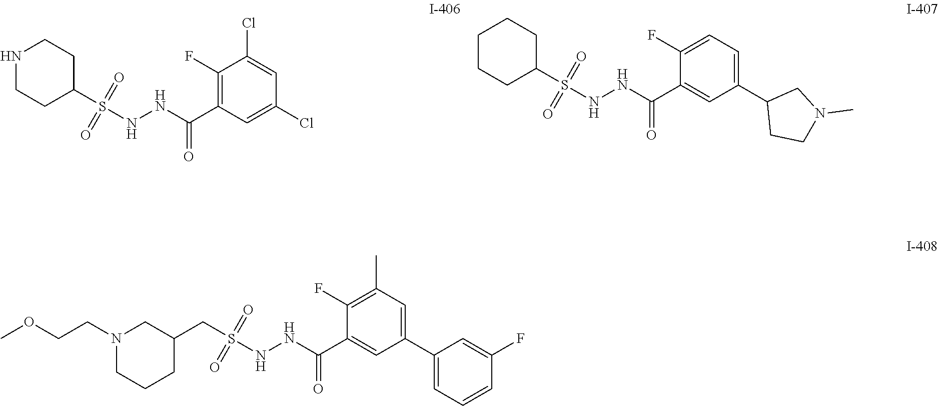 [SMILES: O=C(NNS(=O)(=O)C1CCNCC1)c1cc(Cl)cc(Cl)c1F ; CN1CCC(c2ccc(F)c(C(=O)NNS(=O)(=O)C3CCCCC3)c2)C1 ; COCCN1CCCC(CS(=O)(=O)NNC(=O)c2cc(-c3cccc(F)c3)cc(C)c2F)C1]

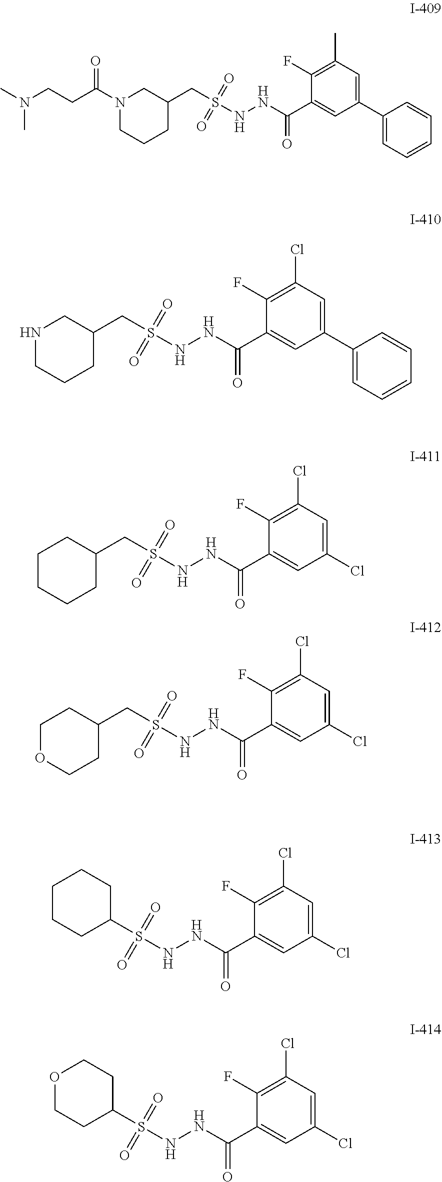 [SMILES: Cc1cc(-c2ccccc2)cc(C(=O)NNS(=O)(=O)CC2CCCN(C(=O)CCN(C)C)C2)c1F ; O=C(NNS(=O)(=O)CC1CCCNC1)c1cc(-c2ccccc2)cc(Cl)c1F ; O=C(NNS(=O)(=O)CC1CCCCC1)c1cc(Cl)cc(Cl)c1F ; O=C(NNS(=O)(=O)CC1CCOCC1)c1cc(Cl)cc(Cl)c1F ; O=C(NNS(=O)(=O)C1CCCCC1)c1cc(Cl)cc(Cl)c1F ; O=C(NNS(=O)(=O)C1CCOCC1)c1cc(Cl)cc(Cl)c1F]

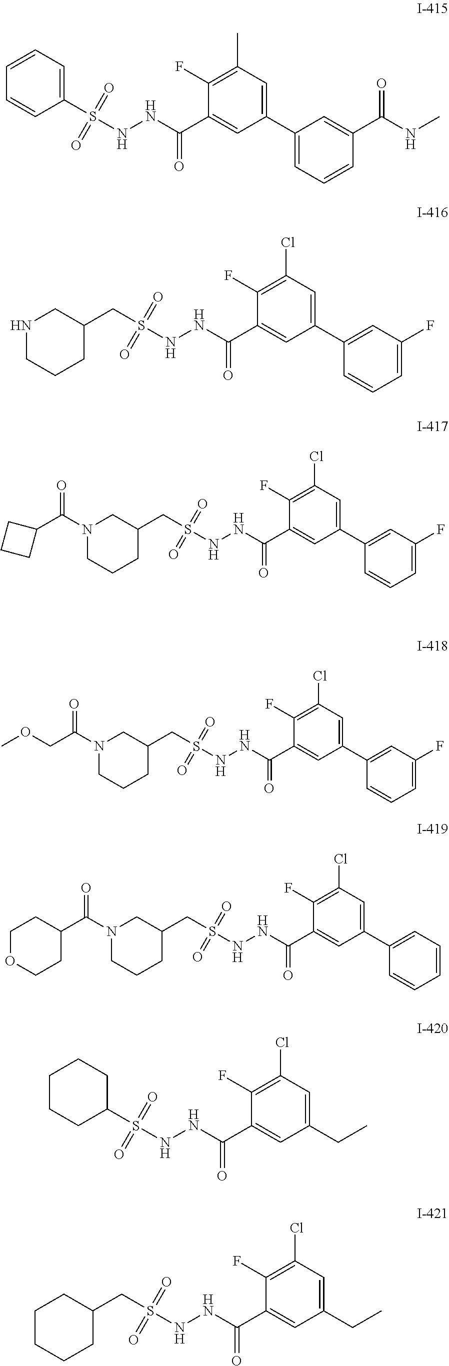

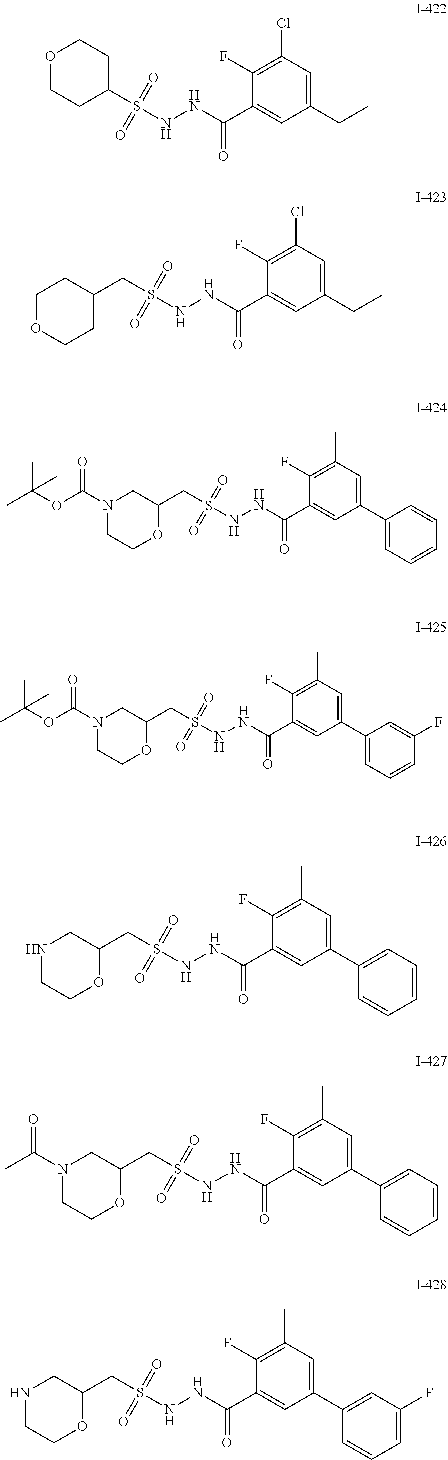

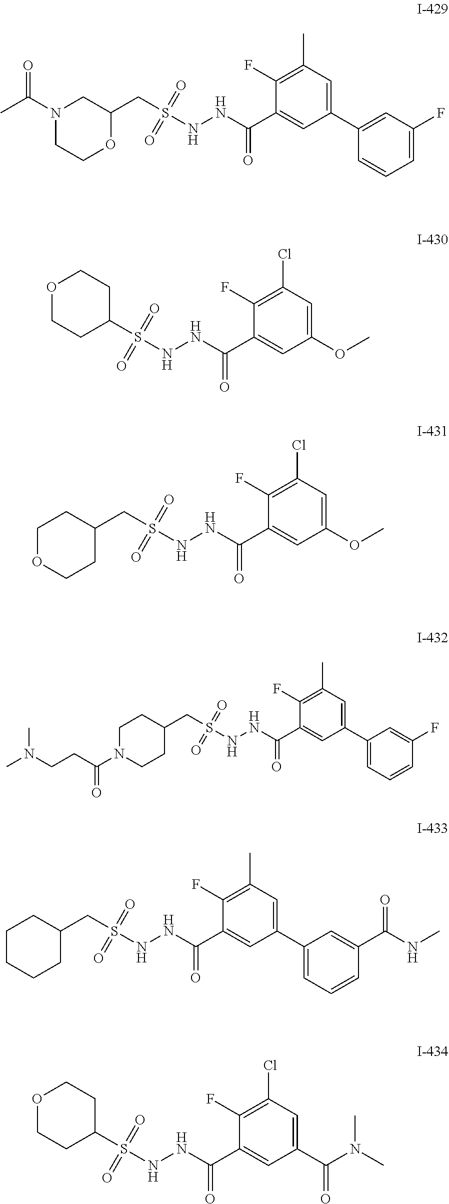

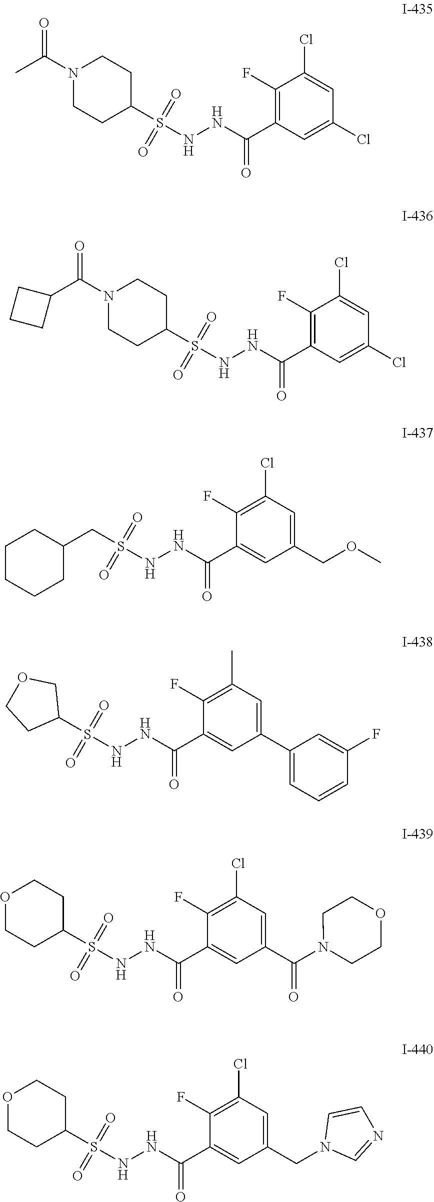 [SMILES: CC(=O)N1CCC(S(=O)(=O)NNC(=O)c2cc(Cl)cc(Cl)c2F)CC1 ; O=C(NNS(=O)(=O)C1CCN(C(=O)C2CCC2)CC1)c1cc(Cl)cc(Cl)c1F ; COCc1cc(Cl)c(F)c(C(=O)NNS(=O)(=O)CC2CCCCC2)c1 ; Cc1cc(-c2cccc(F)c2)cc(C(=O)NNS(=O)(=O)C2CCOC2)c1F ; O=C(NNS(=O)(=O)C1CCOCC1)c1cc(C(=O)N2CCOCC2)cc(Cl)c1F ; O=C(NNS(=O)(=O)C1CCOCC1)c1cc(Cn2ccnc2)cc(Cl)c1F]

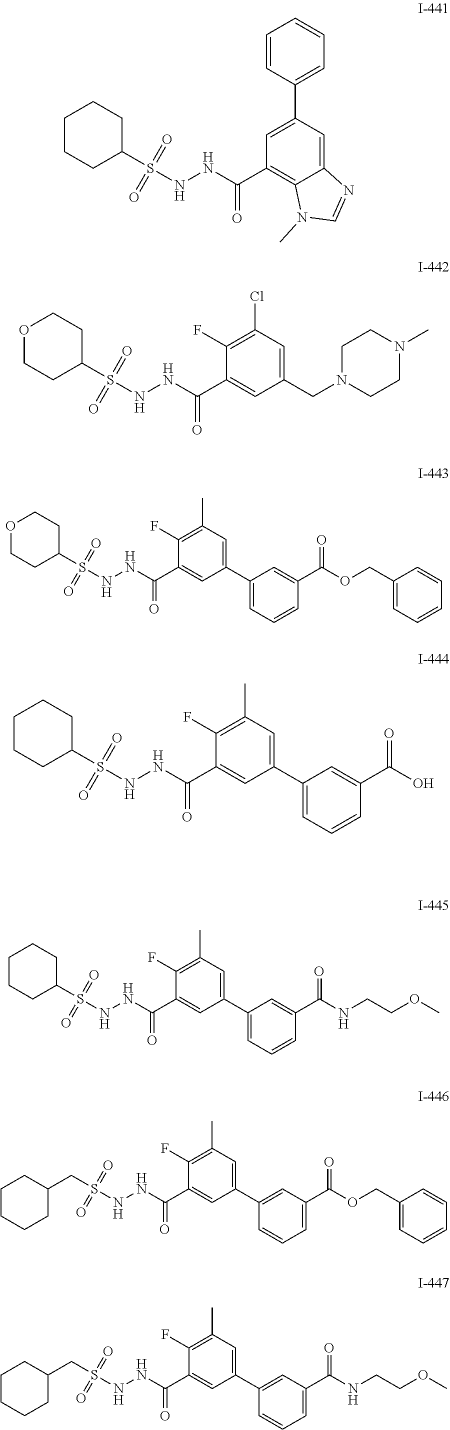

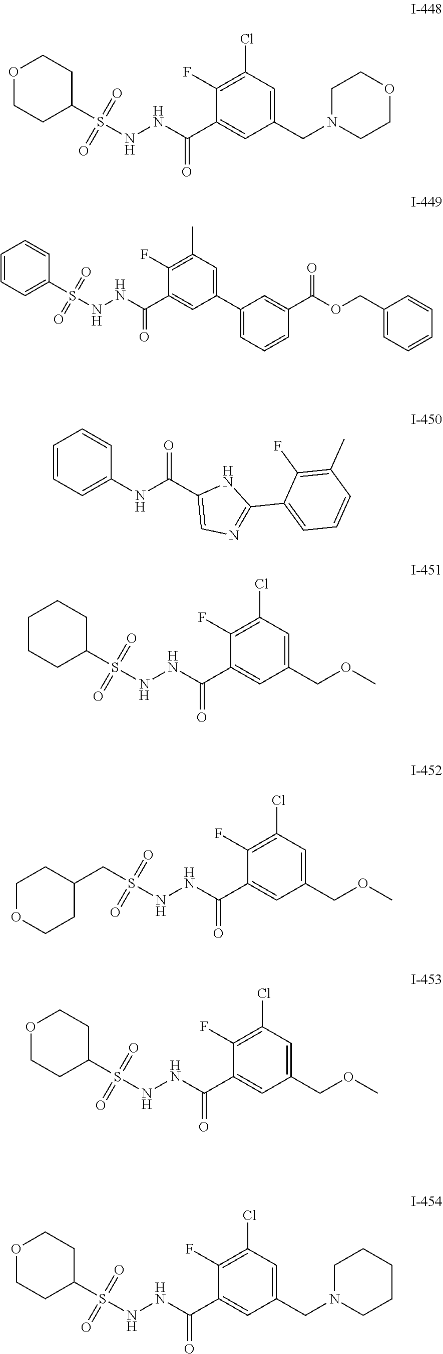

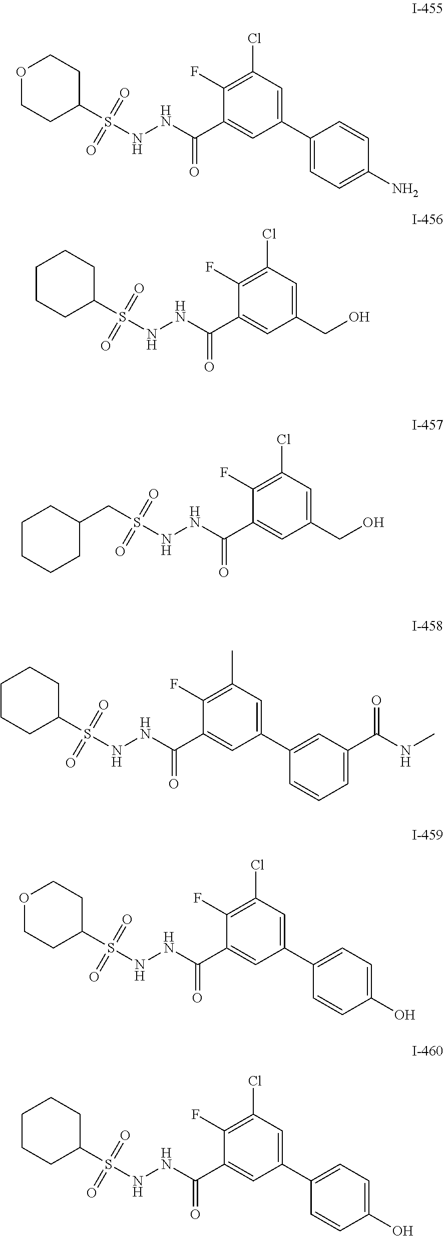 [SMILES: Nc1ccc(-c2cc(Cl)c(F)c(C(=O)NNS(=O)(=O)C3CCOCC3)c2)cc1 ; O=C(NNS(=O)(=O)C1CCCCC1)c1cc(CO)cc(Cl)c1F ; O=C(NNS(=O)(=O)CC1CCCCC1)c1cc(CO)cc(Cl)c1F ; CNC(=O)c1cccc(-c2cc(C)c(F)c(C(=O)NNS(=O)(=O)C3CCCCC3)c2)c1 ; O=C(NNS(=O)(=O)C1CCOCC1)c1cc(-c2ccc(O)cc2)cc(Cl)c1F ; O=C(NNS(=O)(=O)C1CCCCC1)c1cc(-c2ccc(O)cc2)cc(Cl)c1F]

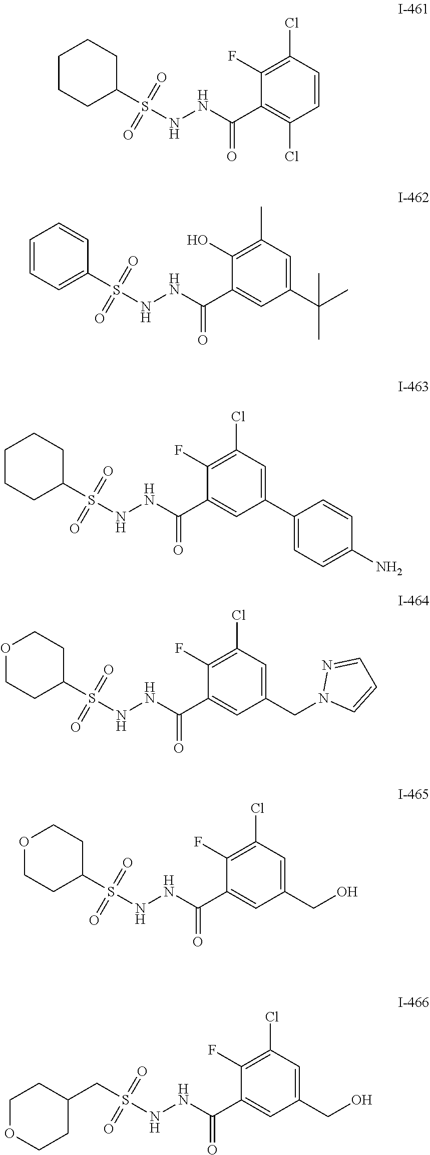 [SMILES: O=C(NNS(=O)(=O)C1CCCCC1)c1c(Cl)ccc(Cl)c1F ; Cc1cc(C(C)(C)C)cc(C(=O)NNS(=O)(=O)c2ccccc2)c1O ; Nc1ccc(-c2cc(Cl)c(F)c(C(=O)NNS(=O)(=O)C3CCCCC3)c2)cc1 ; O=C(NNS(=O)(=O)C1CCOCC1)c1cc(Cn2cccn2)cc(Cl)c1F ; O=C(NNS(=O)(=O)C1CCOCC1)c1cc(CO)cc(Cl)c1F ; O=C(NNS(=O)(=O)CC1CCOCC1)c1cc(CO)cc(Cl)c1F]

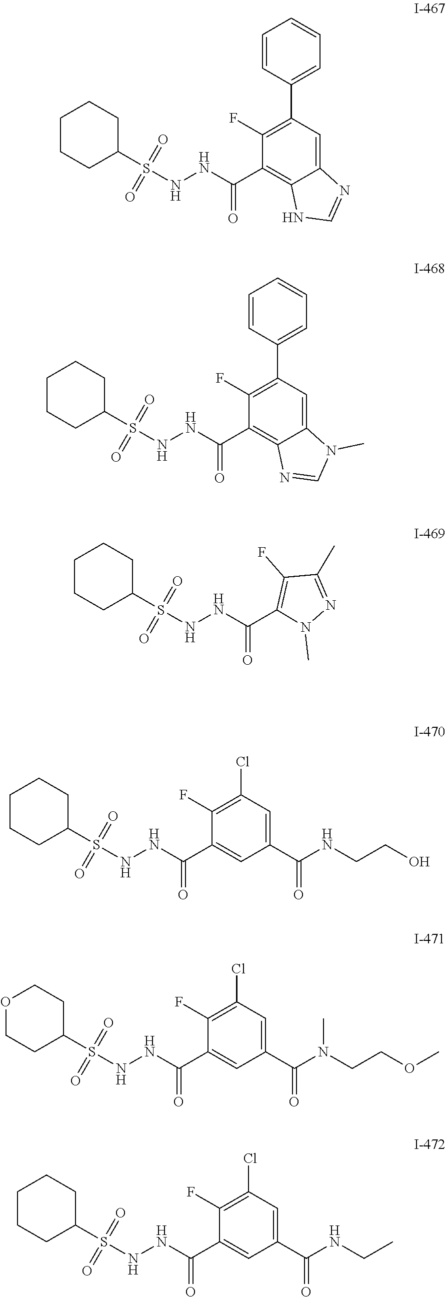 [SMILES: O=C(NNS(=O)(=O)C1CCCCC1)c1c(F)c(-c2ccccc2)cc2nc[nH]c12 ; Cn1cnc2c(C(=O)NNS(=O)(=O)C3CCCCC3)c(F)c(-c3ccccc3)cc21 ; Cc1nn(C)c(C(=O)NNS(=O)(=O)C2CCCCC2)c1F ; O=C(NCCO)c1cc(Cl)c(F)c(C(=O)NNS(=O)(=O)C2CCCCC2)c1 ; COCCN(C)C(=O)c1cc(Cl)c(F)c(C(=O)NNS(=O)(=O)C2CCOCC2)c1 ; CCNC(=O)c1cc(Cl)c(F)c(C(=O)NNS(=O)(=O)C2CCCCC2)c1]

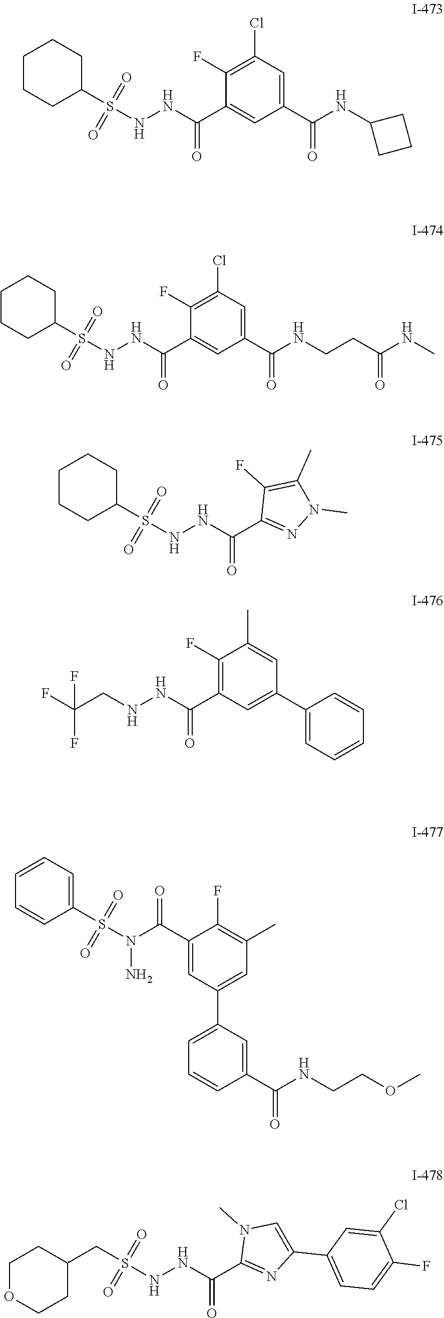

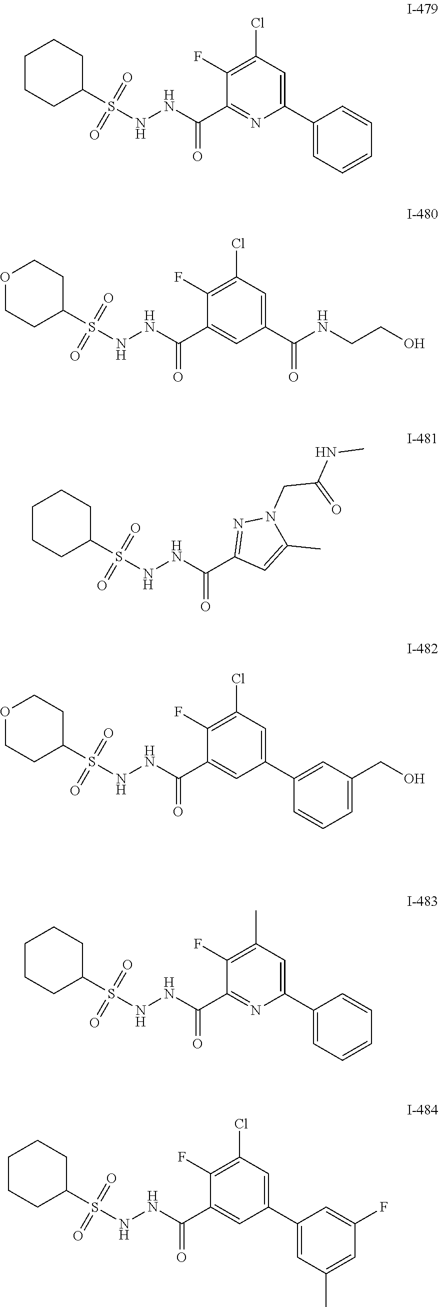 [SMILES: O=C(NNS(=O)(=O)C1CCCCC1)c1nc(-c2ccccc2)cc(Cl)c1F ; O=C(NCCO)c1cc(Cl)c(F)c(C(=O)NNS(=O)(=O)C2CCOCC2)c1 ; CNC(=O)Cn1nc(C(=O)NNS(=O)(=O)C2CCCCC2)cc1C ; O=C(NNS(=O)(=O)C1CCOCC1)c1cc(-c2cccc(CO)c2)cc(Cl)c1F ; Cc1cc(-c2ccccc2)nc(C(=O)NNS(=O)(=O)C2CCCCC2)c1F ; Cc1cc(F)cc(-c2cc(Cl)c(F)c(C(=O)NNS(=O)(=O)C3CCCCC3)c2)c1]

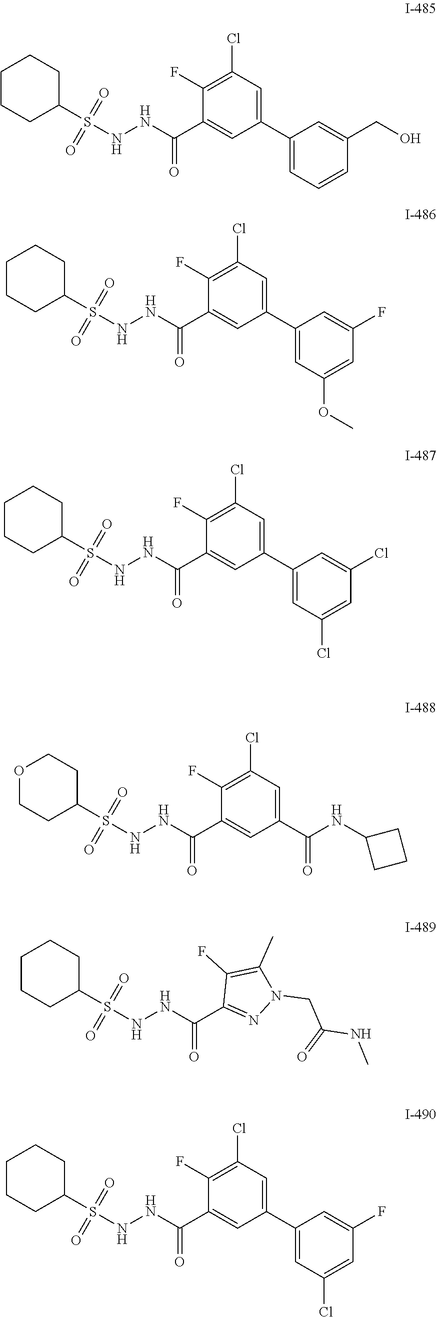 [SMILES: O=C(NNS(=O)(=O)C1CCCCC1)c1cc(-c2cccc(CO)c2)cc(Cl)c1F ; COc1cc(F)cc(-c2cc(Cl)c(F)c(C(=O)NNS(=O)(=O)C3CCCCC3)c2)c1 ; O=C(NNS(=O)(=O)C1CCCCC1)c1cc(-c2cc(Cl)cc(Cl)c2)cc(Cl)c1F ; O=C(NC1CCC1)c1cc(Cl)c(F)c(C(=O)NNS(=O)(=O)C2CCOCC2)c1 ; CNC(=O)Cn1nc(C(=O)NNS(=O)(=O)C2CCCCC2)c(F)c1C ; O=C(NNS(=O)(=O)C1CCCCC1)c1cc(-c2cc(F)cc(Cl)c2)cc(Cl)c1F]

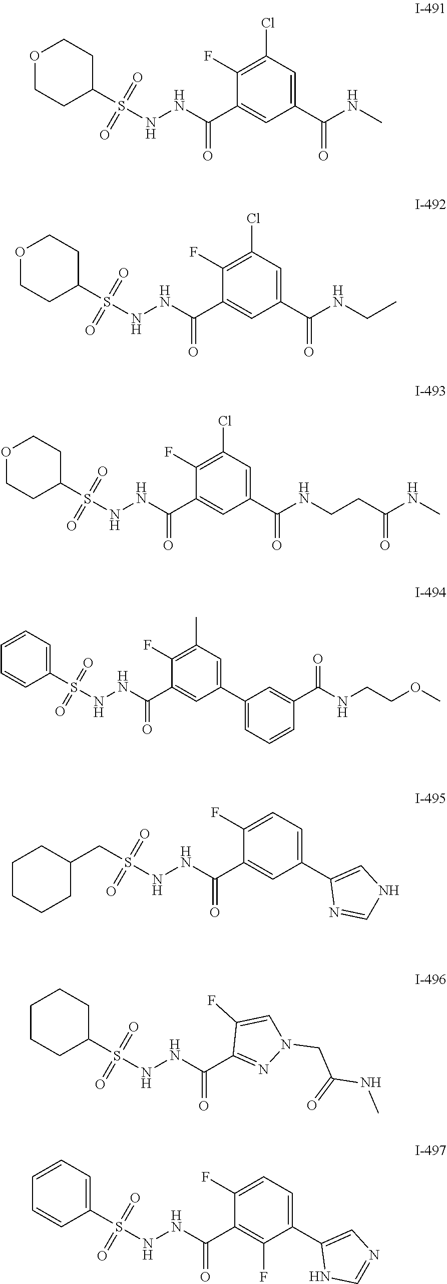

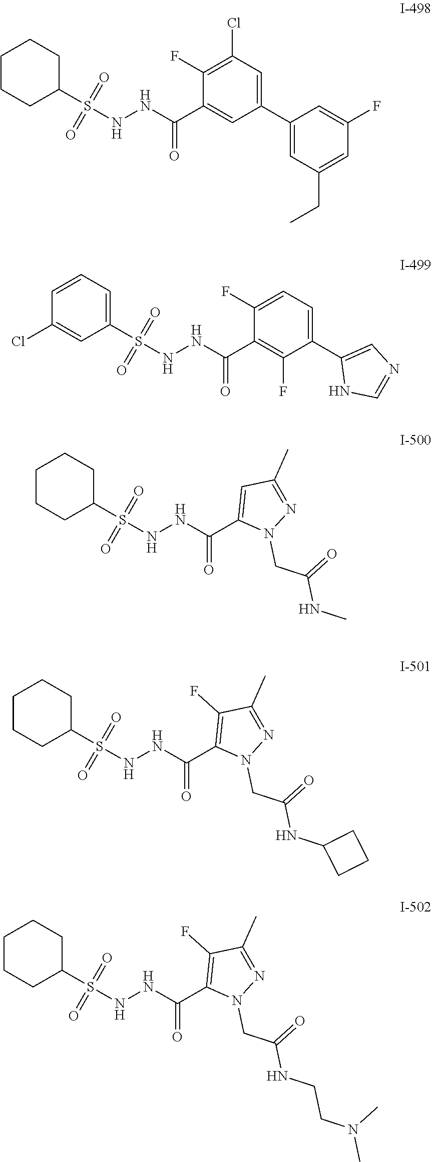 [SMILES: CCc1cc(F)cc(-c2cc(Cl)c(F)c(C(=O)NNS(=O)(=O)C3CCCCC3)c2)c1 ; O=C(NNS(=O)(=O)c1cccc(Cl)c1)c1c(F)ccc(-c2cnc[nH]2)c1F ; CNC(=O)Cn1nc(C)cc1C(=O)NNS(=O)(=O)C1CCCCC1 ; Cc1nn(CC(=O)NC2CCC2)c(C(=O)NNS(=O)(=O)C2CCCCC2)c1F ; Cc1nn(CC(=O)NCCN(C)C)c(C(=O)NNS(=O)(=O)C2CCCCC2)c1F]

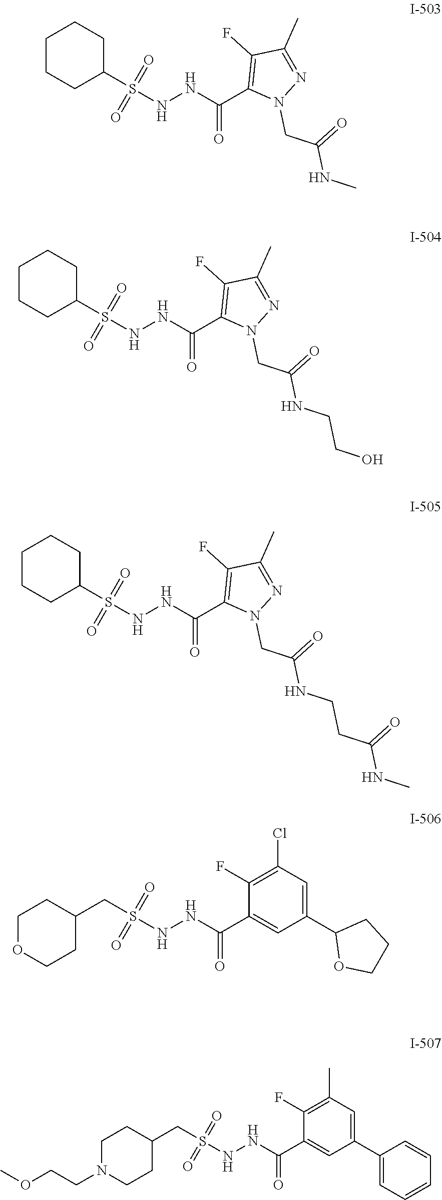

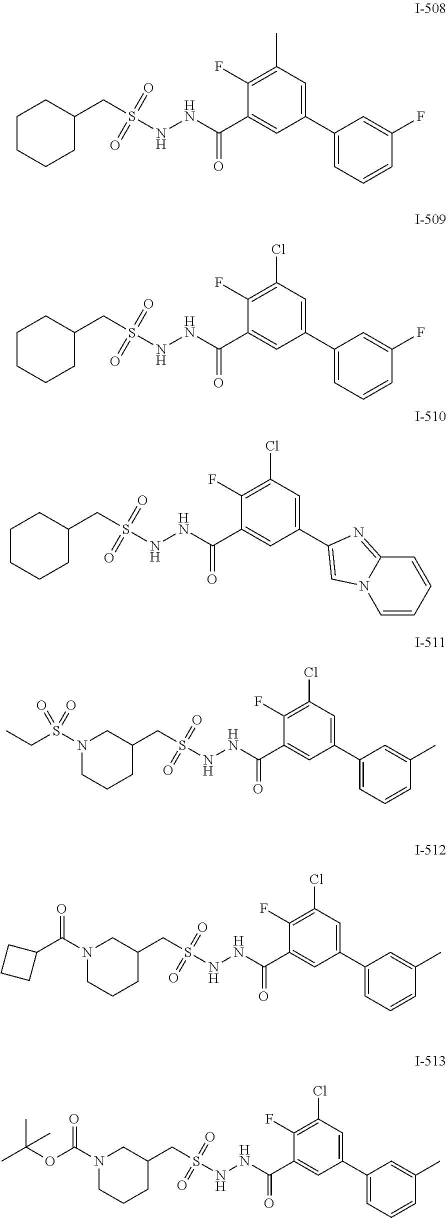

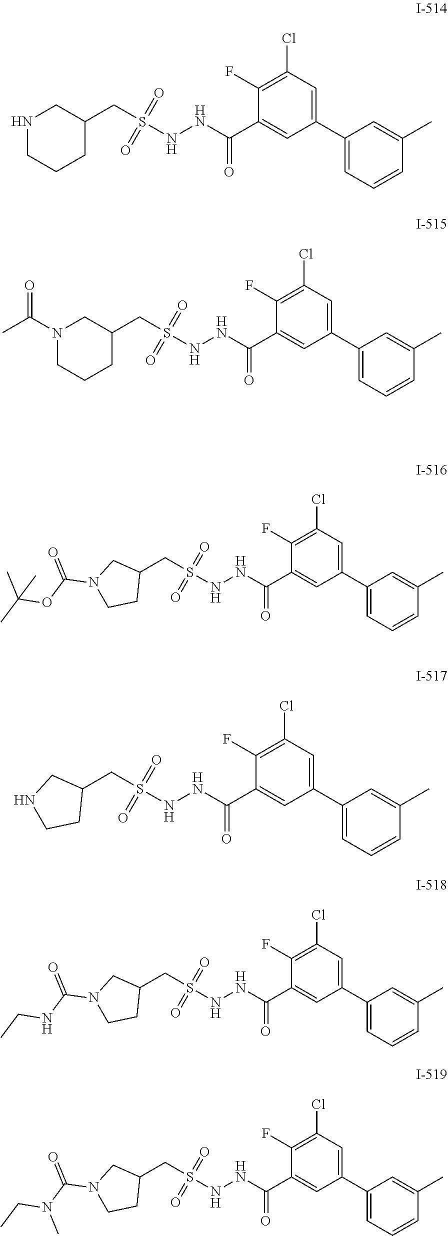

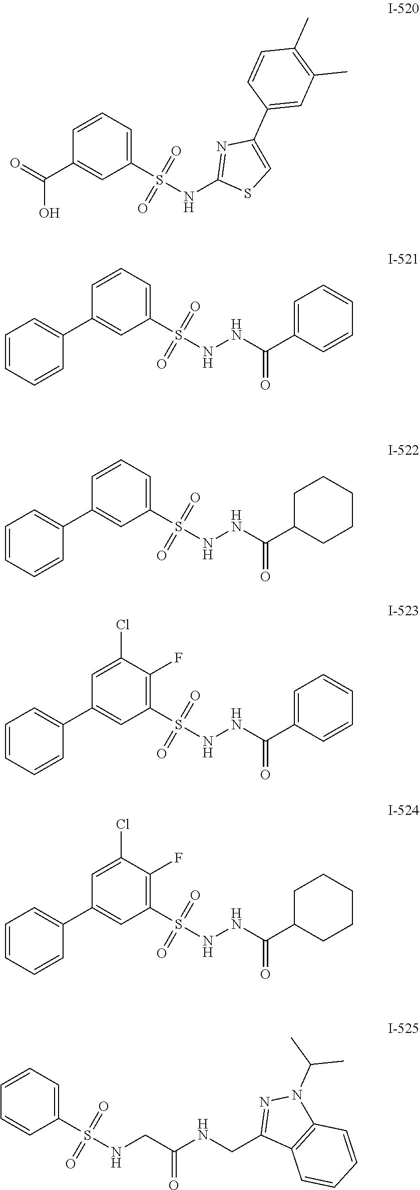 [SMILES: Cc1ccc(-c2csc(NS(=O)(=O)c3cccc(C(=O)O)c3)n2)cc1C ; O=C(NNS(=O)(=O)c1cccc(-c2ccccc2)c1)c1ccccc1 ; O=C(NNS(=O)(=O)c1cccc(-c2ccccc2)c1)C1CCCCC1 ; O=C(NNS(=O)(=O)c1cc(-c2ccccc2)cc(Cl)c1F)c1ccccc1 ; O=C(NNS(=O)(=O)c1cc(-c2ccccc2)cc(Cl)c1F)C1CCCCC1 ; CC(C)n1nc(CNC(=O)CNS(=O)(=O)c2ccccc2)c2ccccc21]

or a pharmaceutically acceptable salt thereof.

### Acetyl Transferases

Histone acetylation and deacetylation are processes by which lysine residues within the N-terminal tail protruding from histone cores of the nucleosome are acetylated and deacetylated. Without wishing to be hound by any particular theory, it is believed that histone acetylation is a part of gene regulation. Histone Acetyltransferases, also known as HATs, are a family of enzymes that acetylate the histone tails of the nucleosome among other nuclear and cytoplasmic non-histone targets. Some HATS acetylate a lysine residue, and such Lysine Acetyltransferases are also referred to as KATs.

KATs can be divided into families based on their structure and sequence similarity. KAT families include, for example, the Gcn5-related N-acetyltransferase (GNAT) family, which includes GCN5 and PCAF, the CREBBP/EP300 family and the MYST (MOZ, Ybf2/Sas3, Sas2, Tip60) family, which includes Tat interacting protein, 60 kDa (Tip60), monocytic leukemia zinc finger protein/MOZ-related factor protein (MOZ/MORF). Different KATs may contain various other domains in addition to the HAT domain which facilitate interactions with other proteins, including reader domains for acetylation and other modifications. See, e.g., Farria et al. Oncogene (2015) 34, 4901-4913, incorporated herein by reference. Some KATs, for example those in the GNAT and CREBBP/EP300 families, contain bromodomains. Bromodomains help KATs recognize and bind to acetylated lysine residues on histone substrates. Together these domains allow for specificity and diversity in KAT substrates. All KATs examined to date have important functions in cellular differentiation and embryo development. Several KATs have also been associated with oncogenesis. For example, CREBBP/EP300, have been implicated in cancer development and progression. See, e.g., Farria et al. Oncogene (2015) 34, 4901-4913; Lee et al, Nat. Rev. Mol. Cell Biol. 8 (4): 284-95; and Avvakumov et al. Oncogene (2007) 26, 5395-5407, the entire contents of each of which are incorporated herein by reference, Inhibitors of KATs and histone deacetylase inhibitors (HDACs) have potential as anti-cancer therapies.

KAT-5, also known as Lysine Acetyltransferase 5, TIP60, or HTATIP, belongs to the MYST family of histone acetyl transferases and was originally isolated as an HIV-1 TAT-interactive protein. KAT-5 has been reported to play important roles in regulating chromatin remodeling, transcription. DNA repair, and apoptosis, and is also thought to play an important role in signal transduction. Alternative splicing of this gene results in multiple transcript variants. The protein sequences of exemplary KAT-5 proteins have been reported. Exemplary human KAT-5 protein sequences include, for example, and without limitation, the sequences provided below. Additional KAT-5 sequences, including KAT5-sequences from other species and additional human KAT-5 sequences will be apparent to those of ordinal), skill in the art, and include, for example, and without limitation, those KAT-5 sequences listed in the NCBI and ENSEMBL gene databases.

>NP_874369. 1 histone acetyltransferase KAT5
isoform 1 [Homo sapiens]
(SEQ ID NO: 1)
MAEVVSPVPGAGRREPGEVGRARGPPVADPGVALSPQGEIIEGCRLPVLR
RNQDNEDEWPLAEILSVKDISGRKLFYVHYIDFNKRLDEWVTHERLDLKK
IQFPKKEAKTPTKNGLPGSRPGSPEREVPASAQASGKTLPIPVQITLRFN
LPKEREAIPGGEPDQPLSSSSCLQPNHRSTKRKVEVVSPATPVPSETAPA
SVFPQNGAARRAVAAQPGRKRKSNCLGTDEDSQDSSDGIPSAPRMTGSLV
SDRSHDDIVTRMKNIECIELGRHRLKTWYFSPYPQELTTLPVLYLCEFCL
KYGRSLKCLQRHLTKCDLRHPPGNEIYRKGTISFFEIDGRKNKSYSQNLC
LLAKCFLDHKTLYYDTDPFLFYVMTEYDCKGFHIVGYFSKEKESTEDYNV
ACILTLPPYQRRGYGKLLIEFSYELSKVEGKTGTPEKPLSDLGLLSYRSY
WSQTILEILMGLKSESGERPQITINEISEITSLKKEDVISTLQYLNLINY
YKGQYILTLSEDIVDGHERAMLKRLLRIDSKCLHFTPKDWSKRGKW
>NP_006379.2 histone acetyltransferase KAT5
isoform 2 [Homo sapiens]
(SEQ ID NO: 2)
MAEVGEIIEGCRLPVLRRNQDNEDEWPLAEILSVKDISGRKLFYVHYIDF
NKRLDEWVTHERLDLKKIQFPKKEAKTPTKNGLPGSRPGSPEREVPASAQ
ASGKTLPIPVQITLRFNLPKEREAIPGGEPDQPLSSSSCLQPNHRSTKRK
VEVVSPATPVPSETAPASVFPQNGAARRAVAAQPGRKRKSNCLGTDEDSQ
DSSDGIPSAPRMTGSLVSDRSHDDIVTRMKNIECIELGRHRLKPWYFSPY
PQELTTLPVLYLCEFCLKYGRSLKCLQRHLTKCDLRHPPGNEIYRKGTIS
FFEIDGRKNKSYSQNLCLLAKCFLDHKTLYYDTDPFLFYVMTEYDCKGFH
IVGYFSKEKESTEDYNVACILTLPPYQRRGYGKLLIEFSYELSKVEGKTG
TPEKPLSDLGLLSYRSYWSQTILEILMGLKSESGERPQITINEISEITSI
KKEDVISTLQYLNLINYVKGQYILTLSEDIVDGHERAMLKRLLRIDSKCL
HFTPKDWSKRGKW
>NP_874368.1 histone acetyltransferase KAT5
isoform 3 [Homo sapiens]
(SEQ ID NO: 3)
MAEVGEIIEGCRLPVLRRNQDNEDEWPLAEILSVKDISGRKLFYVHYIDF
NKRLDEWVTHERLDLKKIQFPKKEAKTPTKNGLPGSRPGSPEREVKRKVE
VVSPATPVPSETAPASVFPQNGAARRAVAAQPGRKRKSNCLGTDEDSQDS
SDGIPSAPRMTGSLVSDRSHDDIVTRMKNIECIELGRHRLKPWYFSPYPQ
ELTTLPVLYLCEFCLKYGRSLKCLQRHLTKCDLRHPPGNEIYRKGTISFF
EIDGRKNKSYSQNLCLLAKCFLDHKTLYYDTDPFLFYVMTEYDCKGFHIV
GYFSKEKESTEDYNVACILTLPPYQRRGYGKLLIEFSYELSKVEGKTGTP
EKPLSDLGLLSYRSYWSQTILEILMGLKSESGERPQITINEISEITSIKK
EDVISTLQYLNLINYYKGQYILTLSEDIVDGHERAMLKRLLRIDSKCLHF
TPKDWSKRGKW
>NP_001193762.1 histone acelyltransferase KAT5
isoform 4 [Homo sapiens]
(SEQ ID NO: 4)
MAEVVSPVPGAGRREPGEVGRARGPPVADPGVALSPQGEIIEGCRLPVLR
RNQDNEDEWPLAEILSVKDISGRKLFYVHYIDFNKRLDEWVTHERLDLKK
IQFPKKEAKTPTKNGLPGSRPGSPEREVKRKVEVVSPATPVPSETAPASV
FPQNGAARRAVAAQPGRKRKSNCLGTDEDSQDSSDGIPSAPRMTGSLVSD
RSHDDIVTRMKNIECIELGRHRLKPWYFSPYPQELTTLPVLYLCEFCLKY
GRSLKCLQRHLTKCDLRHPPGNEIYRKGTISFFEIDGRKNKSYSQNLCLL
AKCFLDHKTLYYDTDPFLFYVMTEYDCKGFHIVGYFSKEKESTEDYNVAC
ILTLPPYQRRGYGKLLIEFSYELSKVEGKTGTPEKPLSDLGLLSYRSYWS
QTILEILMGLKSESGERPQITINEISEITSIKKEDVISTLQYLNLINYYK
GQYILTLSEDIVDGHERAMLKRLLRIDSKCLHFTPKDWSKRGKW

In some embodiments, the present invention provides inhibitors of KATs, and in particular, KAT-5, for use as histone acetyltransferase inhibitors, e.g., in vitro or in vivo. In certain embodiments, the present invention provides inhibitors of KATs, e.g., KAT-5, for use in treating diseases or disorders that are characterized by an abnormal KAT-5 activity, e.g., certain cancers.

Some aspects of this disclosure provide methods for modulating protein acetylation, e.g., histone acetylation, e.g., in a cell or tissue, by contacting a histone acetylase, e.g., KAT-5, or a cell or tissue expressing such a histone acetylase, e.g., KAT-5, with a compound of formulae I, I′ or I″ in an amount sufficient to modulate the activity of the histone acetylase, e.g., of KAT-5, e.g., as measured by a reduction in the acetylation of a target protein of the histone acetyltransferase, e.g., a histone acetylated by KAT-5 activity. In some embodiments, the contacting is in vitro. In some embodiments, the contacting is in vivo, e.g., by administering the compound of formulae I, I′ or I″, or a pharmaceutically acceptable salt thereof, to a subject, e.g., a human subject. In some embodiments, the subject is a subject having or diagnosed with a cancer or a precancerous condition.

### Cancers and Tumors

The present disclosure provides, inter alia, compounds and compositions useful in the treatment of cancer, e.g., for the treatment of a tumor in a subject.

In some embodiments, the present invention provides a method of treating a disease or disorder associated with KAT-5. In certain embodiments, the disease or disorder is a KAT-5-mediated disorder.

Cancers that can be treated with the methods and compositions provided herein, e.g., include, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, and myeloid leukemia), lymphoma (e.g., Burkitt lymphoma (non-Hodgkin lymphoma), cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

In some embodiments, the present disclosure provides methods and compositions for treating a tumor in a subject. In some embodiments, the tumor is a solid tumor. In some embodiments, the tumor is a liquid or disperse tumor. In some embodiments, the tumor is associated with a hematologic malignancy, including but not limited to, acute lymphoblastic leukemia, acute myeloid leukemia, chronic lymphocytic leukemia, chronic myelogenous leukemia, hairy cell leukemia, AIDS-related lymphoma, Hodgkin lymphoma, non-Hodgkin lymphoma, follicular lymphoma, diffuse large B-cell lymphoma, Langerhans cell histiocytosis, multiple myeloma, or myeloproliferative neoplasms.

In some embodiments, a tumor comprises a solid tumor. In some embodiments, solid tumors include but are not limited to tumors of the bladder, breast, central nervous system, cervix, colon, esophagus, endometrium, head and neck, kidney liver, lung, ovary, pancreas, skin, stomach, uterus, or upper respiratory tract. In some embodiments, a tumor that may be treated by the compositions and methods of the present disclosure is a breast tumor. In some embodiments, a tumor that may be treated by the compositions and methods of the present disclosure is not a lung tumor.

In some embodiments, a tumor or cancer suitable for treatment with the methods and compositions provided herein includes, for example, Acute Lymphoblastic Leukemia (ALL), Acute Myeloid Leukemia (AML), Adrenal Cortex Cancer, Adrenocortical Carcinoma, AIDS-Related Cancer (e.g., Kaposi Sarcoma, AIDS-Related Lymphoma, Primary CNS Lymphoma), Anal Cancer, Appendix Cancer, Astrocytoma, Atypical Rhabdoid Tumor, Basal Cell. Carcinoma, Bile Duct Cancer, Bladder Cancer, Bone Cancer Brain Tumor, Breast Cancer, Bronchial Tumor, Burkitt Lymphoma, Carcinoid Tumor, Carcinoma, Cardiac (Heart) Tumor, Central Nervous System Tumor, Cervical Cancer, Cholangiocarcinoma, Chordoma, Chronic Lymphocytic Leukemia (CLL), Chronic Myelogenous Leukemia (CML), Chronic Myeloproliferative Neoplasm, Colorectal Cancer, Craniopharyngioma, Cutaneous T-Cell Lymphoma, Ductal Carcinoma In Situ (DCIS), Embryonal Tumor, Endometrial Cancer, Endometrial Sarcoma, Ependymoma, Esophageal, Esthesioneuroblastoma, Ewing Sarcoma, Extracranial Germ Cell Tumor, Extragonadal Germ Cell Tumor, Eye Cancer, Fallopian Tube Cancer, Gallbladder Cancer, Gastric (Stomach) Cancer, Gastrointestinal Carcinoid Tumor, Gastrointestinal Stromal Tumor (GIST), Germ Cell Tumor, Gestational Trophoblastic Disease, Glioma, Hairy Cell Leukemia, Head and Neck Cancer, Hepatocellular (Liver) Cancer. Hodgkin Lymphoma, Hypopharyngeal Cancer, Intraocular Melanoma, Islet Cell Tumor, Kaposi Sarcoma, Kidney Tumor, Langerhans Cell Histiocytosis, Laryngeal Cancer, Leukemia, Lip and Oral Cavity Cancer, Liver Cancer, Lung Cancer, Lymphoma, Male Breast Cancer, Malignant Fibrous Histiocytoma, Melanoma, Merkel Cell Carcinoma, Mesothelioma, Mouth Cancer, Multiple Endocrine Neoplasia Syndrome, Multiple Myeloma, Plasma Cell Neoplasm, Mycosis Fungoides, Myelodysplastic Syndrome Myelodysplastic/Myeloproliferative Neoplasm, Nasal Cavity Cancer, Nasopharyngeal Cancer, Neuroblastoma, Non-Hodgkin Lymphoma, Non-Small Cell Lung Cancer, Oral Cancer, Oral Cavity Cancer, Oropharyngeal Cancer, Osteosarcoma, Ovarian Cancer, Pancreatic Cancer, Pancreatic Neuroendocrine Tumor (Islet Cell Tumor), Paraganglioma, Paranasal Sinus Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Primary Peritoneal Cancer, Prostate Cancer, Rectal Cancer, Renal Cell (Kidney) Cancer, Retinoblastoma Retinoblastoma, Rhabdomyosarcoma, Rhabdomyosarcoma, Salivary Gland Cancer, Sarcoma, Sézary Syndrome, Skin Cancer, Small Intestine Cancer, Soft Tissue Sarcoma, Squamous Cell Carcinoma, Squamous Neck Cancer, Stomach (Gastric) Cancer. T-Cell Lymphoma, Testicular Cancer, Testicular Cancer, Throat Cancer, Thymic Carcinoma, Thymoma, Thyroid Cancer, Urethral Cancer, Uterine Sarcoma, Uterine Sarcoma, Vaginal Cancer, Vascular Tumor, Vulvar Cancer, Waldenstrom Macroglobulinemia, Wilms' Tumor.

### Pharmaceutical Compositions

In some embodiments, the present invention provides a pharmaceutical composition comprising an inhibitor of KAT-5 as described herein. In some embodiments, a KAT-5 inhibitor, e.g., a compound of formulae I, I′ or I″ provided herein, can be administered to a subject, e.g., to a human patient, alone, e.g., in the form of a pharmaceutically acceptable salt, a solvated or hydrated form of a compound of formulae I, I′ or I″, and any polymorph or crystal form thereof. In some embodiments, a KAT-5 inhibitor, e.g., a compound of formulae I, I′ or I″, can be administered in the form of a pharmaceutical composition, e.g., where the compound of formulae I, I′ or I″ is admixed with a suitable carrier or excipient. A pharmaceutical composition typically comprises or can be administered at a dose sufficient to treat or ameliorate a disease or condition in the recipient subject, e.g., to treat or ameliorate a cancer as described herein. Accordingly, a pharmaceutical composition is formulated in a manner suitable for administration to a subject, e.g., in that it is free from pathogens and formulated according to the applicable regulatory standards for administration to a subject, e.g., for administration to a human subject. As an example, a formulation for injection is typically sterile and essentially pyrogen-free.

A compound of formulae I, I′ or I″ can also be administered to a subject as a mixture with other agents, e.g., with one or more additional therapeutic agents), e.g., in a suitably formulated pharmaceutical composition. For example, some aspects of the present disclosure relate to pharmaceutical compositions comprising a therapeutically effective dose of a compound of formulae I, I′ or I″, or a pharmaceutically acceptable salt, hydrate, enantiomer or stereoisomer thereof; and a pharmaceutically acceptable diluent or carrier.

Techniques for formulation and administration of a compound of formulae I, I′ or I″ may be found in references well known to one of ordinary skill in the art, such as Remington's “The Science and Practice of Pharmacy.” 21st ed., Lippincott Williams & Wilkins 2005, the entire contents of which are incorporated herein by reference.

Pharmaceutical compositions as provided herein are typically formulated for a suitable route of administration. Suitable routes of administration may, for example, include enteral administration, e.g., oral, rectal, or intestinal administration; parenteral administration, e.g., intravenous, intramuscular, intraperitoneal, subcutaneous, or intramedullary injection, as well as intrathecal, direct intraventricular, or intraocular injections; topical delivery, including eyedrop and transdermal; and intranasal and other transmucosal delivery, or any suitable route provided herein or otherwise apparent to those of ordinary skill in the art.

The pharmaceutical compositions provided herein may be manufactured, e.g., by mixing, dissolving, granulating, dragee-making, levigating, emulsifying, encapsulating, entrapping, or lyophilizing processes, or by any other suitable processes known to those of ordinary skill in the art.

Pharmaceutical compositions for use in accordance with the present invention may be formulated using one or more physiologically acceptable carriers comprising excipients and auxiliaries which facilitate processing of a compound of formulae I, I′ or I″ into preparations which can be used pharmaceutically. Proper formulation is dependent upon the route of administration chosen.

For injection, the agents of the invention may be formulated in aqueous solutions, preferably in physiologically compatible buffers such as Flanks' solution, Ringer's solution, or physiological saline buffer. For transmucosal administration, penetrants are used in the formulation appropriate to the barrier to be permeated. Such penetrants are generally known in the alt.

For oral administration, a compound of formulae I, I′ or I″ can be formulated readily by combining the compound with pharmaceutically acceptable carriers known in the art. Such carriers enable a compound of formulae I, I′ or I″ to be formulated as tablets, pills, dragees, capsules, liquids, gels, syrups, slurries, suspensions and the like, for oral ingestion by a patient to be treated. Pharmaceutical preparations for oral use can be obtained by combining the compound of formulae I, I′ or I″ with a solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredient(s), e.g., a compound of formulae I, I′ or I″, in admixture with filler such as lactose, binders such as starches, and/or lubricants such as talc or magnesium stearate and, optionally, stabilizers. In soft capsules, the compound of formulae I, I′ or I″ may be dissolved or suspended in suitable liquids, such as fatty oils, liquid paraffin, or liquid polyethylene glycols. In addition, stabilizers may be added.

For buccal administration, the compositions may take the form of tablets or lozenges formulated in conventional manner.

For administration by inhalation, a compound of formulae I, I′ or I″ for use according to the present disclosure is conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g., gelatin for use in an inhaler or insufflator may be formulated containing a powder mix of the compound of formulae I, I′ or I″ and a suitable powder base such as lactose or starch.

Suitable compound(s) of formulae I, I′ or I″ can be formulated for parenteral administration by injection, e.g., bolus injection or continuous infusion. Formulations for injection may be presented in unit dosage form, e.g., in ampoules, or in multi-dose containers, and, in some embodiments, may contain an added preservative. The compositions may take such forms as suspensions, solutions or emulsions in oily or aqueous vehicles, and may contain formulators, agents such as suspending, stabilizing and/or dispersing agents.

Pharmaceutical formulations for parenteral administration include aqueous solutions of compounds) of formulae I, I′ or I″ in water-soluble form. Additionally, suspensions of compound(s) of formulae I, I′ or I″ may be prepared as appropriate injection suspensions, e.g., aqueous or oily injection suspensions. Suitable lipophilic solvents or vehicles include fatty oils such as sesame oil, or synthetic fatty acid esters, such as ethyl oleate or triglycerides, or liposomes, Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of compound(s) of formulae I, I′ or I″ to allow for the preparation of highly concentrated solutions.

Alternatively, the active ingredient(s), e.g., compounds) of formulae I, I′ or I″, may be in powder form for reconstitution before use with a suitable vehicle, e.g., sterile pyrogen-free water.

Compound(s) of formulae I, I′ or I″ may also be formulated in rectal compositions such as suppositories or retention enemas, e.g., containing conventional suppository bases, such as cocoa butter or other glycerides.

In addition to the formulations described previously, a compound of formulae I, I′, or I″ may also be formulated as a depot preparation. Such long acting formulations may be administered by implantation (for example, subcutaneously or intramuscularly or by intramuscular injection). Thus, for example, a compound of formulae I, I′ or I″ may be formulated with suitable polymeric or hydrophobic materials (for example as an emulsion in an acceptable oil) or ion exchange resins, or as sparingly soluble derivatives (for example, as a sparingly soluble salt).

Alternatively, other delivery systems for hydrophobic pharmaceutical compound(s) of formulae I, I′ or I″ may be employed. Liposomes and emulsions are examples of delivery vehicles or carriers for hydrophobic drugs. Certain organic solvents such as dimethysulfoxide also may be employed. Additionally, a compound of formulae I, I′ or I″ may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compound(s) of formulae I, I′ or I″ for a few hours, a few days, a few weeks, or a few months, e.g., up to over 100 days.

The pharmaceutical compositions may also comprise suitable solid or gel phase carriers or excipients. Examples of such carriers or excipients include but are not limited to calcium carbonate, calcium phosphate, various sugars, starches, cellulose derivatives, gelatin, and polymers, such as polyethylene glycols.

Additional suitable pharmaceutical compositions and processes and strategies for formulating a suitable compound of formulae I, I′ or I″ will be apparent to the skilled artisan based on the present disclosure. The disclosure is not limited in this respect.

### Methods of Treatment

Some aspects of this disclosure provide methods for modulating protein acetylation, e.g., histone acetylation, in a subject in need thereof by administering a compound of formulae I, I′ or I″ to the subject in an amount sufficient to modulate acetylation of a target protein, e.g., a histone acetylated by KAT-5 activity. In some embodiments, the subject is a subject having or diagnosed with a cancer or a precancerous condition.

Provided herein are methods of treating, preventing or alleviating a symptom of conditions and diseases, such as cancers and precancerous conditions, the course of which can be influenced by modulating the acetylation status of histones or other proteins that are acetylated by KAT-5, wherein said acetylation status is mediated at least in part by the activity of CREBBP. Modulation of the acetylation status of histones can in turn influence the level of expression of target genes activated by acetylation, and/or target genes suppressed by acetylation.

For example, some aspects of the invention provide methods for treating or alleviating a symptom of cancer or precancerous condition. In some embodiments, the method comprises the step of administering to a subject having a cancer or a precancerous condition a compound of formulae I, I′ or I″, e.g., in the form of a pharmaceutical composition, at a therapeutically effective amount.

In some embodiments, compound of formulae I, I′ or I″ inhibits histone acetyltransferase activity of KAT-5. In some embodiments, compound of formulae I, I′ or I″ selectively inhibits histone acetyltransferase activity of KAT-5.

In some embodiments, the subject is diagnosed with a disease or disorder known to be associated with a dysregulation of histone acetylation, e.g., with a dysfunction, of KAT-5. In some embodiments, the subject is diagnosed with a disease or disorder mediated by KAT-5. In some embodiments, the subject has been diagnosed with a cancer.

Dysregulated histone acetylation has been reported to be involved in aberrant expression of certain genes in cancers and other diseases. Compounds described herein can be used to treat such histone acetylation-associated diseases, e.g., to inhibit KAT-5-mediated histone acetylation in affected cells, tissues, or subjects.

Modulators of histone acetylation can be used for modulating cell proliferation, of cells harboring a mutation resulting in aberrant histone acetylation, or for inducing cell death in cells depending on KAT-5 histone acetylation for survival or proliferation. Accordingly, diseases that may be treated with compound(s) of formulae I, I′ or I″ include hyperproliferative diseases, such as benign cell growth and malignant cell growth (cancer).

Exemplary cancers that may be treated with compound provided herein include, without limitation, lymphomas, including non-Hodgkin lymphoma, follicular lymphoma (FL) and diffuse large B-cell lymphoma (DLBCL); melanoma; and leukemia, including CML; Acute Lymphoblastic Leukemia; Acute Myeloid Leukemia; Adrenocortical Carcinoma; AIDS-Related. Cancers; AIDS-Related Lymphoma; Anal Cancer; Astrocytoma, Childhood Cerebellar; Astrocytoma, Childhood Cerebral; Basal Cell Carcinoma, see Skin Cancer (non-Melanoma); Bile Duct Cancer, Extrahepatic; Bladder Cancer; Bone Cancer, osteosarcoma/Malignant Fibrous Histiocytoma; Brain Stem Glioma; Brain Tumor; Brain Tumor; Cerebellar Astrocytoma; Brain Tumor, Cerebral Astrocytoma/Malignant Glioma; Brain Tumor, Ependymoma; Brain Tumor, Medulloblastoma; Brain Tumor, Supratentorial Primitive Neuroectodermal Tumors; Brain Tumor, Visual Pathway and Hypothalamic Glioma; Breast Cancer; Bronchial Adenomas/Carcinoids; Burkitt's Lymphoma; Carcinoid Tumor; Carcinoid Tumor, Gastrointestinal; Carcinoma of Unknown Primary; Central Nervous System Lymphoma, Primary; Cerebellar Astrocytoma; Cervical Cancer; Childhood Cancers; Chronic Lymphocytic Leukemia; Chronic Myelogenous Leukemia; Chronic Myelogenous Leukemia, Hairy Cell; Chronic Myeloproliferative Disorders; Colon Cancer; Colorectal Cancer; Cutaneous T-Cell Lymphoma, see Mycosis Fungoides and Sezary Syndrome; Endometrial Cancer; Esophageal Cancer; Ewing's Family of Tumors; Extrahepatic Bile Duct Cancer; Eye Cancer, Intraocular Melanoma; Eye Cancer, Retinoblastoma; Gallbladder Cancer; Gastric (Stomach) Cancer; Gastrointestinal Carcinoid Tumor; Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma; Hodgkin's Lymphoma During Pregnancy; Hypopharyngeal Cancer; Hypothalamic and Visual Pathway Glioma; Intraocular Melanoma; Islet Cell Carcinoma (Endocrine Pancreas); Kaposi's Sarcoma; Kidney (Renal Cell) Cancer; Kidney Cancer; Laryngeal Cancer; Leukemia; Lip and Oral Cavity Cancer; Liver Cancer, Adult (Primary); Liver Cancer, Childhood (Primary); Lung Cancer, Non-Small Cell; Lung Cancer, Small Cell; Lymphoma, Primary Central Nervous System; Macroglobulinemia, Waldenstrom's; Malignant Fibrous Histiocytoma of Bone/Osteosarcoma; Medulloblastoma; Melanoma; Merkel Cell Carcinoma; Mesothelioma; Mesothelioma, Adult Malignant; Metastatic Squamous Neck Cancer with Occult Primary; Multiple Endocrine Neoplasia Syndrome; Multiple Myeloma; Multiple Myeloma/Plasma Cell Neoplasm. Mycosis Fungoides; Myelodysplastic Syndromes; Myelodysplastic/Myeloproliferative Diseases; Myeloid Leukemia, Adult Acute; Myeloid Leukemia, Childhood Acute; Myeloproliferative Disorders, Chronic; Nasal Cavity and Paranasal Sinus Cancer; Nasopharyngeal Cancer; Neuroblastoma; Non-Hodgkin's Lymphoma; Non-Hodgkin's Lymphoma During Pregnancy; Oral Cancer; Oral Cavity Cancer, Lip and; Oropharyngeal Cancer; Osteosarcoma/Malignant Fibrous Histiocytoma of Bone; Ovarian Cancer; Ovarian Epithelial Cancer; Ovarian Low Malignant Potential Tumor; Pancreatic Cancer; Pancreatic Cancer, Islet Cell; Paranasal Sinus and Nasal Cavity Cancer; Parathyroid Cancer; Penile Cancer; Pheochromocytoma; Pineoblastoma and Supratentorial Primitive Neuroectodermal Tumors; Pituitary Tumor; Plasma Cell Neoplasm/Multiple Myeloma; Pleuropulmonary Blastoma; Pregnancy and Breast Cancer; Prostate Cancer; Rectal Cancer; Retinoblastoma; Rhabdomyosarcoma; Salivary Gland Cancer; Sarcoma, Ewing's Family of Tumors; Sarcoma, Soft Tissue; Sarcoma, Uterine; Sezary Syndrome; Skin Cancer; Skin Cancer (non-Melanoma); Small Intestine Cancer; Soft Tissue Sarcoma; Squamous Cell Carcinoma, see Skin Cancer (non-Melanoma); Squamous Neck Cancer with Occult Primary, Metastatic; Stomach (Gastric) Cancer; Testicular Cancer; Thymoma; Thymoma and Thymic Carcinoma; Thyroid Cancer; Transitional Cell Cancer of the Renal Pelvis and Ureter; Trophoblastic Tumor, Gestational; Unknown Primary Site, Cancer of; Unusual Cancers of Childhood; Urethral Cancer; Uterine Cancer, Endometrial; Uterine Sarcoma; Vaginal Cancer; Visual Pathway and Hypothalamic Glioma; Vulvar Cancer; Waldenstrom's Macroglobulinemia; Wilms' Tumor; and Women's Cancers, Exemplary precancerous conditions that can be treated with compound(s) of formulae I, I′ or I″ include myelodisplastic syndrome (MDS; formerly known as preleukemia).

Any other disease in which histone acetylation mediated by KAT-5 plays a role may be treatable or preventable using compounds and methods described herein.

### Administration

In some embodiments, an active agent for use in accordance with the present disclosure is formulated, dosed, and/or administered in a therapeutically effective amount using pharmaceutical compositions and dosing regimens that are consistent with good medical practice and appropriate for the relevant agent(s) and subject(s). In principle, therapeutic compositions can be administered by any appropriate method known in the art, including, without limitation, oral, mucosal, by-inhalation, topical, buccal, nasal, rectal, or parenteral (e.g. intravenous, infusion; intratumoral, intranodal, subcutaneous, intraperitoneal, intramuscular, intradermal, transdermal, or other kinds of administration involving physical breaching of a tissue of a subject and administration of the therapeutic composition through the breach in the tissue).

In some embodiments, a dosing regimen for a particular active agent may involve intermittent or continuous (e.g., by perfusion or other slow release system) administration, for example to achieve a particular desired pharmacokinetic profile or other pattern of exposure in one or more tissues or fluids of interest in the subject receiving therapy.

In some embodiments, different agents administered in combination may be administered via different routes of delivery and/or according to different schedules. Alternatively or additionally, in some embodiments, one or more doses of a first active agent is administered substantially simultaneously with, and in some embodiments via a common route and/or as part of a single composition with, one or more other active agents.

Factors to be considered when optimizing routes and/or dosing schedule for a given therapeutic regimen may include, for example, the particular indication being treated, the clinical condition of a subject (e.g., age, overall health, prior therapy received and/or response thereto) the site of delivery of the agent, the nature of the agent (e.g. small molecule, an antibody or other polypeptide-based compound), the mode and/or route of administration of the agent, the presence or absence of combination therapy, and other factors known to medical practitioners. For example, in the treatment of cancer, relevant features of the indication being treated may include, for example, one or more of cancer type, stage, location.

In some embodiments, one or more features of a particular pharmaceutical composition and/or of a utilized dosing regimen may be modified over time (e.g., increasing or decreasing the amount of active agent in any individual dose, increasing or decreasing time intervals between doses), for example in order to optimize a desired therapeutic effect or response.

In general, type, amount, and frequency of dosing of active agents in accordance with the present invention are governed by safety and efficacy requirements that apply when one or more relevant agent(s) is/are administered to a mammal, preferably a human. In general, such features of dosing are selected to provide a particular, and typically detectable, therapeutic response as compared to what is observed absent therapy.

In the context of the present invention, an exemplary desirable therapeutic response may involve, but is not limited to, inhibition of and/or decreased tumor growth, tumor size, metastasis, one or more of the symptoms and side effects that are associated with a tumor, as well as increased apoptosis of cancer cells, therapeutically relevant decrease or increase of one or more cell marker or circulating markers. Such criteria can be readily assessed by any of a variety of immunological, cytological, and other methods that are disclosed in the literature.

In some embodiments, an effective dose (and/or a unit dose) of an active agent, may be at least about 0.01 μg/kg body weight, at least about 0.05 μg/kg body weight; at least about 0.1 μg/kg body weight, at least about 1 μg/kg body weight, at least about 2.5 μg/kg body weight, at least about 5 μg/kg body weight, and not more than about 100 μg/kg body weight. It will be understood by one of skill in the art that in some embodiments such guidelines may be adjusted for the molecular weight of the active agent. The dosage may also be varied for route of administration, the cycle of treatment, or consequently to dose escalation protocol that can be used to determine the maximum tolerated dose and dose limiting toxicity (if any) in connection to the administration of a compound of formulae I, I′ or I″ and/or an additional therapeutic agent at increasing doses. Consequently, the relative amounts of the each agent within a pharmaceutical composition may also vary, for example, each composition may comprise between 0001% and 100% (w/w) of the corresponding agent.

In some embodiments, a “therapeutically effective amount” or “therapeutically effective dose” is an amount of a compound of formulae I, I′ or I″, or a combination of two or more compounds of formulae I, I′ or I″, or a combination of a compound of formulae I, I′ or I″ with one or more additional therapeutic agent(s), which inhibits, totally or partially, the progression of the condition or alleviates, at least partially, one or more symptoms of the condition. In some embodiments, a therapeutically effective amount can be an amount which is prophylactically effective. In some embodiments, an amount which is therapeutically effective may depend upon a patient's size and/or gender, the condition to be treated, severity of the condition and/or the result sought. In some embodiments, a therapeutically effective amount refers to that amount of a compound of formulae I, I′ or I″ that results in amelioration of at least one symptom in a patient. In some embodiments, for a given patient, a therapeutically effective amount may be determined by methods known to those of skill in the art.

In some embodiments, toxicity and/or therapeutic efficacy of a compound of formulae I, I′ or I″ can be determined by standard pharmaceutical procedures in cell cultures or experimental animals, e.g., for determining the maximum tolerated dose (MTD) and the ED₅₀ (effective dose for 50% maximal response). Typically, the dose ratio between toxic and therapeutic effects is the therapeutic index; in some embodiments, this ratio can be expressed as the ratio between MTD and ED₅₀. Data obtained from such cell culture assays and animal studies can be used in formulating a range of dosage for use in humans.

In some embodiments, dosage may be guided by monitoring the effect of a compound of formulae I, I′ or I″ on one or more pharmacodynamic markers of enzyme inhibition (e.g., histone acetylation or target gene expression) in diseased or surrogate tissue. For example, cell culture or animal experiments can be used to determine the relationship between doses required for changes in pharmacodynamic markers and doses required for therapeutic efficacy can be determined in cell culture or animal experiments or early stage clinical trials. In some embodiments, dosage of a compound of formulae I, I′ or I″ lies preferably within a range of circulating concentrations that include the ED₅₀ with little or no toxicity. In some embodiments, dosage may vary within such a range, for example depending upon the dosage form employed and/or the route of administration utilized. The exact formulation, route of administration and dosage can be chosen by the individual physician in view of the patient's condition. In the treatment of crises or severe conditions, administration of a dosage approaching the MTD may be required to obtain a rapid response.

In some embodiments, dosage amount and/or interval may be adjusted individually, for example to provide plasma levels of an active moiety which are sufficient to maintain, for example a desired effect, or a minimal effective concentration (MEC) for a period of time required to achieve therapeutic efficacy. In some embodiments, MEC for a particular compound of formulae I, I′ or I″ can be estimated, for example, from in vitro data and/or animal experiments. Dosages necessary to achieve the MEC will depend on individual characteristics and route of administration. In some embodiments, high pressure liquid chromatography (HPLC) assays or bioassays can be used to determine plasma concentrations.

In some embodiments, dosage intervals can be determined using the MEC value. In certain embodiments, compound(s) of formulae I, I′ or I″ should be administered using a regimen which maintains plasma levels above the MEC for 10-90% of the time, preferably between 30-90% and most preferably between 50-90% until the desired amelioration of a symptom is achieved. In other embodiments, different MEC plasma levels will be maintained for differing amounts of time. In cases of local administration or selective uptake, the effective local concentration of the drug may not be related to plasma concentration.

One of skill in the art can select from a variety of administration regimens and will understand that an effective amount of a particular compound of formulae I, I′ or I″ may be dependent on the subject being treated, on the subject's weight, the severity of the affliction, the manner of administration and/or the judgment of the prescribing physician.

### Combination Therapy

In some embodiments, a compound of formulae I, I′ or I″ can be used in combination with another therapeutic agent to treat diseases such as cancer. In some embodiments, a compound of formulae I, I′ or I″, or a pharmaceutical composition thereof, can optionally be administered in combination with one or more additional therapeutic agents, such as a cancer therapeutic agent, e.g., a chemotherapeutic agent or a biological agent. An additional agent can be, for example, a therapeutic agent that is art-recognized as being useful to treat the disease or condition being treated by a compound of formulae I, I′ or I″ e.g., an anti-cancer agent, or an agent that ameliorates a symptom associated with the disease or condition being treated. The additional agent also can be an agent that imparts a beneficial attribute to the therapeutic composition (e.g., an agent that affects the viscosity of the composition). For example, in some embodiments, a compound of formulae I, I′ or I″ is administered to a subject who has received, is receiving, and/or will receive therapy with another therapeutic agent or modality (e.g., with a chemotherapeutic agent, surgery, radiation, or a combination thereof).

Some embodiments of combination therapy modalities provided by the present disclosure provide, for example, administration of a compound of formulae I, I′ or I″ and additional agent(s) in a single pharmaceutical formulation. Some embodiments provide administration of a compound of formulae I, I′ or I″ and administration of an additional therapeutic agent in separate pharmaceutical formulations.

Examples of chemotherapeutic agents that can be used in combination with a compound of formulae I, I′ or I″ described herein include platinum compounds (e.g., cisplatin, carboplatin, and oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, and bendamustine), antitumor antibiotics (e.g., daunorubicin, doxorubicin, idarubicin, epirubicin, mitoxantrone, bleomycin, mytomycin C, plicamycin, and dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, and sunitinib), nitrosoureas (e.g., carmustine, fotemustine, and lomustine), hexamethylmelamine, mitotane, angiogenesis inhibitors (e.g., thalidomide and lenalidomide), steroids (e.g., prednisone, dexamethasone, and prednisolone), hormonal agents (e.g., tamoxifen, raloxifene, leuprolide, bicaluatmide, granisetron, and flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, and oxaprozin), selective cyclooxygenase-2 (COX-2) inhibitors, or any combination thereof.

Examples of biological agents that can be used in the compositions and methods described herein include monoclonal antibodies (e.g., rituximab, cetuximah, panetumumab, tositumomab, trastuzumab, alemtuzumab, gemtuzumab ozolzarnicin, bevacizumab, catumaxomab, denosumab, obinutuzumab, ofatwnumab, ramucirumab, pertuzumab, ipilimumab, nivolumab, nimotuzumab, lambrolizumab, pidilizumab, siltuximab, BMS-936559, RG7446/MPDL3280A, MEDI41736, tremeliniumab, or others known in the art), enzymes (e.g., L-asparaginase), cytokines (e.g., interferons and interleukins), growth factors (e.g., colony stimulating factors and erythropoietin), cancer vaccines, gene therapy vectors, or any combination thereof.

In some embodiments, a compound of formulae I, I′ or I″ is administered to a subject in need thereof in combination with another agent for the treatment of cancer, either in the same or in different pharmaceutical compositions. In some embodiments, the additional agent is an anticancer agent. In some embodiments, the additional agent affects (e.g., inhibits) histone modifications, such as histone acetylation or histone methylation. In certain embodiments, an additional anticancer agent is selected from the group consisting of chemotherapeutics (such as 2CdA, 6-Mercaptopurine, 6-TG, Abraxane™ Accutane®, Actinomycin-D, Adriamycin®, Alimta®, all-trans retinoic acid, amethopterin, Ara-C, Azacitadine, BCNU, Blenoxane®, Camptosar®, CeeNU®, Clofarabine, Clolar™, Cytoxan®, daunorubicin hydrochloride, DaunoXome®, Dacogen®, DIC, Doxil®, Ellence®, Eloxatin®, Emcyt®, etoposide phosphate, Fludara®, FUDR®, Gemzar®, Gleevec®, hexamethylmelamine, Hycamtin®, Hydrea®, Idamycin®, ixabepilone, Ixempra®, L-asparaginase, Leukeran®, liposomal Ara-C, L-PAM, Lysodren, Matulane®, mithracin, Mitontycin-C, Myleran®, Navelbine®, Neutrexin®, nitotinib, Nipent®, Nitrogen Mustard, Novantrone®, Oncaspar®, Panretin®, Paraplatin®, Platinol®, prolifeprospan 20 with carmustine implant, Sandostatin®, Targretin®, Tasigna®, Taxotere®, Temodar®, TESPA, Trisenox®, Valstar®, Velban®, Vidaza™, vincristine sulfate, VM 26, Xeloda® and Zanosar®; biologics (such as Alpha Interferon, Bacillus Calmette-Guerin, Bexxar®, Campath®, Ergamisol®, Eriotinib, Herceptin®, Interleukin-2, Iressa®, lenalidomide, Mylotarg®, Ontak®, Pegasys®, Revlimid®, Rituxan®, Tarceva™, Thalomid®, Velcade® and Zevalin™); small molecules (such as Tykerb®); corticosteroids (such as dexamethasone sodium phosphate, DeltaSone® and Delta-Cortef®); hormonal therapies (such as Arimidex®, Aromasin®, Casodex®, Cytadren®, Eligord®, Eulexin®, Evista®, Falodex®, Fermara®, Halotestin®, Megace®, Nilandron®, Nolvadex®, Plenaxis™ and Zoladex®); and radiopharmaceuticals (such as Iodotope®, Metastron®, Phosphocol® and Samarium SM-153).

The additional agents that can be used in combination with a compound of formulae I, I′ or I″ as set forth above are for illustrative purposes and not intended to be limiting. The combinations embraced by this disclosure, include, without limitation, one or more compounds of formulae I, I′ or I″ as provided herein and at least one additional agent selected from the lists above or otherwise provided herein. Compounds of formulae I, I′ or I′ can also be used in combination with one or with more than one additional agent, e.g., with two, three, four, five, or six, or more, additional agents.

In some embodiments, treatment methods described herein are performed on subjects for which other treatments of the medical condition have failed or have had less success in treatment through other means, e.g., in subjects having a cancer refractory to standard-of-care treatment. Additionally, the treatment methods described herein can be performed in conjunction with one or more additional treatments of the medical condition, e.g., in addition to or in combination with standard-of-care treatment. For instance, the method can comprise administering a cancer-therapeutic regimen, e.g., nonmyeloablative chemotherapy, surges r, hormone therapy, and/or radiation, prior to, substantially simultaneously with, or after the administration of a compound of formulae I, I′ or I″ described herein, or composition thereof. In certain embodiments, a subject to which compound of formula I, I′ or I″ described herein is administered can also be treated with antibiotics and/or one or more additional pharmaceutical agents.

### EXAMPLES

### Synthetic Experimentals

Materials and Methods

Equipment: ¹H NMR Spectra were recorded at 400 MHz using a Bruker AVANCE 400 MHz spectrometer. LC-MS equipment and conditions are as follows:

LC-MS (Agilent): LC: Agilent Technologies 1290 series, Binary Pump, Diode Array Detector. Agilent. Poroshell 120 EC—C18, 2.7 μm, 41.6×50 mm column. Mobile phase: A: 0.05% Formic acid in water (v/v), B: 0.05% Formic acid in ACN (v/v). Flow Rate: 1 mL/min at 25° C. Detector: 214 nm, 254 nm. Gradient stop time, 5 min, Timetable:

| T (min) | A(%) | B(%) |
|---|---|---|
| 0.0 | 90 | 10 |
| 0.5 | 90 | 10 |
| 4.5 | 0 | 100 |
| 4.51 | 90 | 10 |
| 5.0 | 90 | 10 |

MS: G6120A, Quadrupole LC/MS, ion Source: ES-API, TIC: 70˜1000 m/z, Fragmentor: 60, Drying gas flow: 10 L/min, Nebulizer pressure: 35 psi, Drying gas temperature: 350° C., Vcap: 3000V.

Sample preparation: samples were dissolved in ACN or methanol at ˜100 μg/mL, then filtered through a 0.22 μm filter membrane. Injection volume: 1˜10 μL.

Definitions: Boc (tert-butoxycarbonyl); CDCl₃ (deuterated chloroform); DMF (NA-dimethylformamide): DMSO (dimethylsulfoxide); DMSO-d₆ (deuterated dimethylsulfoxide); EDCI (1-ethyl-3-(3-dimethylaminopropyl) carbodiimide); eq (equivalent); ES-API (electrospray atmospheric pressure ionization); Et₃N (triethylamine); Et₂O (diethyl ether); EtOAc (ethyl acetate); g (gram); h (hour); HATU (2-(7-aza-1H-benzotriazole-1-yl)-1,1,3,3-tetramethyluronium hexafluorophosphate); NMR (proton nuclear magnetic resonance); HOBt (hydroxybenzotriazole); Hz (hertz); L (litre); LC-MS (liquid chromatography-mass spectrometry); M (molar); MeOH (methanol); mg (milligrams); MHz (megahertz); min (minutes); mL (millilitres), mmol (millimoles); Pet. ether or PE (petroleum ether); ppm (parts per million); psi (pounds per square inch); Rₜ (retention time); RT (room temperature); THF (tetrahydrofuran); TLC (thin layer chromatography); v/v (volume/volume).

### Synthesis of Intermediate A (4-fluoro-5-methyl-[1,1′-biphenyl]-3-carbohydrazide)

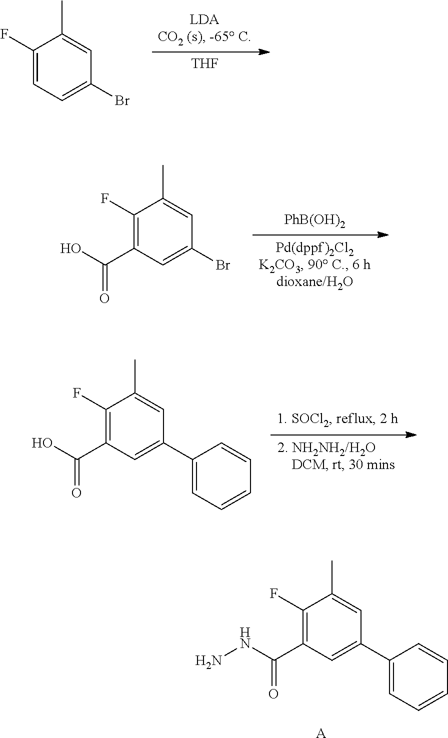

### Step 1: 5-bromo-2-fluoro-3-methylbenzoic acid

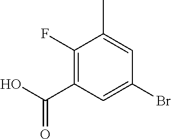

To a solution of 4-bromo-1-fluoro-2-methylbenzene (5.0 g, 26.4 mmol) in THF was added dropwise LDA (2 M in THF, 29.0 mmol) at −65° C. under N₂ atmosphere. The resulting mixture was stirred at −65° C. for 2 h, after which excess solid carbon dioxide was added. The mixture was stirred for 30 min and warmed to room temperature. The reaction mixture was diluted with water (50 mL) and extracted with EtOAc (50 mL×2). The aqueous layer was acidified to pH 3 by 2 M HCl and extracted with EtOAc (50 mL×2), The combined organic layers were washed with brine (50 mL), dried over Na₂SO₄ and concentrated to give 5-bromo-2-fluoro-3-methylbenzoic acid (3.4 g, 52%), which was used for the next step without further purification.

¹H NMR (400 MHz, DMSO-d₆) δ (ppm): 13.47 (s, 1H), 7.75 (d, J=6.0 Hz, 2H), 2.26 (d, 1.6 Hz, 3H).

### Step 2: 4-fluoro-5-methyl-[1,1′-biphenyl]-3-carboxylic acid

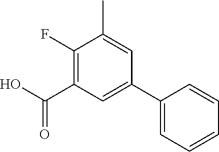 [SMILES: Cc1cc(-c2ccccc2)cc(C(=O)O)c1F]

To a mixture of 5-bromo-2-fluoro-3-methylbenzoic acid (3.5 g, 15.0 mmol) and phenylboronic acid (2.19 g, 18.0 mmol) in dioxane/water (60 mL, 5:1) were added PdCl₂(dppf)₂ (1.09 g, 1.50 mmol) and potassium carbonate (8.29 g, 60.0 mmol) at room temperature under N₂ atmosphere. After heating at 90° C. for 6 h, the reaction mixture was poured into 1 M HCl solution and extracted with EA (150 mL×2). The combined organic layers were washed with 1 M HCl (100 mL×2), water (100 mL) and brine (100 mL), dried over Na₂SO₄ and concentrated. The residue was recrystallized from hexane to give 4-fluoro-5-methyl-[1,1′-biphenyl]-3-carboxylic acid (3 g, 82%) as a yellow solid.

LC-MS (Agilent): Rₜ 3.51 min; m/z calculated for C₁₄H₁₁FO₂ [M−H]⁻ 229.1, found 229.1.

¹H NMR (400 MHz, CDCl₃) δ (ppm): 8.06 (d, J=6.0 Hz, 1H), 7.64 (d, J=6.0 Hz, 1H), 7.57 (d, J=7.6 Hz, 2H), 7.45 (t, J=7.6 Hz, 2H), 7.37 (t, J=7.2 Hz, 1H), 2.40 (s, 3H).

### Step 3: 4-fluoro-5-methyl-[1,1′-biphenyl]-3-carbohydrazide

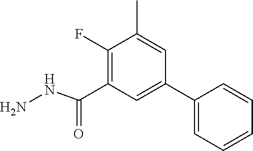 [SMILES: Cc1cc(-c2ccccc2)cc(C(=O)NN)c1F]

A solution of 4-fluoro-5-methyl-[1,1′-biphenyl]-3-carboxylic acid (3 g, 13.0 mmol) in thionyl chloride (15.4 g, 130 mmol) was heated to reflux for 2 h. The reaction mixture was concentrated in vacuo. The residue was dissolved in DCM (50 mL) and hydrazine hydrate (26.0 g, 650 mmol) was added dropwise. The mixture was stirred at room temperature for 30 min. The reaction mixture was diluted with water (100 mL) and extracted with DCII (150 mL×2). The combined organic layers were washed with water (200 mL) and brine (200 mL), dried over Na₂SO₄ and concentrated. The residue was purified by silica gel column (CH₂Cl₂/MeOH=50:1 to 20:1, v/v) to give 4-fluoro-5-methyl-[1,1′-biphenyl]-3-carbohydrazide (1.7 g, 50%) as a yellow solid.

LC-MS (Agilent): Rₜ 2.87 min; m/z calculated for C₁₄H₁₃FN₂O [M+H]⁺ 245.1, found 245.1.

¹H NMR (400 MHz, CDCl₃) δ (ppm): 8.11 (d, J=6.4 Hz, 1H), 7.57-7.53 (m, 3H), 7.43 (t, J=7.6 Hz, 2H), 7.35 (t, J=7.2 Hz, 1H), 2.37 (s, 3H).

General Procedure 1

To a mixture of 4-fluoro-5-methyl[1,1″-biphenyl]-3-carbohydrazide (1.0 eq) and sodium carbonate (2.0 eq) in DCM at 0° C. under N₂ atmosphere was added sulfonyl chloride (1.2 eq). After stirring at room temperature for 18 h, the reaction mixture was diluted with water and extracted with DCM. The combined organic layers were washed with water and brine, dried over Na₂SO₄ and concentrated. The residue was purified by Prep-TLC to afford the desired product.

General Procedure 2

To a solution of 4-fluoro-5-methyl[1,1′-biphenyl]-3-carbohydrazide (1.0 eq) and triethylamine (2.0 eq) in DCM was added sulfonyl chloride (1.0-1.2 eq) at 0° C. under N₂ atmosphere. After stirring at room temperature for 18 h, the reaction mixture was diluted with water and extracted with DCM, me combined organic layers were washed with water and brine, dried over Na₂SO₄ and concentrated. The residue was purified by Prep-TLC to afford the desired product.

The following compounds were synthesized via the general procedures

| Compound | Procedure | LCMS | ¹HNMR |
|---|---|---|---|
| 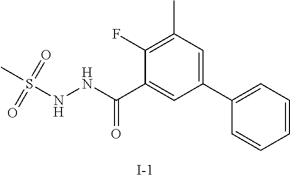 [SMILES: Cc1cc(-c2ccccc2)cc(C(=O)NNS(C)(=O)=O)c1F ; [I-]] | 1 | [M − 1] = 321.1 | ¹H NMR (400 MHz, DMSO-d₆) δ (ppm): 10.67 (s, 1H), 9.72 (s, 1H), 7.77 (d, J = 5.2 Hz, 1H), 7.69 (d, J = 7.6 Hz, 2H), 7.63-7.61 (m, 1H), 7.48 (t, J = 7.6 Hz, 2H), 7.39 (t, J = 7.2 Hz, 1H), 3.04 (s, 3H), 2.35 (s, 3H). |
| 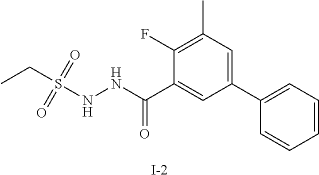 | 2 | [M − 1] = 335.1 | ¹H NMR (400 MHz, DMSO-d₆) δ (ppm): 10.60 (s, 1H), 9.67 (s, 1H), 7.77 (d, J = 6.4 Hz, 1H), 7.68 (d, J = 8.0 Hz, 2H), 7.61-7.59 (m, 1H), 7.48 (t, J = 7.6 Hz, 2H), 7.39 (t, J = 7.2 Hz, 1H), 3.28 (q, J = 7.2 Hz, 2H), 2.34 (s, 3H), 1.33 (t, J = 7.2 Hz, 3H). |
| 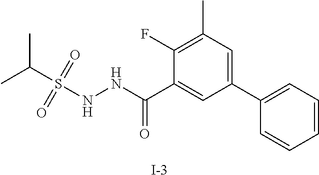 | 2 | [M − 1] = 349.2 | ¹H NMR (400 MHz, DMSO-d₆) δ (ppm): 10.53 (s, 1H), 9.58 (s, 1H), 7.77 (d, J = 6.4 Hz, 1H), 7.68 (d, J = 7.6 Hz, 2H), 7.59-7.57 (m, 1H), 7.48 (t, J = 7.6 Hz, 2H), 7.39 (t, J = 7.2 Hz, 1H), 3.28 (m, 1H), 2.34 (s, 3H), 1.36 (s, 3H), 1.33 (s, 3H). |
| 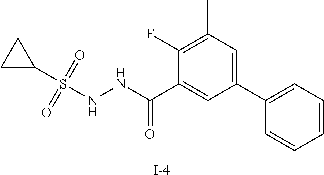 | 2 | [M − 1] = 347.1 | ¹H NMR (400 MHz, DMSO-d₆) δ (ppm): 10.63 (s, 1H), 9.70 (s, 1H), 7.77 (d, J = 6.8 Hz, 1H), 7.68 (d, J = 8.0 Hz, 2H), 7.59-7.57 (m, 1H), 7.48 (t, J = 7.2 Hz, 2H), 7.39 (t, J = 7.2 Hz, 1H), 2.63-2.57 (m, 1H), 2.35 (s, 3H), 1.00-0.98 (m, 4H) |
| 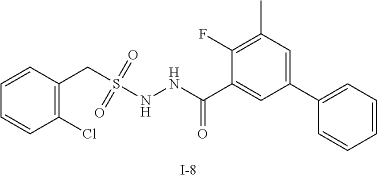 | 2 | [M − 1] = 431.1 | ¹H NMR (400 MHz, DMSO-d₆) δ (ppm): 10.77 (s, 1H), 10.03 (s, 1H), 7.79 (d, J = 5.2 Hz, 1H), 7.70-7.65 (m, 4H), 7.47-7.53 (m, 3H), 7.39-7.41 (m, 3H), 4.67 (s, 2H), 2.36 (s, 3H). |
| 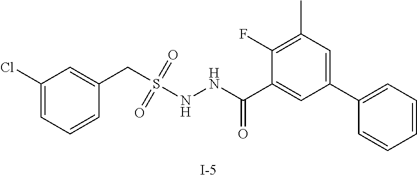 | 2 | [M − 1] = 431.0 | ¹H NMR (400 MHz, DMSO-d₆) δ ppm: 10.68 (s, 1H), 9.86 (s, 1H), 7.78- 7.79 (m, 1H), 7.69 (d, J = 7.6 Hz, 2H), 7.63-7.65 (m, 2H), 7.47-7.51 (m, 3H), 7.39-7.43 (m, 3H), 4.49 (s, 2H), 2.36 (s, 3H). |
| 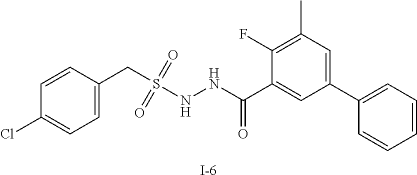 | 2 | [M − 1] = 431.1 | ¹H NMR (400 MHz, DMSO-d₆) δ (ppm): 10.64 (s, 1H), 9.74 (s, 1H), 7.77 (d, J = 5.2 Hz, 1H), 7.69 (d, J = 7.6 Hz, 2H), 7.65-7.62 (m, 1H), 7.55- 7.34 (m, 7H), 4.47 (s, 2H), 2.35 (s, 3H). |
| 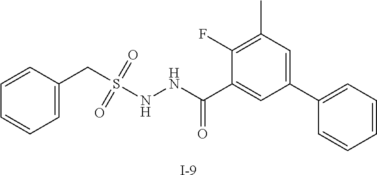 | 2 | [M − 1] = 397.2 | ¹H NMR (400 MHz, DMSO-d₆) δ ppm: 10.67 (s, 1H), 9.77 (s, 1H), 7.79 (d, J = 6.7 Hz, 1H), 7.70 (d, J = 7.3 Hz, 2H), 7.65 (d, J = 5.2 Hz, 1H), 7.55-7.45 (m, 4H), 7.37-7.39 (m, 4H), 4.46 (s, 2H), 2.36 (s, 3H) |
| 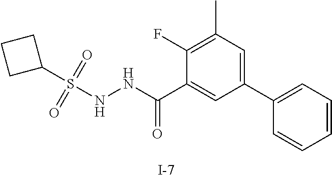 | 2 | [M − 1] = 361.1 | ¹H NMR (400 MHz, DMSO-d₆) δ (ppm): 10.56 (brs, 1H), 9.62 (brs, 1H); 7.76 (d, J = 6.4 Hz, 1H), 7.67 (d, J = 8.0 Hz, 2H), 7.58 (d, J = 6.0 Hz, 1H), 7.48 (t, J = 7.6 Hz, 2H), 7.39 (t, J = 7.2 Hz, 1H), 3.95 (m, 1H), 2.40-2.29 (m, 7H), 1.97-1.87 (m, 2H). |
| 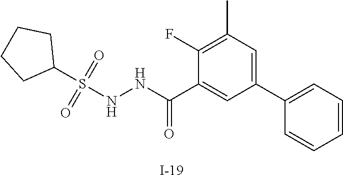 | 2 | [M − 1] = 375.2 | ¹H NMR (400 MHz, DMSO-d₆) δ (ppm): 10.55 (s, 1H), 9.60 (s, 1H), 7.76 (d, J = 6.4 Hz, 1H), 7.67 (d, J = 7.2 Hz, 2H), 7.59 (d, J = 6.0 Hz, 1H), 7.48 (t, J = 7.6 Hz, 2H), 7.39 (t, J = 7.2 Hz, 1H), 3.66-3.58 (m, 1H), 2.34 (s, 3H), 2.01-1.91 (m, 4H), 1.72-1.64 (m, 2H), 1.59-1.57 (m, 2H). |
| 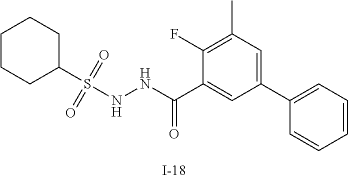 | 2 | [M − 1] = 389.2 | ¹H NMR (400 MHz, DMSO-d₆) δ (ppm): 10.50 (s, 1H), 9.56 (s, 1H), 7.76 (d, J = 6.0 Hz, 1H), 7.67 (d, J = 7.2 Hz, 2H), 7.57 (d, J = 6.0 Hz, 1H), 7.48 (t, J = 7.6 Hz, 2H), 7.39 (t, J = 7.2 Hz, 1H), 3.02 (t, J = 11.6 Hz, 1H), 2.35 (s, 3H), 2.26 (d, J = 12.0 Hz, 2H), 1.80 (d, J = 12.0 Hz, 2H), 1.65- 1.40 (m, 6H). |

### Synthesis of Benzyl 3-((2-(4-fluoro-5-methyl-[1,1′-biphenyl]-3-carbonyl)hydrazinyl)sulfonyl)azetidine-1-carboxylate (I-121)

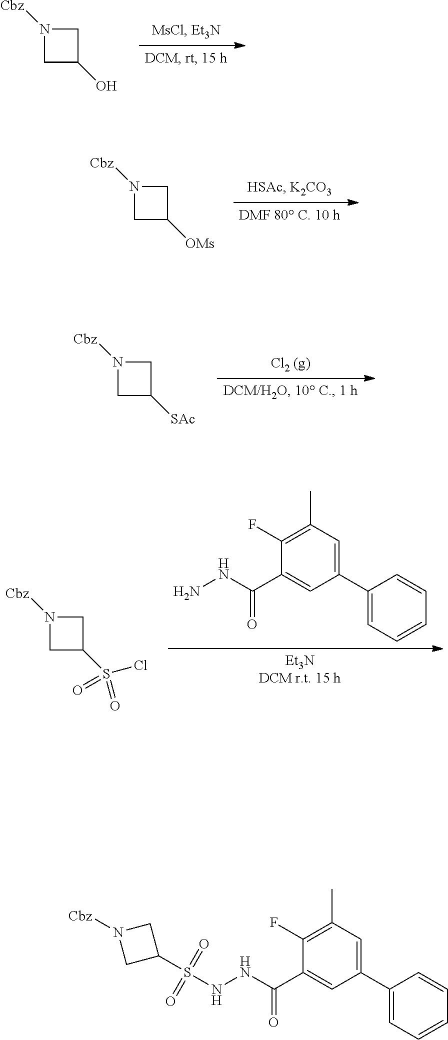

### Step 1: Benzyl 3-((methylsulfonyl)oxy)azetidine-1-carboxylate

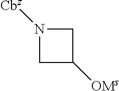 [SMILES: CS(=O)(=O)OC1CN(C(=O)OCc2ccccc2)C1]

Methanesulfonyl chloride (13.6 g, 119 mmol) was added to a solution of benzyl 3-hydroxyazetidine-1-carboxylate (20.7 g, 99.8 mmol) and triethylamine (15.0 g, 149 mmol) in DCM (200 mL) at 0° C. After stirring at room temperature for 15 h, the reaction mixture was washed with 1 M HCl (50 mL) and the aqueous layer extracted with DCM (100 mL×2). The combined organic layers were dried over Na₂SO₄ and concentrated to give benzyl 3-((methylsulfonyl)oxy)azetidine-1-carboxylate (28 g, 98%) as a colourless oil, which was used for the next step without further purification.

LC-MS (Agilent): 3.02 min; m/z calculated for C₁₂H₁₅NO₅S [M+1]⁺ 286.1, found 286.1.

¹H NMR (400 MHz, CDCl₃) δ (ppm): 7.38-7.31 (m, 5H), 5.25-5.20 (m, 1H), 5.11 (s, 2H), 4.38-4.34 (m, 2H), 4.19-4.16 (m, 2H), 3.06 (s, 3H).

### Step 2: Benzyl 3-(acetylthio)azetidine-1-carboxylate

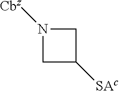

Thioacetic acid (5.99 g, 78.7 mmol) was added dropwise to a mixture of potassium carbonate (10.8 g, 78.7 mmol) and benzyl 3-((methylsulfonyl)oxy)azetidine-1-carboxylate (15 g, 52.5 mmol) in DMF (100 mL) at 10° C. After heating at 80° C. for 10 h, the reaction mixture was diluted with H₂O (300 mL) and extracted with EA (150 mL×3). The combined organic layers were washed with brine (200 mL), dried over Na₂SO₄ and concentrated. The residue was purified by column chromatography (PE/EA=5:1, v/v) to afford benzyl 3-(acetylthio)azetidine-1-carboxylate (9.5 g, 68%) as an off-white solid.

LC-MS (Agilent): Rₜ 3.45 min, m/z calculated for C₁₃H₁₅NO₃S [M+1]⁺ 266.1, found 266.1.

### Step 3: Benzyl 3-(chlorosulfonyl)azetidine-1-carboxylate

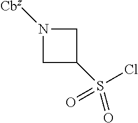 [SMILES: O=C(OCc1ccccc1)N1CC(S(=O)(=O)Cl)C1]

H₂O (4 mL) was added to a solution of benzyl 3-(acetylthio)azetidine-1-carboxylate. (300 mg, 1.13 mmol) in DCM (8 mL) and chlorine was bubbled through the mixture at 0-10° C. with stirring for 1 h. The organic phase was separated, washed with H2O 2O (8 mL), sat. NaHCO₃ (10 mL) and brine (10 mL), dried over Na₂SO₄ and concentrated to afford benzyl 3-(chlorosulfonyl)azetidine-1-carboxylate (250 mg, 76%) as a colourless oil, which was used for the next step directly.

¹H NMR (400 MHz, CDCl₃) δ (ppm): 7.39-7.34 (m, 5H), 5.13 (s, 2H), 4.56-4.50 (m, 1H), 4.49-4.39 (m, 4H).

### Step 4: Benzyl 3-((2-(4-fluoro-5-methyl-[1,1′-biphenyl]-3-carbonyl)hydrazinyl)sulfonyl)azetidine-1-carboxylate (I-121)

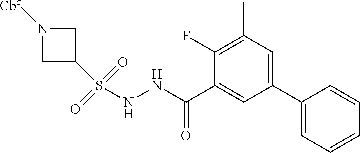 [SMILES: Cc1cc(-c2ccccc2)cc(C(=O)NNS(=O)(=O)C2CN(C(=O)OCc3ccccc3)C2)c1F]

To a solution of 4-fluoro-5-methyl-[1,1′-biphenyl]-3-carbohydrazide (1.05 g, 4.30 mmol) and triethylamine (871 mg, 8.61 mmol) in DCM (70 mL) was added a solution of benzyl 3-(chlorosulfonyl)azetidine-1-carboxylate (1.5 g, 5.17 mmol) in DCM (80 mL) under N₂ atmosphere at 0° C. After warming to room temperature and stirring for overnight, the mixture was diluted with water (150 mL) and extracted with DCM (150 mL×2). The combined organic layers were washed with H₂O (150 mL) and brine (150 mL), dried (Na₂SO₄) and concentrated. The residue was purified by column chromatography (PE/EA=3:1, v/v) to afford benzyl 3-((2-(4-fluoro-5-methyl-[1,1′-biphenyl]-3-carbonyl)hydrazinyl)sulfonyl)azetidine-1-carboxylate (500 mg, 21%) as a white solid.

LC-MS (Agilent): Rₜ 3.84 min; m/z calculated for C₂₅H₂₄FN₃O₅S [M−1]⁻ 496.2, found 496.2.

¹H NMR: (400 MHz, DMSO-d₆) δ (ppm): 10.77 (s, 1H), 10.11 (s, 1H), 7.82-7.74 (m, 1H), 7.72-7.64 (m, 2H), 7.63-7.57 (m, 1H), 7.54-7.27 (m, 8H), 5.07 (s, 2H), 4.37-4.06 (m, 5H), 2.34 (s, 3H).

### Synthesis of tert-Butyl 3-((2-(4-fluoro-5-methyl-[1,1′-biphenyl]-3-carbonyl)hydrazinyl)sulfonyl)azetidine-1-carboxylate (I-119) and N′-(4-fluoro-5-methyl-[1,1′-biphenyl]-3-carbonyl)azetidine-3-sulfonohydrazide hydrochloride (I-120)

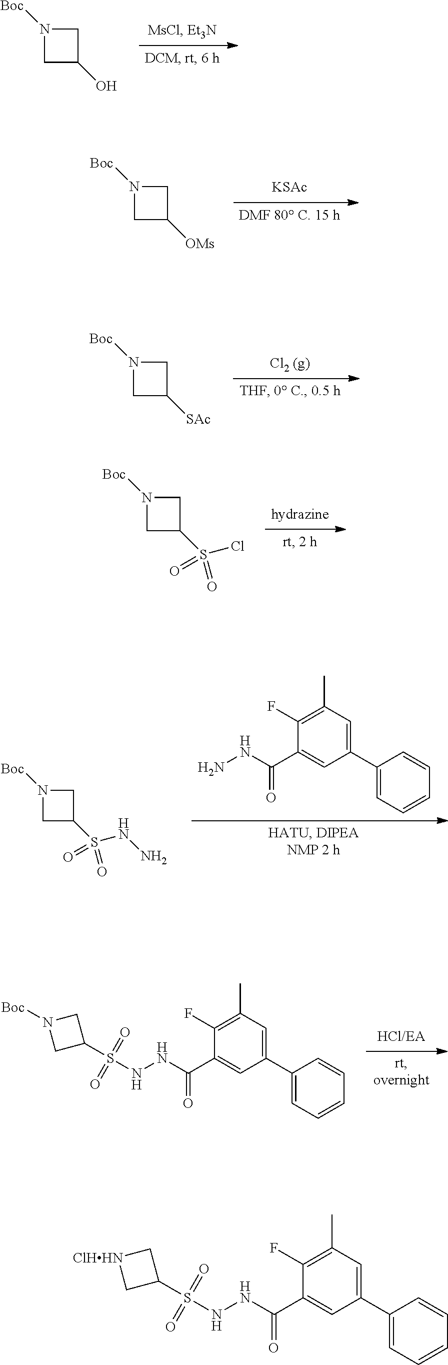

### Step 1: tert-Butyl 3-((methylsulfonyl)oxy)azetidine-1-carboxylate

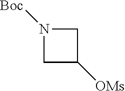 [SMILES: CC(C)(C)OC(=O)N1CC(OS(C)(=O)=O)C1]

Methanesulfonyl chloride (21.4 g, 187 mmol) was added to a solution of tert-butyl 3-hydroxyazetidine-1-carboxylate (25 g, 144 mmol) and triethylamine (21.8 g, 216 mmol) in DCM (500 mL) at 0° C. After stirring at room temperature for 6 h, the reaction mixture was washed with 1 M HCl (50 mL) and the aqueous layer was extracted with DCM (100 mL×2). The combined organic layers were dried over Na₂SO₄ and concentrated to give tert-butyl 3-((methylsulfonyl)oxy)azetidine-1-carboxylate (36 g, 99%) as a colourless oil.

¹H NMR (400 MHz, CDCl₃) δ(ppm): 5.21-5.16 (m, 1H), 4.28-424 (m, 2H), 4.10-4.07 (m, 2H), 3.05 (s, 3H), 1.43 (s, 9H).

### Step 2: tert-Butyl 3-(acetylthio)azetidine-1-carboxylate

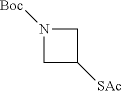

Potassium thioacetate (19.5 g, 171 mmol) was added to a solution of tert-butyl 3-((methylsulfonyl)oxy)azetidine-1-carboxylate (36 g, 143 mmol) in DMF (500 ML). After heating at 80° C. for 15 h, the reaction mixture was diluted with H₂O (1 L) and extracted with EA (250 mL×3). The combined organic layers were washed with brine (300 mL), dried over Na₂SO₄ and concentrated. The residue was purified by column chromatography (PE/EA=10:1, v/v) to afford tert-butyl 3-(acetylthio) azetidine-1-carboxylate (9.5 g, 28%) as a brown oil.

LC-MS (Agilent): Rₜ 3.42 min: m/z calculated for C₁₀H₁₇NO₃S [M+1]⁺ 232.1, found [M+1-56]⁺ 176.1.

¹H NMR (400 MHz, CDCl₃) δ (ppm): 4.36 (t, J=8.8 Hz, 2H), 4.18-4.13 (m, 1H), 3.83-3.79 (m, 2H), 2.22 (s, 3H), 1.43 (s, 9H).

### Step 3: tert-Butyl 3-(chlorosulfonyl) azetidine-1-carboxylate

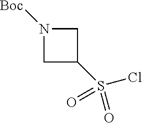 [SMILES: CC(C)(C)OC(=O)N1CC(S(=O)(=O)Cl)C1]

H₂O (5 mL) was added to a solution of tert-butyl 3-(acetylthio) azetidine-1-carboxylate (3.6 g, 15.5 mmol) in DCM (30 mL), and chlorine was bubbled through the mixture at 0° C. with stirring for 0.5 h. The organic phase was separated, washed with sat. NaHCO₃ (20 mL) and brine (15 mL), dried over Na₂SO₄ and concentrated to afford tert-butyl 3-(chlorosulfonyl)azetidine-1-carboxylate (3.7 g, 93%) as a colourless oil, which was used for the next step directly.

¹H NMR (400 MHz, CDCl₃) δ (ppm): 4.52-4.48 (m, 1H), 4.40-4.33 (m, 4H), 1.45 (s, 9H).

### Step 4: tert-Butyl 3-(hydrazinylsulfonyl) azetidine-1-carboxylate

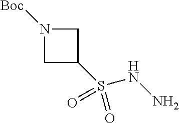 [SMILES: CC(C)(C)OC(=O)N1CC(S(=O)(=O)NN)C1]

80% hydrazine hydrate (1.65 g, 33.1 mmol) was added into a solution of tert-butyl 3-(chlorosulfonyl)azetidine-1-carboxylate (3.7 g, 14.4 mmol) in THF (40 mL) at 0° C., After stirring at room temperature for 2 h, the reaction mixture was concentrated and the residue was purified by column chromatography (DCM/MeOH=20:1, v/v) to afford tert-butyl 3-(hydrazinylsulfonyl) azetidine-1-carboxylate (3 g, 83%) as a yellow oil.

¹H NMR (400 MHz, CDCl₃) δ (ppm): 4.28-4.17 (m, 5H), 1.43 (s, 9H).

### Step 5: tert-Butyl 3-((2-(4-fluoro-5-methyl-[1,1′-biphenyl]-3-carbonyl)hydrazinyl)sulfonyl)azetidine-1-carboxylate (I-119)

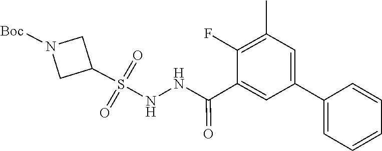 [SMILES: Cc1cc(-c2ccccc2)cc(C(=O)NNS(=O)(=O)C2CN(C(=O)OC(C)(C)C)C2)c1F]

To a solution of 4-fluoro-5-methyl-[1,1′-biphenyl]-3-carboxylic acid (695 mg, 3.02 mmol) in NMP (30 mL) was added diisopropylethylamine (780 mg, 6.04 mmol) and HATU (1.72 g, 4.53 mmol). After stirring at room temperature for 1 h, tert-butyl 3-(hydrazinylsulfonyl) azetidine-1-carboxylate (760 mg, 3.02 mmol) was added. After stirring at rt for 2 h, the reaction mixture was diluted with water (80 mL) and extracted with EA (40 mL×3). The combined organic layers were washed by brine (50 mL), dried and concentrated. The crude product was purified by column chromatography (PE:EA:=3:1, v/v) to give tert-butyl 3-((2-(4-fluoro-5-methyl-[1,1′-biphenyl]-3-carbonyl)hydrazinyl)sulfonyl)azetidine-1-carboxylate (1.4 g, 90%) as a yellow solid.

LC-MS (Agilent): Rₜ 3.83 min; m/z calculated for C₂₂H₂₆FN₃O₅S [M+1]⁺ 464.1, found [M+1−56]⁺ 408.1.

¹H NMR: (400 MHz, DMSO-d₆) δ (ppm): 10.77 (s, 1H), 10.08 (s, 1H), 7.82-7.76 (m, 1H), 7.72-7.65 (m, 2H), 7.64-7.56 (m, 1H), 7.53-7.44 (m, 2H), 7.44-7.34 (m, 1H), 4.25-3.99 (m, 5H), 2.34 (s, 3H), 1.38 (s, 9H).

### Step 6: N′-(4-Fluoro-5-methyl-[1,1′-biphenyl]-3-carbonyl)azetidine-3-sulfonohydrazide hydrochloride (I-120)

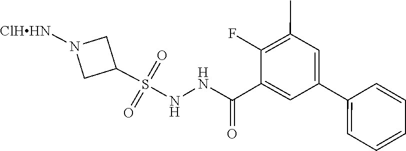

To a solution of 3-((2-(4-fluoro-5-methyl-[1,1′-biphenyl]-3-carbonyl)hydrazinyl)sulfonyl)azetidine-1-carboxylate (800 mg, 1.72 mmol) in EA (20 mL) was added HCl (2) in EA solution (10 mL). After stirring at room temperature overnight, the resulting solid was filtered and washed by EA (5 mL) to give N′-(4-fluoro-5-methyl-[1,1′-biphenyl]-3-carbonyl)azetidine-3-sulfonohydrazide hydrochloride (660 mg, 91%) as a white solid.

LC-MS (Agilent): Rₜ 2.31 min; m/z calculated for C₁₇H₁₉ClFN₃O₃S [M+1−36.5]⁺ 364.1, found 364.1

¹H NMR: (400 MHz, DMSO-d₆) δ (ppm): 10.90 (s, 1H), 10.35 (s, 1H), 9.62 (brs, 1H), 9.39 (brs, 1H), 7.82-7.77 (m, 1H), 7.72-7.66 (m, 2H), 7.66-7.61 (m, 1H), 7.51-7.45 (m, 2H), 7.43-7.36 (m, 1H), 4.54-4.42 (m, 4.35-4.24 (m, 2H), 4.23-4.10 (m, 2H), 2.35 (s, 3H).

### Synthesis of tert-Butyl 3-((2-(4-fluoro-5-methyl-[1,1′-biphenyl]-3-carbonyl)hydrazinyl) sulfonyl)pyrrolidine-1-carboxylate (I-118)

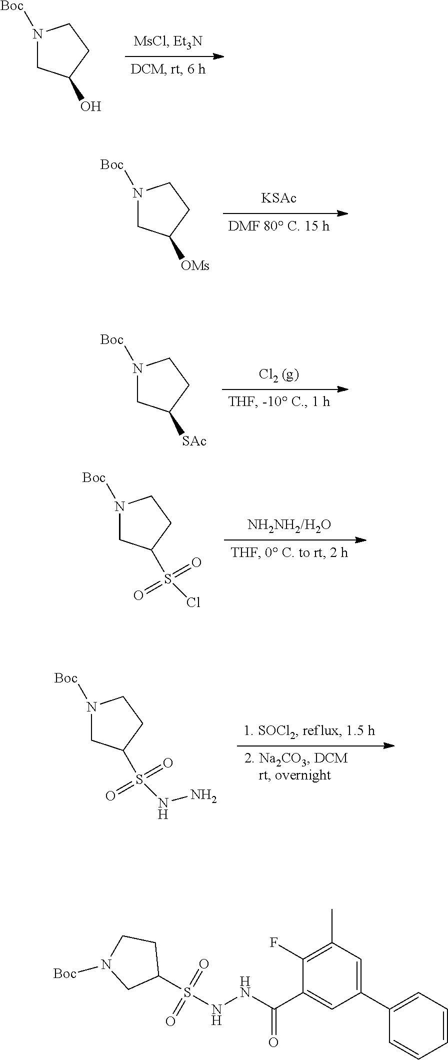

### Step 1: (R)-tert-Butyl 3-((methylsulfonyl)oxy)pyrrolidine-1-carboxylate

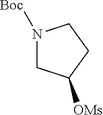 [SMILES: CC(C)(C)OC(=O)N1CC[C@@H](OS(C)(=O)=O)C1]

Methanesulfonyl chloride (19.7 g, 172 mmol) was added to a solution of (R)-tert-butyl 3-hydroxypyrrolidine-1-carboxylate (25 g, 133 mmol) and triethylamine (20.1 g, 199 mmol) in DCM (500 mL) at ° C. After stirring at room temperature for 6 it, the reaction mixture was washed with 1 M HCl (50 mL) and the aqueous layer was extracted with DCM (100 mL×2). The combined organic layers were dried over Na₂SO₄ and concentrated to give (R)-tert-butyl 3-((methylsulfonyl)oxy)pyrrolidine-1-carboxylate (35 g, 99%) as a yellow oil.

¹H NMR (400 MHz, CDCl₃) δ (ppm): 5.28-5.23 (m, 1H), 3.70-3.40 (m, 4H), 3.04 (s, 3H), 2.34-2.07 (m, 2H), 1.46 (s, 9H).

### Step 2: (R)-tert-Butyl 3-acetylthio) pyrrolidine-1-carboxylate

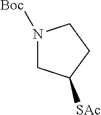

Potassium thioacetate 17.9 g, 157 mmol) was added to a solution of (R)-tert-butyl 3-((methylsulfonyl)oxy) pyrrolidine-1-carboxylate (35 g, 131 mmol) in DMF (500 mL) After heating at 80° C. for 16 h, the reaction mixture was diluted with H₂O (1 L) and extracted with EA (250 mL×3). The combined organic layers were washed with brine (300 mL), dried over Na₂SO₄ and concentrated. The crude product was purified by column chromatography (PE/EA=10:1, v/v) to afford (R)-tert-butyl 3-acetylthio)pyrrolidine-1-carboxylate (4.1 g pure and 9.5 g 90% purity) as a brown oil.

¹H NMR (400 MHz, CDCl₃) δ (ppm): 4.00-3.93 (m, 1H), 3.78-3.73 (m, 1H), 3.42-3.21 (m, 3H), 2.33 (s, 3H), 2.28-2.21 (m, 1H), 1.92-1.82 (m, 1H), 1.45 (s, 9H).

### Step 3: tert-Butyl 3-(chlorosulfonyl) pyrrolidine-1-carboxylate

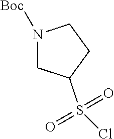 [SMILES: CC(C)(C)OC(=O)N1CCC(S(=O)(=O)Cl)C1]

Chlorine (g) was bubbled through a pre-cooled solution of (S)-tert-butyl 3-(acetylthio)pyrrolidine-1-carboxylate (4.1 g, 16.7 mmol) in THF (150 mL) at −10° C. with stirring for 1 h. The reaction mixture was concentrated to afford tert-butyl 3-(chlorosulfonyl)pyrrolidine-1-carboxylate (4.4 g, 97%) as a yellow oil, which was used for the next step directly.

### Step 4: tert-Butyl 3-(hydrazinylsulfonyl) pyrrolidine-1-carboxylate

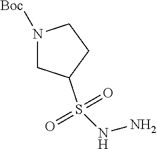 [SMILES: CC(C)(C)OC(=O)N1CCC(S(=O)(=O)NN)C1]

80% Hydrazine hydrate (2.33 g, 37.4 mmol) was added into a solution of tert-butyl 3-(chlorosulfonyl)pyrrolidine-1-carboxylate (4.4 g, 16.3 mmol) in TI-IF (50 mL) at 0° C. After stirring at room temperature for 30 min, the reaction mixture was concentrated. The residue was diluted with DCVI (60 mL) and washed with water (20 mL), brine (20 mL), dried over Na₂SO₄ and concentrated. The crude product was purified by column chromatography (PE:EA=2:1 to DCM:MeOH=10:1) to give tert-butyl 3-(hydrazinylsulfonyl)pyrrolidine-1-carboxylate (1.80 g, 41%) as a white solid.

LC-MS (Agilent): Rₜ 2.20 min; m/z calculated for C₉H₁₉N₃O₄S [M+H]⁺ 265.1, found [M+H−56]⁺ 210.1.

### Step 5: tert-Butyl 3-((2-(4-fluoro-5-methyl-[1,1′-biphenyl]-3-carbonyl)hydrazinyl) sulfonyl)pyrrolidine-1-carboxylate (I-118)

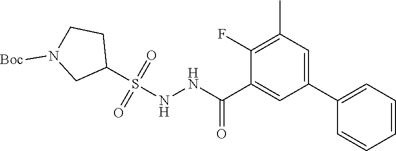 [SMILES: Cc1cc(-c2ccccc2)cc(C(=O)NNS(=O)(=O)C2CCN(C(=O)OC(C)(C)C)C2)c1F]

4-fluoro-5-methyl-[1,1′-biphenyl]-3-carboxylic acid (1.37 g, 5.99 mmol) was dissolved in Thionyl chloride (14.1 g, 119 mmol). After heating at reflux for 1 h, SOCl₂ was removed under reduced pressure. The residue was dissolved in DCM (20 mL) and added dropwise to a suspension of tert-butyl 3-(hydrazinylsulfonyl)pyrrolidine-1-carboxylate (1.59 g, 5.99 mmol) and sodium carbonate (1.26 g, 11.9 mmol) in DCM (10 mL). After stirring at room temperature for overnight, the reaction mixture was diluted with wafer (50 mL) and extracted with EA (80 mL×2). The combined organic layers were washed with water (60 mL) and brine (50 mL), dried and concentrated. The crude product was washed with (PE:EA=2:1, v/v) to give tert-butyl 3-((2-(4-fluoro-5-methyl-[1,1′-biphenyl]-3-carbonyl)hydrazinyl)sulfonyl)pyrrolidine-1-carboxylate (1.67 g, 58%) as a white solid.

LC-MS (Agilent): Rₜ 3.86 min; m/z calculated for C₂₃H₂₈FN₃O₅S [M+H]⁺ 478.1, found [M+1−100]⁺ 378.1.

¹H NMR (400 MHz, DMSO-d₆) δ (ppm): 8.71 (dd, J=12.8, 4.4 Hz, 1H), 8.08-8.09 (m, 1H), 7.37-7.64 (m, 7H), 3.43-3.88 (m, 5H), 2.59-2.33 (m, 5H), 1.44 (5, 9H).

### Synthesis of N′-(4-fluoro-5-methyl-[1,1′-biphenyl]-3-carbonyl)-1-(2-methoxybenzyl)azetidine-3-sulfonohydrazide (I-22)

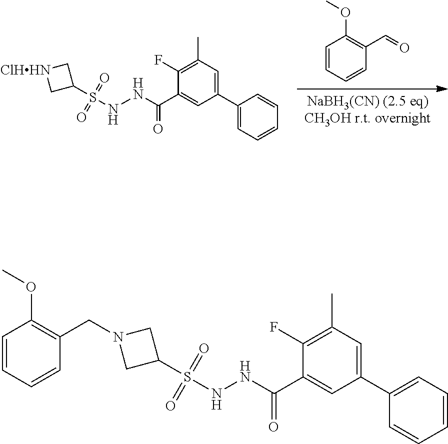

### Step 1: N′-(4-fluoro-5-methyl-[1,1′-biphenyl]-3-carbonyl)-1-(2-methoxybenzyl) azetidine-3-sulfonohydrazide

To a solution of N′-(4-fluoro-5-methyl-[1,1′-biphenyl]-3-carbonyl)azetidine-3-sulfonohydrazide hydrochloride (55 mg, 0.137 mmol) in MeOH (5 mL) was added 2-methoxybenzaldehyde (30 mg, 0.2203 mmol). After stirring at room temperature for 1 h, Sodium cyanoborohydride (21.6 mg, 0.344 mmol) was added. After stirring at room temperature overnight, the reaction mixture was quenched with Sat. NaHCO₃ solution (10 mL) and extracted with EA (30 mL×2). The combined organic layers were washed with H₂O (20 mL) and brine, dried and concentrated. The crude product was purified by column chromatography (PE:EA=4:1) to give N′-(4-fluoro-5-methyl-[1,1′-biphenyl]-3-carbonyl)-1-(2-methoxybenzyl)azetidine-3-sulfonohydrazide (20 mg, 29%) as a white solid.

LC-MS (Agilent): Rₜ 2.80 min; m/z calculated for C₂₅H₂₆FN₃O₄S [M+1]⁺ 484.2, found 484.2.

¹H NMR: (400 MHz, DMSO-d₆) δ (ppm): 10.65 (s, 1H), 9.82 (s, 1H), 7.79-7.74 (m, 1H), 7.67 (d, J=7.2 Hz, 2H), 7.60-7.56 (m, 1H), 7.48 (t, J=7.6 Hz, 2H), 7.39 (t, J=7.2 Hz, 1H), 7.21 (t, J=8.0 Hz, 2H), 6.97-6.92 (m, 1H), 6.89 (t, J=7.2 Hz, 1H), 4.17-4.12 (m, 1H), 3.75 (s, 3H), 3.62-3.54 (m, 4H), 3.43 (t, J=7.2 Hz, 2H), 2.33 (s, 3H).

The following compounds were prepared according to the procedures above:

| | MS | MS found | Detection | Mass Ion |
|---|---|---|---|---|
| Compound | (calc.) | (ESI) | Method | Specks |
| I-122 | 370.11 | 370.43 | ESI | M − 1 |
| I-117 | 390.1413 | 390.47 | ESI | M + 1 |
| I-123 | 384.0944 | 384.43 | ES-API | M − 1 |
| I-124 | 356.0943 | 356.4 | ESI | M − 1 |
| I-125 | 384.0944 | 384.43 | | +H |
| I-126 | 384.0944 | 384.43 | ES-API | M − 1 |
| I-127 | 328.0085 | 328.74 | ESI | M + 1 |
| I-98 | 405.1522 | 405.49 | ES-API | M + 1 |
| I-128 | 418.0554 | 418.87 | ESI | M − 1 |
| I-129 | 400.0893 | 400.42 | ES-API | M + 1 |
| I-130 | 328.0085 | 328.74 | ESI | M + 1 |
| I-131 | 384.0944 | 384.43 | | +H |
| I-97 | 391.1366 | 391.46 | ES-API | M + 1 |
| I-132 | 389.0646 | 389.38 | ES-API | M + 1 |
| I-133 | 278.0525 | 278.3 | ESI | M + 23 |
| I-134 | 312.0886 | 312.27 | ESI | M + 1 |
| I-61 | 453.1522 | 453.53 | ES-API | M + 1 |
| I-135 | 308.0631 | 308.33 | ESI | M + 1 |
| I-23 | 377.1209 | 377.43 | ES-API | M + 1 |
| I-81 | 428.1206 | 428.48 | ES-API | M + 1-100 |
| I-136 | 422.0303 | 422.83 | ES-API | M − 1 |
| I-116 | 300.0944 | 300.35 | ESI | M + 1 |
| I-137 | 517.2047 | 517.62 | ES-API | M + 1-100 |
| I-138 | 258.0805 | 258.25 | ESI | M + 1 |
| I-105 | 447.1628 | 447.53 | ES-API | M + 1 |
| I-139 | 384.0944 | 384.43 | ESI | M + 1 |
| I-27 | 497.1785 | 497.59 | ES-API | M + 1 |
| I-140 | 455.1315 | 455.5 | ES-API | M − 1 |
| I-92 | 433.1835 | 433.54 | ES-API | M + 1 |
| I-141 | 405.1522 | 405.49 | ES-API | M + 1 |
| I-53 | 392.0943 | 392.43 | ES-API | M + 1 |
| I-14 | 391.0991 | 391.45 | ES-API | M + 1 |
| I-109 | 477.1734 | 477.55 | ES-API | M + 1 |
| I-142 | 384.0944 | 384.43 | | +H |
| I-143 | 328.0085 | 328.74 | ESI | M + 1 |
| I-144 | 352.0882 | 352.41 | ESI | M + 1 |
| I-16 | 391.0991 | 391.45 | ES-API | M − 1 |
| I-20 | 406.11 | 406.46 | ES-API | M + 1 |
| I-145 | 306.0674 | 306.34 | ESI | M + 1 |
| I-146 | 326.0725 | 326.37 | ESI | M + 1 |
| I-147 | 414.105 | 414.45 | ES-API | M − 1 |
| I-148 | 290.0725 | 290.34 | ESI | M + 1 |
| I-15 | 311.1304 | 311.4 | ES-API | M + 1 |
| I-149 | 310.0179 | 310.75 | ESI | M + 1 |
| I-17 | 405.1147 | 405.47 | ES-API | M − 1 |
| I-150 | 350.1543 | 350.4 | ES-API | M + 1 |
| I-101 | 449.1785 | 449.54 | ES-API | M + 1 |
| I-151 | 362.11 | 362.42 | ES-API | M + 1 |
| I-152 | 404.0398 | 404.84 | ES-API | M − 1 |
| I-153 | 328.0085 | 328.74 | ES-API | M − 1 |
| I-154 | 388.0693 | 388.39 | ES-API | M − 1 |
| I-155 | 402.085 | 402.42 | ES-API | M − 1 |
| I-156 | 422.0303 | 422.83 | ES-API | M − 1 |
| I-157 | 384.0944 | 384.43 | | +H |
| I-158 | 315.0678 | 315.35 | ESI | M − 1 |
| I-159 | 276.0569 | 276.31 | ESI | M + 1 |
| I-160 | 328.0085 | 328.74 | ES-API | M − 1 |
| I-161 | 422.0303 | 422.83 | ES-API | M − 1 |
| I-162 | 401.0846 | 401.41 | ES-API | M + 1 |
| I-42 | 407.1315 | 407.46 | ES-API | M + 1 |
| I-12 | 288.0681 | 288.33 | ES-API | M + 1 |
| I-163 | 294.0474 | 294.3 | ESI | M + 1 |
| I-164 | 438.085 | 438.45 | ES-API | M − 1 |
| I-13 | 302.0837 | 302.35 | ES-API | M + 1 |
| I-165 | 294.0474 | 294.3 | ESI | M + 1 |
| I-166 | 312.038 | 312.29 | ES-API | M − 1 |
| I-167 | 378.0598 | 378.35 | ES-API | M + 1 |
| I-80 | 428.1206 | 428.48 | ES-API | M + 1 |
| I-102 | 433.1472 | 433.5 | ES-API | M + 1 |
| I-37 | 487.1133 | 487.97 | ES-API | M + 1 |
| I-168 | 369.0835 | 369.41 | ES-API | M + 1 |
| I-38 | 483.1628 | 483.56 | ES-API | M + 1 |
| I-169 | 244.1012 | 244.27 | ESI | M + 1 |
| I-91 | 419.1679 | 419.52 | ES-API | M + 1 |
| I-170 | 412.0208 | 412.8 | ES-API | M + 1 |
| I-94 | 433.1472 | 433.5 | ES-API | M + 1 |
| I-171 | 348.1274 | 348.38 | ESI | M + 1 |
| I-172 | 392.0755 | 392.38 | ES-API | M + 1 |
| I-173 | 309.1277 | 309.34 | ES-API | M + 1 |
| I-174 | 324.058 | 324.33 | ESI | M + 1 |
| I-175 | 385.0896 | 385.41 | ES-API | M − 1 |
| I-176 | 414.105 | 414.45 | ES-API | M + 1 |
| I-177 | 324.058 | 324.33 | ESI | M + 1 |
| I-178 | 349.1478 | 349.4 | ES-API | M + 1 |
| I-10 | 387.0741 | 387.4 | ES-API | M + 1 |
| I-179 | 324.058 | 324.33 | ESI | M + 1 |
| I-180 | 326.0725 | 326.37 | ES-API | M + 1 |
| I-181 | 310.0179 | 310.75 | ESI | M + 1 |
| I-36 | 481.1835 | 481.59 | ES-API | M + 1 |
| I-39 | 322.0787 | 322.35 | ES-API | M + 1 |
| I-55 | 364.0893 | 364.39 | ES-API | M + 1 |
| I-182 | 306.0674 | 306.34 | ESI | M + 1 |
| I-183 | 308.0631 | 308.33 | ES-API | M + 1 |
| I-184 | 417.1522 | 417.5 | ES-API | M + 1 |
| I-185 | 300.0569 | 300.33 | ESI | M − 1 |
| I-186 | 414.105 | 414.45 | ESI | M + 1 |
| I-187 | 418.0554 | 418.87 | ES-API | M − 1 |
| I-188 | 398.11 | 398.45 | ES-API | M − 1 |
| I-189 | 278.0474 | 278.29 | ESI | M + 1 |
| I-190 | 310.0179 | 310.75 | ESI | M − 1 |
| I-191 | 294.0474 | 294.3 | ESI | M − 1 |
| I-192 | 306.0674 | 306.34 | ESI | M − 1 |
| I-193 | 294.0474 | 294.3 | ESI | M + 1 |
| I-194 | 277.0521 | 277.3 | ESI | M + 1 |
| I-195 | 385.0896 | 385.41 | ES-API | M + 1 |
| I-196 | 316.063 | 316.34 | ESI | M + 1 |
| I-197 | 342.0787 | 342.37 | ESI | M + 1 |
| I-110 | 447.1628 | 447.53 | ES-API | M − 1 |
| I-114 | 433.1472 | 433.5 | ES-API | M + 1 |
| I-113 | 419.1315 | 419.47 | ES-API | M + 1 |
| I-62 | 467.1679 | 467.56 | ES-API | M + 1 |
| I-198 | 379.9601 | 381.17 | ES-API | M − 1 |
| I-199 | 409.1148 | 409.48 | ES-API | M + 1 |
| I-200 | 380.1195 | 380.46 | ES-API | M − 1 |
| I-40 | 405.1159 | 405.44 | ES-API | M + 1 |
| I-65 | 317.0634 | 317.34 | ES-API | M + 1 |
| I-99 | 419.1679 | 419.52 | ES-API | M + 1 |
| I-201 | 424.1257 | 424.49 | ES-API | M + 1 |
| I-100 | 433.1835 | 433.54 | ES-API | M + 1 |
| I-106 | 463.1577 | 463.52 | ES-API | M + 1 |
| I-202 | 385.0896 | 385.41 | ES-API | M + 1 |
| I-203 | 323.0507 | 323.77 | ES-API | M + 1 |
| I-204 | 385.0896 | 385.41 | ES-API | M + 1 |
| I-90 | 405.1522 | 405.49 | ES-API | M + 1 |
| I-205 | 384.0944 | 384.43 | ES-API | M − 1 |
| I-107 | 447.1628 | 447.53 | ES-API | M + 1 |
| I-104 | 433.1472 | 433.5 | ES-API | M − 1 |
| I-206 | 491.189 | 491.58 | ES-API | M + 1-100 |
| I-207 | 477.1734 | 477.55 | ES-API | M + 1-100 |
| I-208 | 435.1628 | 435.51 | ES-API | M + 1 |
| I-103 | 345.1159 | 345.39 | ES-API | M + 1 |
| I-47 | 378.115 | 378.45 | ES-API | M + 1 |
| I-67 | 393.0947 | 393.44 | ES-API | M + 1 |
| I-21 | 364.1257 | 364.44 | ES-API | M + 1 |
| I-209 | 385.0896 | 385.41 | ES-API | M + 1 |
| I-108 | 461.1785 | 461.55 | ES-API | M + 1 |
| I-210 | 490.1363 | 490.55 | ES-API | M − 1 |
| I-211 | 400.0893 | 400.42 | ES-API | M − 1 |
| I-88 | 491.189 | 491.58 | ES-API | M + 1-100 |
| I-212 | 388.0693 | 388.39 | ES-API | M + 1 |
| I-89 | 391.1366 | 391.46 | ES-API | M + 1 |
| I-119 | 463.1577 | 463.52 | ES-API | M + 1-100 |
| I-111 | 461.1785 | 461.55 | ES-API | M + 1 |
| I-63 | 487.1133 | 487.97 | ES-API | M + 1 |
| I-213 | 295.1121 | 295.32 | ES-API | M + 1 |
| I-115 | 449.1421 | 449.5 | ES-API | M + 1 |
| I-214 | 402.085 | 402.42 | ES-API | M − 1 |
| I-82 | 416.1006 | 416.44 | ES-API | M − 1 |
| I-215 | 398.11 | 398.45 | ES-API | M + 1 |
| I-216 | 398.11 | 398.45 | ES-API | M + 1 |
| I-217 | 418.0554 | 418.87 | ES-API | M + 1 |
| I-31 | 391.1366 | 391.46 | ES-API | M + 1 |
| I-32 | 481.1472 | 481.54 | ES-API | M − 1 |
| I-218 | 426.1413 | 426.51 | ES-API | M + 1 |
| I-30 | 501.1289 | 502 | ES-API | M + 1 |
| I-219 | 441.1159 | 441.48 | ES-API | M − 1 |
| I-29 | 501.1289 | 502 | ES-API | M + 1 |
| I-43 | 421.1472 | 421.49 | ES-API | M + 1 |
| I-220 | 404.0398 | 404.84 | ES-API | M − 1 |
| I-221 | 412.1257 | 412.48 | ES-API | M − 1 |
| I-222 | 498.1173 | 498.5 | ES-API | M + 1 |
| I-73 | 423.1304 | 423.5 | ES-API | M + 1 |
| I-223 | 402.085 | 402.42 | ES-API | M + 1 |
| I-224 | 505.2047 | 505.61 | ES-API | M + 1-100 |
| I-83 | 416.1006 | 416.44 | ES-API | M + 1 |
| I-225 | 418.0554 | 418.87 | ESI | M − 1 |
| I-226 | 384.0944 | 384.43 | | +H |
| I-227 | 404.157 | 404.5 | ES-API | M − 1 |
| I-228 | 336.0944 | 336.38 | ES-API | M − 1 |
| I-84 | 416.1006 | 416.44 | ES-API | M + 1 |
| I-229 | 384.0944 | 384.43 | | +H |
| I-230 | 356.0943 | 356.4 | ESI | M + 1 |
| I-231 | 350.11 | 350.41 | ES-API | M − 1 |
| I-232 | 414.105 | 414.45 | ES-API | M + 1 |
| I-233 | 388.0693 | 388.39 | ESI | M − 1 |
| I-234 | 370.0787 | 370.4 | | +H |
| I-235 | 400.0893 | 400.42 | ES-API | M − 1 |
| I-236 | 344.0631 | 344.36 | ES-API | M + 1 |
| I-26 | 497.1785 | 497.59 | ES-API | M + 1 |
| I-237 | 414.105 | 414.45 | ESI | M + 1 |
| I-238 | 344.0631 | 344.36 | ESI | M + 1 |
| I-239 | 427.1002 | 427.45 | ES-API | M − 1 |
| I-240 | 434.11 | 434.49 | ESI | M + 1 |
| I-241 | 418.0554 | 418.87 | ESI | M − 1 |
| I-242 | 398.11 | 398.45 | ES-API | M + 1 |
| I-243 | 362.11 | 362.42 | ES-API | M − 1 |
| I-244 | 441.1159 | 441.48 | ES-API | M − 1 |
| I-245 | 280.0682 | 280.32 | ESI | M + 1 |
| I-246 | 402.1038 | 402.47 | ESI | M − 1 |
| I-247 | 512.1518 | 512.58 | ES-API | M + 1 |
| I-248 | 512.1518 | 512.58 | ES-API | M + 1 |
| I-249 | 376.1257 | 376.45 | ES-API | M − 1 |
| I-112 | 477.1734 | 477.55 | ES-API | M + 1 |
| I-28 | 487.1133 | 487.97 | ES-API | M + 1 |
| I-250 | 388.0693 | 388.39 | ES-API | M − 1 |
| I-251 | 384.0944 | 384.43 | ESI | M + 1 |
| I-252 | 295.0427 | 295.29 | ESI | M + 1 |
| I-253 | 370.0787 | 370.4 | ESI | M + 1 |
| I-254 | 283.0991 | 283.35 | ES-API | M + 1 |
| I-255 | 455.1315 | 455.5 | ES-API | M − 1 |
| I-256 | 428.0842 | 428.43 | ES-API | M − 1 |
| I-93 | 449.1785 | 449.54 | ES-API | M + 1 |
| I-257 | 350.1794 | 350.44 | ES-API | M + 1 |
| I-258 | 404.157 | 404.5 | ES-API | M − 1 |
| I-24 | 501.1289 | 502 | ES-API | M + 1 |
| I-25 | 497.1785 | 497.59 | ES-API | M + 1 |
| I-259 | 392.0755 | 392.38 | ES-API | M + 1 |
| I-33 | 467.1679 | 467.56 | ES-API | M + 1 |
| I-260 | 346.1351 | 346.45 | ES-API | M + 1 |
| I-261 | 414.105 | 414.45 | ES-API | M − 1 |
| I-262 | 344.0631 | 344.36 | ES-API | M + 1 |
| I-263 | 418.0554 | 418.87 | ES-API | M + 1 |
| I-264 | 378.0598 | 378.35 | ES-API | M + 1 |
| I-35 | 483.1628 | 483.56 | ES-API | M + 1 |

### Biochemical Assays.

KAT5. Enzyme assay buffer was 50 mM Tris pH 8.0, 0.002% Tween20, 0.005% bovine skin gelatin, and 1 mM dithiothreitol (DTT). For determination of IC₅₀ values, compounds were serially diluted with 2% (v/v) DMSO in the final reaction, pre-incubating each dilution of each compound with 40 μL of assay buffer containing KAT5 enzyme (9 nM final concentration). 10 μL of assay buffer containing 1 μM peptide substrate and 0.5 μM acetyl coenzyme A (final concentrations) was added. Reactions (50 μL total) were then carried out at 25° C. for 90 minutes. Reactions were terminated by the addition of 0.5% formic acid (final concentration), and a sample of each reaction was analyzed by SAMDI Tech, Inc. (Chicago, Ill.) using self-assembled monolayer desorption/ionization time-of-flight mass spectrometry (Mrksich, M. (2008) Mass spectrometry of self-assembled monolayers: a new tool for molecular surface science, ACS Nano 2, 7-18).

KAT6A. Enzyme assay buffer was 50 mM Tris pH 8.0, 0.002% Tween20, 0.005% bovine skin gelatin, and 1 mM dithiothreitol (DTT). For determination of IC₅₀ values, compounds were serially diluted with 2% (v/v) DMSO in the final reaction, pre-incubating each dilution of each compound with 40 μL of assay buffer containing KAT6A enzyme (12.5 nM final concentration). 10 μL of assay buffer containing 1 μM peptide substrate and 1 μM acetyl coenzyme. A (final concentrations) was added. Reactions (50 μL total) were then carried out at 25° C. for 90 minutes. Reactions were terminated by the addition of 0.5% formic acid (final concentration), and a sample of each reaction was analyzed by SAMDI Tech, Inc. (Chicago, Ill.) using self-assembled monolayer desorption/ionization time-of-flight mass spectrometry (Mrksich, M, (2008) Mass spectrometry of self-assembled monolayers: a, new tool for molecular surface science. ACS Nano 2, 7-18).

| | | | | | Assay | |
|---|---|---|---|---|---|---|
| | | Assay | | Assay | [Acetyl | Reaction |
| | Construct/ | [Enz] | | [Peptide] | CoA] | Time |
| Enzyme | amino acids | (nM) | Peptide substrate | (μM) | (μM) | (min) |
| KAT5 | Full length | 9 | H4 1-20 K5R K8R | 1 | 0.5 | 90 |
| | | | K16R | | | |
| | | | SGRGRGGRGLGKG | | | |
| | | | GARRHRK(Biotin)- | | | |
| | | | NH₂ (SEQ ID NO: 9) | | | |
| KAT6A | 501-784 | 12.5 | H4 1-26 K20Me1 | 1 | 1 | 90 |
| | | | SGRGKGGKGLGKG | | | |
| | | | GAKRHRK(Me1)VLR | | | |
| | | | GGK(Biotin)-NH₂ | | | |
| | | | (SEQ ID NO: 10) | | | |

### Enzyme Constructs

KAT5FL:
Original protein before affinity tag cleavage:
(SEQ ID NO: 5)
MHHHKHHSSGVDLGTENLYFQSNAMAEVGEIIEGCRLPVLRRNQDNEDEW
PLAEILSVKDISGRKLFYVHYIDFNKRLDEWVTHERLDLKKIQFPKKEAK
TPTKNGLPGSRPGSPEREVPASAQASGKTLPIPVQITLRFNLPKEREAIP
GGEPDQPLSSSSCLQPNHRSTKRKVEVVSPATPVPSETAPASVFPQNGAA
RRAVAAQPGRKRKSNCLGTDEDSQDSSDGIPSAPRMTGSLVSDRSHDDIV
TRMKNIECIELGRHRLKPWYFSPYPQELTTLPVLYLCEFCLKYGRSLKCL
QRHLTKCDLRHPFGNEIYRKGTISFFEIDGRKNKSYSQNLCLLAKCFLDH
KTLYYDTDPFLFYVMT3YDCKGFHIVGYFSKEKESTEDYNVACILTLPPY
QRRGYGKLLIEFSYELSKVEGKTGTPEKPLSDLGLLSYRSYWSQTILEIL
MGLKSESGERPQITINEISEITSIKKEDVISTLQVLNLINYYKGQYILTL
SEDIVDGHERAMLKRLLRIDSKCLHFTPKDHSKRGKWDYKDDDDK
Final protein after affinity tag cleavage:
(SEQ ID NO: 6)
SNAMAEVGEIIEGCRLPVLRRNQDNEDEWPLAEILSVKDISGRKLFYVHY
IDFNKRLDEWVTHERLDLKKIQFPKKEAKTPTKNGLPGSRPGSPEREVPA
SAQASGKTLPIPVQITLRFNLPKEREAIPGGEPDQPLSSSSCLQPNHRST
KRKVEVVSPATPVPSETAPASVFPQNGAARRAVAAQPGRKRKSNCLGTDE
DSQDSSDGIPSAPRMTGSLVSDRSHDDIVTSMKNIECISLGRHRLKPWYF
SPYPQELTTLPVLYLCEFCLKYGRSLKCLQRHLTKCDLRHPPGNSIYRKG
TISFFEIDGRKNXSYSQNLCLLAKCFLDHXTLYYDTDPFLFYVMTEYDCK
GFHIVGYFSKEKESTEDYNVACILTLPPYQRRGYGKLLIEFSYELSKVEG
KTGTPEKPLSDLGLLSYRSYWSQTILEILMGLKSESGERPQITINEISEI
TSIKKEDVISTLQYLNLINYYKGQYILTLSEDIVDGHERAMLKKLLRIDS
KCLHFTPKDWSKRGKWDYKDDDDK
KAT6A 501-784
Original protein before affinity tag cleavage:
(SEQ ID NO: 7)
MHHHHHHSSGVDLGTENLYFQSNAPPPPQVRCPSVIEFGKYEIHTWYSSP
YPQEYSRLPKLYLCEFCLKYMKSRTILQQHMKKCGWFHPPANEIYRKNNI
SVFEVDGNVSTIYCQNLCLLAKLFLDHKTLYYDVEPFLFYVLTQNDVKGC
HLVGYFSKEKHCQQKYNVSCIMILPQYQRKGYGRFLIDFSYLLSKREGQA
GSPEKPLSDLGRLSYMAYWKSVILECLYHQNDKQISIKKLSKLTGICPQD
ITSTLHHLRMLDFRSDQFVIIRREKLIQDHMAKLQLNLRPVDVDPECLRW
TPVIVSNS
Final protein after affinity tag cleavage.
(SEQ ID NO: 8)
SNAPPDPQVRCPSVIEFGKYEIHTWYSSPYPQEYSRLPKLYLCEFCLKYM
KSRTILQQHMKKCGWFHPPANEIYRKNNISVFEVDGNVSTIYCQNLCLLA
KLFLDHKTLYYDVEPFLFYVLTQNDVKGCHLVGYFSKEKHCQQKYNVSCI
MILPQYQRKGYGRFLIDFSYLLSKREGQAGSPEKPLSDLGRLSYMAYWKS
VILECLYHQNDKQISIKKLSKLTGICPQDITSTLHHLRMLDFRSDQFVII
RREKLIQDHMAKLQLNLRPVDVDEKCLRWTPVIVSNS
underlined residues: His-TEV tag
italicized residues: Flag tag

underlined residues: His-TEV tag

italicized residues: Flag tag

Table 6 shows the activity of selected compounds of this invention in the KAT5 and/or KAT6A inhibition assays. The compound numbers correspond to the compound numbers above. Compounds having an activity designated as “A” provided an IC₅₀≤10 μM; compounds having an activity designated as “B” provided an IC₅₀ 10.01-50 μM; compounds having an activity designated as “C” provided an IC₅₀ of 50.01-100 μM: and compounds having an activity designated as “D” provided an IC₅₀ of >100 μM.

TABLE 6

| | IC₅₀ His-TEV-FL | IC₅₀ His-TEV- |
|---|---|---|
| # | KAT5-Flag (μM) | KAT6A 501-784 (μM) |
| I-1 | C | B |
| I-2 | B | B |
| I-3 | B | C |
| I-4 | C | C |
| I-5 | A | A |
| I-6 | A | A |
| I-7 | B | B |
| I-8 | A | A |
| I-9 | A | A |
| I-10 | D | D |
| I-12 | D | D |
| I-13 | D | D |
| I-14 | B | B |
| I-15 | D | D |
| I-16 | D | C |
| I-17 | C | D |
| I-18 | A | A |
| I-19 | A | B |
| I-20 | D | D |
| I-21 | D | D |
| I-22 | B | B |
| I-23 | B | B |
| I-24 | B | B |
| I-25 | B | B |
| I-26 | A | A |
| I-27 | B | B |
| I-28 | D | D |
| I-29 | A | A |
| I-30 | B | B |
| I-31 | C | B |
| I-32 | B | A |
| I-33 | B | B |
| I-34 | B | B |
| I-35 | B | B |
| I-36 | B | B |
| I-37 | B | B |
| I-38 | B | B |
| I-39 | B | D |
| I-40 | C | B |
| I-42 | C | B |
| I-43 | C | B |
| I-44 | D | D |
| I-45 | D | D |
| I-46 | D | D |
| I-47 | D | D |
| I-48 | D | D |
| I-49 | D | D |
| I-50 | C | D |
| I-53 | B | B |
| I-55 | B | B |
| I-61 | D | B |
| I-62 | D | B |
| I-63 | D | D |
| I-65 | D | D |
| I-67 | D | D |
| I-73 | D | D |
| I-75 | C | B |
| I-79 | A | A |
| I-80 | A | A |
| I-81 | C | B |
| I-82 | A | A |
| I-83 | A | A |
| I-84 | A | A |
| I-88 | A | A |
| I-89 | D | D |
| I-90 | B | B |
| I-91 | B | C |
| I-92 | D | D |
| I-93 | B | B |
| I-94 | B | B |
| I-97 | D | C |
| I-98 | D | B |
| I-99 | D | B |
| I-100 | D | C |
| I-101 | B | B |
| I-102 | B | B |
| I-103 | D | D |
| I-104 | C | B |
| I-105 | B | B |
| I-106 | B | B |
| I-107 | B | B |
| I-108 | A | B |
| I-109 | B | B |
| I-110 | C | A |
| I-111 | B | A |
| I-112 | B | B |
| I-113 | C | B |
| I-114 | D | B |
| I-115 | C | B |
| I-116 | D | D |
| I-117 | D | C |
| I-118 | B | A |
| I-119 | B | B |
| I-120 | D | D |
| I-121 | B | B |
| I-122 | D | D |
| I-123 | D | D |
| I-124 | D | D |
| I-125 | B | A |
| I-126 | C | A |
| I-127 | D | B |
| I-128 | C | B |
| I-129 | B | A |
| I-130 | D | D |
| I-131 | D | A |
| I-132 | C | A |
| I-133 | D | D |
| I-134 | D | D |
| I-135 | D | D |
| I-136 | A | A |
| I-137 | B | B |
| I-138 | D | D |
| I-139 | D | D |
| I-140 | B | A |
| I-141 | C | D |
| I-142 | B | A |
| I-143 | D | B |
| I-144 | D | A |
| I-145 | D | D |
| I-146 | D | B |
| I-147 | B | A |
| I-148 | D | D |
| I-149 | D | D |
| I-150 | D | D |
| I-151 | D | D |
| I-152 | B | A |
| I-153 | B | A |
| I-154 | B | A |
| I-155 | B | A |
| I-156 | B | A |
| I-157 | A | A |
| I-158 | D | D |
| I-159 | D | C |
| I-160 | B | A |
| I-161 | B | A |
| I-162 | D | A |
| I-163 | D | B |
| I-164 | A | A |
| I-165 | D | B |
| I-166 | D | B |
| I-167 | C | B |
| I-168 | C | C |
| I-169 | D | D |
| I-170 | D | C |
| I-171 | D | D |
| I-172 | D | B |
| I-173 | D | D |
| I-174 | D | D |
| I-175 | A | A |
| I-176 | A | A |
| I-177 | D | B |
| I-178 | D | D |
| I-179 | D | — |
| I-180 | D | D |
| I-181 | D | — |
| I-182 | D | — |
| I-183 | B | A |
| I-184 | D | D |
| I-185 | D | D |
| I-186 | C | B |
| I-187 | A | A |
| I-188 | A | A |
| I-189 | D | D |
| I-190 | C | B |
| I-191 | D | C |
| I-192 | D | D |
| I-193 | D | C |
| I-194 | D | D |
| I-195 | B | A |
| I-196 | D | C |
| I-197 | D | D |
| I-198 | C | A |
| I-199 | D | D |
| I-200 | D | D |
| I-201 | D | — |
| I-202 | D | B |
| I-203 | D | D |
| I-204 | A | A |
| I-205 | D | C |
| I-206 | A | A |
| I-207 | A | A |
| I-208 | B | B |
| I-209 | A | A |
| I-210 | A | A |
| I-211 | A | A |
| I-212 | D | C |
| I-213 | D | D |
| I-214 | A | A |
| I-215 | D | B |
| I-216 | A | A |
| I-217 | A | A |
| I-218 | D | — |
| I-219 | A | A |
| I-220 | A | A |
| I-221 | B | A |
| I-222 | C | A |
| I-223 | A | A |
| I-224 | A | A |
| I-225 | C | B |
| I-226 | D | A |
| I-227 | A | A |
| I-228 | D | D |
| I-229 | B | B |
| I-230 | D | D |
| I-231 | D | D |
| I-232 | A | A |
| I-233 | B | A |
| I-234 | D | A |
| I-235 | A | A |
| I-236 | D | D |
| I-237 | D | C |
| I-238 | C | B |
| I-239 | A | A |
| I-240 | B | B |
| I-241 | C | B |
| I-242 | D | B |
| I-243 | D | D |
| I-244 | B | A |
| I-245 | D | D |
| I-246 | C | A |
| I-247 | C | B |
| I-248 | C | B |
| I-249 | D | D |
| I-250 | A | A |
| I-251 | C | A |
| I-252 | D | C |
| I-253 | C | B |
| I-254 | D | D |
| I-255 | A | A |
| I-256 | C | A |
| I-257 | D | D |
| I-258 | C | C |
| I-259 | B | A |
| I-260 | D | D |
| I-261 | A | A |
| I-262 | C | A |
| I-263 | A | A |
| I-264 | D | C |
| I-265 | D | D |
| I-266 | D | D |
| I-267 | A | A |
| I-268 | B | B |
| I-269 | A | B |
| I-270 | A | B |
| I-271 | A | A |
| I-272 | B | C |
| I-273 | D | C |
| I-274 | A | A |
| I-275 | D | D |
| I-276 | A | B |
| I-277 | B | B |
| I-278 | C | D |
| I-279 | B | A |
| I-280 | A | A |
| I-281 | A | A |
| I-282 | C | B |
| I-283 | D | D |
| I-284 | D | D |
| I-285 | A | A |
| I-286 | C | A |
| I-287 | D | D |
| I-288 | B | B |
| I-289 | A | A |
| I-290 | D | D |
| I-291 | A | A |
| I-292 | A | A |
| I-293 | A | C |
| I-294 | A | A |
| I-295 | D | D |
| I-296 | D | D |
| I-297 | B | A |
| I-298 | A | B |
| I-299 | B | C |
| I-300 | B | C |
| I-301 | B | C |
| I-302 | C | C |
| I-303 | B | A |
| I-304 | C | A |
| I-305 | D | B |
| I-306 | A | A |
| I-307 | B | A |
| I-308 | C | D |
| I-309 | D | D |
| I-310 | B | A |
| I-311 | D | D |
| I-312 | D | D |
| I-313 | A | B |
| I-314 | D | D |
| I-315 | B | B |
| I-316 | D | D |
| I-317 | A | B |
| I-318 | C | C |
| I-319 | B | c |
| I-320 | B | B |
| I-321 | A | B |
| I-322 | A | A |
| I-323 | A | A |
| I-324 | D | D |
| I-325 | A | B |
| I-326 | A | A |
| I-327 | A | A |
| I-328 | B | B |
| I-329 | B | B |
| I-330 | A | A |
| I-331 | A | A |
| I-332 | D | D |
| I-333 | B | B |
| I-334 | B | B |
| I-335 | A | A |
| I-336 | B | B |
| I-337 | B | B |
| I-338 | D | D |
| I-339 | B | B |
| I-340 | A | A |
| I-341 | A | A |
| I-342 | C | C |
| I-343 | B | B |
| I-344 | A | A |
| I-345 | D | D |
| I-346 | A | A |
| I-347 | B | D |
| I-348 | B | B |
| I-349 | B | B |
| I-350 | D | A |
| I-351 | B | D |
| I-352 | A | A |
| I-353 | A | A |
| I-354 | D | D |
| I-355 | B | B |
| I-356 | A | A |
| I-357 | C | D |
| I-358 | A | B |
| I-359 | A | A |
| I-360 | B | B |
| I-361 | C | D |
| I-362 | A | A |
| I-363 | D | D |
| I-364 | C | C |
| I-365 | C | D |
| I-366 | B | C |
| I-367 | — | — |
| I-368 | C | C |
| I-369 | A | A |
| I-370 | B | D |
| I-371 | A | A |
| I-372 | A | A |
| I-373 | C | C |
| I-374 | B | B |
| I-375 | C | C |
| I-376 | B | B |
| I-377 | A | A |
| I-378 | C | D |
| I-379 | A | A |
| I-380 | D | C |
| I-381 | A | B |
| I-382 | C | C |
| I-383 | A | A |
| I-384 | D | A |
| I-385 | A | A |
| I-386 | D | B |
| I-387 | D | D |
| I-388 | A | A |
| I-389 | B | B |
| I-390 | A | A |
| I-391 | A | D |
| I-392 | A | B |
| I-393 | A | D |
| I-394 | A | B |
| I-395 | A | A |
| I-396 | A | A |
| I-397 | A | A |
| I-398 | C | B |
| I-399 | C | C |
| I-400 | A | A |
| I-401 | A | A |
| I-402 | D | D |
| I-403 | D | D |
| I-404 | B | B |
| I-405 | B | D |
| I-406 | D | D |
| I-407 | D | D |
| I-408 | A | B |
| I-409 | B | B |
| I-410 | B | B |
| I-411 | A | B |
| I-412 | B | D |
| I-413 | B | D |
| I-414 | D | D |
| I-415 | B | A |
| I-416 | C | C |
| I-417 | A | A |
| I-418 | A | A |
| I-419 | A | A |
| I-420 | B | D |
| I-421 | A | B |
| I-422 | D | D |
| I-423 | B | D |
| I-424 | A | A |
| I-425 | A | A |
| I-426 | B | A |
| I-427 | A | A |
| I-428 | B | A |
| I-429 | A | A |
| I-430 | C | D |
| I-431 | B | C |
| I-432 | B | B |
| I-433 | A | B |
| I-434 | D | D |
| I-435 | D | D |
| I-436 | C | D |
| I-437 | A | A |
| I-438 | B | B |
| I-439 | D | D |
| I-440 | D | D |
| I-441 | D | D |
| I-442 | D | D |
| I-443 | A | A |
| I-444 | A | A |
| I-445 | B | D |
| I-446 | B | C |
| I-447 | A | B |
| I-448 | D | D |
| I-449 | C | C |
| I-450 | A | A |
| I-451 | A | B |
| I-452 | A | A |
| I-453 | D | D |
| I-454 | C | B |
| I-455 | B | D |
| I-456 | D | D |
| I-457 | A | A |
| I-458 | D | D |
| I-459 | D | A |
| I-460 | A | B |
| I-461 | D | D |
| I-462 | D | D |
| I-463 | A | D |
| I-464 | D | D |
| I-465 | D | C |
| I-466 | B | B |
| I-467 | D | D |
| I-468 | D | D |
| I-469 | D | D |
| I-470 | C | D |
| I-471 | D | D |
| I-472 | D | D |
| I-473 | D | D |
| I-474 | D | D |
| I-475 | D | D |
| I-476 | D | D |
| I-477 | D | D |
| I-478 | D | D |
| I-479 | D | C |
| I-480 | D | D |
| I-481 | D | D |
| I-482 | B | B |
| I-483 | C | D |
| I-484 | B | B |
| I-485 | A | B |
| I-486 | B | B |
| I-487 | B | B |
| I-488 | D | D |
| I-489 | D | D |
| I-490 | C | B |
| I-491 | D | D |
| I-492 | D | D |
| I-493 | D | D |
| I-494 | B | A |
| I-495 | B | B |
| I-496 | D | D |
| I-497 | C | B |
| I-498 | B | B |
| I-499 | D | D |
| I-500 | D | D |
| I-501 | D | D |
| I-502 | D | D |
| I-503 | D | D |
| I-504 | D | D |
| I-505 | D | D |
| I-506 | A | — |
| I-507 | A | A |
| I-508 | — | — |
| I-509 | — | — |
| I-510 | A | D |
| I-511 | A | D |
| I-512 | A | D |
| I-513 | A | D |
| I-514 | D | D |
| I-515 | A | D |
| I-516 | A | D |
| I-517 | D | D |
| I-518 | D | D |
| I-519 | A | A |
| I-520 | D | D |
| I-521 | D | D |
| I-522 | D | D |
| I-523 | B | B |
| I-524 | C | B |
| I-525 | D | — |

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one to indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

1. A compound of formula I: 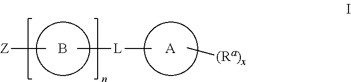
or a pharmaceutically acceptable salt thereof, wherein:
Ring A is selected from phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, an 8-10 membered bicyclic heterocyclic ring having 1-3 heteroatoms independently selected from nitrogen oxygen and sulfur, and an 8-10 membered bicyclic heteroaryl ring having 1-3 heteroatoms independently selected from nitrogen oxygen and sulfur;
L is a 3- to 6-atom linker comprising at least one —S(O)₂— group and 1-4 additional groups independently selected from —C(O)—, —NH—, —O—, and C₁₋₃ aliphatic; wherein:
two atoms of L may, together with their intervening atoms, form a 4-6 membered saturated or partially unsaturated heterocyclic ring having 1-2 heteroatoms independently selected from nitrogen, oxygen and sulfur, or a 5-6 membered heteroaryl ring having 1-2 heteroatoms independently selected from nitrogen, oxygen and sulfur;
Ring B is an optionally substituted group selected from phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, and an 8-10 membered bicyclic heteroaryl ring having 1-3 heteroatoms independently selected from nitrogen oxygen and sulfur;
R^{a} is selected from halogen, —CN, —NO₂, —OR, —SR, —N(R)₂, —C(O)R, —C(O)₂R, —OC(O)R, —C(O)N(R)₂, —N(R)C(O)R, —Cy, or optionally substituted C₁₋₄ aliphatic;
Z is selected from halogen, —CN, —NO₂, —OR, —SR, —N(R)₂, —C(O)R, —C(O)₂R, —OC(O)R, —C(O)N(R)₂, —N(R)C(O)R, —Cy, —(C₁₋₃ aliphatic)-Cy or optionally substituted C₁₋₄ aliphatic;
Cy is an optionally substituted group selected from phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 6-8 membered bridged bicyclic heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, and an 8-10 membered bicyclic heteroaryl ring having 1-3 heteroatoms independently selected from nitrogen oxygen and sulfur;
each R is independently hydrogen or an optionally substituted group selected from C₁₋₄ aliphatic, phenyl, a 3-7 membered saturated or partially unsaturated carbocyclic ring, a 3-7 membered saturated or partially unsaturated heterocyclic ring having 1-2 heteroatoms independently selected from nitrogen, oxygen and sulfur, a 5-6 membered heteroaryl ring having 1-2 heteroatoms independently selected from nitrogen, oxygen and sulfur, an 8-10 membered bicyclic aryl ring, and an 8-10 membered bicyclic heteroaryl ring having 1-3 heteroatoms independently selected from nitrogen oxygen and sulfur;
n is 0 or 1; and
x is 0, 1, 2, or 3.

2. The compound according to claim 1, wherein L is a 3-atom linker.

3. The compound according to claim 2, wherein L is

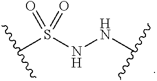 [SMILES: *NNS(*)(=O)=O]

4. The compound according to claim 1, wherein L is a 4-atom linker.

5. The compound according to claim 4, wherein L is selected from the group consisting of

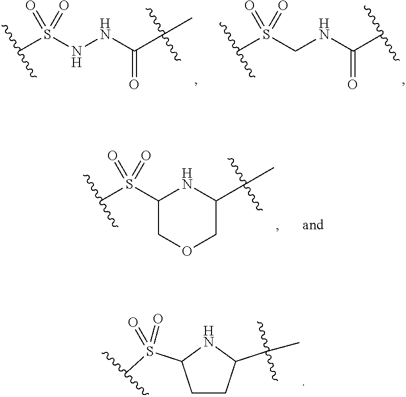 [SMILES: *C(=O)NNS(*)(=O)=O ; *C(=O)NCS(*)(=O)=O ; *C1COCC(S(*)(=O)=O)N1 ; *C1CCC(S(*)(=O)=O)N1]

6. (canceled)

7. The compound according to claim 1, wherein L is a 5-atom linker.

8. The compound according to claim 7, wherein L is selected from the group consisting of

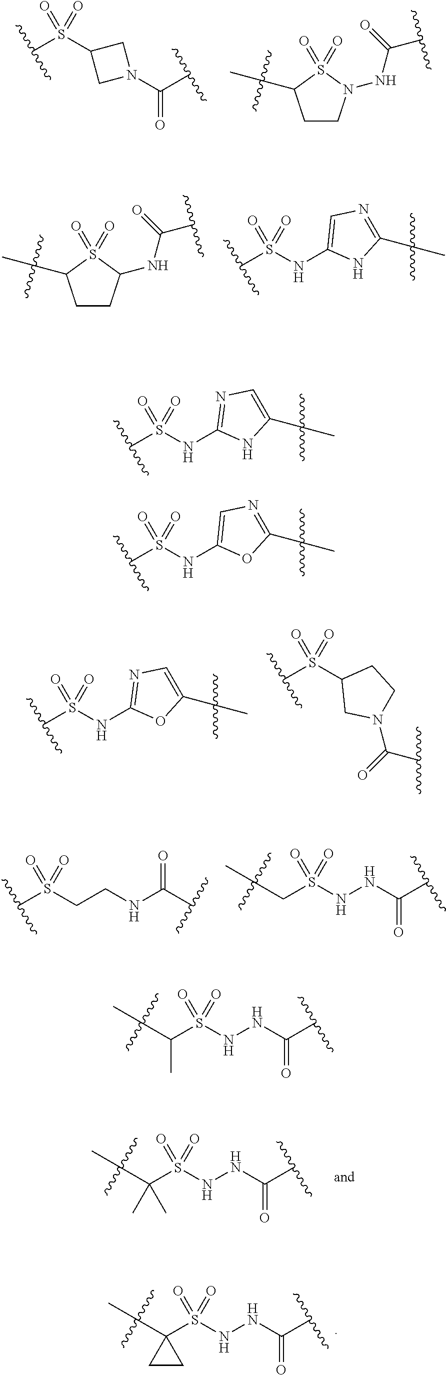

9. The compound according to claim 1, wherein Z is optionally substituted C₁₋₄ aliphatic.

10. (canceled)

11. The compound according to claim 1, wherein Z is —Cy.

12-14. (canceled)

15. The compound according to claim 1, wherein Ring B is an optionally substituted 3-7 membered saturated or partially unsaturated heterocyclic ring having 1-2 heteroatoms independently selected from nitrogen, oxygen and sulfur.

16-22. (canceled)

23. The compound according to claim 1, wherein Ring A is phenyl.

24. The compound according to claim 1, wherein Ring A is a 8-10 membered bicyclic heteroaryl ring having 1-3 heteroatoms independently selected from nitrogen oxygen and sulfur.

25. The compound according to claim 1, wherein Ring A is a 3-7 membered saturated or partially unsaturated heterocyclic ring having 1-2 heteroatoms independently selected from nitrogen, oxygen and sulfur.

26. The compound according to claim 1, wherein the compound is selected from formulae I-a, I-b, I-c, I-d, I-e, I-f, I-g, I-h, I-j, I-k, I-l, I-m, I-n, I-o, I-p, I-q, I-r, or I-s: 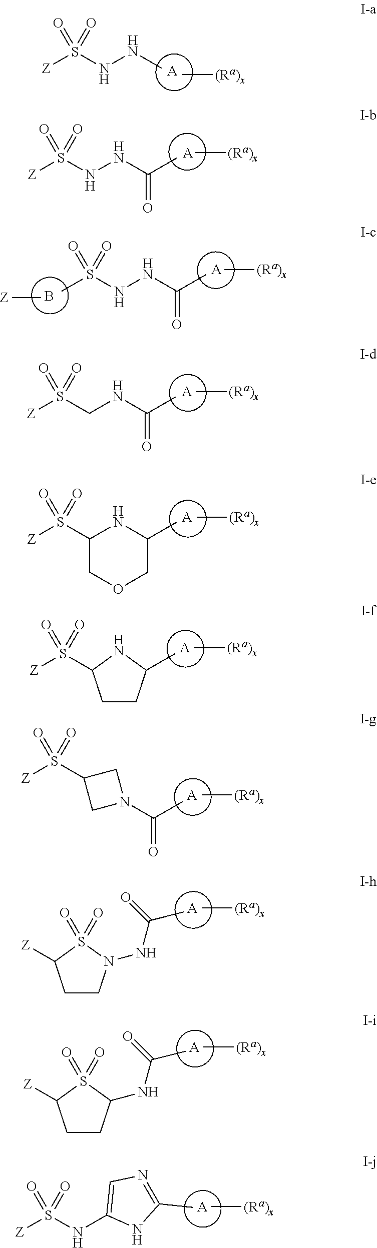 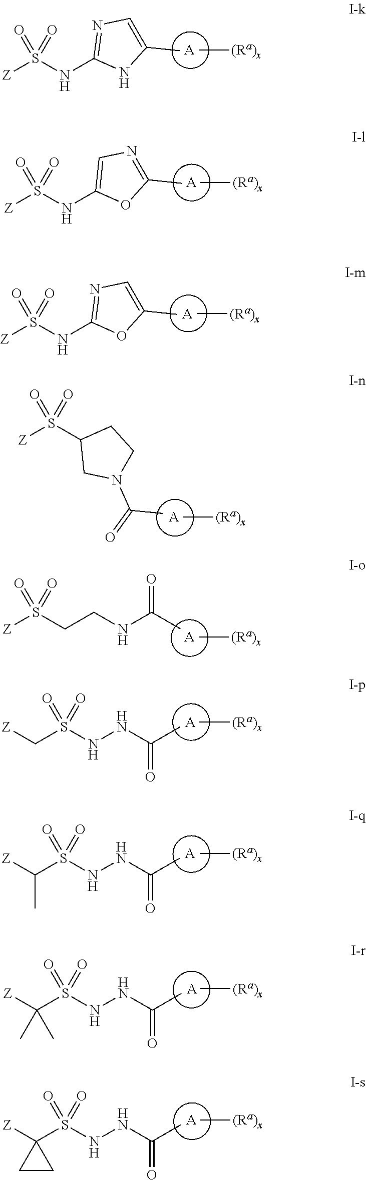
or a pharmaceutically acceptable salt thereof.

27. The compound according to claim 1, wherein the compound is selected from a compound of formulae II, III, IV or V: 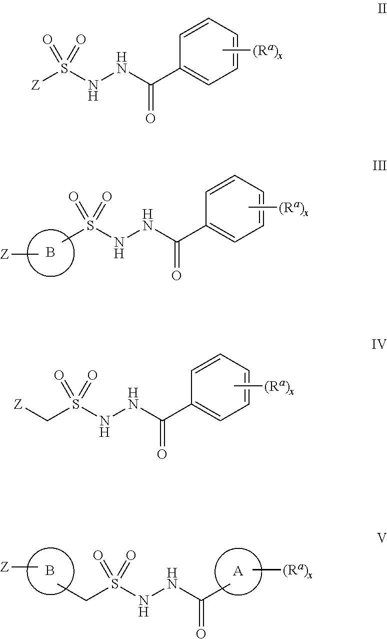
or a pharmaceutically acceptable salt thereof.

28. A pharmaceutical composition comprising a compound or pharmaceutically acceptable salt thereof according to claim 1, and a pharmaceutically acceptable excipient.

29. A method of treating a disease or disorder associated with KAT-5 in a subject in need thereof, comprising administering to the subject an effective amount of a compound or pharmaceutically acceptable salt thereof according to claim 1.

30. A method of modulating protein acetylation in a subject in need thereof, comprising administering to the subject an effective amount of a compound or pharmaceutically acceptable salt thereof according to claim 1.

31. A method of treating cancer in a subject, comprising administering to the subject an effective amount of a compound or pharmaceutically acceptable salt thereof according to claim 1.

32. The method of claim 31, further comprising administering to the subject an additional therapeutic agent.
